(19) United States
(12) Patent Application Publication
(10) Pub. No.: US 2025/0295028 A1
(43) Pub. Date: Sep. 18, 2025

(54) CYCLIC COMPOUNDS FOR ORGANIC ELECTROLUMINESCENT DEVICES

(71) Applicant: Merck Patent GmbH, Darmstadt (DE)

(72) Inventor: Philipp STOESSEL, Darmstadt (DE)

(21) Appl. No.: 18/862,215

(22) PCT Filed: May 3, 2023
(86) PCT No.: PCT/EP2023/061596

§ 371 Date: Nov. 1, 2024

(30) Foreign Application Priority Data
May 6, 2022 (EP) 22172121.0

(51) Int. Cl. H10K 85/60; C07B 59/00; C07C 211/54; C07C 211/60; C07D 209/86; C07D 209/94; C07D 239/26; C07D 239/28; C07D 239/74; C07D 241/42; C07D 251/24; C07D 403/04; C07D 403/10; C07D 403/14; C07D 405/04; C07D 405/10; C07D 409/04; C07D 487/04; C09K 11/02; H10K 50/11; H10K 101/20

(52) U.S. Cl. CPC H10K 85/633; C07B 59/001; C07B 59/002; C07C 211/54; C07C 211/60; C07D 209/86; C07D 209/94; C07D 239/26; C07D 239/28; C07D 239/74; C07D 241/42; C07D 251/24; C07D 403/04; C07D 403/10; C07D 403/14; C07D 405/04; C07D 405/10; C07D 409/04; C07D 487/04; C09K 11/02; H10K 85/622; H10K 85/636; H10K 85/654; H10K 85/6572; C07B 2200/05; C07C 2602/08; H10K 50/11; H10K 85/615; H10K 85/626; H10K 85/6574; H10K 2101/20

(57) ABSTRACT

The present invention relates to cyclic compounds which are suitable for use in electronic devices, and to electronic devices, in particular organic electroluminescent devices, containing said compounds.

The present invention relates to cyclic compounds for use in electronic devices, especially in organic electroluminescent devices, and to electronic devices, especially organic electroluminescent devices comprising these materials.

Emitting materials used in organic electroluminescent devices are frequently phosphorescent organometallic complexes. For quantum-mechanical reasons, up to four times the energy efficiency and power efficiency is possible using organometallic compounds as phosphorescent emitters. In electroluminescent devices, especially also in electroluminescent devices that exhibit triplet emission (phosphorescence), there is generally still a need for improvement. The properties of phosphorescent electroluminescent devices are determined not only by the triplet emitters used. More particularly, the other materials used, such as matrix materials, are also of particular significance here. Improvements in these materials can thus also lead to distinct improvements in the properties of the electroluminescent devices.

The above-detailed electroluminescent devices are among the subject matter described in document WO 2014/015938 A1.

In general terms, in the case of these materials, for example for use as matrix materials, there is still a need for improvement, particularly in relation to the lifetime, but also in relation to the efficiency and operating voltage of the device.

It is therefore an object of the present invention to provide compounds which are suitable for use in an organic electronic device, especially in an organic electroluminescent device, and which lead to good device properties when used in this device, and to provide the corresponding electronic device.

More particularly, the object addressed by the present invention is that of providing compounds which lead to a high lifetime, good efficiency and low operating voltage. Particularly the properties of the matrix materials too have a major influence on the lifetime and efficiency of the organic electroluminescent device.

It is a further object of the present invention to provide compounds that feature a low refractive index (RI).

A further object of the present invention can be considered that of providing compounds suitable for use in phosphorescent or fluorescent electroluminescent devices, especially as a matrix material. A particular problem addressed by the present invention is that of providing matrix materials that are suitable for red- and yellow-phosphorescing electroluminescent devices, especially for red-phosphorescing electroluminescent devices, and if appropriate also for blue-phosphorescing electroluminescent devices.

In addition, the compounds, especially when they are used as matrix materials, as hole transport materials or as electron blocker materials in organic electroluminescent devices, should lead to devices having excellent color purity.

A further object can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, this object is achieved by particular compounds described in detail below that are of good suitability for use in electroluminescent devices and lead to organic electroluminescent devices that show very good properties, especially in relation to lifetime, color purity, efficiency, operating voltage and refractive index. The present invention therefore provides these compounds and electronic devices, especially organic electroluminescent devices, comprising such compounds.

The present invention provides a compound comprising at least one structure of the formula (I), preferably a compound of the formula (I),

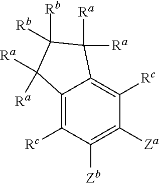

where the symbols are as follows:
Z^{a} is the same or different at each instance and is Ar^{a}, N(Ar^{c})₂ or (Ar)N(Ar^{c})₂;
Z^{b} is the same or different at each instance and is Ar^{b}, N(Ar^{d})₂ or (Ar)N(Ar^{d})₂;
R^{a} is the same or different at each instance and is a straight-chain alkyl, alkoxy or thioalkoxy group having 1 to 10 carbon atoms or a branched or cyclic alkyl, alkoxy or thioalkoxy group having 3 to 10 carbon atoms, each of which may be substituted by one or more R² radicals, preferably a straight-chain alkyl group having 1 to 10 carbon atoms or a branched or cyclic alkyl group having 3 to 10 carbon atoms, each of which may be substituted by one or more R² radicals; at the same time, two or more, preferably adjacent substituents R^{a} together may form a ring system;
R^{b} is the same or different at each instance and is a straight-chain alkyl, alkoxy or thioalkoxy group having 1 to 10 carbon atoms or a branched or cyclic alkyl, alkoxy or thioalkoxy group having 3 to 10 carbon atoms, each of which may be substituted by one or more R² radicals, preferably a straight-chain alkyl group having 1 to 10 carbon atoms or a branched or cyclic alkyl group having 3 to 10 carbon atoms, each of which may be substituted by one or more R² radicals; at the same time, two adjacent substituents R^{b} together may form a ring system;
R^{c} is the same or different at each instance and is H, D, a straight-chain alkyl, alkoxy or thioalkoxy group having 1 to 10 carbon atoms, preferably 1 to 6 carbon atoms, or a branched or cyclic alkyl, alkoxy or thioalkoxy group having 3 to 10 carbon atoms, each of which may be substituted by one or more R² radicals, preferably H, D, a straight-chain alkyl group having 1 to 10 carbon atoms or a branched or cyclic alkyl group having 3 to 10 carbon atoms, each of which may be substituted by one or more R² radicals, preferably H, D, a straight-chain alkyl group having 1 to 10 carbon atoms, preferably 1 to 6 carbon atoms, more preferably H, D;
Ar^{a}, Ar^{b}, Ar^{c}, Ar^{d} is the same or different at each instance and is an aromatic or heteroaromatic ring system which has 6 to 60 aromatic ring atoms and may be substituted by one or more R radicals; at the same time, two Ar^{c}, Ar^{d} radicals bonded to the same nitrogen atom may also be bridged to one another by a single bond or a bridge selected from B(R), C(R)₂, Si(R)₂, C═O, C═NR, C═C(R)₂, RC═CR, O, S, S═O, SO₂, N(R), P(R), P(═O)R and an ortho-linked phenylene group which may be substituted by one or more R radicals, preferably selected from C(R)₂, O, N(R) and an ortho-linked phenylene group which may be substituted by one or more R radicals; preferably, Ar^{a}, Ar^{b}, Ar^{c}, Ar^{d} is the same or different at each instance and is an aryl or heteroaryl group which has 6 to 40 aromatic ring atoms and may be substituted by one or more R radicals; at the same time, two Ar, Ar^{c}, Ar^{d} radicals bonded to the same nitrogen atom may also be bridged to one another by a single bond or a bridge selected from B(R), C(R)₂, Si(R)₂, C═O, C═NR, C═C(R)₂, RC═CR, O, S, S═O, SO₂, N(R), P(R), P(═O)R and an ortho-linked phenylene group which may be substituted by one or more R radicals, preferably selected from C(R)₂, O, N(R) and an ortho-linked phenylene group which may be substituted by one or more R radicals;
Ar is the same or different at each instance and is a connecting aromatic or heteroaromatic ring system which has 5 to 60 aromatic ring atoms and may be substituted by one or more R radicals; at the same time, one Ar radical together with one or both of the Ar^{c}, Ar^{d} radicals bonded to the same nitrogen atom may also be bridged to one another by a single bond or a bridge selected from B(R), C(R)₂, Si(R)₂, C═O, C═NR, C═C(R)₂, RC═CR, O, S, S═O, SO₂, N(R), P(R), P(═O)R and an ortho-linked phenylene group which may be substituted by one or more R radicals, preferably selected from C(R)₂, O, N(R) and an ortho-linked phenylene group which may be substituted by one or more R radicals; preferably, Ar is the same or different at each instance and is an arylene or heteroarylene group which has 5 to 40 aromatic ring atoms and may be substituted by one or more R radicals; at the same time, one Ar radical together with one or both of the Ar^{c}, Ar^{d} radicals bonded to the same nitrogen atom may also be bridged to one another by a single bond or a bridge selected from B(R), C(R)₂, Si(R)₂, C═O, C═NR, C═C(R)₂, RC═CR, O, S, S═O, SO₂, N(R), P(R), P(═O)R and an ortho-linked phenylene group which may be substituted by one or more R radicals, preferably selected from C(R)₂, O, N(R) and an ortho-linked phenylene group which may be substituted by one or more R radicals;
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system together or with a further group;
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be bridged to one another by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more, preferably adjacent R¹ radicals together may form a ring system; at the same time, one or more R¹ radicals may form a ring system with a further part of the compound;
Ar″ is the same or different at each instance and is an aromatic or heteroaromatic ring system which has 5 to 30 aromatic ring atoms and may be substituted by one or more R² radicals; at the same time, it is possible for two Ar″ radicals bonded to the same carbon atom, silicon atom, nitrogen atom, phosphorus atom or boron atom also to be bridged to one another by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, preferably adjacent substituents R² together may form a ring system;
   where the Z^{a} and Z^{b} groups do not form a ring system.

The Z^{a} and Z^{b} groups do not form a ring system. This means that the two groups are connected to one another only by the basic structure shown in formula (I), but not via Ar, Ar^{a}, Ar^{b}, Ar^{c}, Ar^{d} radicals that are used to define the Z^{a} and Z^{b} groups. This includes R, R¹ and R² radicals by which the Ar, Ar^{a}, Ar^{b}, Ar^{c}, Ar^{d} radicals may be substituted.

It may also be the case that the compound is symmetric in relation to the Z^{a} and Z^{b} groups.

It may additionally be the case that the compound is unsymmetric in relation to the Z^{a} and Z^{b} groups.

An aryl group in the context of this invention contains 6 to 40 carbon atoms; a heteroaryl group in the context of this invention contains 3 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 3 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, further preferably F or CN, especially preferably CN.

An aromatic or heteroaromatic ring system which has 5-60 or 5 to 40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

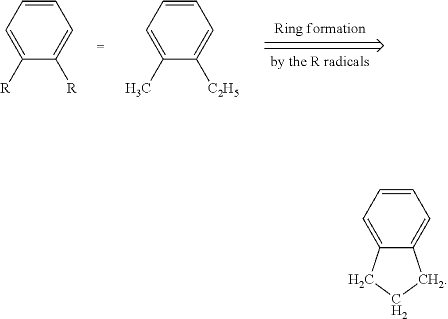

In addition, however, the abovementioned wording shall also be understood to mean that, if one of the two radicals is hydrogen, the second radical binds to the position to which the hydrogen atom was bonded, forming a ring. This will be illustrated by the following scheme:

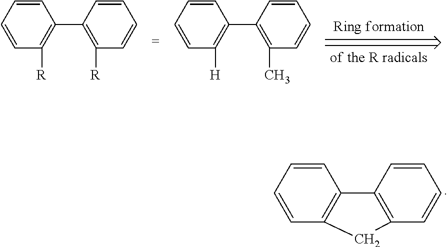

In a preferred configuration, the compounds of the invention may preferably at least one structure of the formulae (I-1) to (I-6) and are more preferably selected from the compounds of the formulae (I-1) to (I-6):

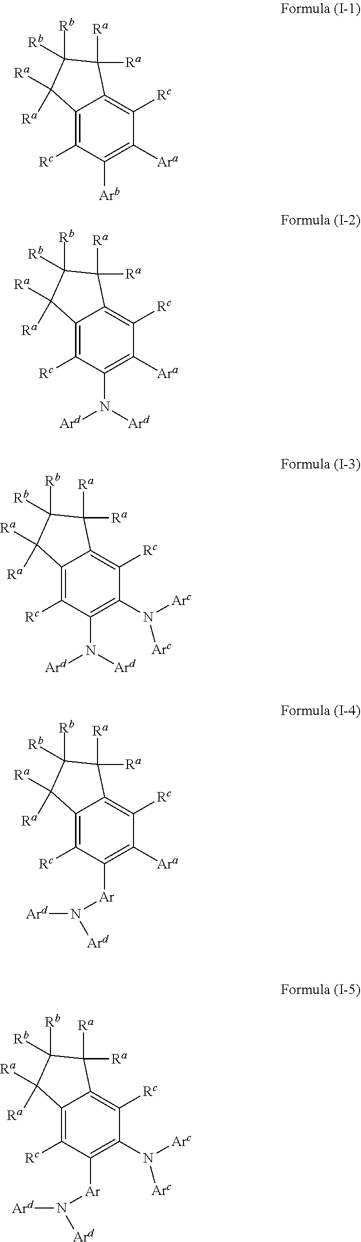

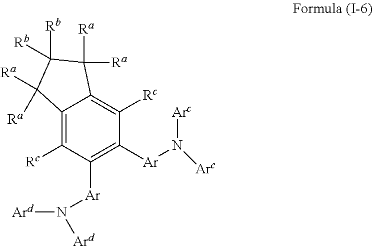

where the symbols Ar, Ar^{a}, Ar^{b}, Ar^{c}, Ar^{d}, R^{a}, R^{b} and Re have the definitions given above, especially for formula (I).

In addition, it may be the case that the Ar, Ar^{a}, Ar^{b}, Ar^{c} and/or Ar^{d} group is the same or different at each instance and is selected from phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, quinoline, isoquinoline, quinazoline, quinoxaline, anthracene, pyrene, perylene, chrysene, phenanthrene or triphenylene, each of which may be substituted by one or more R radicals, preferably phenyl, biphenyl, fluorene, dibenzofuran, triphenylene, carbazole, indolocarbazole.

It may preferably be the case that the Ar^{a}, Ar^{b}, Ar^{c} and/or Ar^{d} group is the same or different at each instance and is selected from structures of the formulae (Ar^{a}-1) to (Ar^{a}-29):

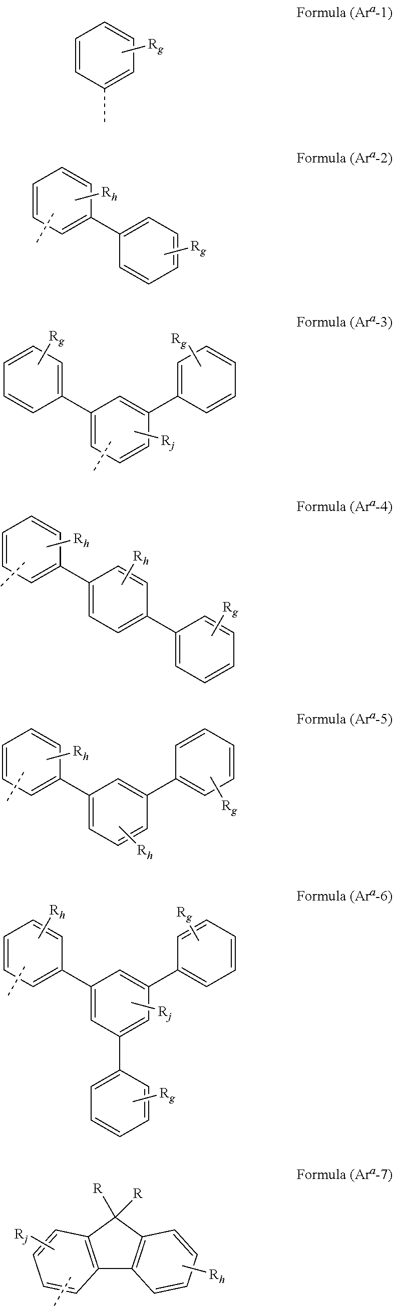

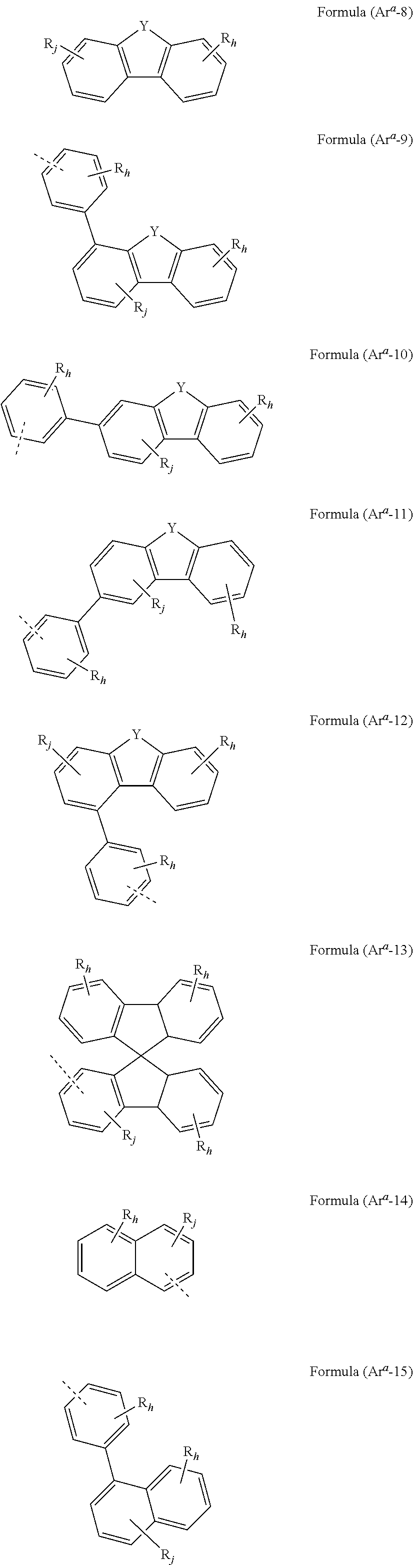

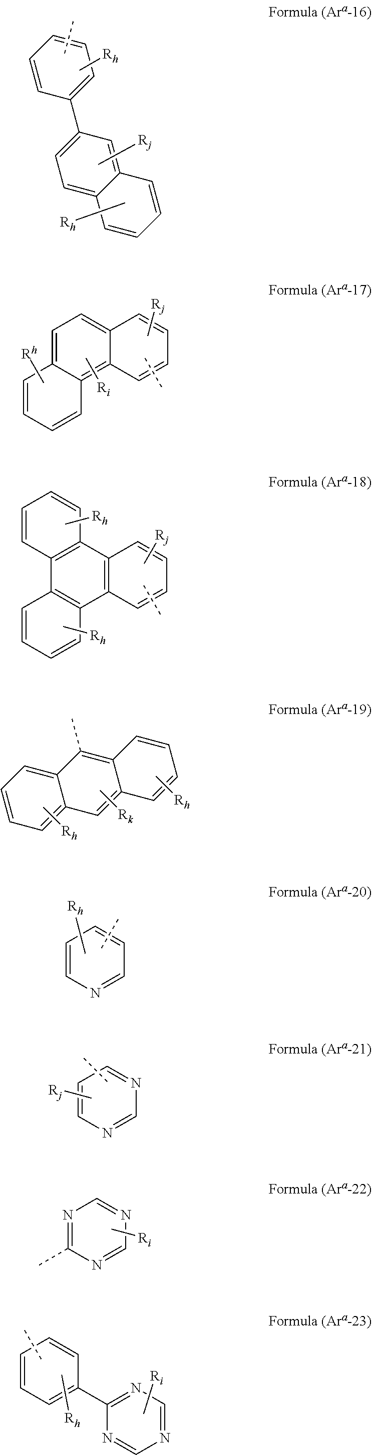

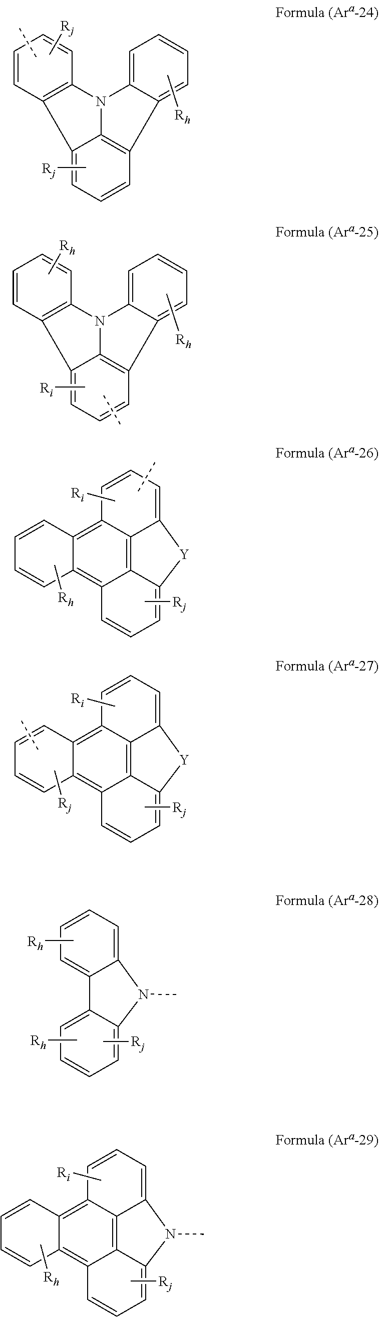

where the symbols used are as follows:
Y is O, S or NR, preferably O or NR;
k at each instance is independently 0 or 1;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5;
R has the definition given above, especially for formula (I), and the dotted bond marks the position of attachment.

It may preferably be the case that the Ar^{a} and/or Ar^{b} group is a structure of the formula —Ar^{e}-Q where Ar^{e} is the same or different at each instance and is an aromatic or heteroaromatic ring system which has 5 to 40 aromatic ring atoms and may be substituted by one or more R radicals, preferably, Ar^{e} is the same or different at each instance and is an aryl or heteroaryl group which has 5 to 30 aromatic ring atoms and may be substituted by one or more R radicals, preferably selected from phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, quinoline, isoquinoline, quinazoline, quinoxaline, anthracene, pyrene, perylene, chrysene, phenanthrene or triphenylene, each of which may be substituted by one or more R radicals, preferably phenyl, biphenyl, fluorene, dibenzofuran, triphenylene, carbazole, indolocarbazole; and Q is an electron transport group, preferably a pyridine, pyrimidine, pyrazine, pyridazine, triazine, quinazoline, quinoxaline, quinoline, isoquinoline, imidazole and/or benzimidazole group which may be substituted by one or more R radicals.

Preferred aromatic or heteroaromatic ring systems Ar and/or Ar^{e} are selected from phenylene, biphenylene, especially ortho-, meta- or para-biphenylene, terphenylene, especially ortho-terphenylene, meta-terphenylene, para-terphenylene or branched terphenylene, quaterphenylene, especially ortho-quaterphenylene, meta-quaterphenylene, para-quaterphenylene or branched quaterphenylene, fluorenylene which may be joined via the 1, 2, 3 or 4 position, spirobifluorenylene which may be joined via the 1, 2, 3 or 4 position, naphthylene, especially naphthylene joined via the 1 or 2 position, indolylene, benzofuranylene, benzothiophenylene, carbazolylene which may be joined via the 1, 2, 3, 4 or 9 position, dibenzofuranylene which may be joined via the 1, 2, 3 or 4 position, benzothiophenylene which may be joined via the 1, 2, 3 or 4 position, carbazolylene, indenocarbazolylene, indolocarbazolylene, pyridinylene, pyrimidinylene, pyrazinylene, pyridazinylene, triazinylene, quinolinylene, isoquinolinylene, quinazolinylene, quinoxalinylene, phenanthrenylene or triphenylenylene, each of which may be substituted by one or more R radicals.

In a further preferred configuration, it may be the case that the Ar and/or Ar^{e} group is the same or different at each instance and is selected from structures of the formulae (Ar^{e}-1) to (Ar^{e}-9):

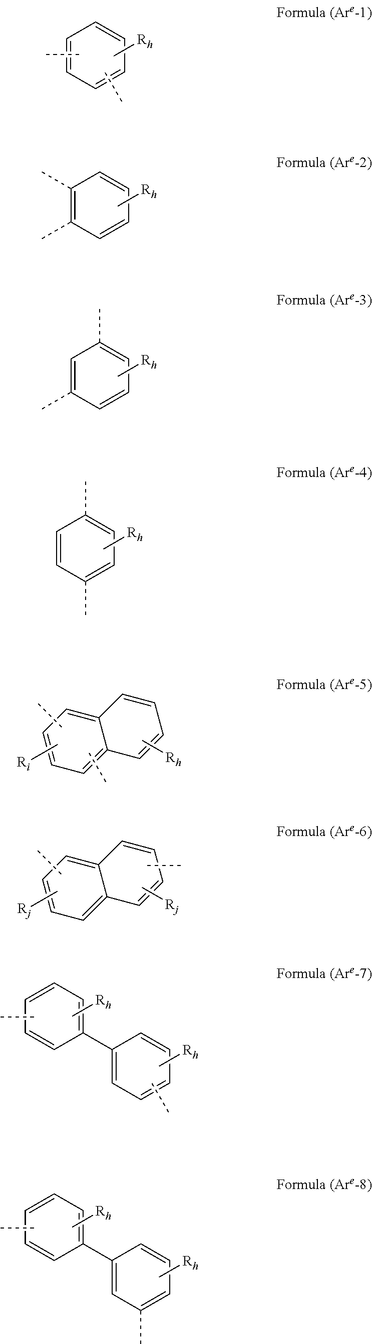

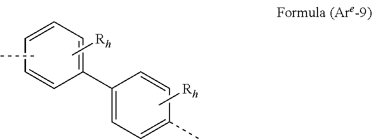

where the symbols used are as follows:
j at each instance is independently 0, 1, 2 or 3;
h at each instance is independently 0, 1, 2, 3 or 4;
R has the definition given above, especially for formula (I), and the dotted bond marks the position of attachment.

In a preferred embodiment, it may be the case that the Q group is the same or different at each instance and is selected from structures of the formulae (Q-1) to (Q-8):

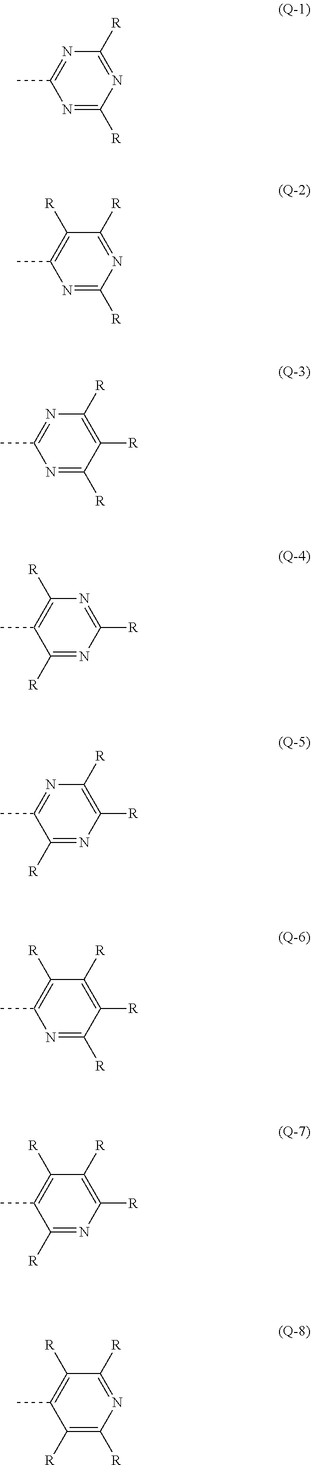

where R has the definition given above, especially for formula (I), and the dotted bond marks the position of attachment.

In an especially preferred configuration, it may be the case that the Q group is the same or different at each instance and is selected from structures of the formulae (Q-1a), (Q-1b), (Q-1c), (Q-1d), (Q-1e), (Q-1f), (Q-1g), (Q-1h), (Q-1i) and/or (Q-1j):

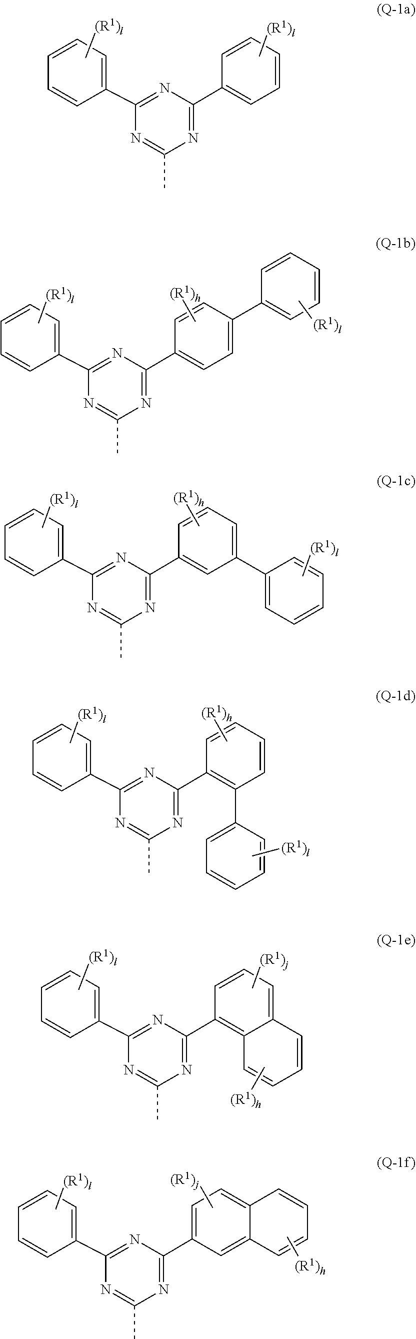

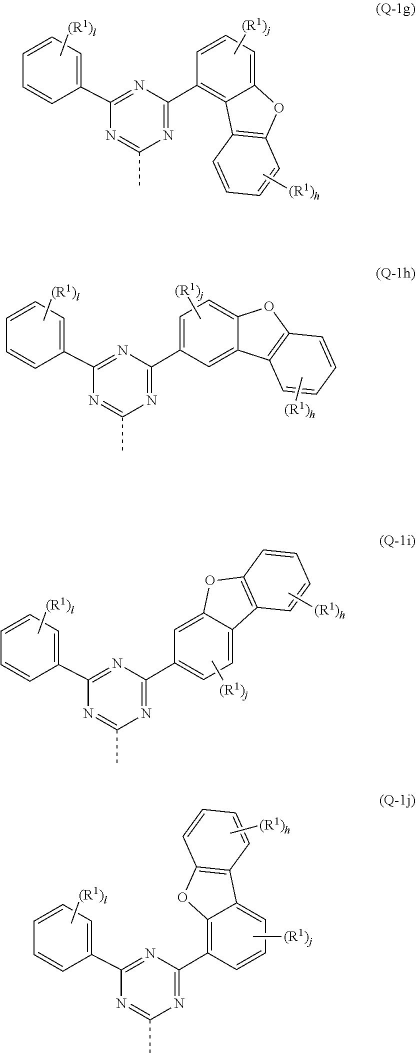

where R¹ has the definition given above, especially for formula (I), the dotted bond marks the position of attachment, the index I is the same or different at each instance and is 0, 1, 2, 3, 4 or 5, preferably 0, 1, 2 or 3, more preferably 0 or 1; the index h is the same or different at each instance and is 0, 1, 2, 3 or 4, preferably 0, 1 or 2, more preferably 0 or 1; the index j is the same or different at each instance and is 0, 1, 2 or 3, preferably 0, 1 or 2, more preferably 0 or 1. Preference is given here to structures of the formula (Q-1a).

In one embodiment, it may be the case that the compound comprises at least one structure of formula (I-2), where the Ar^{a} group is selected from structures of the formulae (Ar^{a}-1) to (Ar^{a}-18), where, in structures of the formulae (Ar^{a}-8) to (Ar^{a}-12), the Y group is preferably O or NR and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is preferably O or NR, where the Ar^{d} groups are the same or different at each instance and are preferably selected from structures of the formulae (Ar^{a}-1), (Ar^{a}-2) and (Ar^{a}-7) to (Ar^{a}-18). In this context, in a preferred configuration, the Ar^{d} groups are preferably connected by a single bond, where these Ar^{d} groups more preferably form a carbazole radical.

In one embodiment, it may be the case that the compound comprises at least one structure of formula (I-4), where the Ar group is selected from structures of the formulae (Ar^{e}-1) to (Ar^{e}-4), the Ar^{a} group is selected from structures of the formulae (Ar^{a}-1) to (Ar^{a}-18), where, in structures of the formulae (Ar^{a}-8) to (Ar^{a}-12), the Y group is preferably O or NR, and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is preferably O or NR, where the Ar^{d} groups are the same or different at each instance and are preferably selected from structures of the formulae (Ar^{a}-1), (Ar^{a}-2), (Ar^{a}-7) to (Ar^{a}-18). In this context, in a preferred configuration, the Ar^{d} groups or at least one of the Ar^{d} groups together with the Ar group are connected by a single bond, where these Ar^{d} groups or at least one of the Ar^{d} groups together with the Ar group more preferably form a carbazole radical.

In one embodiment, it may be the case that the compound comprises at least one structure of formula (I-3), where the Ar^{c} groups are the same or different at each instance and are selected from structures of the formulae (Ar^{a}-1) to (Ar^{a}-23), where, in structures of the formulae (Ar^{a}-8) to (Ar^{a}-12), the Y group is preferably O or NR; preferably, the Ar^{c} groups are the same or different at each instance and are selected from structures of the formulae (Ar^{a}-1), (Ar^{a}-2), (Ar^{a}-7) to (Ar^{a}-18); and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is preferably O or NR, where the Ar^{d} groups are the same or different at each instance and are preferably selected from structures of the formulae (Ar^{a}-1), (Ar^{a}-2), (Ar^{a}-7) to (Ar^{a}-18). In this context, in a preferred configuration, the Ar^{c} groups are preferably connected by a single bond, where these Ar^{c} groups more preferably form a carbazole radical. In an especially preferred configuration, the Ar^{c} groups in each case and the Ar^{d} groups in each case are connected by a single bond, where these Ar^{c} and Ar^{d} groups more preferably each form a carbazole radical.

In one embodiment, it may be the case that the compound comprises at least one structure of formula (I-5) and/or formula (I-6), where the Ar group is the same or different at each instance and is selected from structures of the formulae (Ar^{e}-1) to (Ar^{e}-4), the Ar^{c} groups are the same or different at each instance and are selected from structures of the formulae (Ar^{a}-1) to (Ar^{a}-23), where, in structures of the formulae (Ar^{a}-8) to (Ar^{a}-12), the Y group is preferably O or NR; preferably, the Ar^{c} groups are the same or different at each instance and are selected from structures of the formulae (Ar^{a}-1), (Ar^{a}-2), (Ar^{a}-7) to (Ar^{a}-18); and the Ar^{d} groups are the same or different at each instance and are selected from structures of the formulae (Ar^{a}-1) to (Ar^{a}-18), where, in structures of the formulae (Ar^{a}-8) to (Ar^{a}-12), the Y group is preferably O or NR, where the Ar^{d} groups are the same or different at each instance and are preferably selected from structures of the formulae (Ar^{a}-1), (Ar^{a}-2), (Ar^{a}-7) to (Ar^{a}-18). In this context, in a preferred configuration, the Ar^{c} groups or at least one of the Ar^{c} groups together with the Ar group are preferably connected by a single bond, where these Ar^{e} groups or at least one of the Ar^{c} groups together with the Ar group more preferably form a carbazole radical. In a further preferred configuration, the Ar^{d} groups or at least one of the Ar^{d} groups together with the Ar group are preferably connected by a single bond, where these Ar^{d} groups or at least one of the Ar^{d} groups together with the Ar group more preferably form a carbazole radical. In an especially preferred configuration, the Ar^{c} groups or at least one of the Ar^{c} groups together with the Ar group are connected by a single bond, where these groups preferably form a carbazole radical, and the Ar^{d} groups or at least one of the Ar^{d} groups together with the Ar group are connected by a single bond, where these groups preferably form a carbazole radical. These especially preferred compounds accordingly have two carbazole radicals, where one carbazole radical comprises an Ar^{c} group and one carbazole radical comprises an Ar^{d} group.

It may preferably be the case that the compound comprises at least one structure of formula (I-2), where the Ar^{a} group is a structure of the formula Ar^{e}-Q, where the symbols Ar^{e} and Q have the definition given above, and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is preferably O or NR, where the Ar^{d} groups are the same or different at each instance and are preferably selected from structures of the formulae (Ar^{a}-1), (Ar^{a}-2), (Ar^{a}-7) to (Ar^{a}-18). In this context, in a preferred configuration, the Ar^{d} groups are preferably connected by a single bond, where these Ar^{d} groups more preferably form a carbazole radical.

In addition, it may be the case that the compound comprises at least one structure of formula (I-4), where the Ar group is selected from structures of the formulae (Ar^{e}-1) to (Ar^{e}-4), the Ar^{a} group is a structure of the formula Ar^{e}-Q, where the symbols Ar^{e} and Q have the definition given above, and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is preferably O, where the Ar^{d} groups are the same or different at each instance and are preferably selected from structures of the formulae (Ar^{a}-1), (Ar^{a}-2), (Ar^{a}-7) to (Ar^{a}-18). In this context, in a preferred configuration, the Ar^{d} groups or at least one of the Ar^{d} groups together with the Ar group are connected by a single bond, where these Ar^{d} groups or at least one of the Ar^{d} groups together with the Ar group more preferably form a carbazole radical.

In a further configuration, it may be the case that the compound comprises at least one structure of formula (I-1) and/or formula (I-2), where the Ar^{a} group comprises an electron transport group or is an electron transport group, where the Ar^{a} group preferably comprises a pyridine, pyrimidine, pyrazine, pyridazine, triazine, quinazoline, quinoxaline, quinoline, isoquinoline, imidazole and/or benzimidazole group or is one of the groups mentioned, where these may be substituted by one or more R radicals, where triazine groups are particularly preferred.

It may preferably be the case that the compound comprises at least one structure of formula (I-1), where the Ar^{b} group comprises an electron transport group or is an electron transport group, where the Ar^{b} group preferably comprises a pyridine, pyrimidine, pyrazine, pyridazine, triazine, quinazoline, quinoxaline, quinoline, isoquinoline, imidazole and/or benzimidazole group or is one of the groups mentioned, where these may be substituted by one or more R radicals, where triazine groups are particularly preferred.

It may more preferably be the case that the compound comprises at least one structure of formula (I-1), where the Ar^{a} group is a structure of the formula Ar^{e}-Q and the Ar^{b} group is a structure of the formula Ar^{e}-Q, where the symbols Ar^{e} and Q have the definition given above.

In a further configuration, it may be the case that the compound comprises at least one structure of formula (I-1), where the Ar^{a} group comprises an electron transport group or is an electron transport group, where the Ar^{a} group preferably comprises a pyridine, pyrimidine, pyrazine, pyridazine, triazine, quinazoline, quinoxaline, quinoline, isoquinoline, imidazole and/or benzimidazole group or is one of the groups mentioned, where these may be substituted by one or more R radicals, where triazine groups are particularly preferred, and the Ar^{b} group is selected from structures of the formulae (Ar^{a}-1) to (Ar^{a}-18), where, in structures of the formulae (Ar^{a}-8) to (Ar^{a}-12), the Y group is preferably O or NR; where the Ar^{a} group is preferably a structure of the formula Ar^{e}-Q, where the symbols Ar^{e} and Q have the definition given above.

The present compounds are especially suitable as host material for emitters, preferably as host material for singlet, triplet and TADF emitters, electron transport material, electron injection material, hole conductor material, hole injection material, electron blocker material, hole blocker material, in an electronic device. The specific properties of the compounds depend here on the type and number of the respective functional groups. Compounds comprising one, two or more electron transport groups, but not a hole transport group, are especially suitable as host material, electron transport material, electron injection material and/or hole blocker material. Compounds comprising one, two or more hole transport groups, but not an electron transport group, are especially suitable as host material, hole conductor material, hole injection material and/or electron blocker material. Compounds comprising one, two or more hole transport groups and one, two or more electron transport groups are especially suitable as host material.

Electron transport groups are widely known in the technical field and promote the ability of compounds to transport and/or to conduct electrons. Examples of electron transport groups are pyridine, pyrimidine, pyrazine, pyridazine, triazine, quinazoline, quinoxaline, quinoline, isoquinoline, imidazole and/or benzimidazole groups.

Hole transport groups are likewise known in the technical field, and they preferably include triarylamine or carbazole groups.

It may also be the case that the Ar, Ar^{b}, Ar^{c}, Ar^{d} groups do not comprise any triazine group, preferably any pyridine, pyrimidine, pyrazine, pyridazine, triazine, quinazoline, quinoxaline, quinoline, isoquinoline, imidazole and/or benzimidazole group, and more preferably any electron transport group.

It may additionally be the case that the Ar, Ar^{a}, Ar^{b}, Ar^{c}, Ar^{d} groups do not comprise any triazine group, preferably any pyridine, pyrimidine, pyrazine, pyridazine, triazine, quinazoline, quinoxaline, quinoline, isoquinoline, imidazole and/or benzimidazole group, and more preferably any electron transport group.

It may moreover be the case that the Ar, Ar^{a}, Ar^{c}, Ar^{d} groups do not comprise any carbazole group, preferably any carbazole group and/or any substituents of the formula N(Ar′)₂, N(R¹)₂, and more preferably any hole transport group.

It may further be the case that the Ar, Ar^{b}, Ar^{c}, Ar^{d} groups do not comprise any carbazole group, preferably any carbazole group and/or any substituents of the formula N(Ar′)₂, N(R¹)₂, and more preferably any hole transport group.

It may also be the case that the Ar, Ar^{a}, Ar^{b}, Ar^{c}, Ar^{d} groups do not comprise any carbazole group, preferably any carbazole group and/or any substituents of the formula N(Ar′)₂, N(R¹)₂, and more preferably any hole transport group.

It may additionally be the case that the Ar^{e} group does not comprise any carbazole group, preferably any carbazole group and/or any substituents of the formula N(Ar′)₂, N(R¹)₂, and more preferably any hole transport group.

It may moreover be the case that the Q group does not comprise any carbazole group, preferably any carbazole group and/or any substituents of the formula N(Ar′)₂, N(R¹)₂, and more preferably any hole transport group.

In a further preferred embodiment, it may be the case that the compounds of the invention include a structure of the formulae (II-1) to (II-154), where the compounds of the invention may more preferably be selected from the compounds of the formulae (II-1) to (II-154)

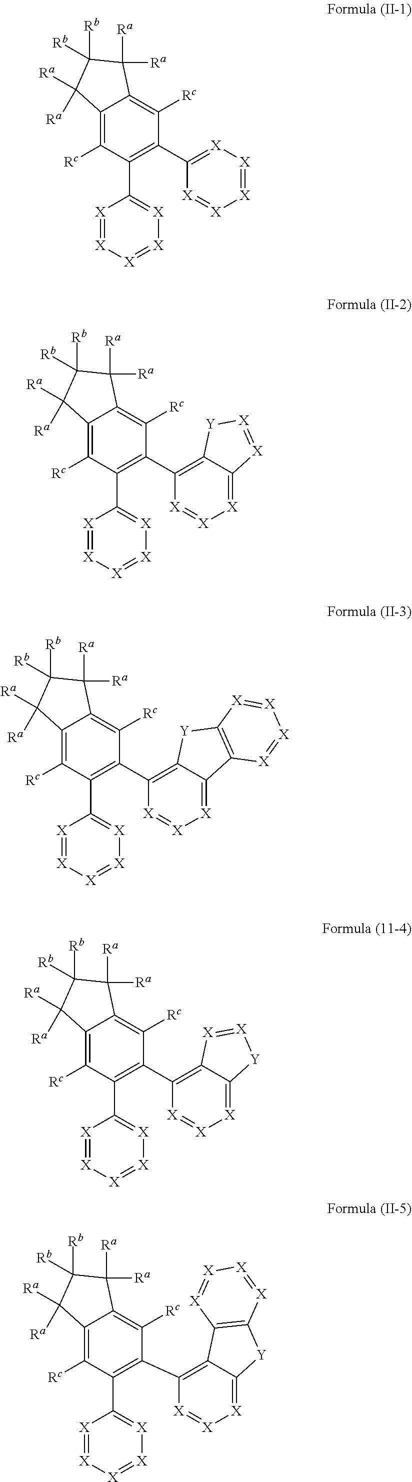

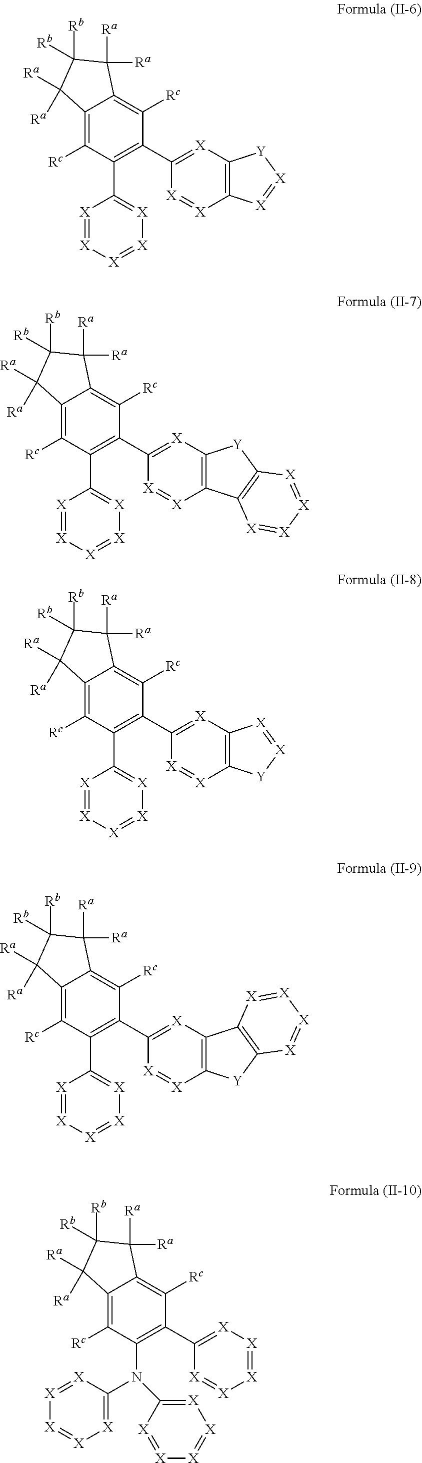

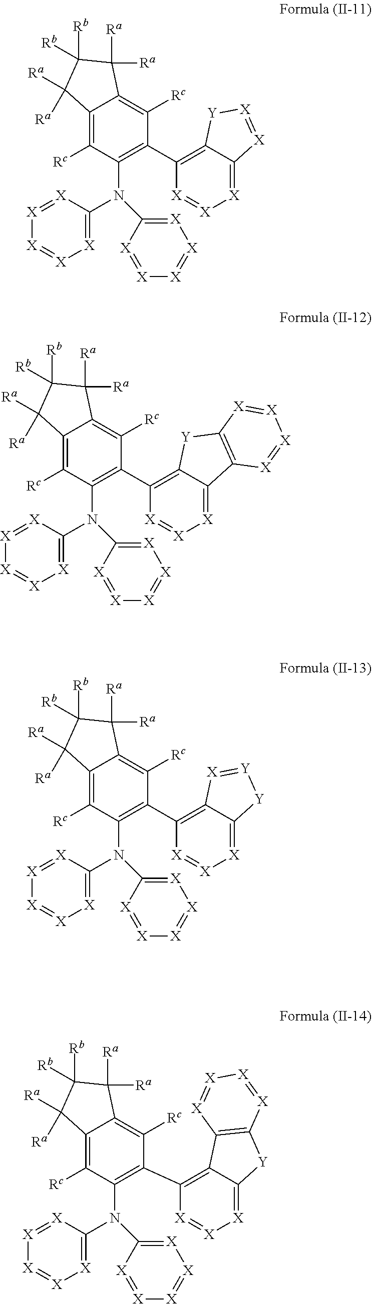

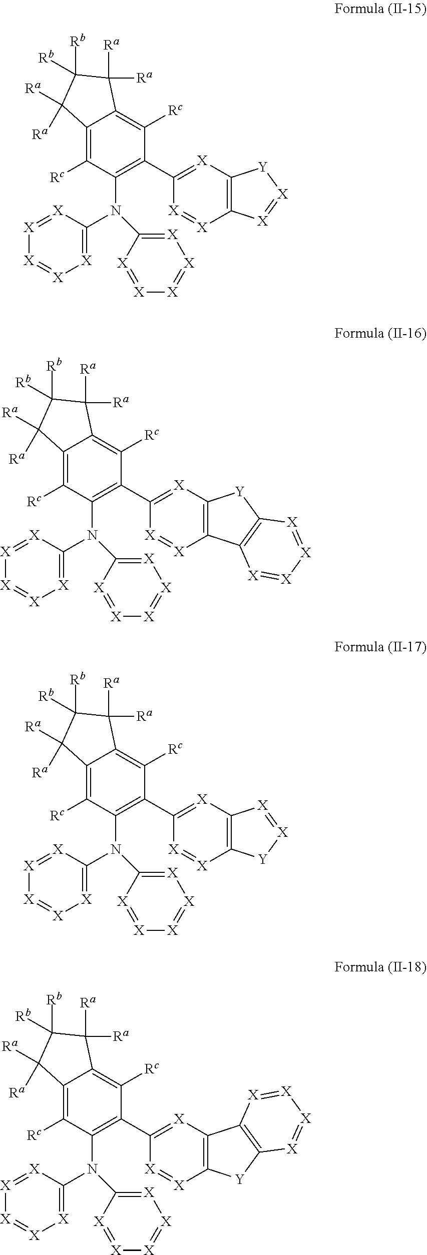

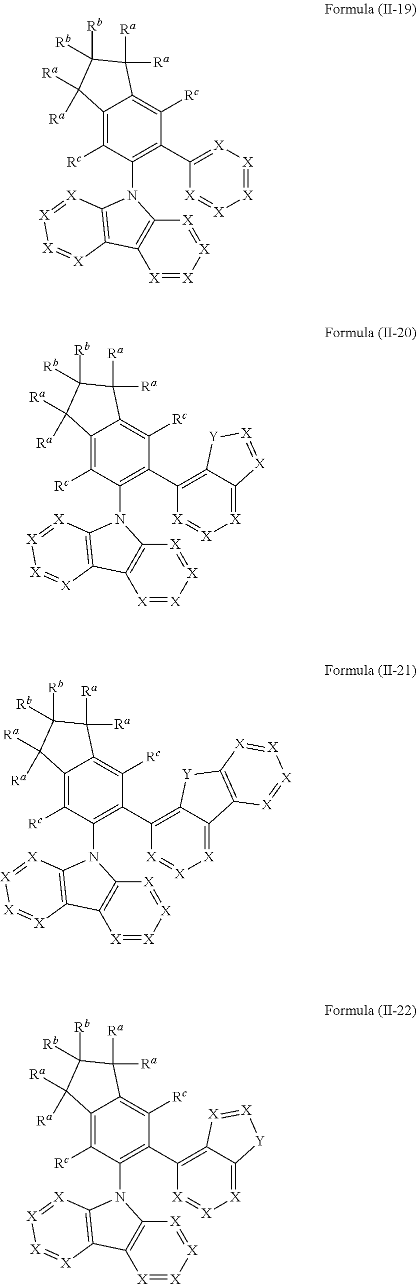

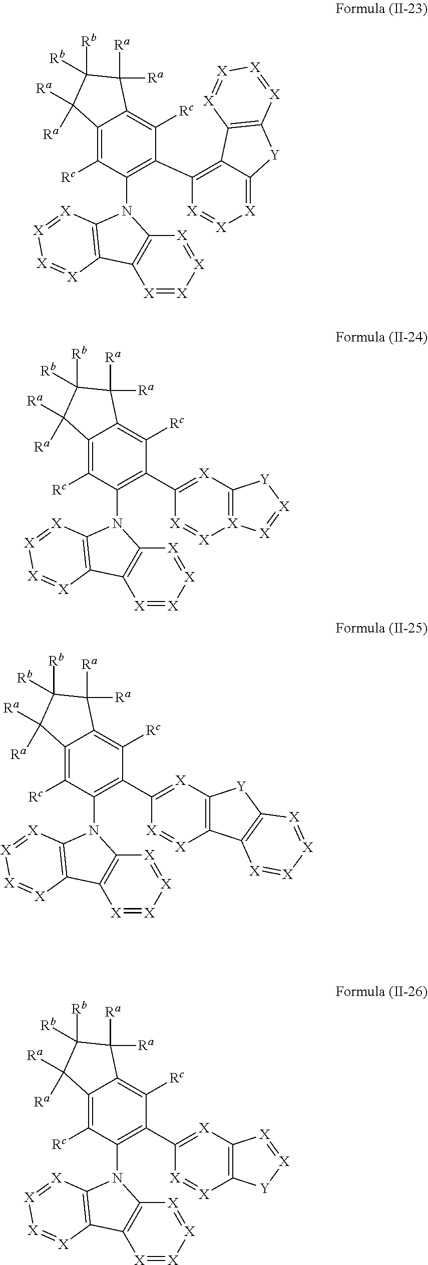

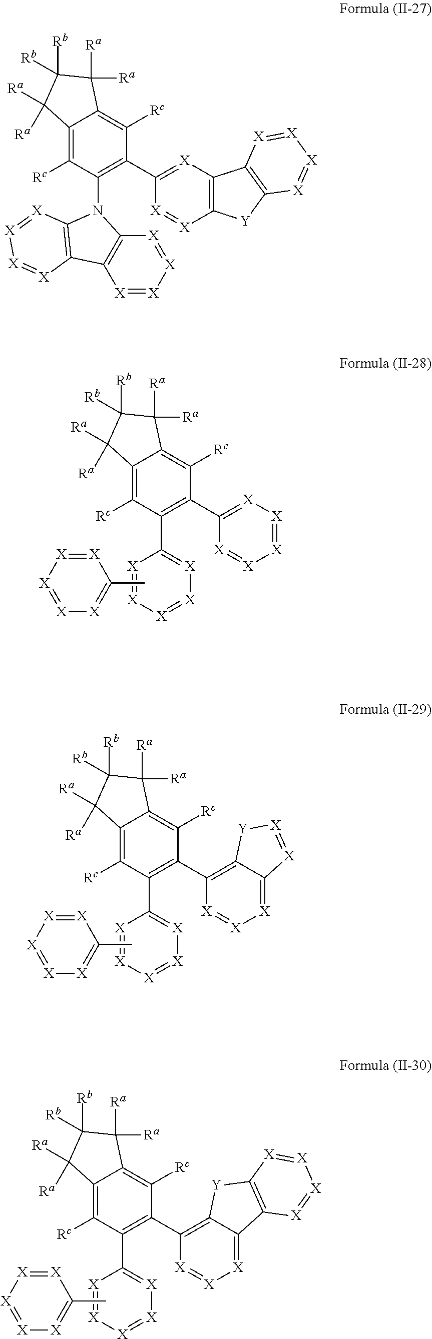

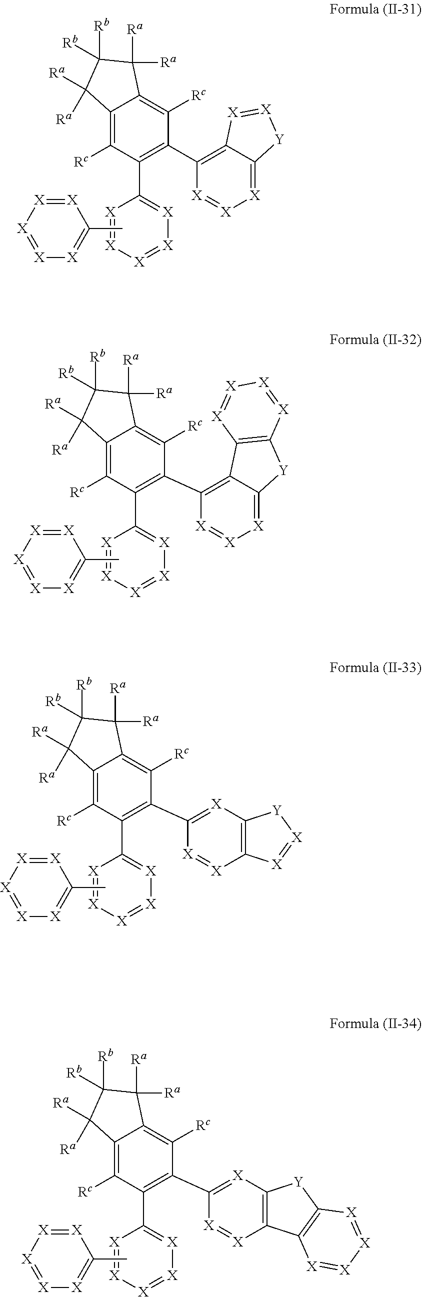

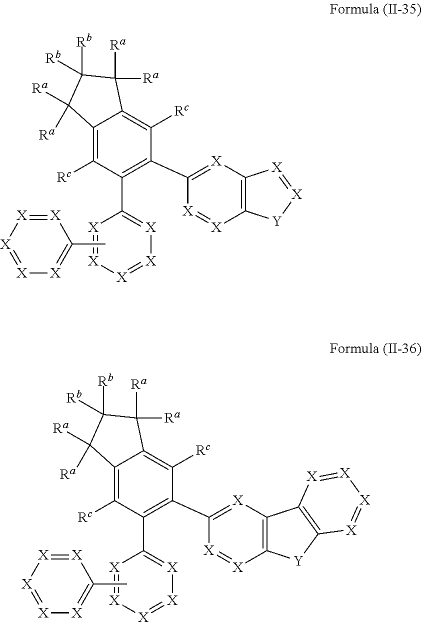

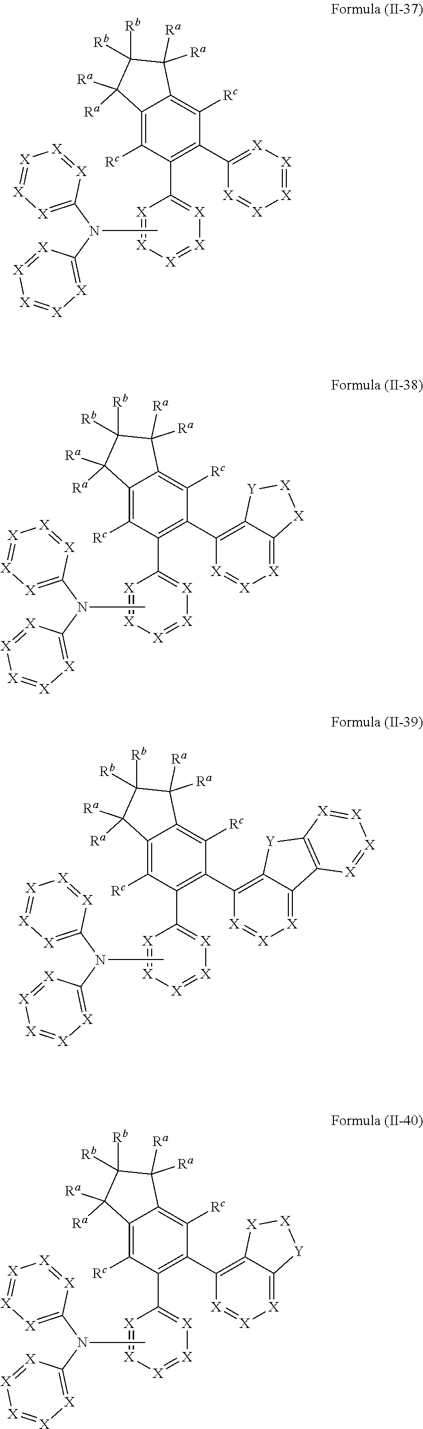

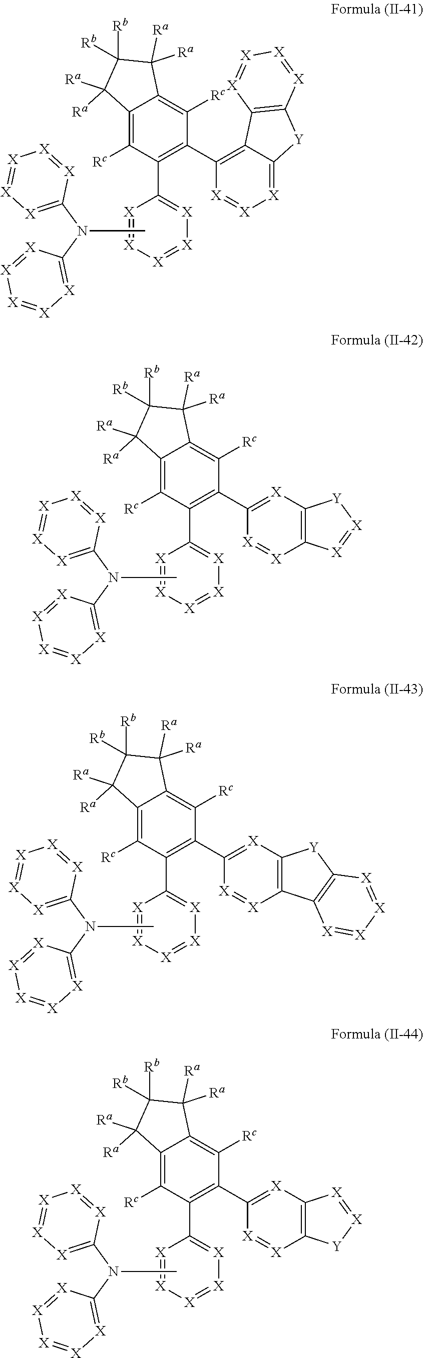

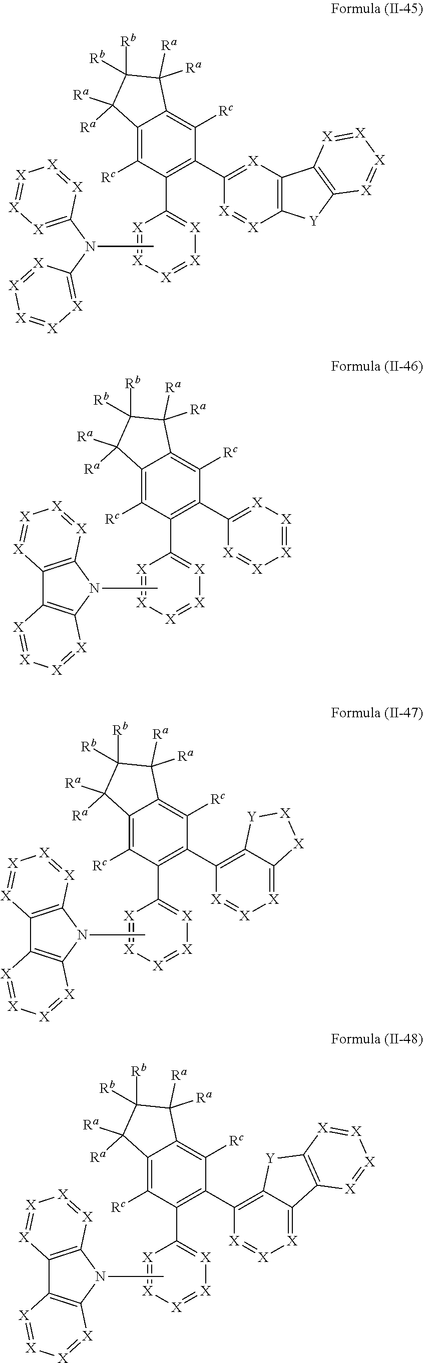

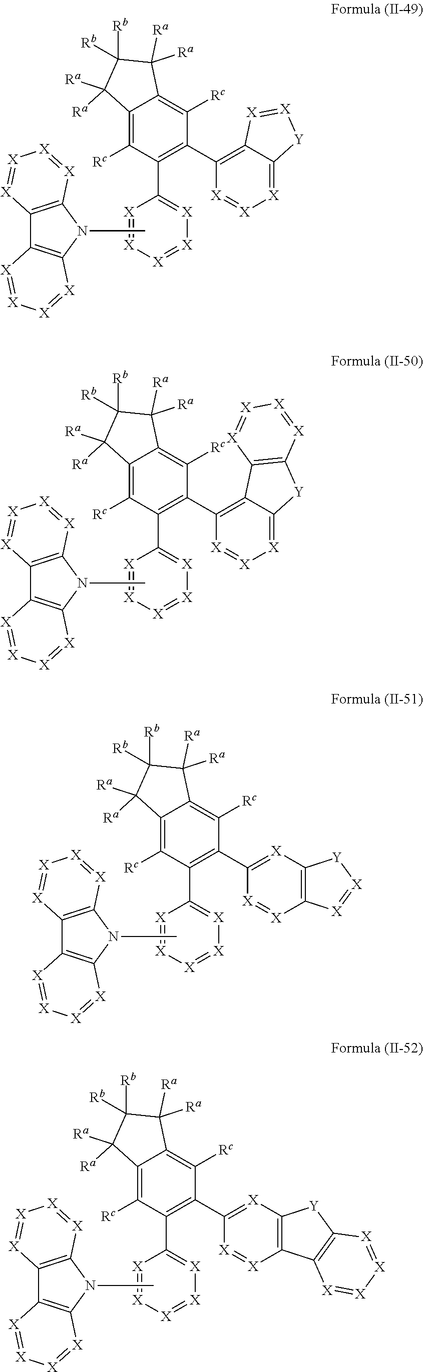

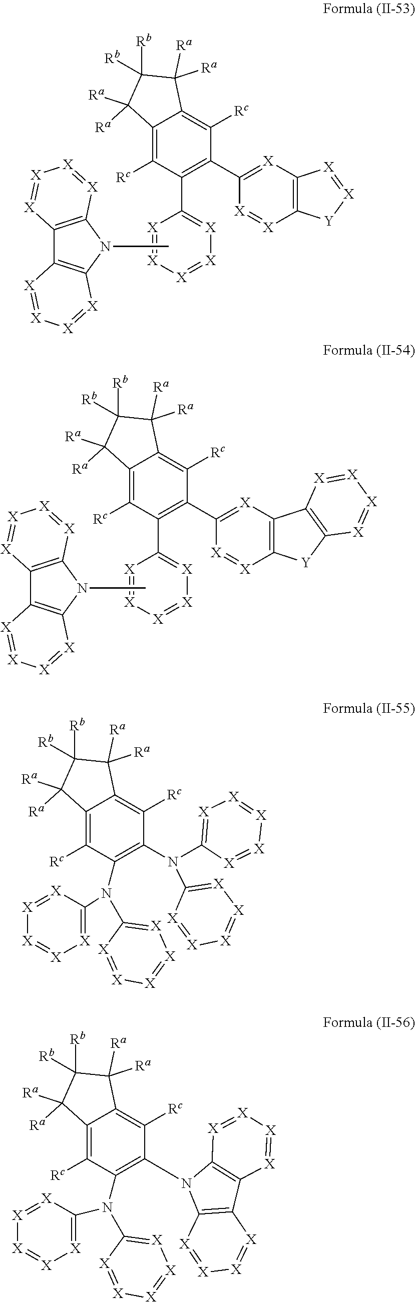

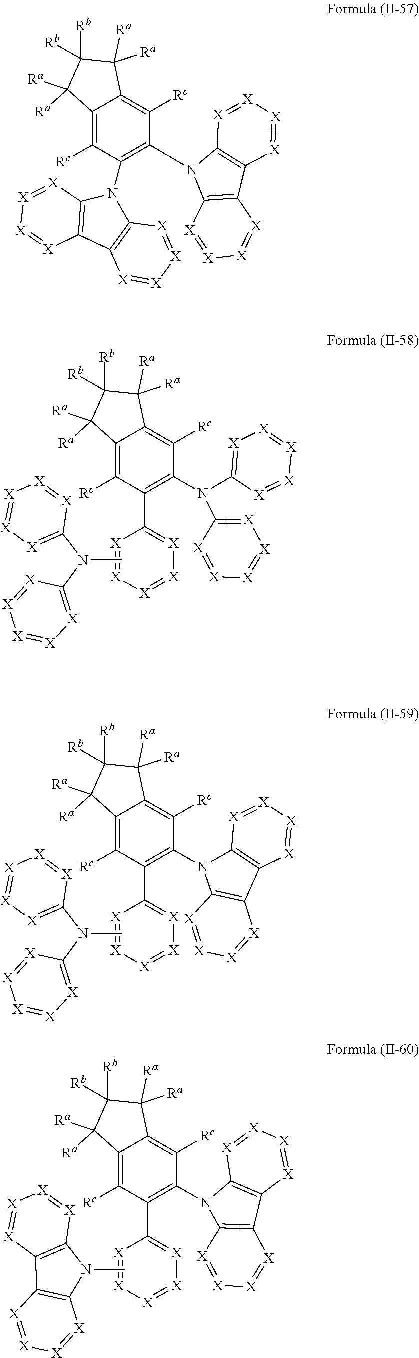

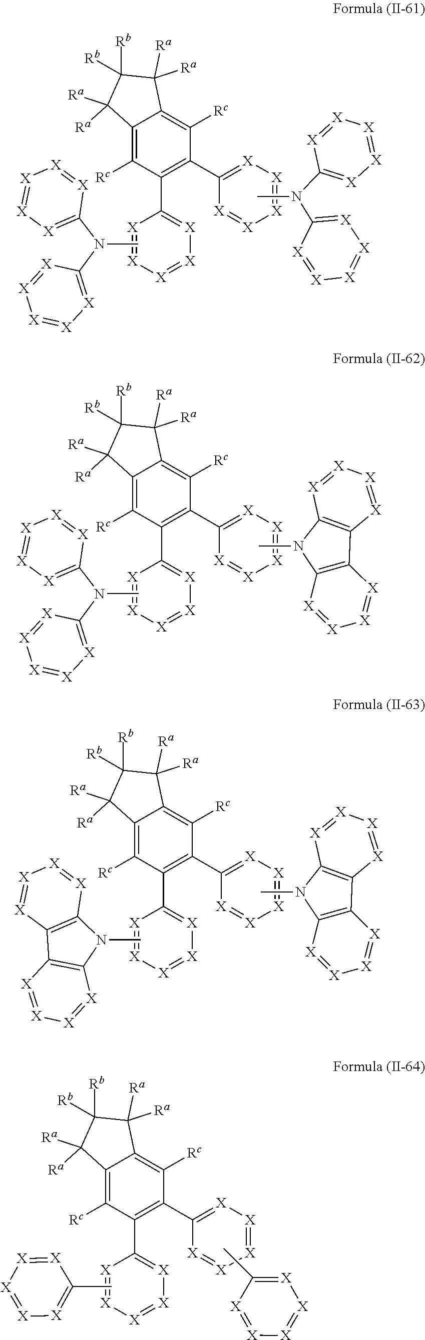

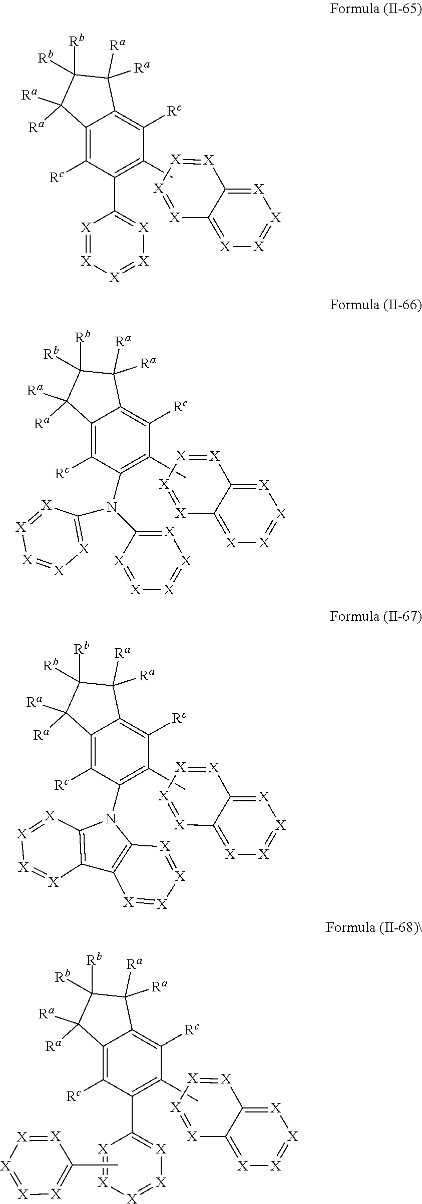

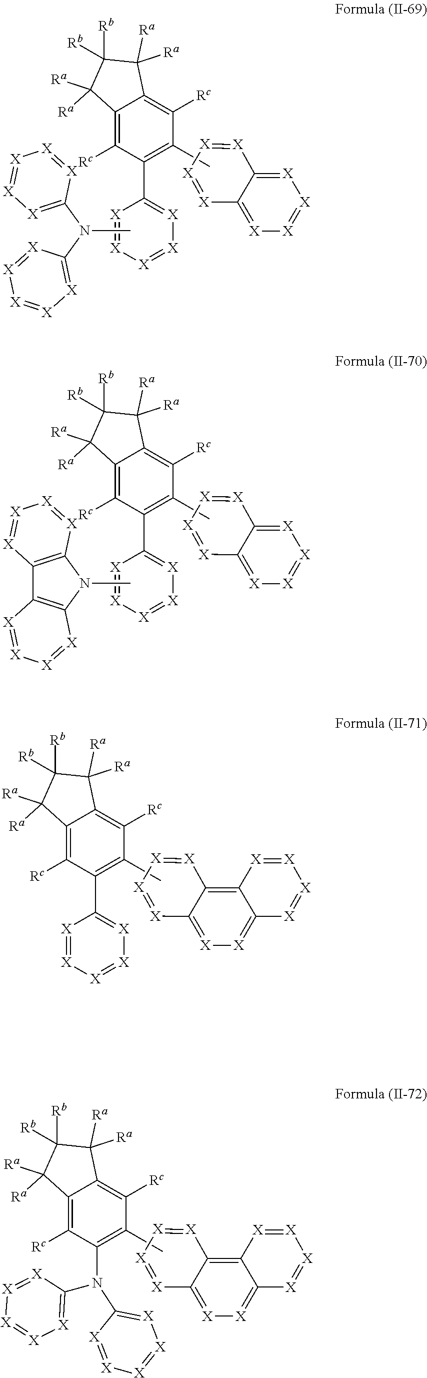

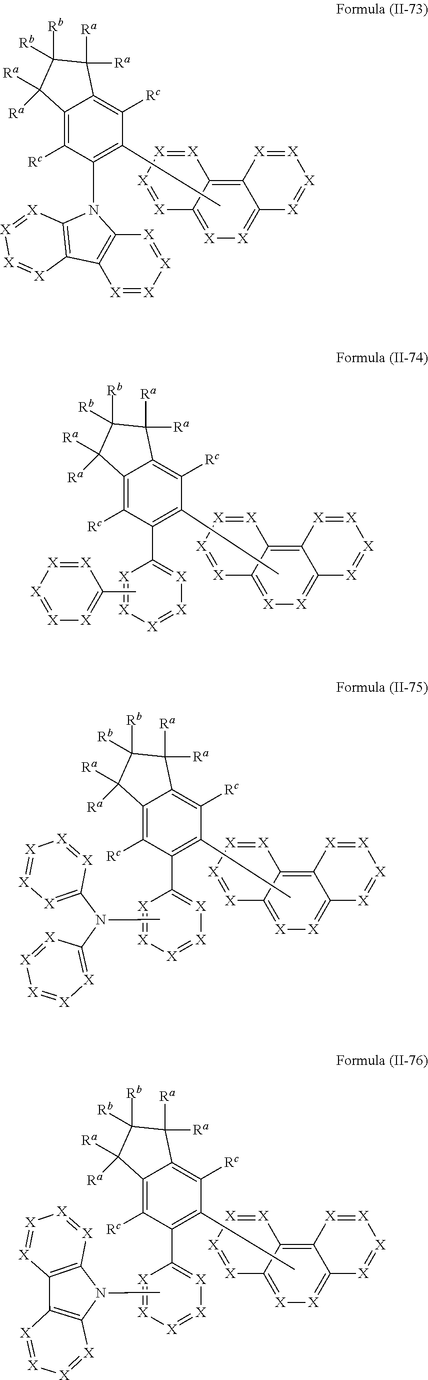

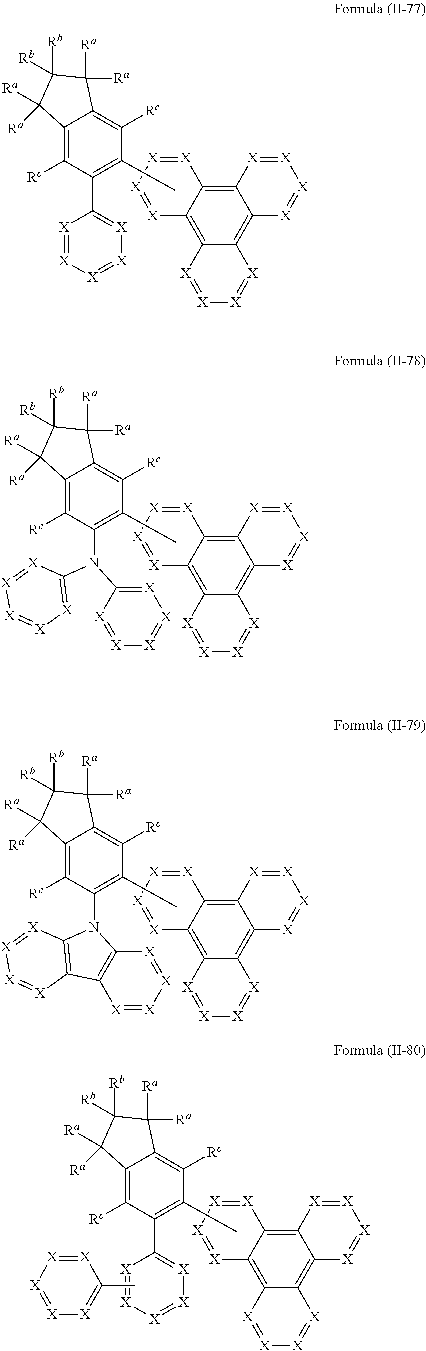

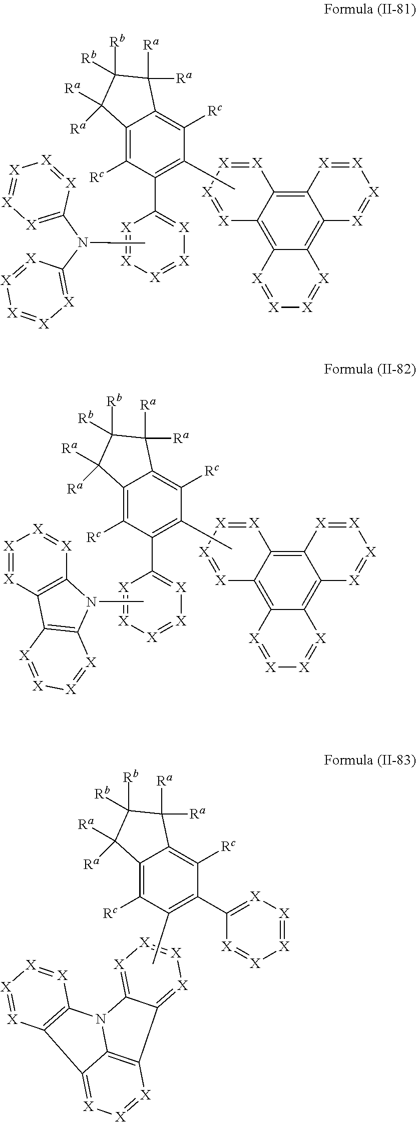

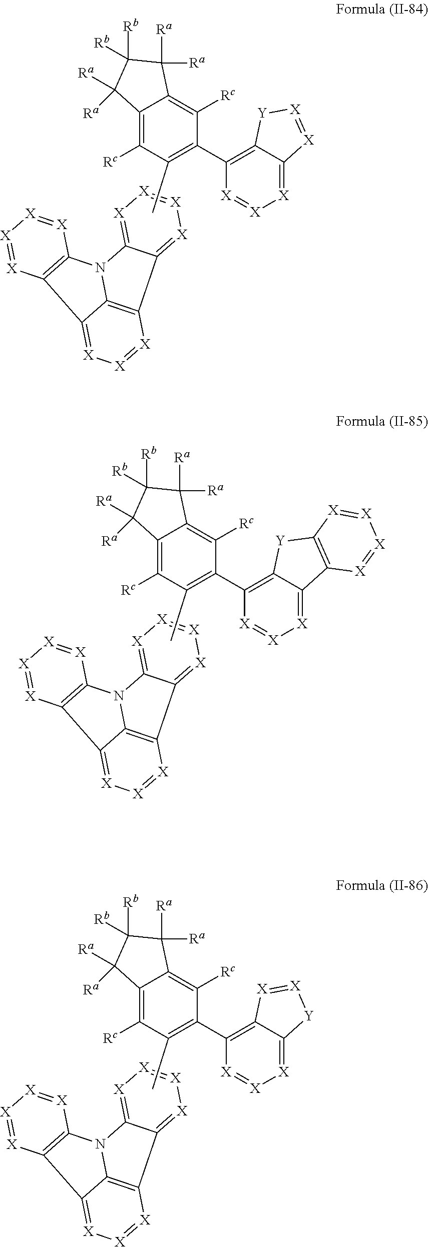

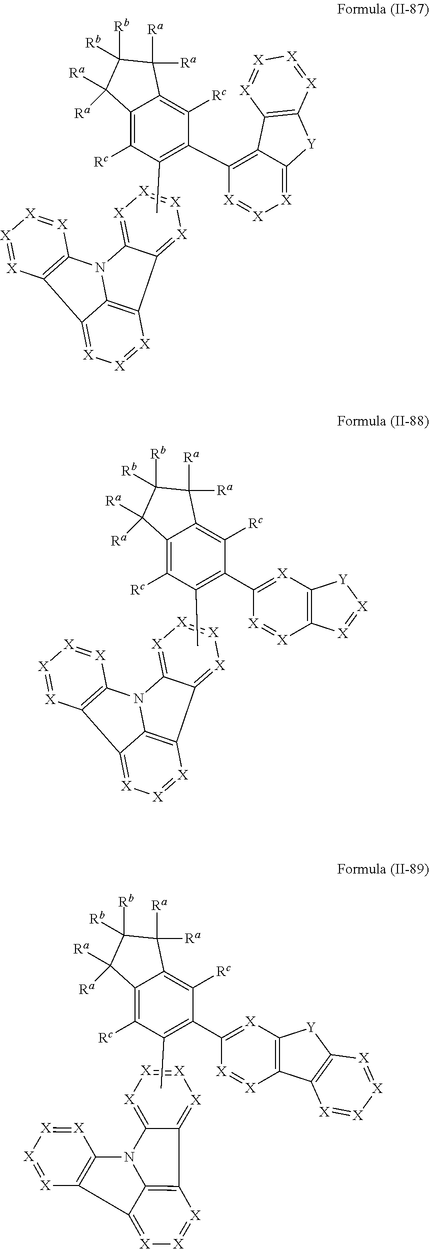

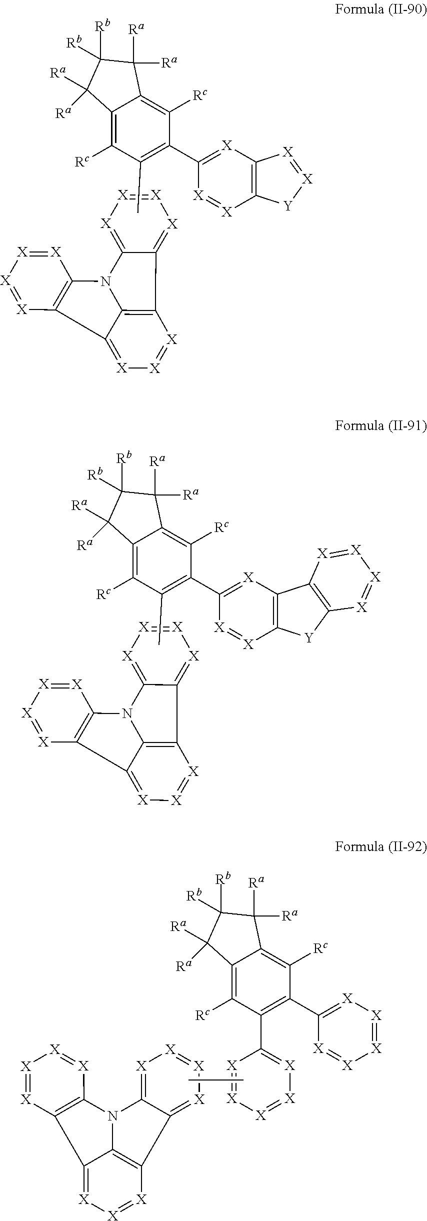

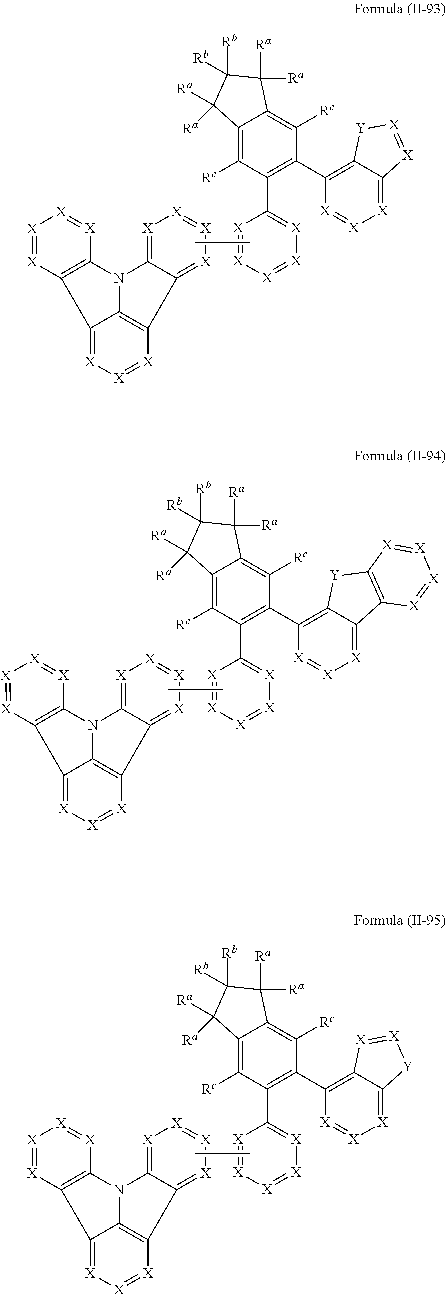

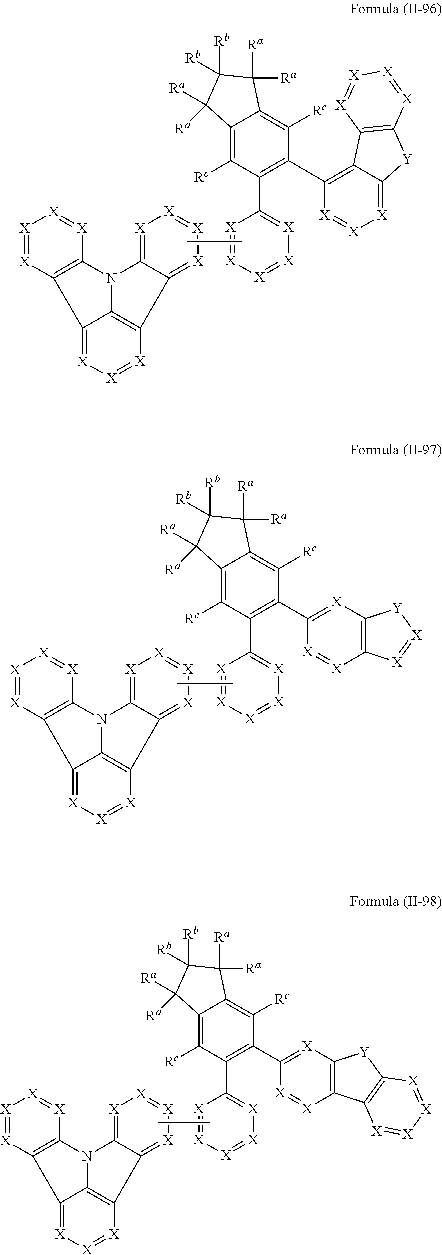

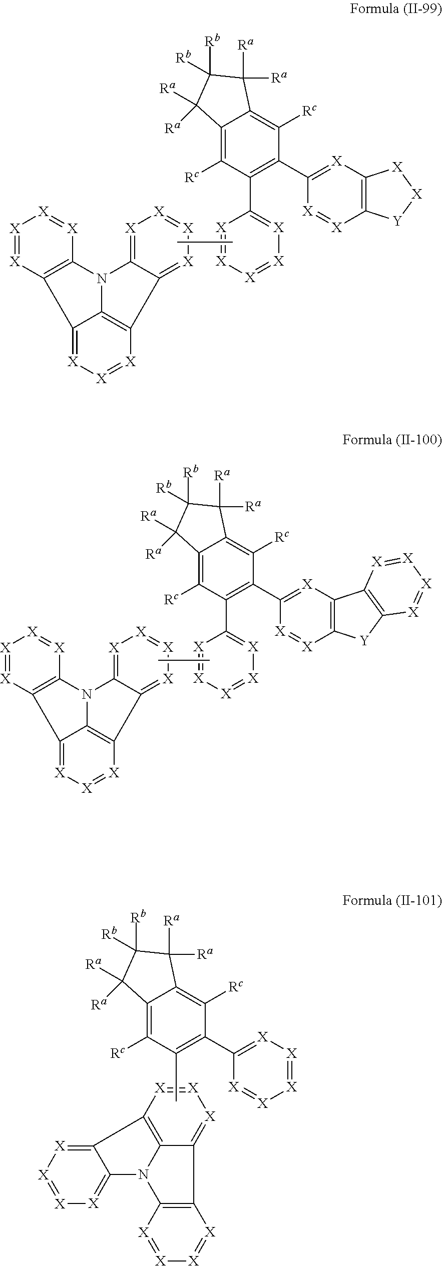

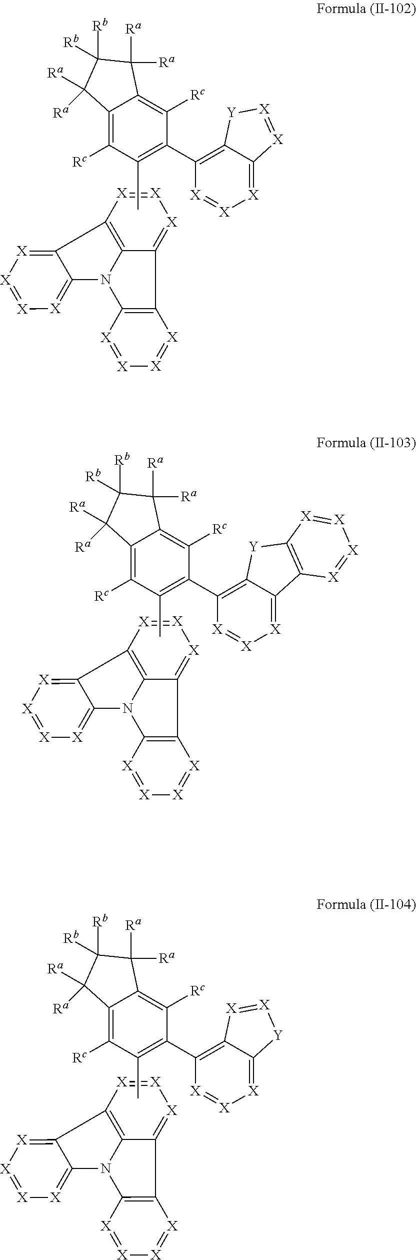

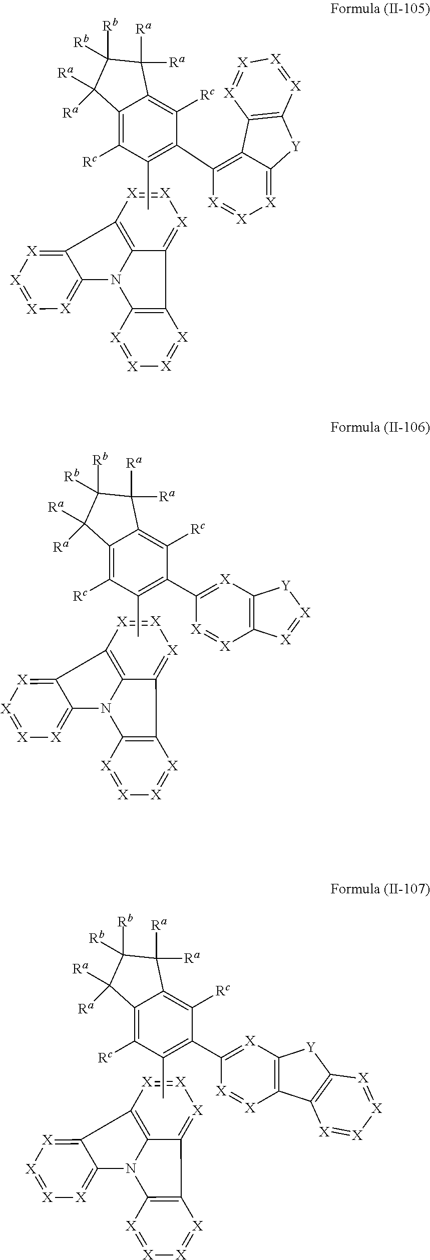

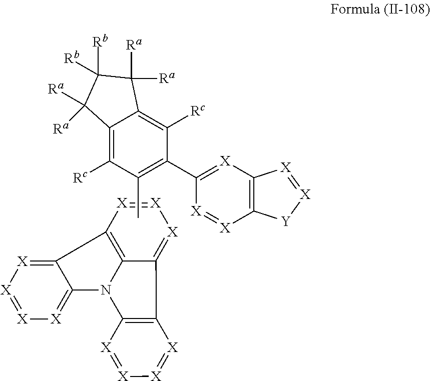

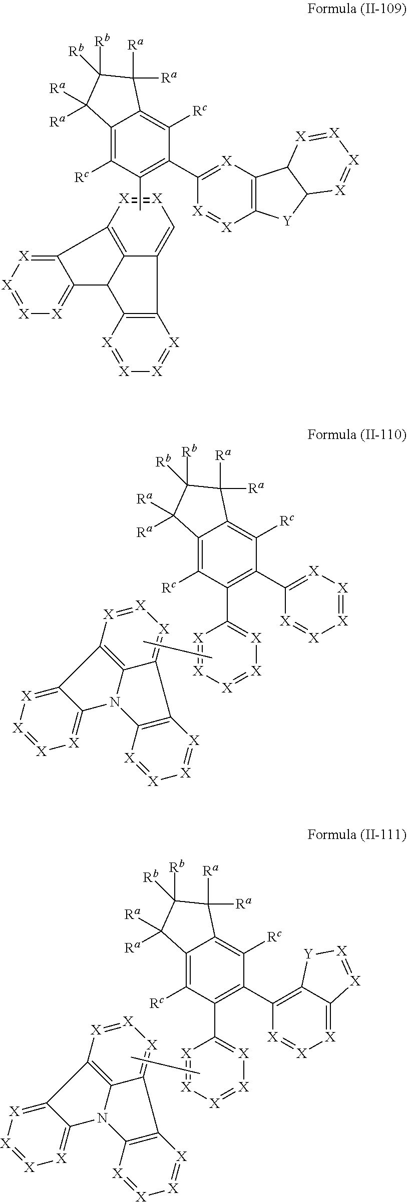

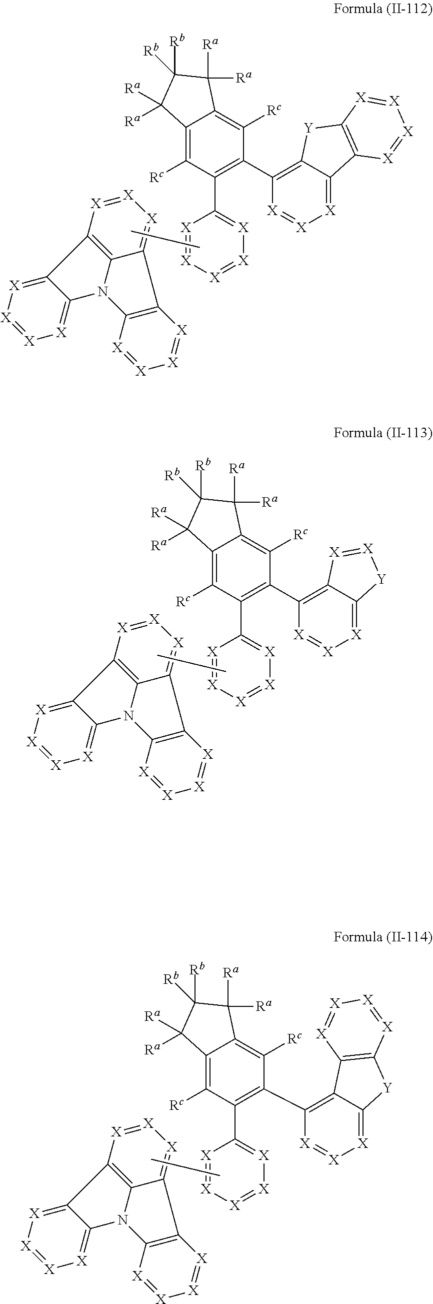

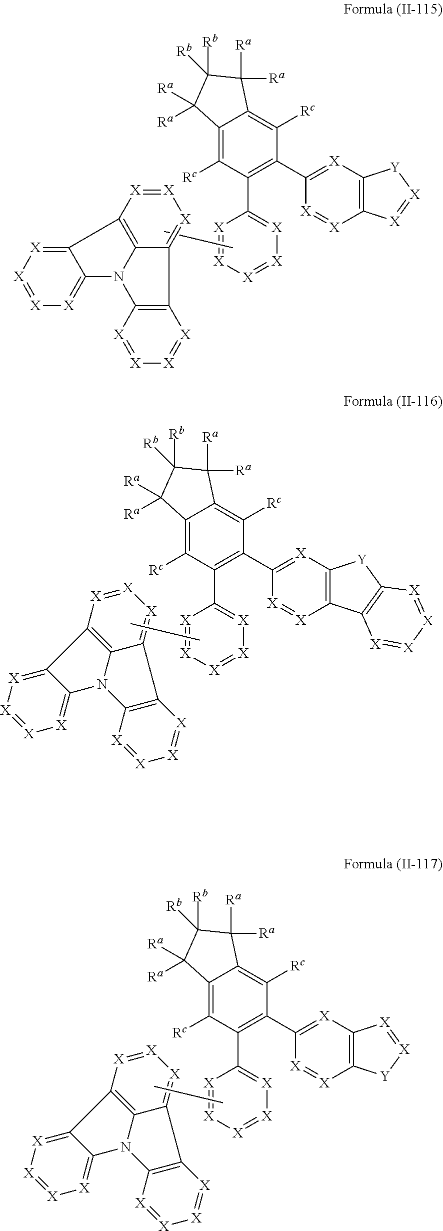

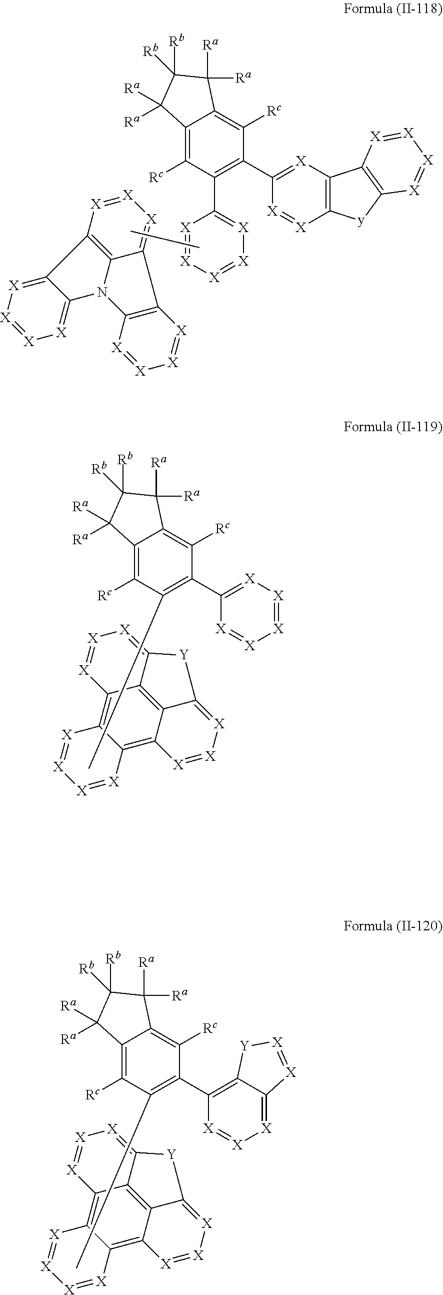

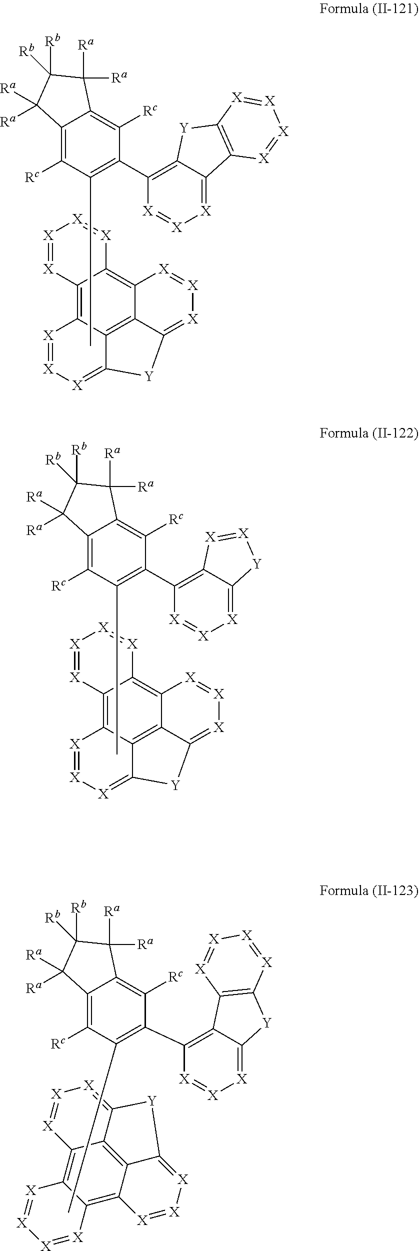

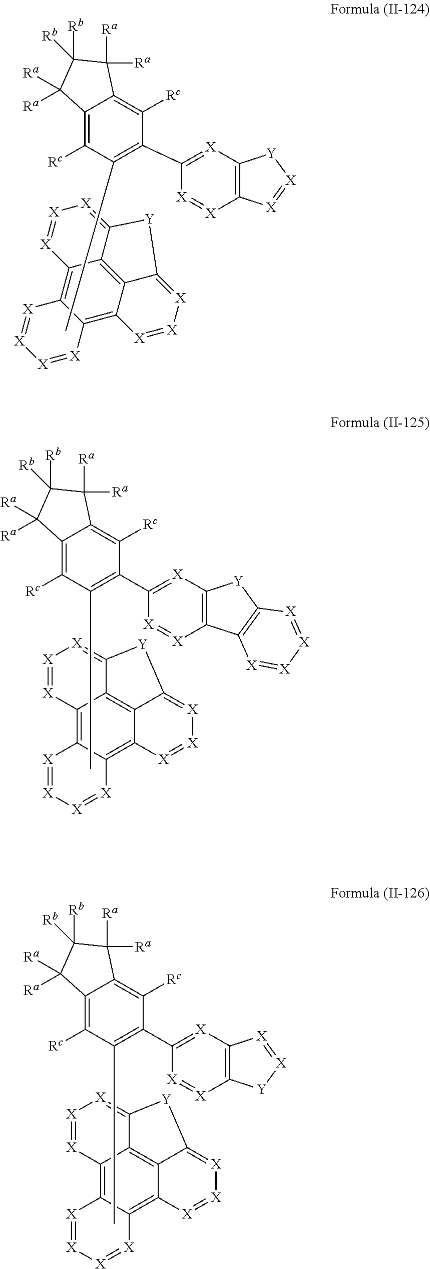

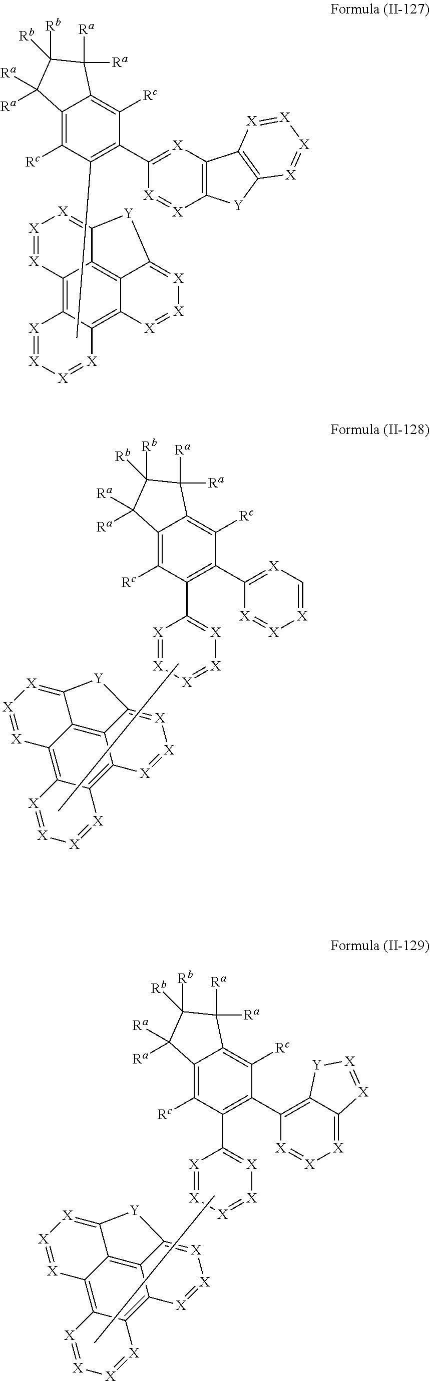

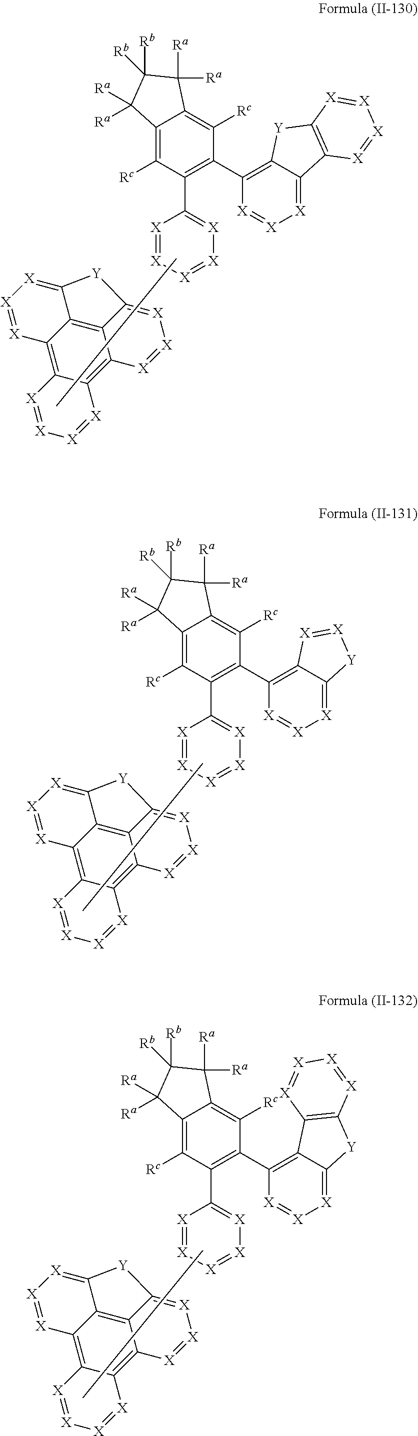

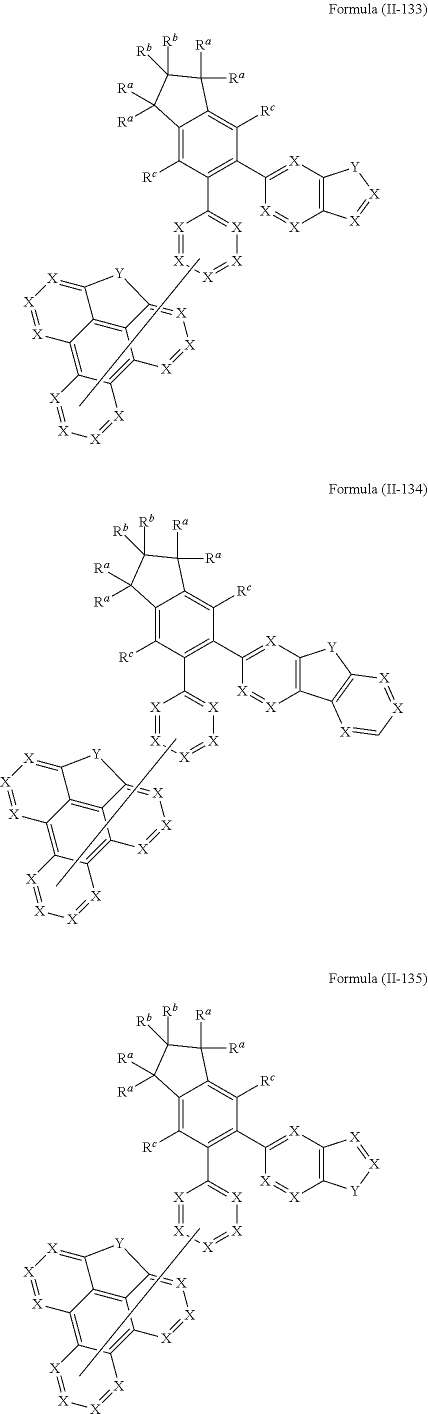

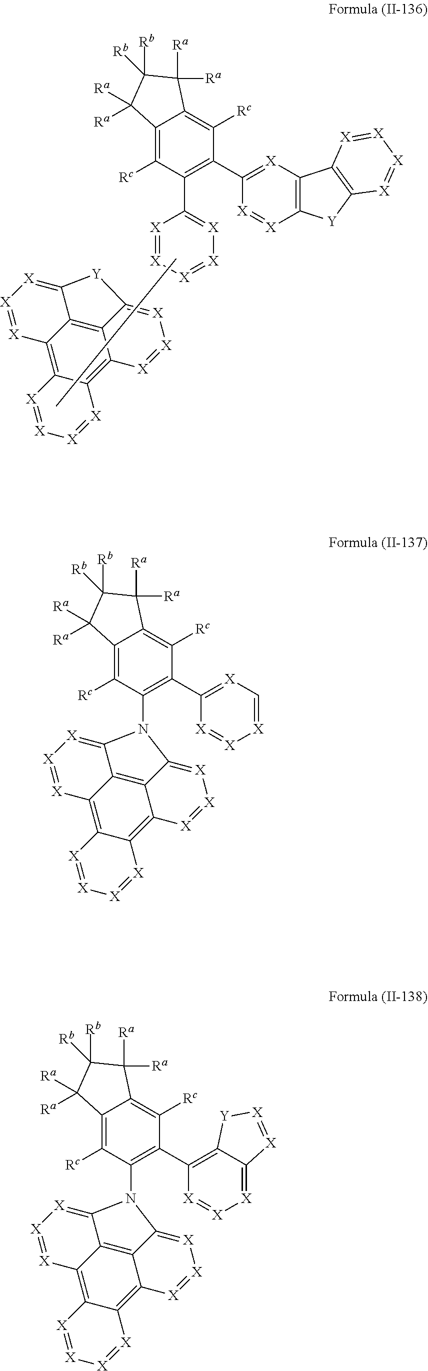

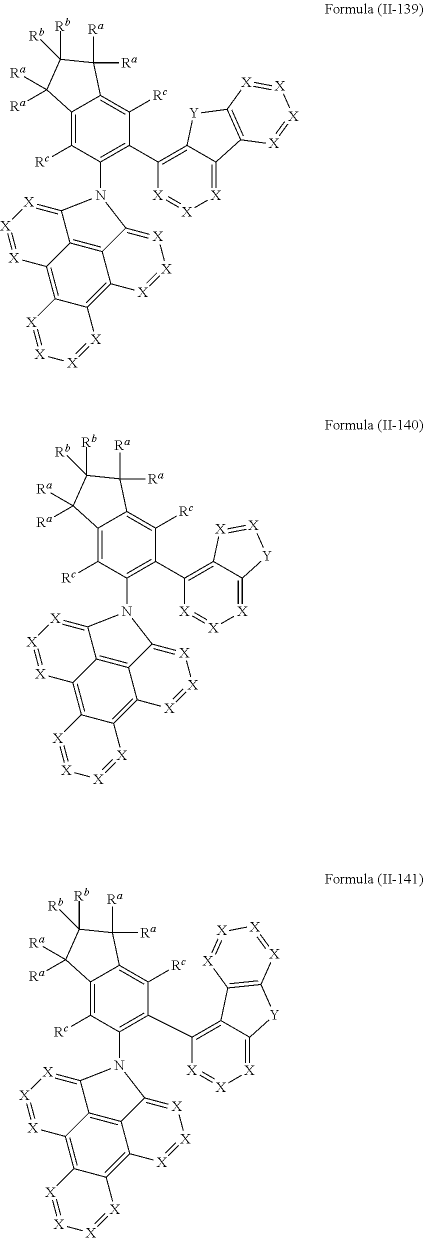

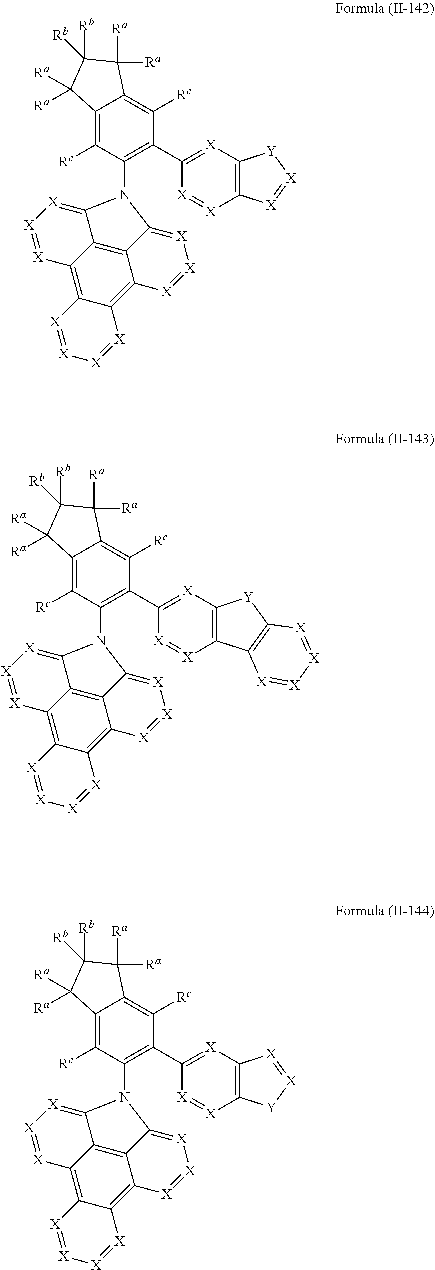

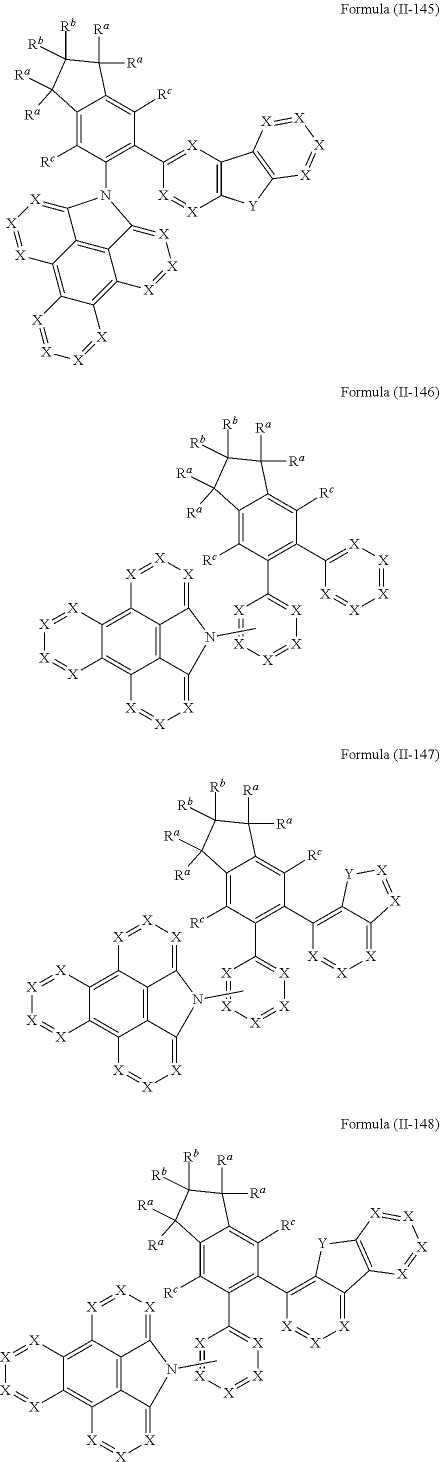

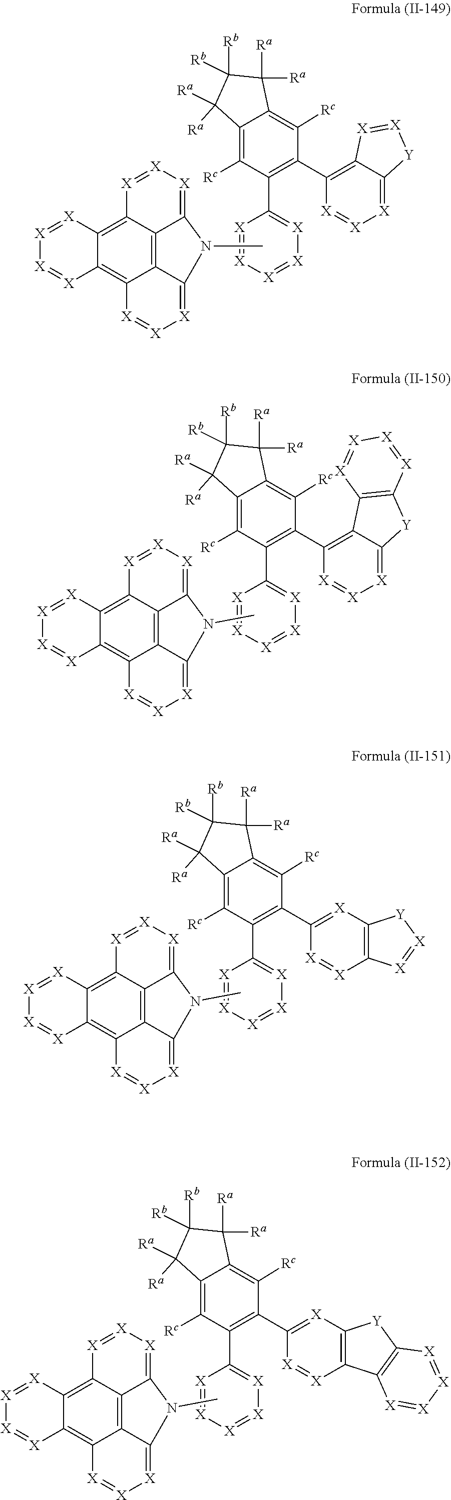

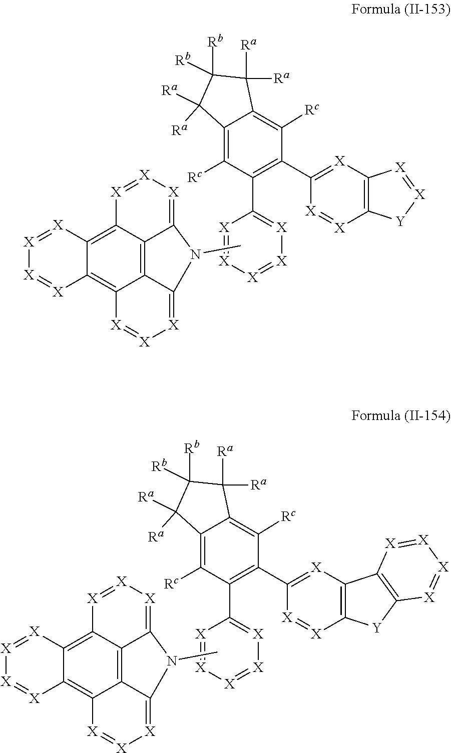

where the symbols R, R^{a}, R^{b} and R^{c} have the definitions given above, especially for formula (I), and the further symbols are as follows:
X is the same or different at each instance and is N, CR, or C if a group binds to the structure; and
Y is O, S, NR or C(R)₂, preferably O, NR or C(R)₂.

It may preferably be the case that, in structures/compounds of the formulae (II-1) to (II-154), not more than two X groups per ring are N, and preferably all X are CR; preferably at least one, more preferably at least two of the X groups per ring are selected from C—H and C-D.

It may also be the case that, in structures/compounds of the formulae (II-1) to (II-154), at least two nonadjacent X groups per ring are N; the Y group is NR and at least one X group in a ring fused to the ring having the Y group is N and/or the Y group in one ring is NR and one X group not adjacent to the Y group in that ring is N. These structures/compounds preferably comprise electron transport groups and are therefore particularly suitable as electron transport materials and/or matrix materials.

In a further embodiment, it may be the case that, in structures/compounds of the formulae (II-1) to (II-154), not more than four, preferably not more than two X groups are N; more preferably, all X groups are CR, where preferably not more than 4, more preferably not more than 3 and especially preferably not more than 2 of the CR groups that X represents are not the CH group. These structures/compounds preferably do not comprise any electron transport groups and are therefore particularly suitable as hole transport materials and/or matrix materials.

In a further preferred embodiment, it may be the case that the compounds of the invention include a structure of the formulae (III-1) to (III-60), where the compounds of the invention may more preferably be selected from the compounds of the formulae (III-1) to (III-60)

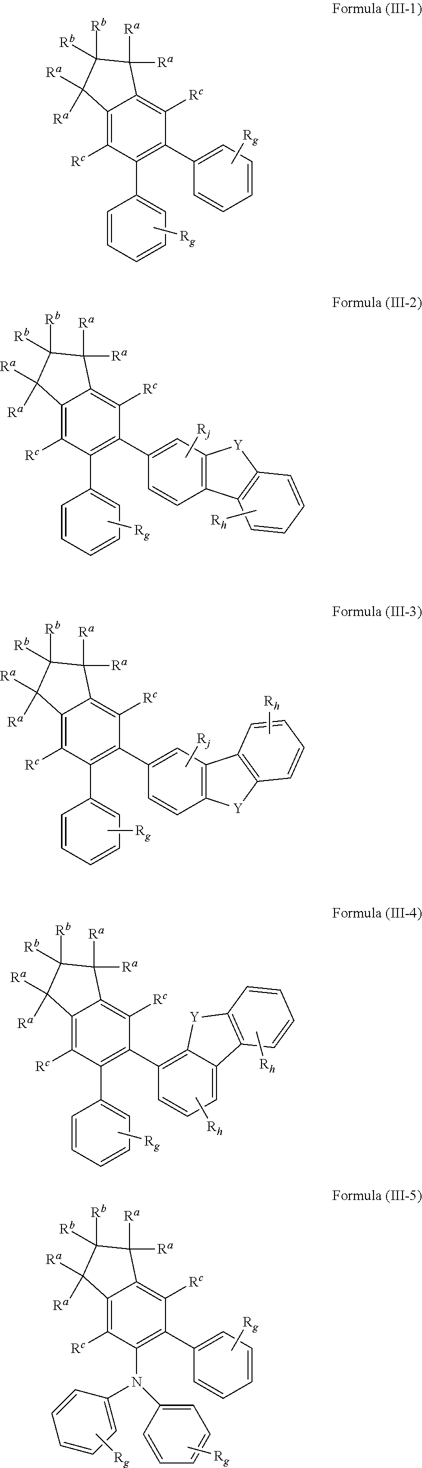

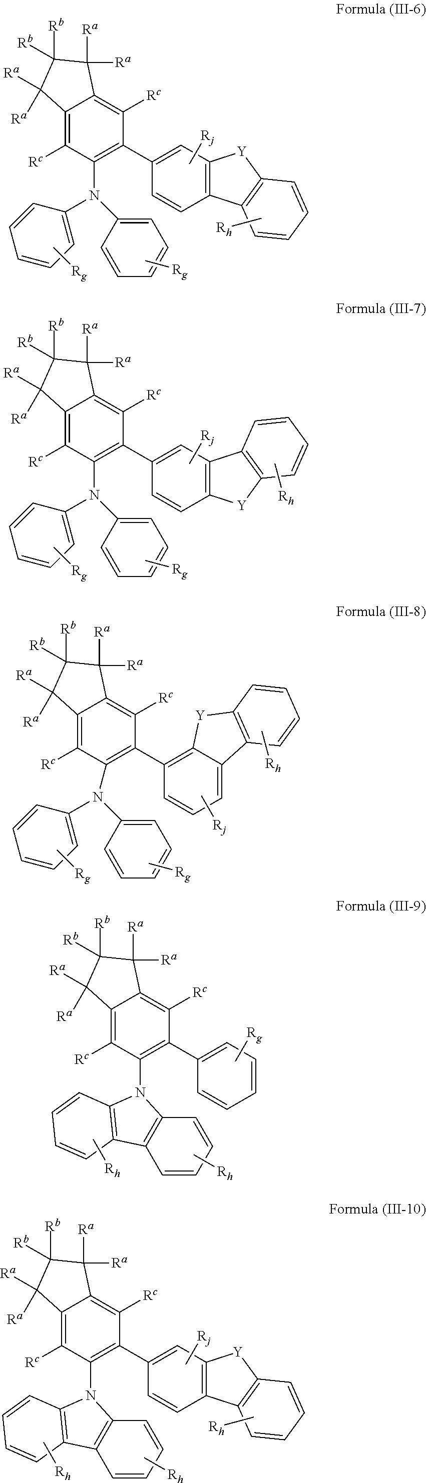

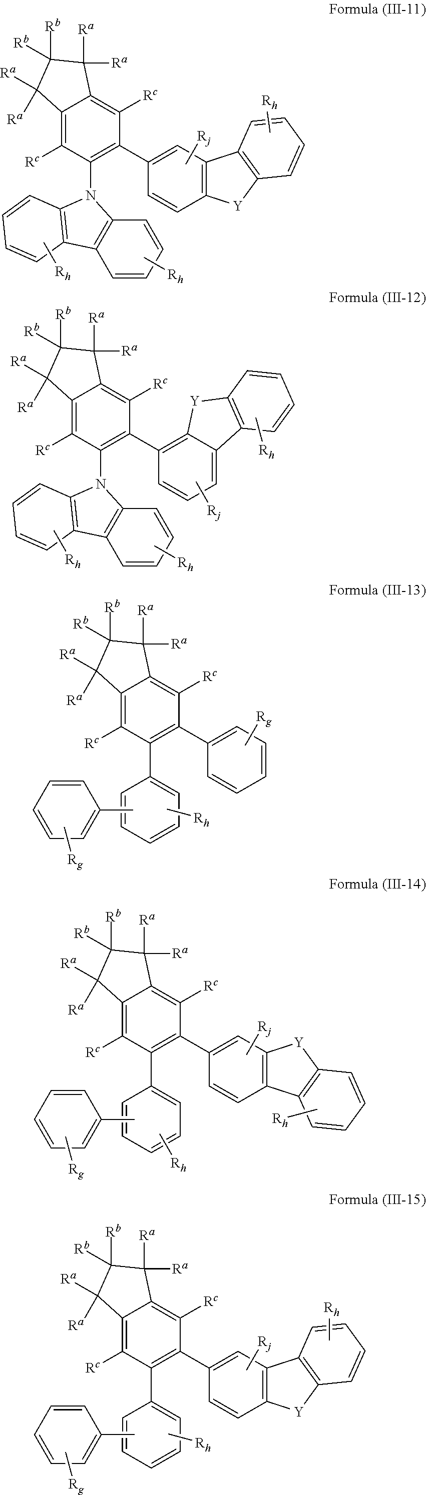

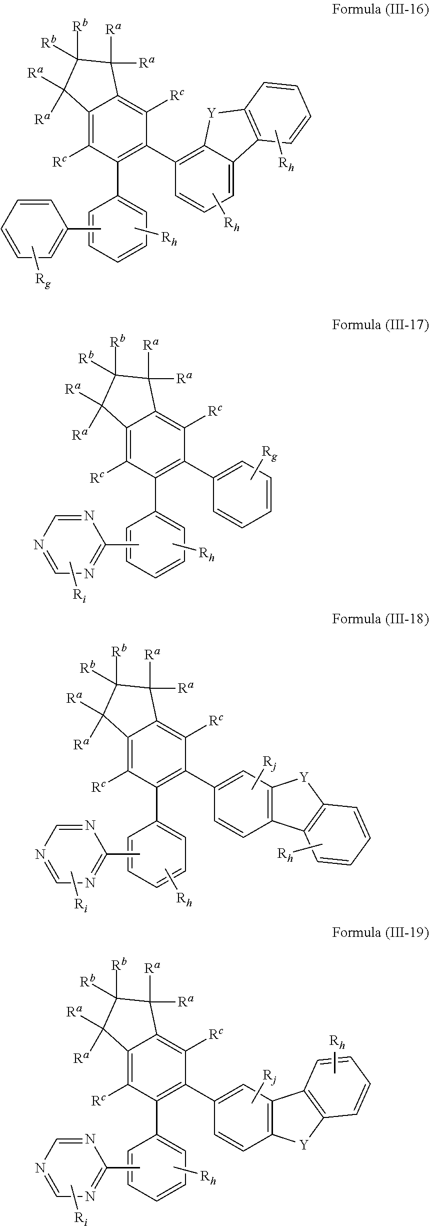

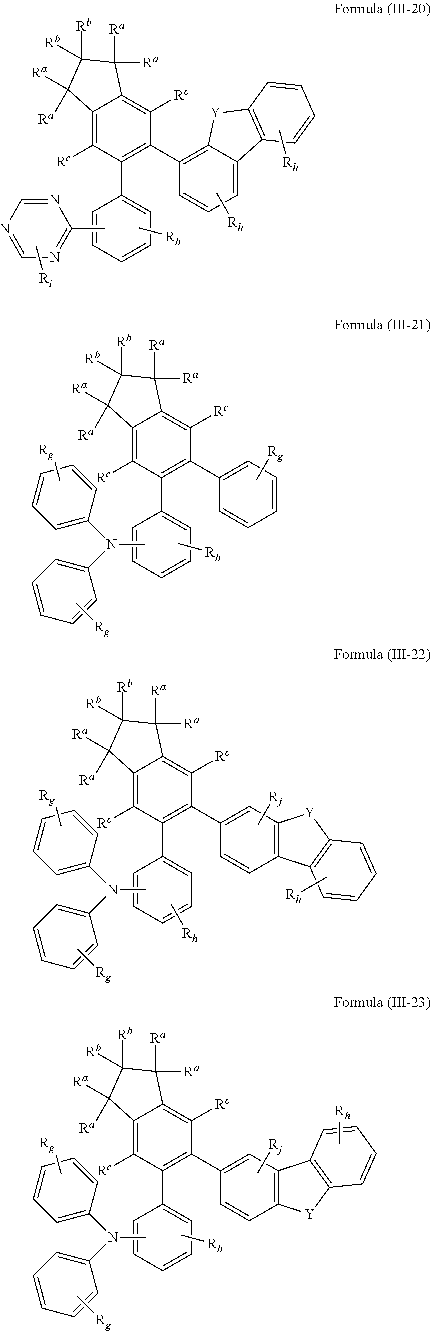

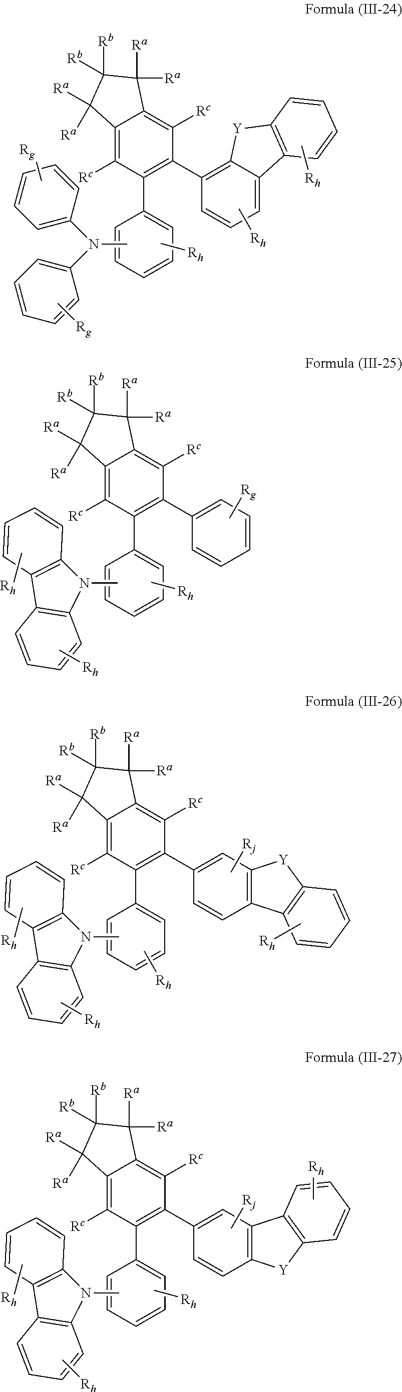

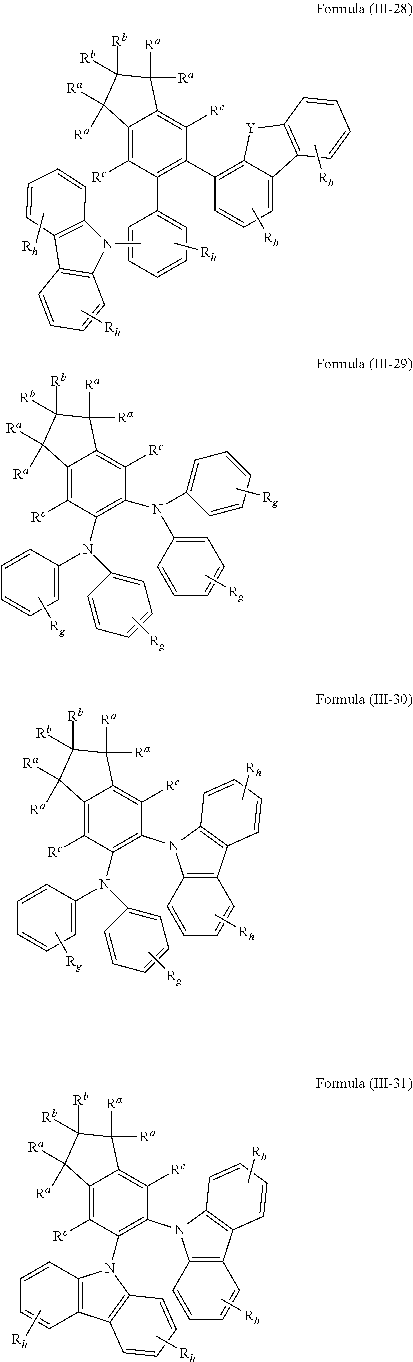

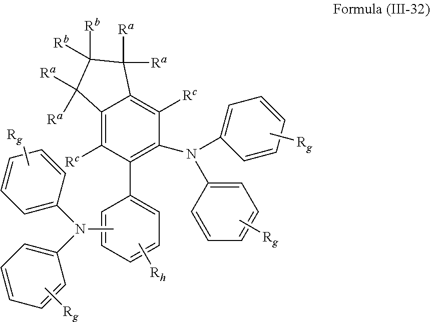

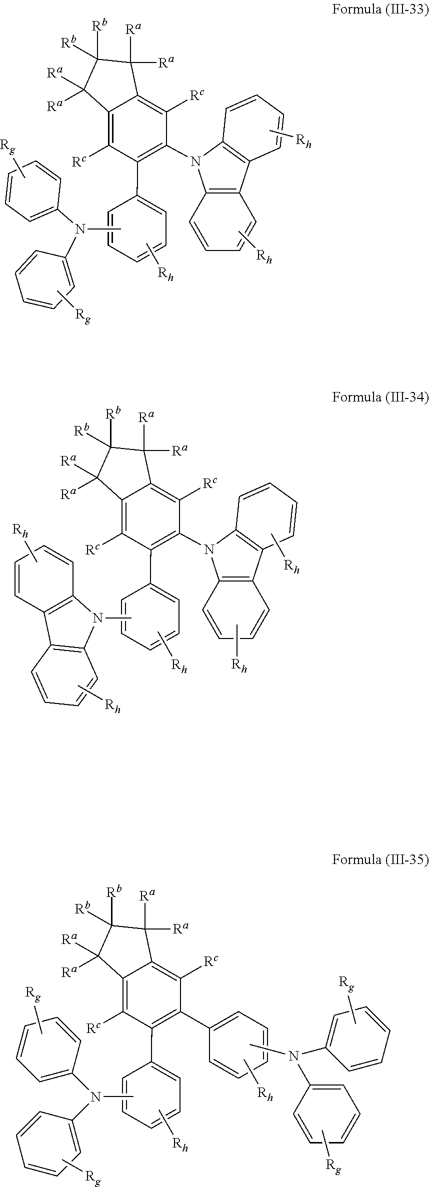

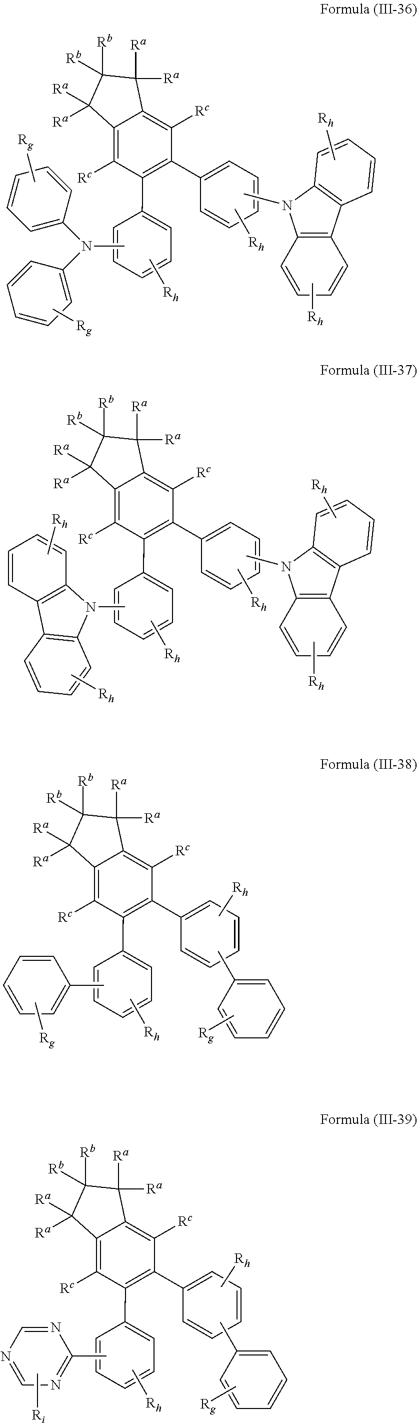

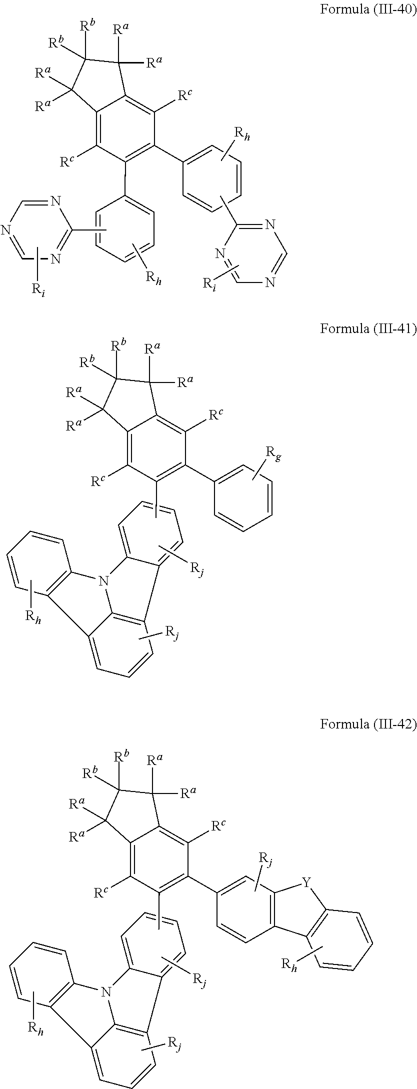

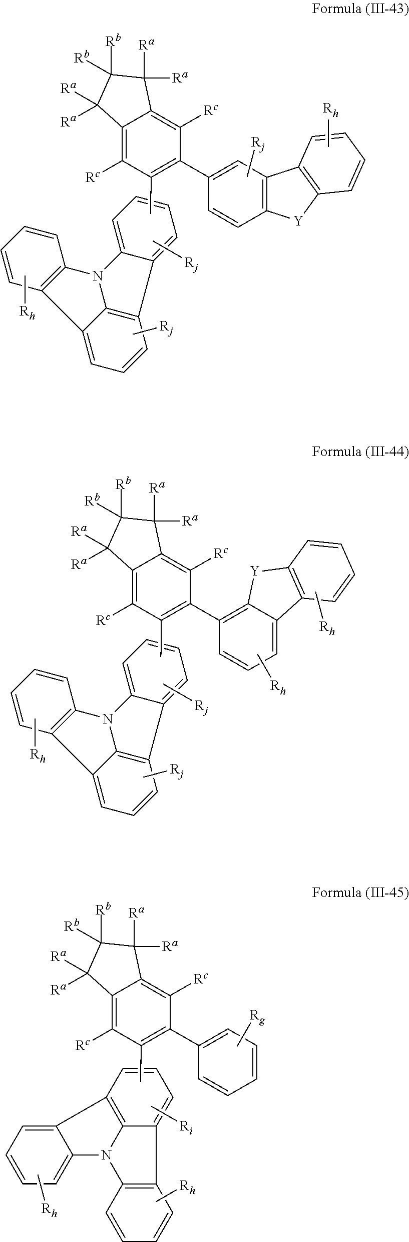

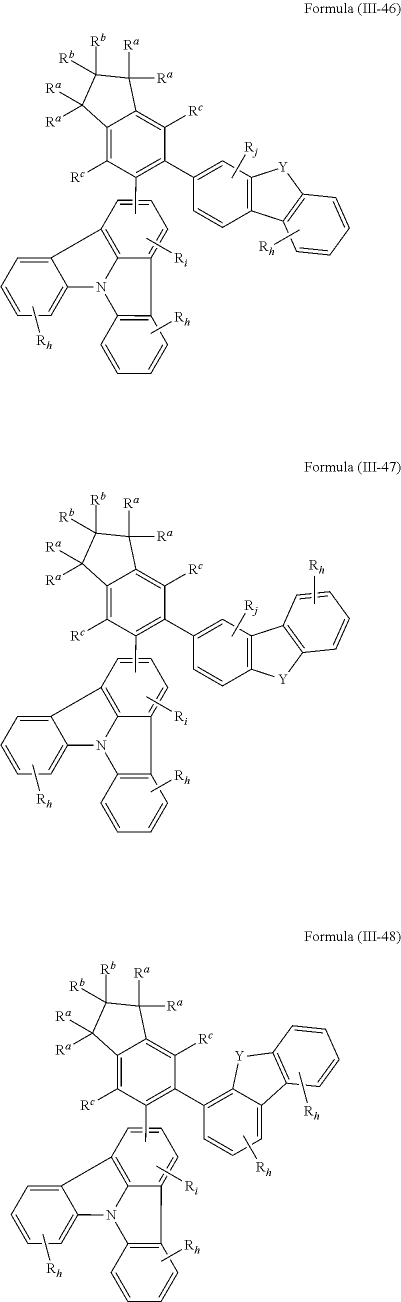

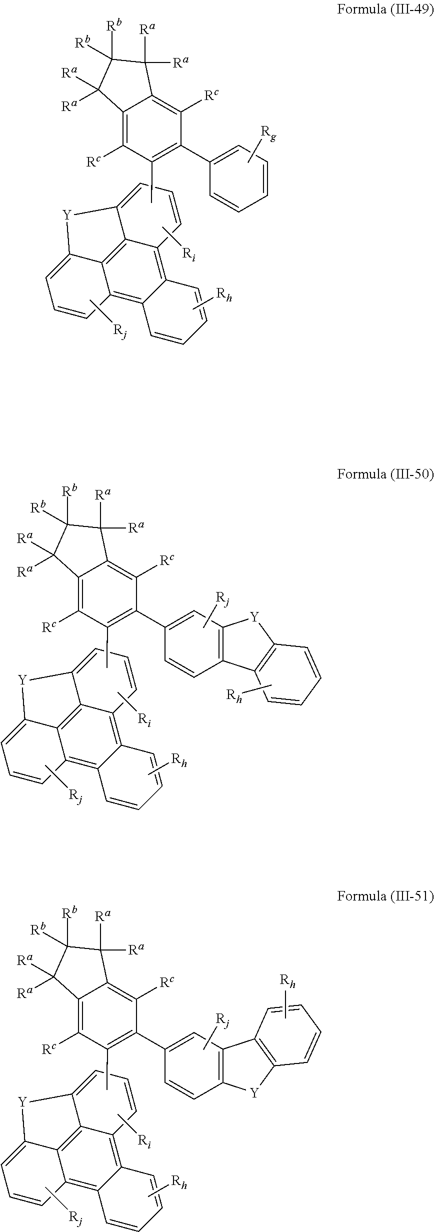

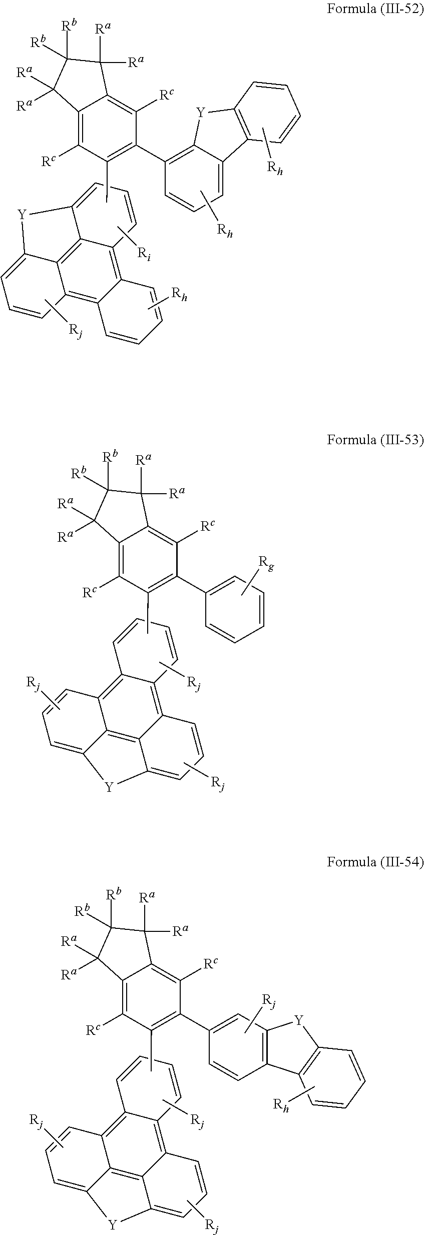

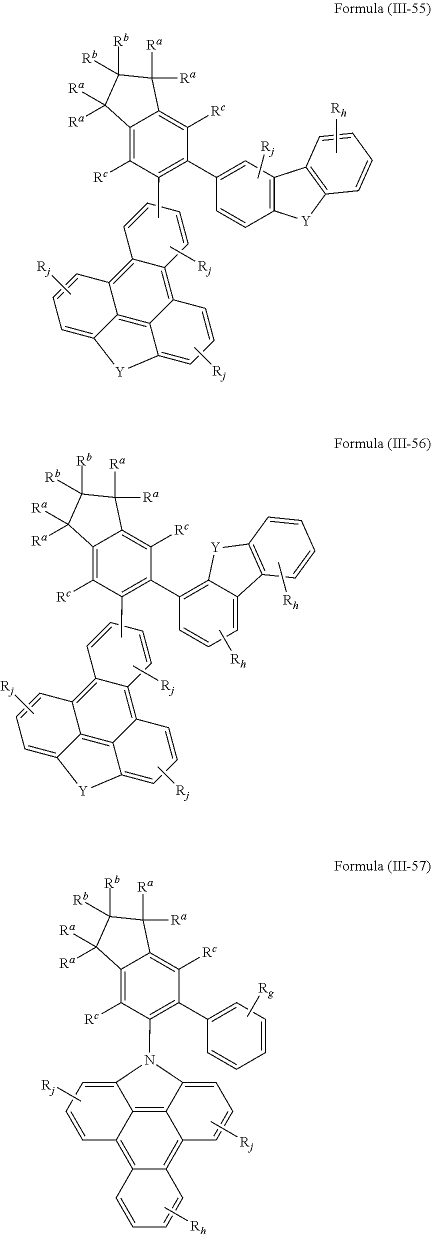

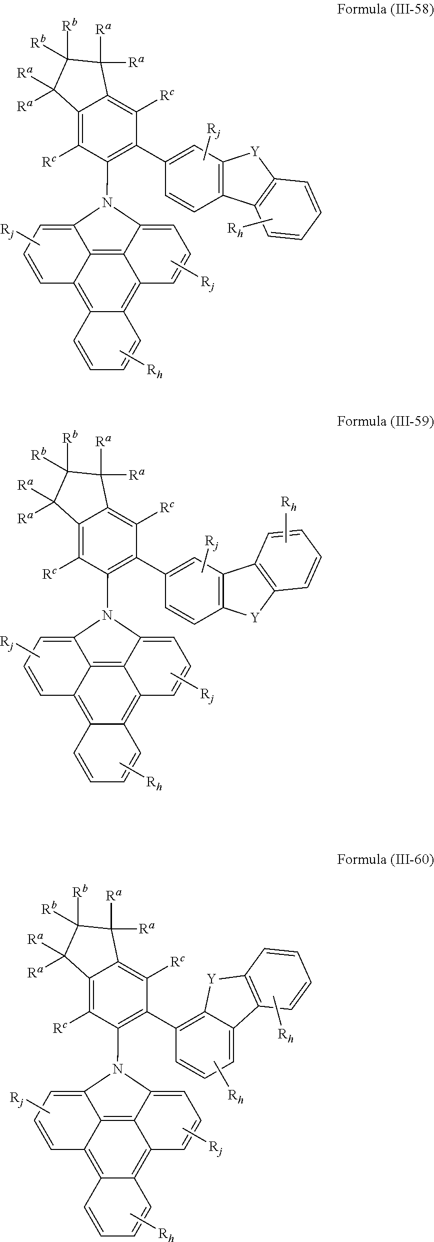

where the symbols R, R^{a}, R^{b} and R^{c} have the definitions given above, especially for formula (I), and the further symbols are as follows:
Y is O, S, NR or C(R)₂, preferably O, NR or C(R)₂;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5.

The sum total of the indices i, j, h and g in structures/compounds of the formulae (III-1) to (III-60) is preferably not more than 6, especially preferably not more than 4 and more preferably not more than 2.

In a preferred development of the present invention, it may be the case that at least two, preferably adjacent R radicals form a fused ring together with the further groups to which the two R radicals bind, where the two R radicals form at least one structure of the formulae (RA-1) to (RA-13):

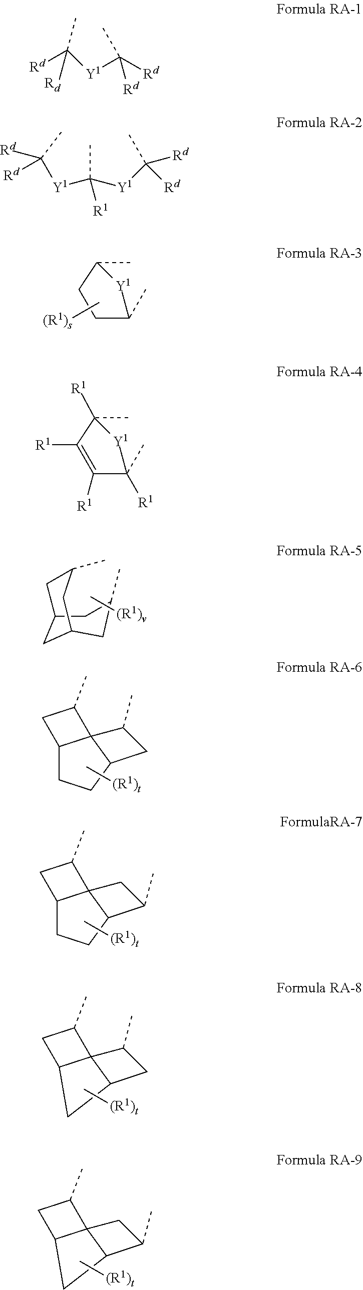

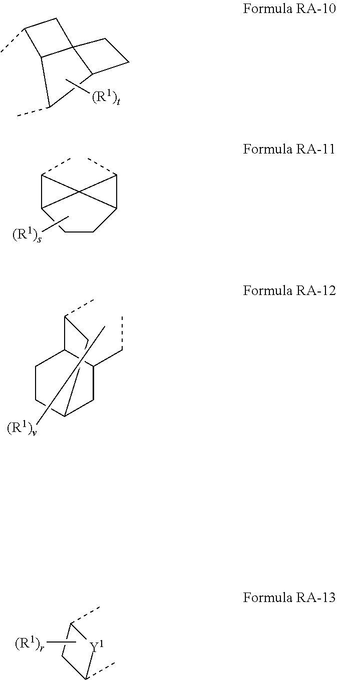

where R¹ has the definition set out above, the dotted bonds represent the sites of attachment to the atoms of the groups to which the two R radicals bind, and the further symbols have the following definition:
Y¹ is the same or different at each instance and is C(R¹)₂, (R¹)₂C—C(R¹)₂, (R¹)C═C(R¹), NR¹, NAr′, O or S, preferably C(R¹)₂, (R¹)₂C—C(R¹)₂, (R¹)C═C(R¹), O or S;
R^{d} is the same or different at each instance and is F, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may be substituted in each case by one or more R² radicals, where one or more nonadjacent CH₂ groups may be replaced by R²C═CR², C≡C, Si(R²)₂, C═O, C═S, C═Se, C═NR², —C(═O)O—, —C(═O)NR²—, NR², P(═O)(R²), —O—, —S—, SO or SO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals; at the same time, it is also possible for two Rd radicals together or one Rd radical together with an R¹ radical or together with a further group to form a ring system, where R² has the definition given above, especially for formula (I);
r is 0, 1, 2, 3 or 4, preferably 0, 1 or 2, more preferably 0 or 1;
s is 0, 1, 2, 3, 4, 5 or 6, preferably 0, 1, 2, 3 or 4, more preferably 0, 1 or 2;
t is 0, 1, 2, 3, 4, 5, 6, 7 or 8, preferably 0, 1, 2, 3 or 4, more preferably 0, 1 or 2;
v is 0, 1, 2, 3, 4, 5, 6, 7, 8 or 9, preferably 0, 1, 2, 3 or 4, more preferably 0, 1 or 2.

Preference is given here to structures of the formulae RA-1, RA-3, RA-4 and RA-5, and particular preference to structures of the formulae RA-4 and RA-5.

In a preferred embodiment of the invention, preferably at least two, preferably adjacent, R radicals form a fused ring together with the further groups to which the two R radicals bind, where the two R radicals form structures of the formulae (RA-1a) to (RA-4f):

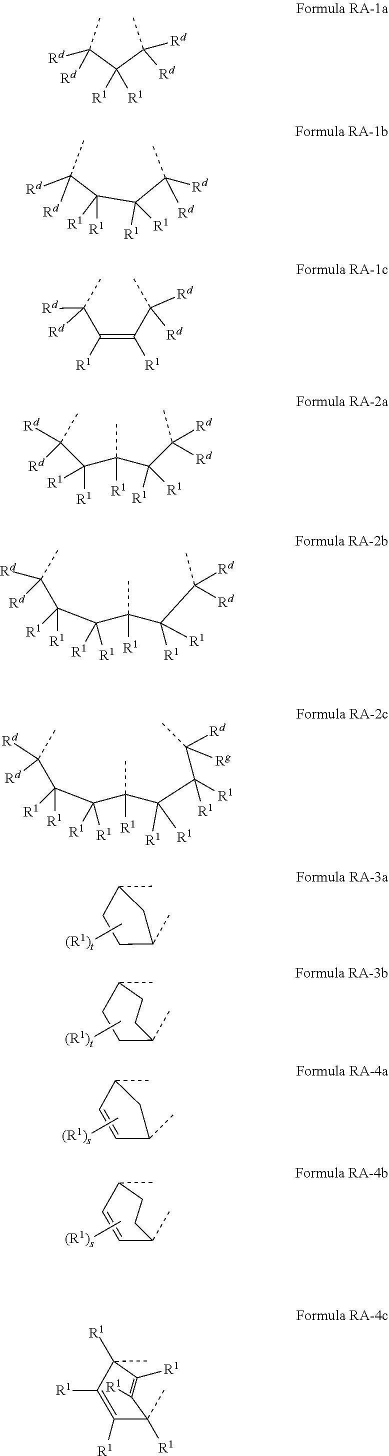

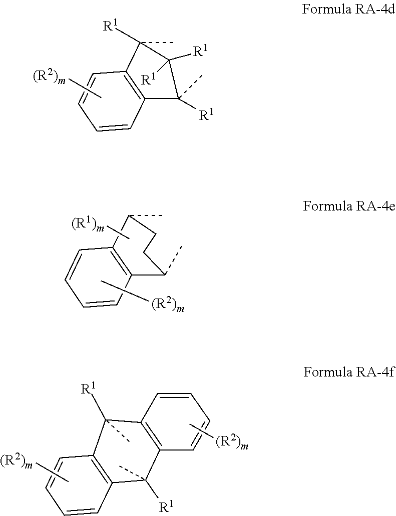

where the dotted bonds represent the sites of attachment to the atoms of the groups to which the two R radicals bind, the index m is 0, 1, 2, 3 or 4, preferably 0, 1 or 2, and the symbols R¹, R², R^{d} and the indices s and t have the definition given above, especially for formula (I) and/or formulae (RA-1) to (RA-13).

Preference is given here to structures of the formula RA-4f.

It may also be the case that the at least two R radicals that form structures of the formulae (RA-1) to (RA-12) and/or (RA-1a) to (RA-4f) and form a fused ring are R radicals from adjacent X groups, or are R radicals that each bind to adjacent carbon atoms, where these carbon atoms are preferably connected via a bond.

In a further-preferred configuration, preferably at least two, preferably adjacent, R radicals form a fused ring together with the further groups to which the two R radicals bind, where the two R radicals form structures of the formula (RB):

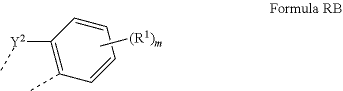

where R¹ has the definition given above, especially for formula (I), the dotted bonds represent the bonding sites via which the two R radicals bind, the index m is 0, 1, 2, 3 or 4, preferably 0, 1 or 2, and Y² is C(R¹)₂, NR¹, NAr′, BR¹, BAr′, O or S, preferably C(R¹)₂, NAr′ or O, more preferably C(R¹)₂ or O, where Ar′ has the definition given above, especially for formula (I).

It may also be the case that the at least two R radicals that form structures of the formula (RB) and form a fused ring are R radicals from adjacent X groups, or are R radicals that each bind to adjacent carbon atoms, where these carbon atoms are preferably joined via a bond.

More particularly, it may be the case that, in preferred structures/compounds, the sum total of the indices r, s, t, v, m and n is preferably 0, 1, 2 or 3, more preferably 1 or 2.

More preferably, the compounds include at least one structure of the formulae (IV-1) to (IV-12); more preferably, the compounds are selected from compounds of the formulae (IV-1) to (IV-12), where the compounds have at least one fused ring:

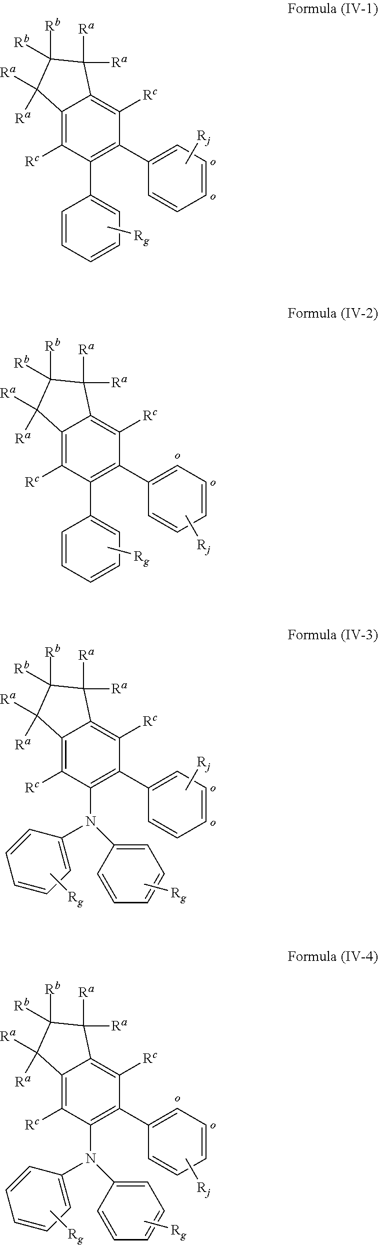

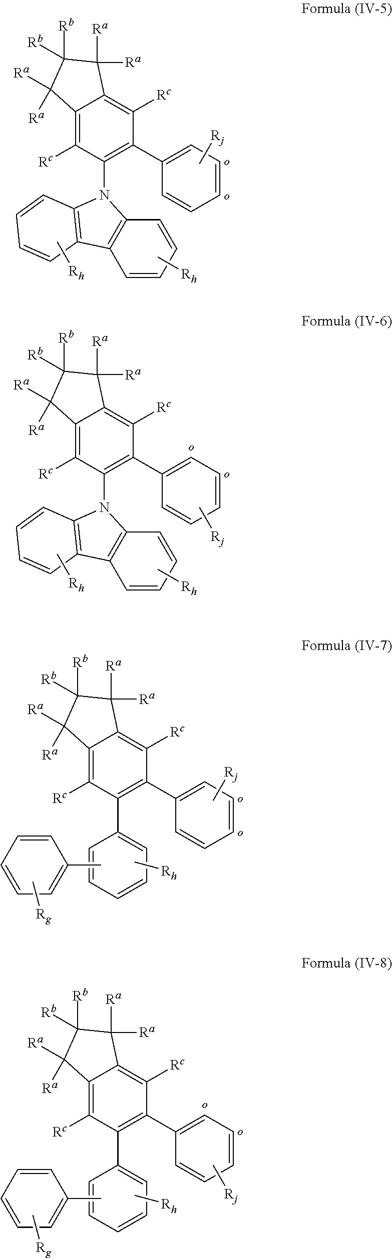

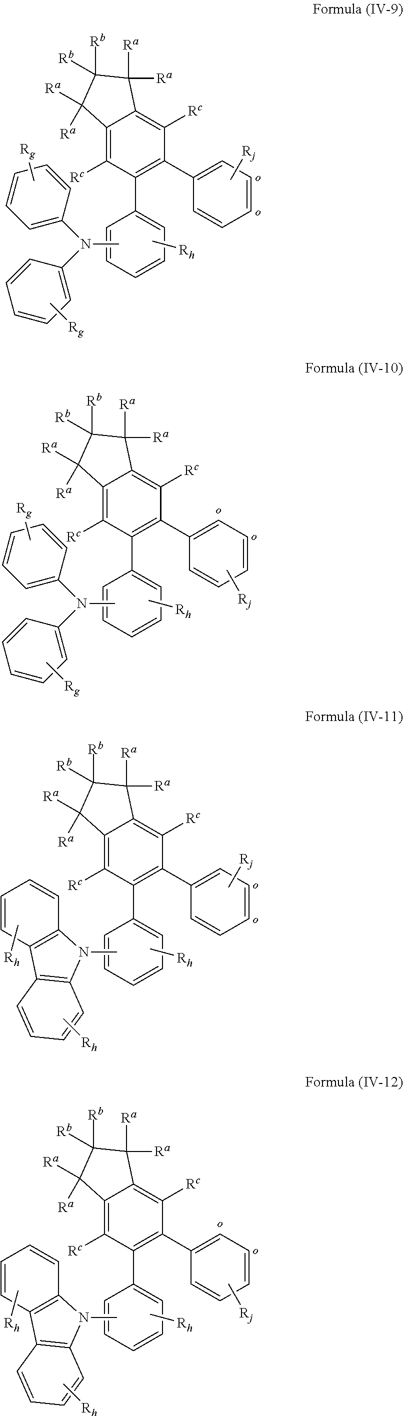

where the symbols R, R^{a}, R^{b} and R^{c} have the definitions given above, especially for formula (I), the symbol o represents the fusion sites of the at least one fused ring, and the other indices used are as follows:
j at each instance is independently 0, 1, 2 or 3;
h at each instance is independently 0, 1, 2, 3 or 4;
g at each instance is independently 0, 1, 2, 3, 4 or 5.

It may also be the case, especially for structures/compounds of the formulae (IV-1) to (IV-12) that the fused ring is formed by structures of the formulae (RA-1) to (RA-13), (RA-1a) to (RA-4f) and/or (RB), as shown above, preferably is formed by structures of the formulae (RA-1) to (RA-13) and/or (RA-1a) to (RA-4f).

It may preferably be the case that the compounds have at least two fused rings, where at least one fused ring is formed by structures of the formulae (RA-1) to (RA-13) and/or (RA-1a) to (RA-4f) and a further ring by structures of the formulae (RA-1) to (RA-13), (RA-1a) to (RA-4f) or (RB).

It may additionally be the case that the substituents R, R^{d} and R¹ in the above formulae do not form a fused aromatic or heteroaromatic ring system with the ring atoms of the ring system to which the substituents R, R^{d} and R¹ bind. This includes the formation of a fused aromatic or heteroaromatic ring system with possible substituents R^{d}, R¹ and R² which may be bonded to the substituents R, R^{d} and R¹.

The R^{a}, R^{b}, R^{c} radicals preferably do not form any ring system with other groups.

When the compound of the invention is substituted by aromatic or heteroaromatic R, R^{d}, R¹ or R² groups, it is preferable when these do not have any aryl or heteroaryl groups having more than two aromatic six-membered rings fused directly to one another. More preferably, the substituents do not have any aryl or heteroaryl groups having six-membered rings fused directly to one another at all. The reason for this preference is the low triplet energy of such structures. Fused aryl groups which have more than two aromatic six-membered rings fused directly to one another but are nevertheless also suitable in accordance with the invention are phenanthrene and triphenylene, since these also have a high triplet level.

It may also be the case that the R radical does not comprise any aromatic or heteroaromatic ring system having three linear-condensed aromatic 6-membered rings, where preferably none of the R radicals comprises an aromatic or heteroaromatic ring system having three linear-condensed aromatic 6-membered rings.

Preferably, the Z^{a}, Z^{b} group may form through-conjugation with the group to which the Z^{a}, Z^{b} group in formula (I) or the preferred embodiments of this formula is bonded. Through-conjugation of the aromatic or heteroaromatic systems is formed as soon as direct bonds are formed between adjacent aromatic or heteroaromatic rings. A further bond between the aforementioned conjugated groups, for example via a sulfur, nitrogen or oxygen atom or a carbonyl group, is not detrimental to conjugation.

It may further be the case that the substituents R and R¹ according to the above formulae do not form a fused aromatic or heteroaromatic ring system, preferably any fused ring system, with the ring atoms of the ring system. This includes the formation of a fused ring system with possible substituents R¹ and R² which may be bonded to the R, R¹ radicals.

When two radicals that may especially be selected from R, R¹ and/or R² form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R, R¹ and/or R² may also be joined to one another via a bond, such that this can bring about a ring closure. In this case, each of the corresponding bonding sites has preferably been provided with a substituent R, R¹ and/or R².

It may also be the case that at least one R radical is the same or different at each instance and is selected from the group consisting of a branched or cyclic alkyl, alkoxy or thioalkoxy group having 3 to 20 carbon atoms or an aromatic or heteroaromatic ring system selected from the groups of the following formulae Ar-1 to Ar-75; preferably, the substituents R either form a fused ring, preferably according to the structures of the formulae (RA-1) to (RA-13) or (RB), or the substituent R is the same or different at each instance and is selected from the group consisting of an aromatic or heteroaromatic ring system selected from the groups of the following formulae Ar-1 to Ar-75, and/or the Ar′ group is the same or different at each instance and is selected from the groups of the following formulae Ar-1 to Ar-75, and/or the Ar^{a}, Ar^{b}, Ar^{c}, Ar^{d} and/or Ar′ group is the same or different at each instance and is selected from the groups of the following formulae Ar-1 to Ar-75:

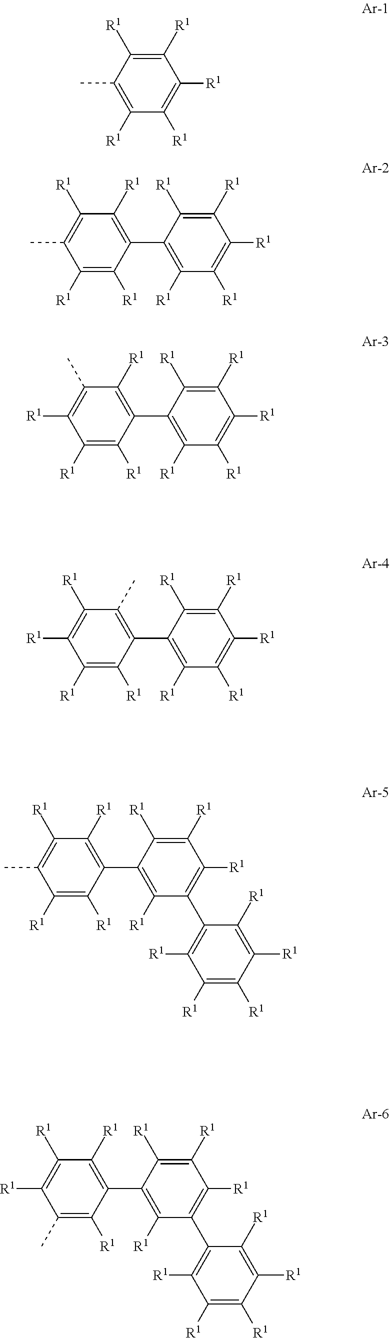

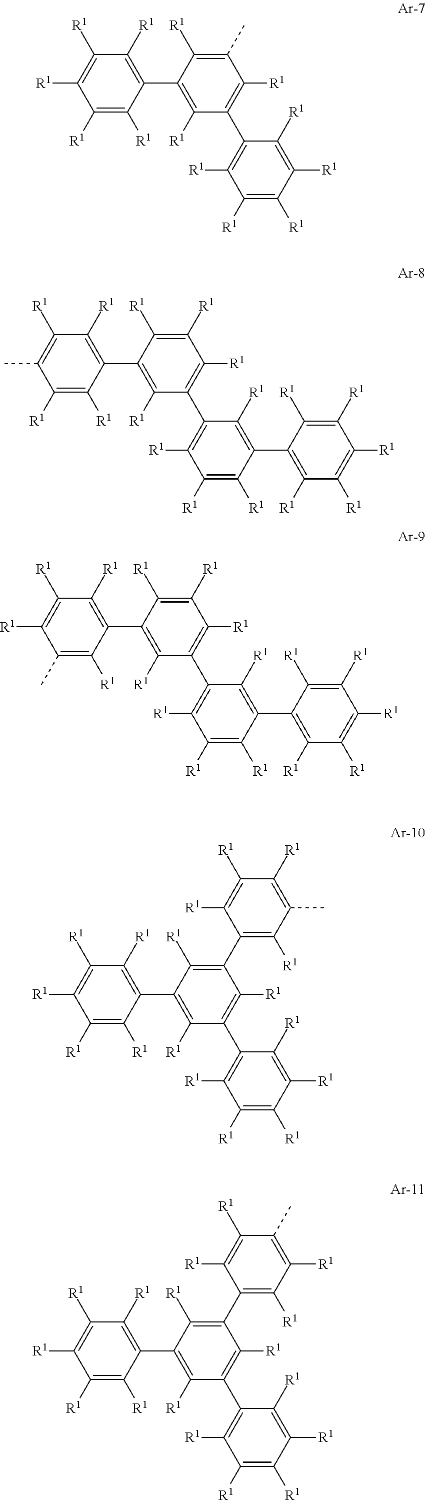

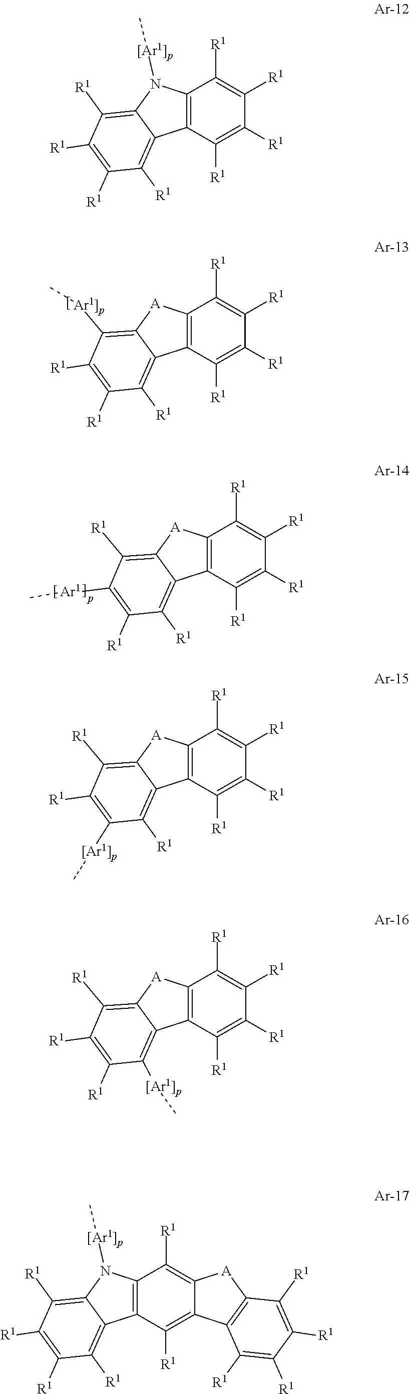

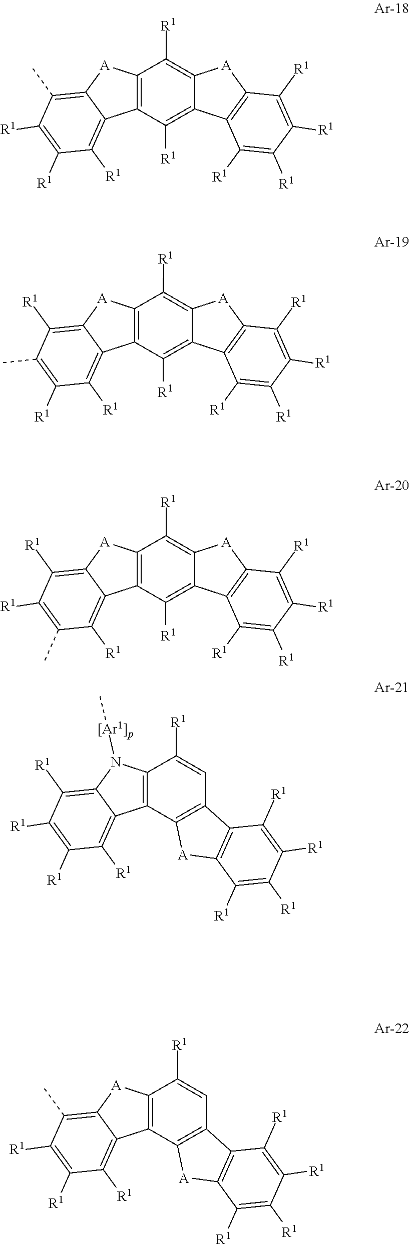

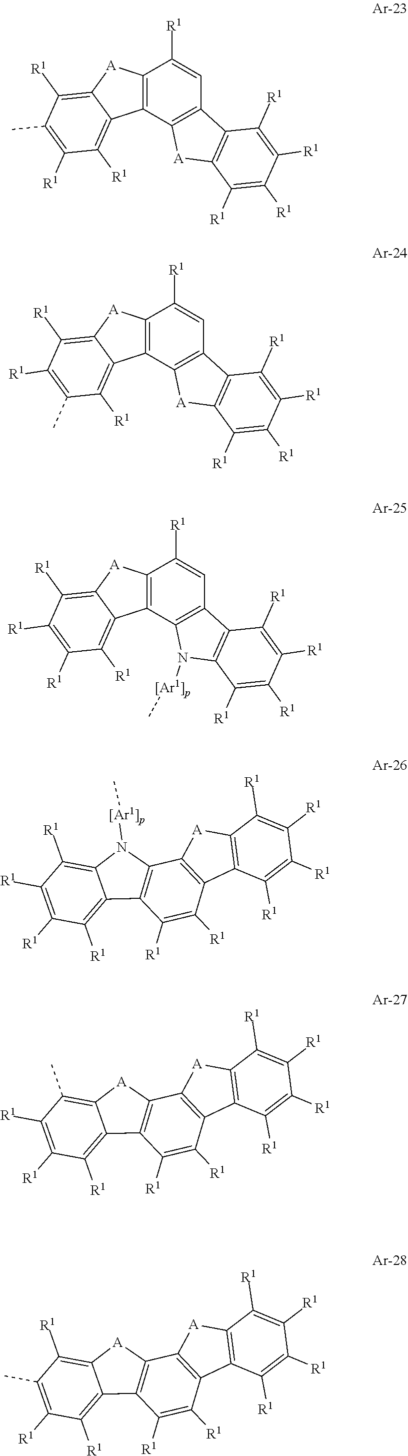

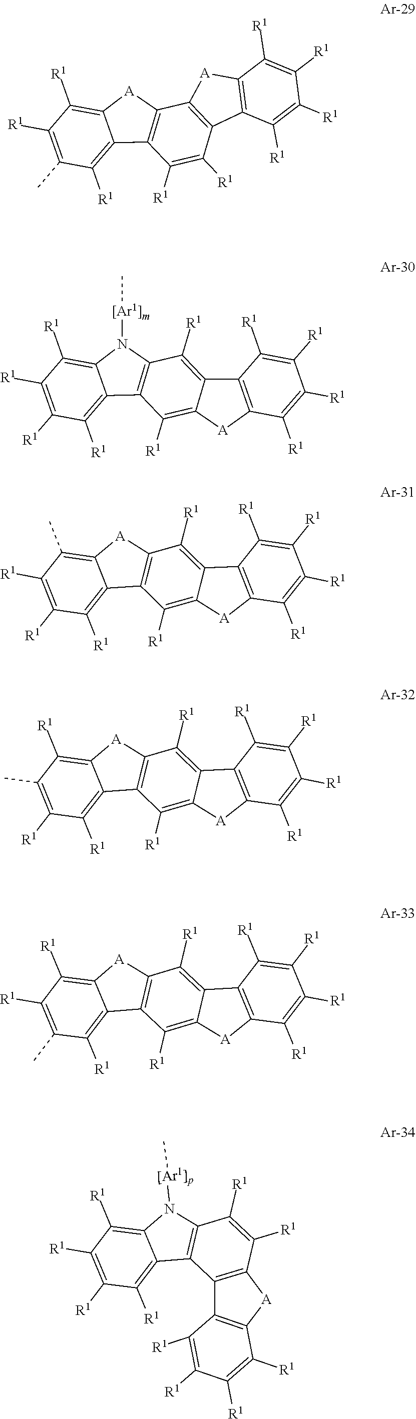

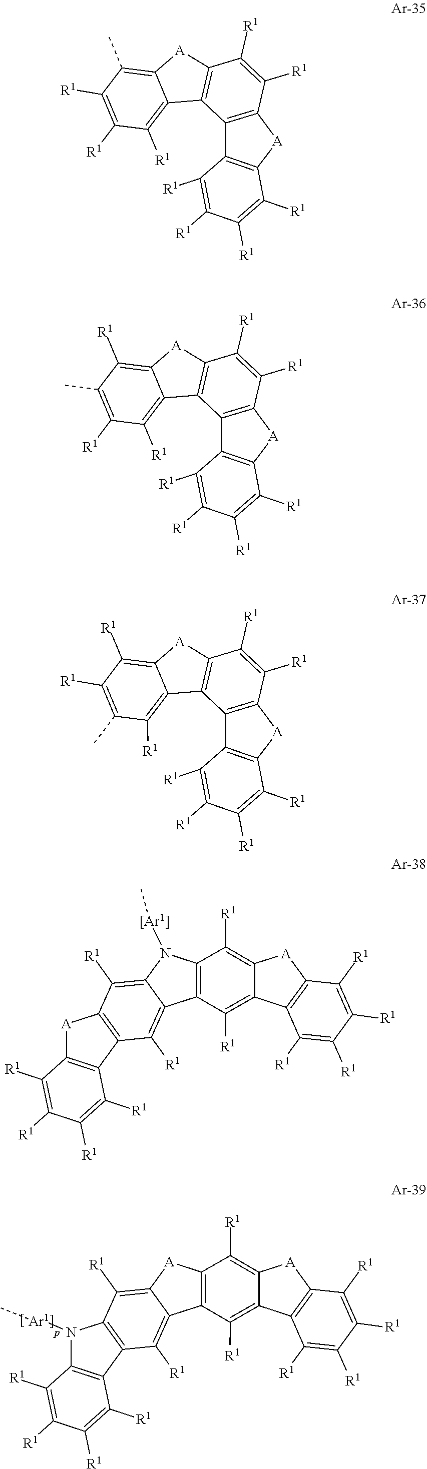

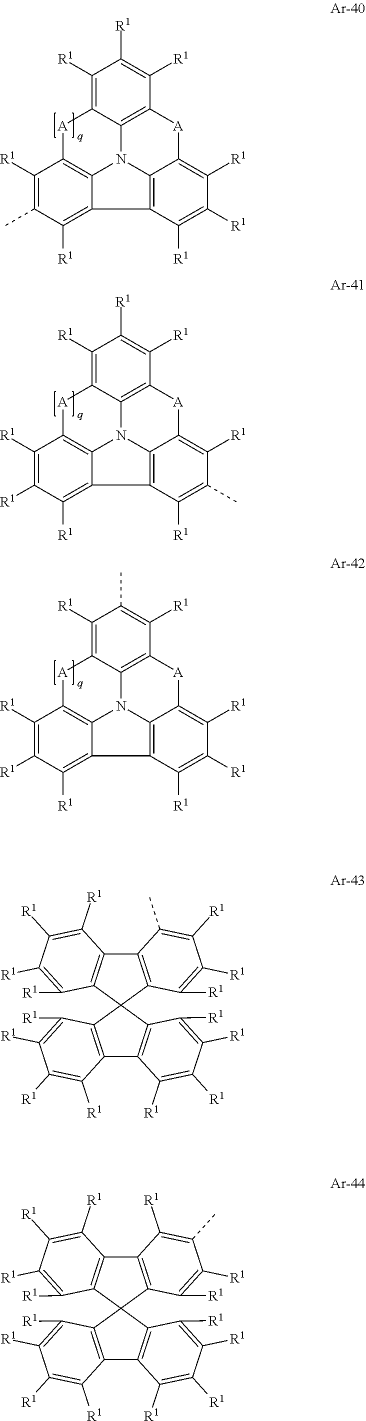

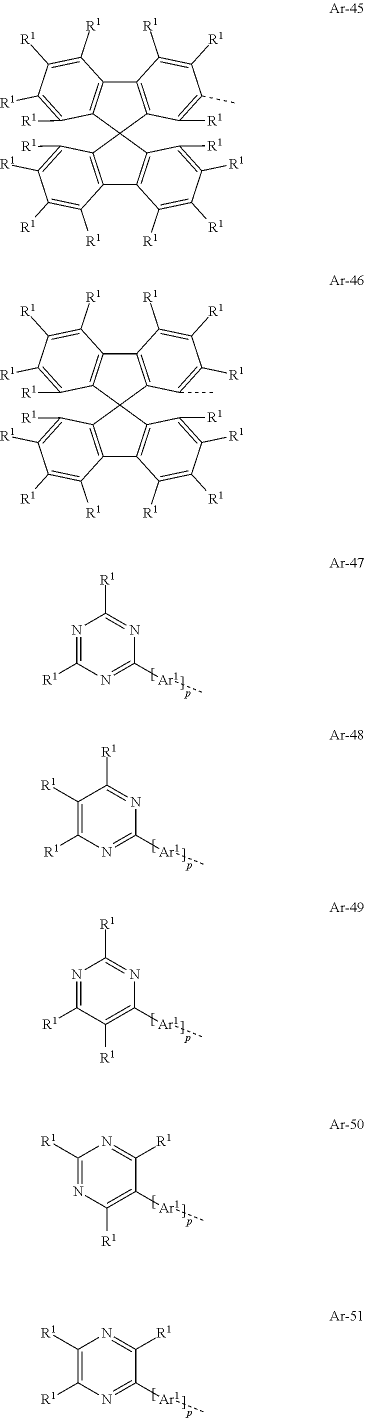

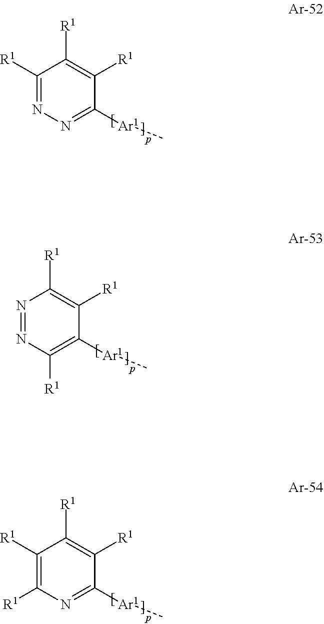

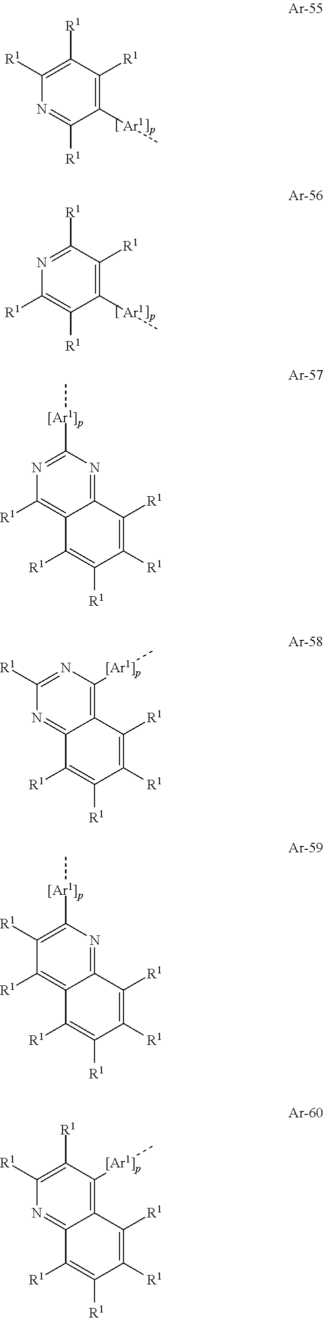

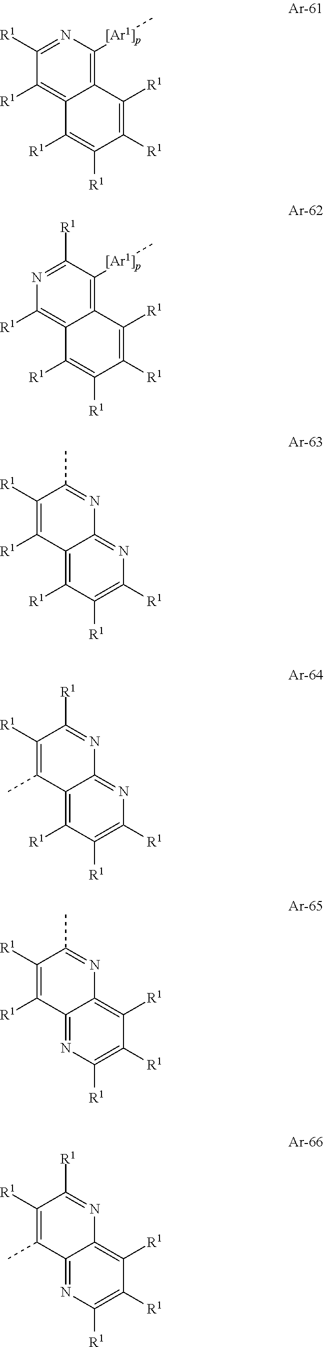

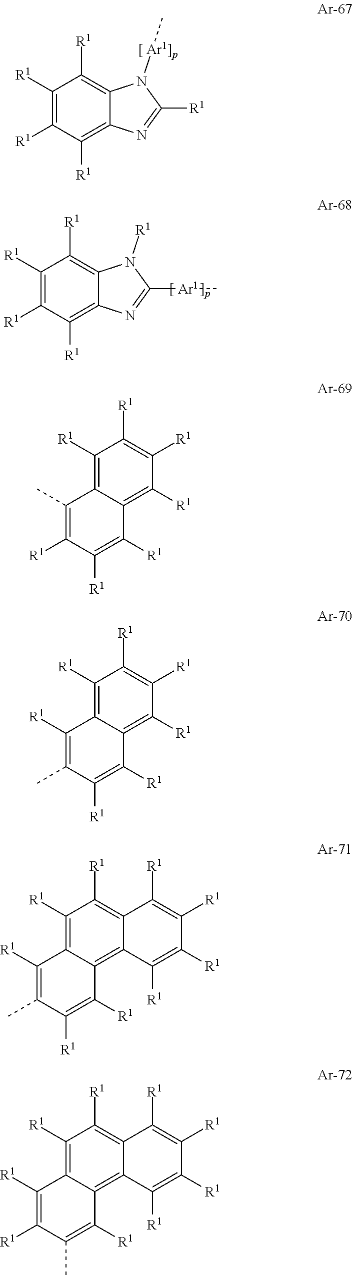

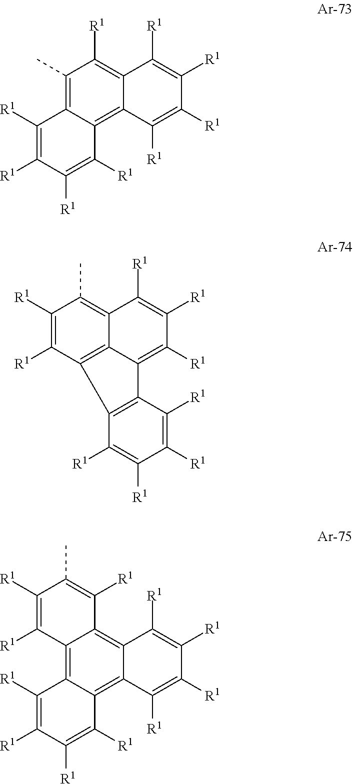

where R¹ has the definitions given above, the dotted bond represents the bond to the corresponding group and in addition:
Ar¹ is the same or different at each instance and is a divalent aromatic or heteroaromatic ring system which has 6 to 18 aromatic ring atoms and may be substituted in each case by one or more R¹ radicals;
A is the same or different at each instance and is C(R¹)₂, NR¹, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R¹ radicals are bonded to the corresponding carbon atoms instead.

The above-detailed structures of the formulae (Ar-1) to (Ar-75) are preferred configurations of the Ar^{a}, Ar^{b}, Ar^{c}, Ar^{d} radical as defined, for example, in structures of the formula (I), in which case the substituents R¹ in formulae (Ar-1) to (Ar-75) should be replaced by R, where R has the definition set out above, especially for formula (I).

The above-detailed structures of the formulae (Ar-1) to (Ar-75) are preferred configurations of the Ar and Ar^{e} radicals as defined, for example, for structures of the formula (I), in which case the substituents R¹ in formulae (Ar-1) to (Ar-75) should be replaced by R, where R has the definition set out above, especially for formula (I). In addition, the Ar and Ar^{e} radicals include a further site of attachment.

Preference is given here to structures of the formulae (Ar-1), (Ar-2), (Ar-3), (Ar-12), (Ar-13), (Ar-14), (Ar-15), (Ar-16), (Ar-40), (Ar-41), (Ar-42), (Ar-43), (Ar-44), (Ar-45), (Ar-46), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16).

When the abovementioned groups for structures of the formulae (Ar-1) to (Ar-75) have two or more A groups, possible options for these include all combinations from the definition of A. Preferred embodiments in that case are those in which one A group is NR¹ and the other A group is C(R¹)₂ or in which both A groups are NR¹ or in which both A groups are O.

When A is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R² radicals. Preference is given to phenyl, biphenyl, terphenyl and quaterphenyl. Preference is further given to triazine, pyrimidine and quinazoline as listed above for Ar-47 to Ar-50, Ar-57 and Ar-58, where these structures, rather than by R¹, may be substituted by one or more R² radicals.

When A is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

There follows a description of preferred substituents R, R^{a}, R^{b}, R^{c} and R^{d}.

In a preferred embodiment of the invention, R is the same or different at each instance and is selected from the group consisting of H, D, F, CN, NO₂, Si(R¹)₃, B(OR¹)₂, a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R¹ radicals.

In a further-preferred embodiment of the invention, substituent R is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R¹ radicals.

It may also be the case that at least one R radical, preferably one substituent R, is the same or different at each instance and is selected from the group consisting of H, D, an aromatic or heteroaromatic ring system which has 6 to 30 aromatic ring atoms and may be substituted by one or more R¹ radicals, or an N(Ar′)₂ group; more preferably, at least one substituent R is the same or different at each instance and is selected from the group consisting of an aromatic or heteroaromatic ring system which has 6 to 30 aromatic ring atoms and may be substituted by one or more R¹ radicals. In a further-preferred embodiment of the invention, the substituents R either form a ring according to the structures of the formulae (RA-1) to (RA-12), (RA-1a) to (RA-4f) or (RB) or the substituent R is the same or different at each instance and is selected from the group consisting of H, D, an aromatic or heteroaromatic ring system which has 6 to 30 aromatic ring atoms and may be substituted by one or more R¹ radicals, or an N(Ar′)₂ group. More preferably, the R radical, preferably the substituent R, is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R¹ radicals.

It may also be the case that at least one R radical is an aromatic or heteroaromatic ring system which has 5 to 13 aromatic ring atoms and may be substituted by one or more R¹ radicals.

It may preferably be the case that at least one radical, preferably one substituent R, is selected from phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹ radicals. What is meant more particularly here by the expression “substituent” is that R is not H. In addition, the substituents R may be the same or different if two or more substituents selected from the aromatic or heteroaromatic groups mentioned are present.

It may preferably be the case that the R^{a} group is methyl, ethyl, propyl, or two R^{a} groups that bind to the same carbon atom form a cycloalkyl radical having 5 or 6, preferably 5, carbon atoms, where the R^{a} group is preferably methyl, where these groups may be deuterated.

It may preferably be the case that the R^{b} group is methyl, ethyl, propyl, or two R^{b} groups that bind to the same carbon atom form a cycloalkyl radical having 5 or 6, preferably 5, carbon atoms, where the R^{b} group is preferably methyl, where these groups may be deuterated.

It may preferably be the case that the R^{c} group is H, D, methyl, ethyl, propyl, where these groups may be deuterated, where the R^{c} group is preferably H or D.

In a preferred embodiment of the invention, R^{d} is the same or different at each instance and is selected from the group consisting of a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R² radicals.

In a further-preferred embodiment of the invention, R^{d} is the same or different at each instance and is selected from the group consisting of a straight-chain alkyl group having 1 to 10 carbon atoms or a branched or cyclic alkyl group having 3 to 10 carbon atoms, where the alkyl group may be substituted in each case by one or more R² radicals, an aromatic or heteroaromatic ring system which has 6 to 30 aromatic ring atoms and may be substituted by one or more R² radicals. More preferably, R^{d} is the same or different at each instance and is selected from the group consisting of a straight-chain alkyl group having 1 to 5 carbon atoms or a branched or cyclic alkyl group having 3 to 5 carbon atoms, where the alkyl group may be substituted in each case by one or more R² radicals, or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R² radicals.

In a preferred embodiment of the invention, R^{d} is the same or different at each instance and is selected from the group consisting of a straight-chain alkyl group having 1 to 6 carbon atoms or a cyclic alkyl group having 3 to 6 carbon atoms, where the alkyl group may be substituted in each case by one or more R² radicals, or an aromatic or heteroaromatic ring system which has 6 to 24 aromatic ring atoms and may be substituted in each case by one or more R² radicals; at the same time, two R^{d} radicals together may also form a ring system. More preferably, R^{d} is the same or different at each instance and is selected from the group consisting of a straight-chain alkyl group having 1, 2, 3 or 4 carbon atoms or a branched or cyclic alkyl group having 3 to 6 carbon atoms, where the alkyl group may be substituted in each case by one or more R² radicals, but is preferably unsubstituted, or an aromatic ring system which has 6 to 12 aromatic ring atoms, especially 6 aromatic ring atoms, and may be substituted in each case by one or more, preferably nonaromatic R² radicals, but is preferably unsubstituted; at the same time, two R^{d} radicals together may form a ring system. Most preferably, R^{d} is the same or different at each instance and is selected from the group consisting of a straight-chain alkyl group having 1, 2, 3 or 4 carbon atoms, or a branched alkyl group having 3 to 6 carbon atoms. Most preferably, R^{d} is a methyl group or is a phenyl group, where two phenyl groups together may form a ring system, preference being given to a methyl group over a phenyl group.

Preferred aromatic or heteroaromatic ring systems represented by substituent R, R^{d}, or Ar^{a}, Ar^{b}, Ar^{c}, Ar^{d} or Ar′, are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, anthracene, pyrene, perylene, chrysene, phenanthrene or triphenylene, each of which may be substituted by one or more R, R¹ or R² radicals. The structures Ar-1 to Ar-75 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). With regard to the structures Ar-1 to Ar-75, it should be stated that these are shown with a substituent R¹. In the case of the ring systems Ar^{a}, Ar^{b}, Ar^{c}, Ar^{d}, these substituents R¹ should be replaced by R, and in the case of R^{d}, these substituents R¹ should be replaced by R².

Further suitable R groups are groups of the formula -Ar⁴-N(Ar²) (Ar³), where Ar², Ar³ and Ar⁴ are the same or different at each instance and are an aromatic or heteroaromatic ring system which has 5 to 24 aromatic ring atoms and may be substituted in each case by one or more R¹ radicals. The total number of aromatic ring atoms in Ar², Ar³ and Ar⁴ here is not more than 60 and preferably not more than 40.

Ar⁴ and Ar² here may also be bonded to one another and/or Ar² and Ar³ to one another by a group selected from C(R¹)₂, NR¹, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R¹ radicals. More preferably, Ar⁴ is selected from the group consisting of ortho-, meta- or para-phenylene or ortho-, meta- or para-biphenyl, each of which may be substituted by one or more R¹ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R¹ radicals. Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a further preferred embodiment of the invention, R¹ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R² radicals. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 13 aromatic ring atoms and may be substituted in each case by one or more R⁵ radicals, but is preferably unsubstituted.

In a further preferred embodiment of the invention, R² is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (I) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. An exception to this is formed by phenanthrene and triphenylene, which, because of their high triplet energy, may be preferable in spite of the presence of fused aromatic six-membered rings.

It may also be the case that the compound includes exactly two or exactly three structures of formula (I), (1-1) to (I-6) (II-1) to (II-154), (III-1) to (III-60) and/or (IV-1) to (IV-12).

In a preferred configuration, the compounds are selected from compounds of the formula (D-1):

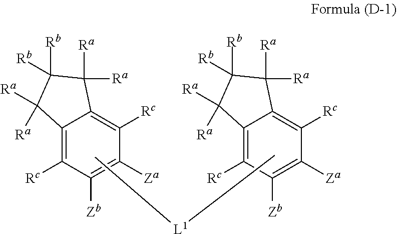

where the L¹ group is a connecting group, preferably a bond or an aromatic or heteroaromatic ring system which has 5 to 40, preferably 5 to 30, aromatic ring atoms and may be substituted by one or more R radicals, and the further symbols used have the definitions given above, especially for formula (I), where the L¹ group forms a bond to the base structure in place of a hydrogen atom or a substituent; preferably, the L¹ group binds to the Z^{a}, Z^{b} radicals.

In a further preferred embodiment of the invention, L¹ is a bond or an aromatic or heteroaromatic ring system which has 5 to 14 aromatic or heteroaromatic ring atoms, preferably an aromatic ring system which has 6 to 12 carbon atoms, and which may be substituted by one or more R radicals, but is preferably unsubstituted, where R may have the definition given above, especially for formula (I). More preferably, L¹ is an aromatic ring system having 6 to 10 aromatic ring atoms or a heteroaromatic ring system having 6 to 13 heteroaromatic ring atoms, each of which may be substituted by one or more R¹ radicals, but is preferably unsubstituted, where R¹ may have the definition given above, especially for formula (I).

Further preferably, the symbol L¹ shown in formula (D1) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L¹ group shown in formula (D1) comprises an aromatic ring system having not more than four, preferably not more than three, more preferably not more than two, fused aromatic and/or heteroaromatic 6-membered rings, and preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L¹ are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R¹ radicals, but are preferably unsubstituted.

In a preferred configuration, a compound of the invention can be represented by at least one of the structures of formulae (I), (I-1) to (I-6) (II-1) to (II-154), (III-1) to (III-60) and/or (IV-1) to (IV-12). Preferably, compounds of the invention, preferably comprising structures of formulae (I), (I-1) to (I-6) (II-1) to (II-154), (III-1) to (III-60) and/or (IV-1) to (IV-12), have a molecular weight of not more than 5000 g/mol, preferably not more than 4000 g/mol, particularly preferably not more than 3000 g/mol, especially preferably not more than 2000 g/mol, more especially preferably not more than 1200 g/mol and most preferably not more than 900 g/mol.

In addition, it is a feature of preferred compounds of the invention that they are sublimable. These compounds generally have a molar mass of less than about 1200 g/mol.

It may also be the case that the compound comprising structures of formula (I), preferably the compound of formula (I) or a preferred embodiment of that structure/compound, is not in direct contact with a metal atom, and is preferably not a ligand for a metal complex.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

| | |
|---|---|
| 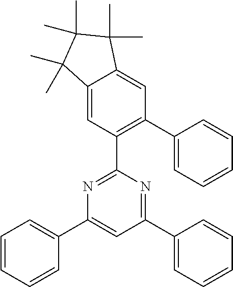 [SMILES: CC1(C)c2cc(-c3ccccc3)c(-c3nc(-c4ccccc4)cc(-c4ccccc4)n3)cc2C(C)(C)C1(C)C] | 1 |
| 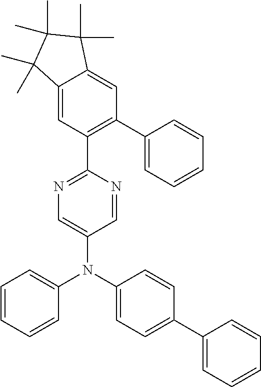 [SMILES: CC1(C)c2cc(-c3ccccc3)c(-c3ncc(N(c4ccccc4)c4ccc(-c5ccccc5)cc4)cn3)cc2C(C)(C)C1(C)C] | 2 |
| 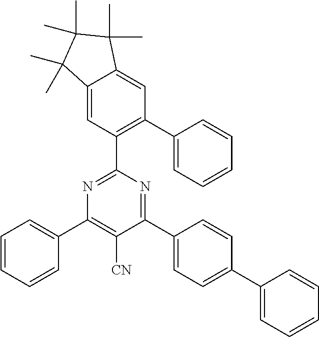 [SMILES: CC1(C)c2cc(-c3ccccc3)c(-c3nc(-c4ccccc4)c(C#N)c(-c4ccc(-c5ccccc5)cc4)n3)cc2C(C)(C)C1(C)C] | 3 |
| 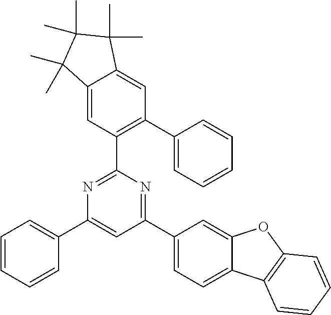 [SMILES: CC1(C)c2cc(-c3ccccc3)c(-c3nc(-c4ccccc4)cc(-c4ccc5c(c4)oc4ccccc45)n3)cc2C(C)(C)C1(C)C] | 4 |
| 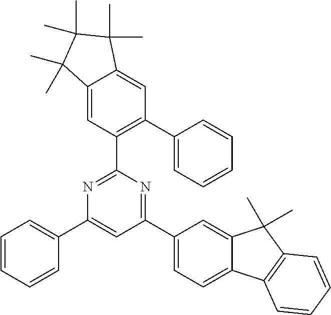 [SMILES: CC1(C)c2ccccc2-c2ccc(-c3cc(-c4ccccc4)nc(-c4cc5c(cc4-c4ccccc4)C(C)(C)C(C)(C)C5(C)C)n3)cc21] | 5 |
| 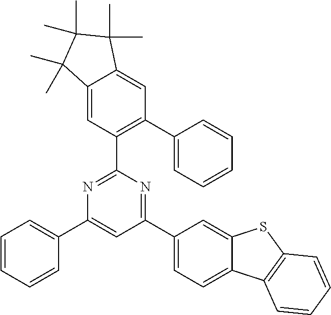 [SMILES: CC1(C)c2cc(-c3ccccc3)c(-c3nc(-c4ccccc4)cc(-c4ccc5c(c4)sc4ccccc45)n3)cc2C(C)(C)C1(C)C] | 6 |
| 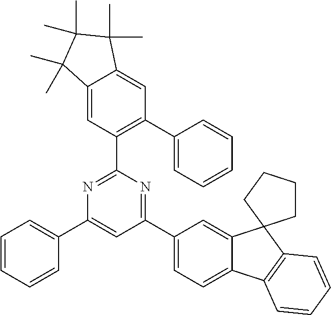 [SMILES: CC1(C)c2cc(-c3ccccc3)c(-c3nc(-c4ccccc4)cc(-c4ccc5c(c4)C4(CCCC4)c4ccccc4-5)n3)cc2C(C)(C)C1(C)C] | 7 |
| 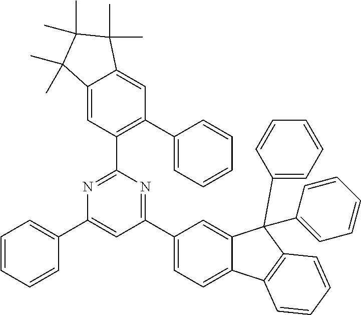 [SMILES: CC1(C)c2cc(-c3ccccc3)c(-c3nc(-c4ccccc4)cc(-c4ccc5c(c4)C(c4ccccc4)(c4ccccc4)c4ccccc4-5)n3)cc2C(C)(C)C1(C)C] | 8 |
| 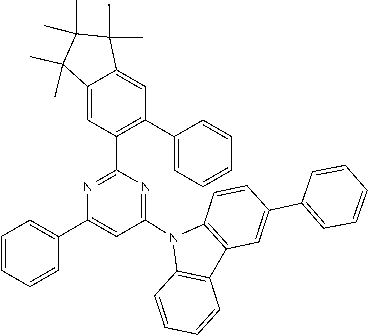 [SMILES: CC1(C)c2cc(-c3ccccc3)c(-c3nc(-c4ccccc4)cc(-n4c5ccccc5c5cc(-c6ccccc6)ccc54)n3)cc2C(C)(C)C1(C)C] | 9 |
| 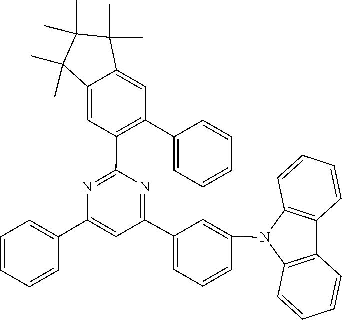 [SMILES: CC1(C)c2cc(-c3ccccc3)c(-c3nc(-c4ccccc4)cc(-c4cccc(-n5c6ccccc6c6ccccc65)c4)n3)cc2C(C)(C)C1(C)C] | 10 |
| 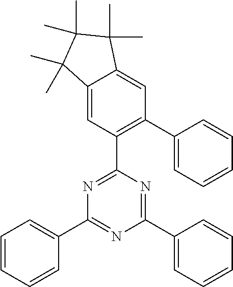 [SMILES: CC1(C)c2cc(-c3ccccc3)c(-c3nc(-c4ccccc4)nc(-c4ccccc4)n3)cc2C(C)(C)C1(C)C] | 11 |
| 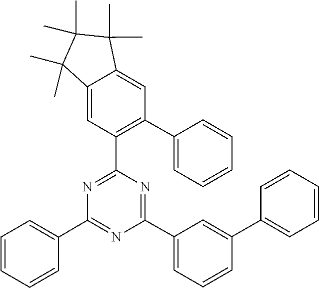 [SMILES: CC1(C)c2cc(-c3ccccc3)c(-c3nc(-c4ccccc4)nc(-c4cccc(-c5ccccc5)c4)n3)cc2C(C)(C)C1(C)C] | 12 |
| 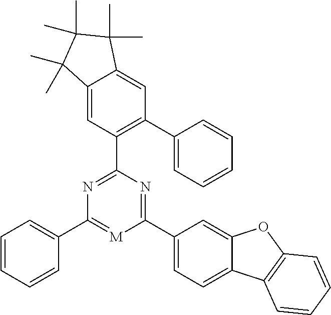 | 13 |
| 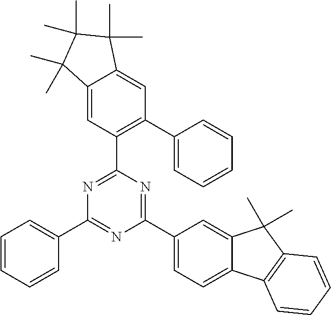 [SMILES: CC1(C)c2ccccc2-c2ccc(-c3nc(-c4ccccc4)nc(-c4cc5c(cc4-c4ccccc4)C(C)(C)C(C)(C)C5(C)C)n3)cc21] | 14 |
| 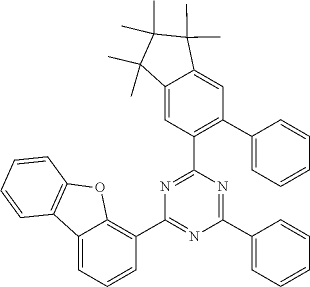 [SMILES: CC1(C)c2cc(-c3ccccc3)c(-c3nc(-c4ccccc4)nc(-c4cccc5c4oc4ccccc45)n3)cc2C(C)(C)C1(C)C] | 15 |
| 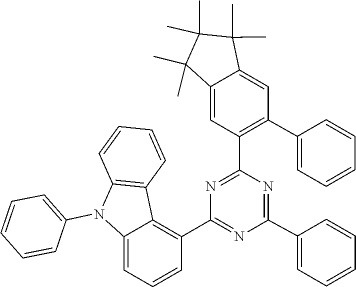 [SMILES: CC1(C)c2cc(-c3ccccc3)c(-c3nc(-c4ccccc4)nc(-c4cccc5c4c4ccccc4n5-c4ccccc4)n3)cc2C(C)(C)C1(C)C] | 16 |
| 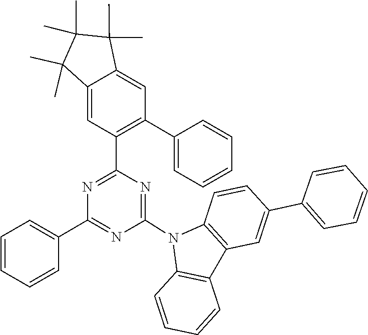 [SMILES: CC1(C)c2cc(-c3ccccc3)c(-c3nc(-c4ccccc4)nc(-n4c5ccccc5c5cc(-c6ccccc6)ccc54)n3)cc2C(C)(C)C1(C)C] | 17 |
| 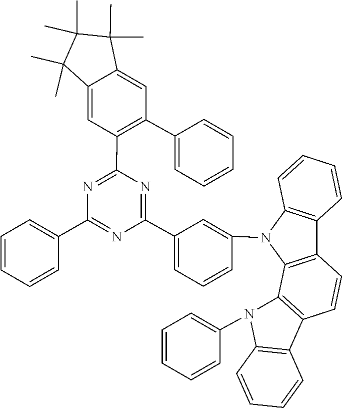 [SMILES: CC1(C)c2cc(-c3ccccc3)c(-c3nc(-c4ccccc4)nc(-c4cccc(-n5c6ccccc6c6ccc7c8ccccc8n(-c8ccccc8)c7c65)c4)n3)cc2C(C)(C)C1(C)C] | 18 |
| 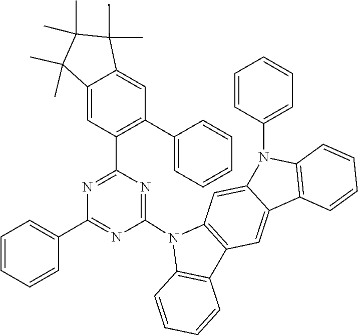 [SMILES: CC1(C)c2cc(-c3ccccc3)c(-c3nc(-c4ccccc4)nc(-n4c5ccccc5c5cc6c7ccccc7n(-c7ccccc7)c6cc54)n3)cc2C(C)(C)C1(C)C] | 19 |
| 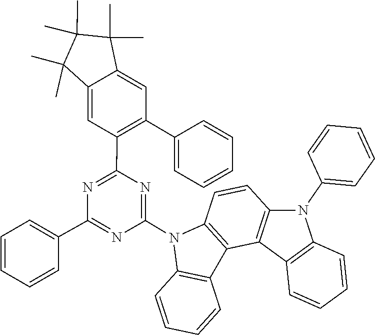 [SMILES: CC1(C)c2cc(-c3ccccc3)c(-c3nc(-c4ccccc4)nc(-n4c5ccccc5c5c6c7ccccc7n(-c7ccccc7)c6ccc54)n3)cc2C(C)(C)C1(C)C] | 20 |
| 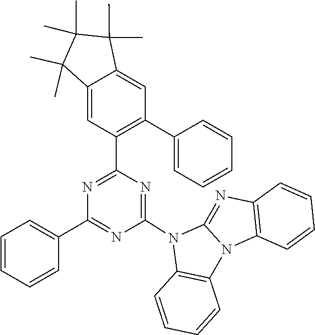 [SMILES: CC1(C)c2cc(-c3ccccc3)c(-c3nc(-c4ccccc4)nc(-n4c5ccccc5n5c6ccccc6nc45)n3)cc2C(C)(C)C1(C)C] | 21 |
| 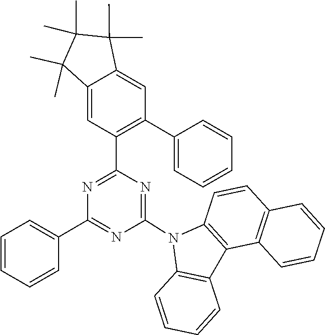 [SMILES: CC1(C)c2cc(-c3ccccc3)c(-c3nc(-c4ccccc4)nc(-n4c5ccccc5c5c6ccccc6ccc54)n3)cc2C(C)(C)C1(C)C] | 22 |
| 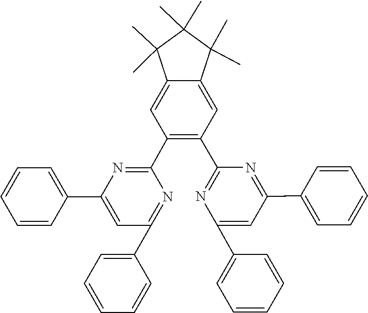 [SMILES: CC1(C)c2cc(-c3nc(-c4ccccc4)cc(-c4ccccc4)n3)c(-c3nc(-c4ccccc4)cc(-c4ccccc4)n3)cc2C(C)(C)C1(C)C] | 23 |
| 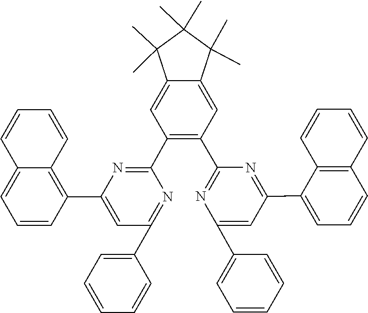 [SMILES: CC1(C)c2cc(-c3nc(-c4ccccc4)cc(-c4cccc5ccccc45)n3)c(-c3nc(-c4ccccc4)cc(-c4cccc5ccccc45)n3)cc2C(C)(C)C1(C)C] | 24 |
| 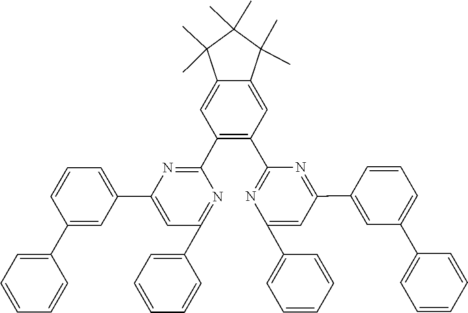 [SMILES: CC1(C)c2cc(-c3nc(-c4ccccc4)cc(-c4cccc(-c5ccccc5)c4)n3)c(-c3nc(-c4ccccc4)cc(-c4cccc(-c5ccccc5)c4)n3)cc2C(C)(C)C1(C)C] | 25 |
| 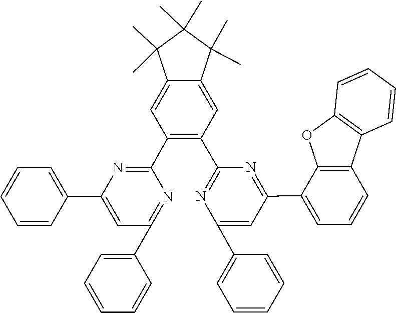 [SMILES: CC1(C)c2cc(-c3nc(-c4ccccc4)cc(-c4ccccc4)n3)c(-c3nc(-c4ccccc4)cc(-c4cccc5c4oc4ccccc45)n3)cc2C(C)(C)C1(C)C] | 26 |
| 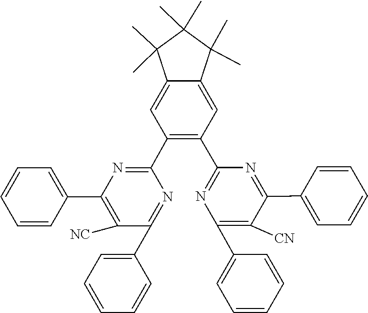 [SMILES: CC1(C)c2cc(-c3nc(-c4ccccc4)c(C#N)c(-c4ccccc4)n3)c(-c3nc(-c4ccccc4)c(C#N)c(-c4ccccc4)n3)cc2C(C)(C)C1(C)C] | 27 |
| 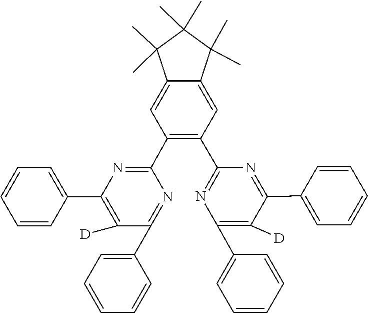 [SMILES: [2H]c1c(-c2ccccc2)nc(-c2cc3c(cc2-c2nc(-c4ccccc4)c([2H])c(-c4ccccc4)n2)C(C)(C)C(C)(C)C3(C)C)nc1-c1ccccc1] | 28 |
| 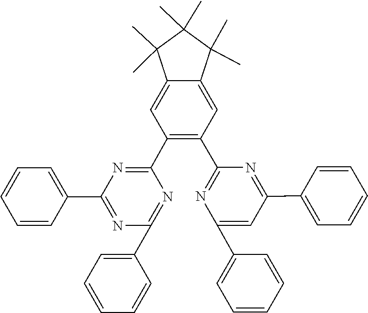 [SMILES: CC1(C)c2cc(-c3nc(-c4ccccc4)cc(-c4ccccc4)n3)c(-c3nc(-c4ccccc4)nc(-c4ccccc4)n3)cc2C(C)(C)C1(C)C] | 29 |
| 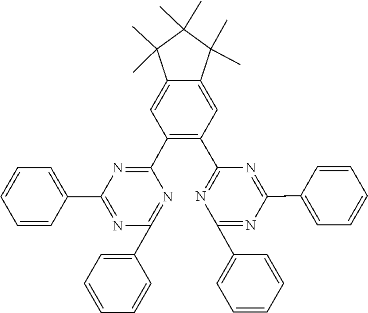 [SMILES: CC1(C)c2cc(-c3nc(-c4ccccc4)nc(-c4ccccc4)n3)c(-c3nc(-c4ccccc4)nc(-c4ccccc4)n3)cc2C(C)(C)C1(C)C] | 30 |
| 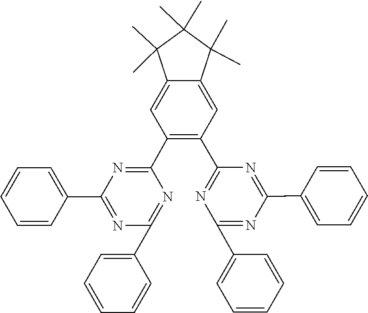 [SMILES: CC1(C)c2cc(-c3nc(-c4ccccc4)nc(-c4ccccc4)n3)c(-c3nc(-c4ccccc4)nc(-c4ccccc4)n3)cc2C(C)(C)C1(C)C] | 31 |
| 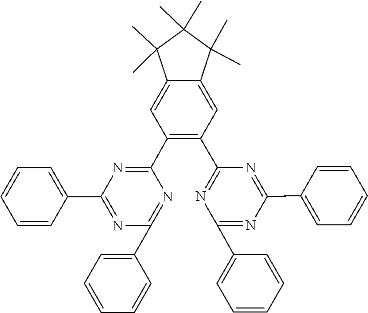 [SMILES: CC1(C)c2cc(-c3nc(-c4ccccc4)nc(-c4ccccc4)n3)c(-c3nc(-c4ccccc4)nc(-c4ccccc4)n3)cc2C(C)(C)C1(C)C] | 30 |
| 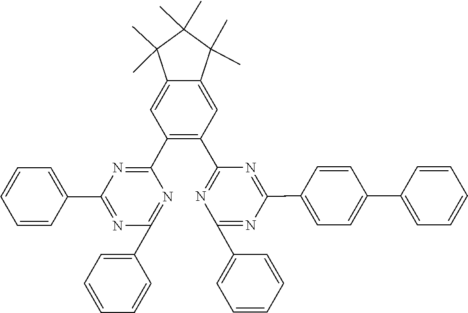 [SMILES: CC1(C)c2cc(-c3nc(-c4ccccc4)nc(-c4ccccc4)n3)c(-c3nc(-c4ccccc4)nc(-c4ccc(-c5ccccc5)cc4)n3)cc2C(C)(C)C1(C)C] | 32 |
| 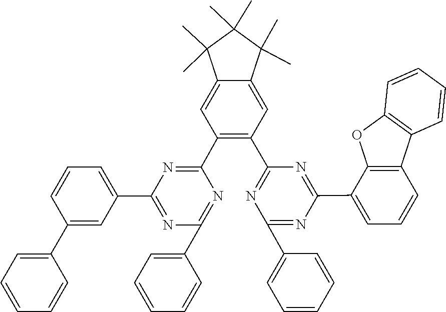 [SMILES: CC1(C)c2cc(-c3nc(-c4ccccc4)nc(-c4cccc(-c5ccccc5)c4)n3)c(-c3nc(-c4ccccc4)nc(-c4cccc5c4oc4ccccc45)n3)cc2C(C)(C)C1(C)C] | 33 |
| 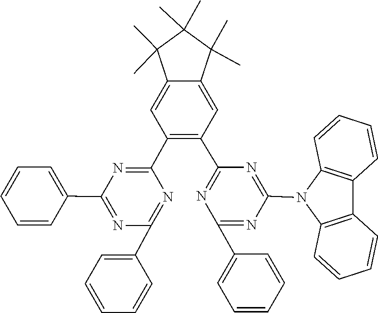 [SMILES: CC1(C)c2cc(-c3nc(-c4ccccc4)nc(-c4ccccc4)n3)c(-c3nc(-c4ccccc4)nc(-n4c5ccccc5c5ccccc54)n3)cc2C(C)(C)C1(C)C] | 34 |
| 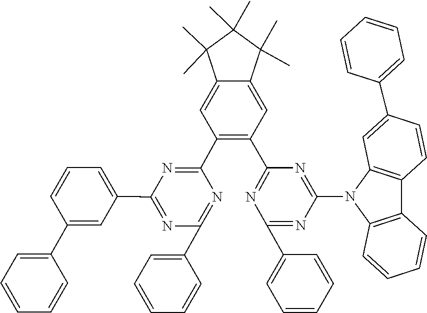 | 35 |
| 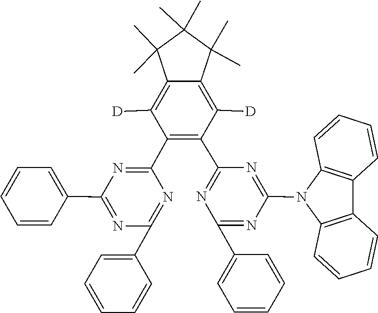 [SMILES: [2H]c1c(-c2nc(-c3ccccc3)nc(-c3ccccc3)n2)c(-c2nc(-c3ccccc3)nc(-n3c4ccccc4c4ccccc43)n2)c([2H])c2c1C(C)(C)C(C)(C)C2(C)C] | 36 |
| 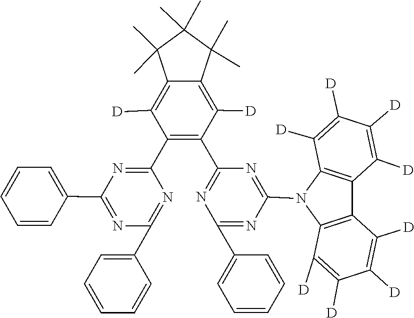 [SMILES: [2H]c1c(-c2nc(-c3ccccc3)nc(-c3ccccc3)n2)c(-c2nc(-c3ccccc3)nc(-n3c4c([2H])c([2H])c([2H])c([2H])c4c4c([2H])c([2H])c([2H])c([2H])c43)n2)c([2H])c2c1C(C)(C)C(C)(C)C2(C)C] | 37 |
| 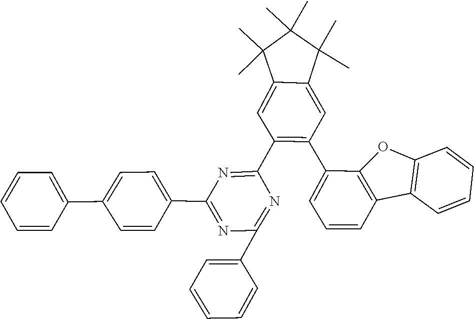 [SMILES: CC1(C)c2cc(-c3nc(-c4ccccc4)nc(-c4ccc(-c5ccccc5)cc4)n3)c(-c3cccc4c3oc3ccccc34)cc2C(C)(C)C1(C)C] | 38 |
| 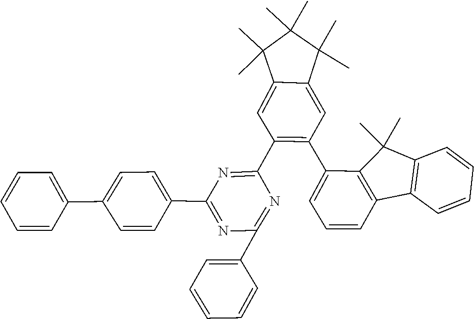 [SMILES: CC1(C)c2ccccc2-c2cccc(-c3cc4c(cc3-c3nc(-c5ccccc5)nc(-c5ccc(-c6ccccc6)cc5)n3)C(C)(C)C(C)(C)C4(C)C)c21] | 39 |
| 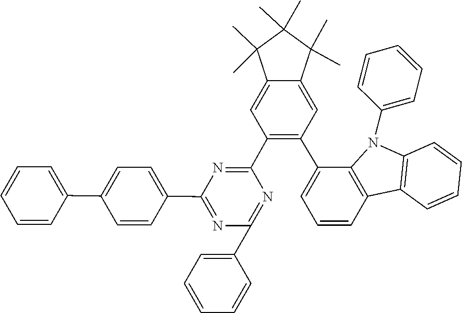 [SMILES: CC1(C)c2cc(-c3nc(-c4ccccc4)nc(-c4ccc(-c5ccccc5)cc4)n3)c(-c3cccc4c5ccccc5n(-c5ccccc5)c34)cc2C(C)(C)C1(C)C] | 40 |
| 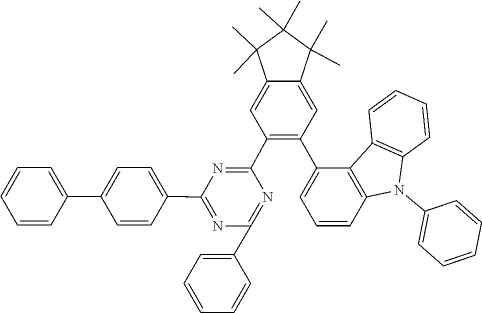 [SMILES: CC1(C)c2cc(-c3nc(-c4ccccc4)nc(-c4ccc(-c5ccccc5)cc4)n3)c(-c3cccc4c3c3ccccc3n4-c3ccccc3)cc2C(C)(C)C1(C)C] | 41 |
| 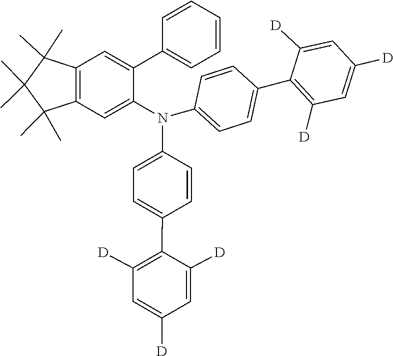 [SMILES: [2H]c1cc([2H])c(-c2ccc(N(c3ccc(-c4c([2H])cc([2H])cc4[2H])cc3)c3cc4c(cc3-c3ccccc3)C(C)(C)C(C)(C)C4(C)C)cc2)c([2H])c1] | 42 |
| 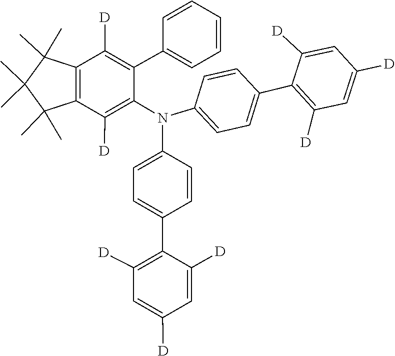 [SMILES: [2H]c1cc([2H])c(-c2ccc(N(c3ccc(-c4c([2H])cc([2H])cc4[2H])cc3)c3c([2H])c4c(c([2H])c3-c3ccccc3)C(C)(C)C(C)(C)C4(C)C)cc2)c([2H])c1] | 43 |
| 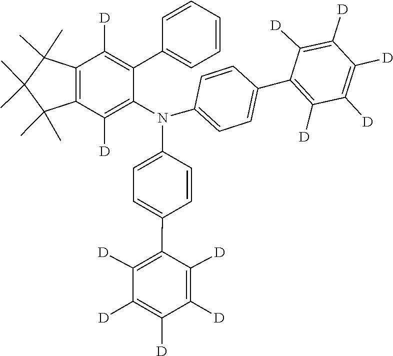 [SMILES: [2H]c1c([2H])c([2H])c(-c2ccc(N(c3ccc(-c4c([2H])c([2H])c([2H])c([2H])c4[2H])cc3)c3c([2H])c4c(c([2H])c3-c3ccccc3)C(C)(C)C(C)(C)C4(C)C)cc2)c([2H])c1[2H]] | 44 |
| 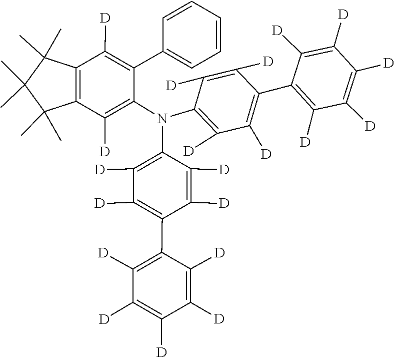 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c(c([2H])c3-c3ccccc3)C(C)(C)C(C)(C)C4(C)C)c([2H])c2[2H])c([2H])c1[2H]] | 45 |
| 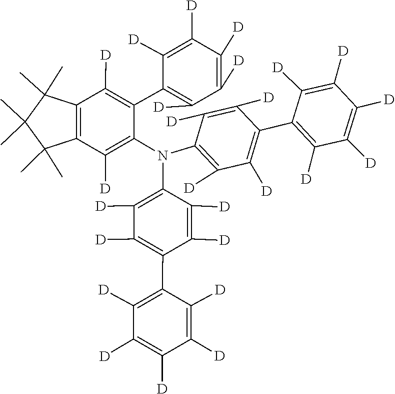 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c(c([2H])c3-c3c([2H])c([2H])c([2H])c([2H])c3[2H])C(C)(C)C(C)(C)C4(C)C)c([2H])c2[2H])c([2H])c1[2H]] | 46 |
| 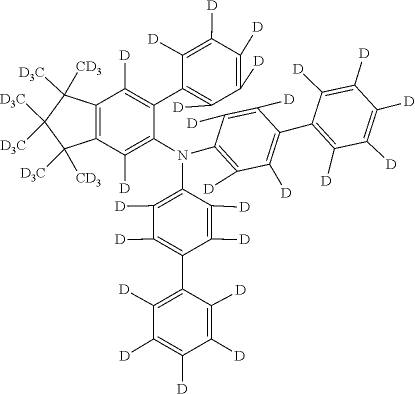 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c(c([2H])c3-c3c([2H])c([2H])c([2H])c([2H])c3[2H])C(C([2H])([2H])[2H])(C([2H])([2H])[2H])C(C([2H])([2H])[2H])(C([2H])([2H])[2H])C4(C([2H])([2H])[2H])C([2H])([2H])[2H])c([2H])c2[2H])c([2H])c1[2H]] | 47 |
| 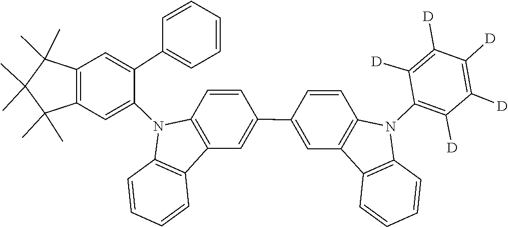 [SMILES: [2H]c1c([2H])c([2H])c(-n2c3ccccc3c3cc(-c4ccc5c(c4)c4ccccc4n5-c4cc5c(cc4-c4ccccc4)C(C)(C)C(C)(C)C5(C)C)ccc32)c([2H])c1[2H]] | 48 |
| 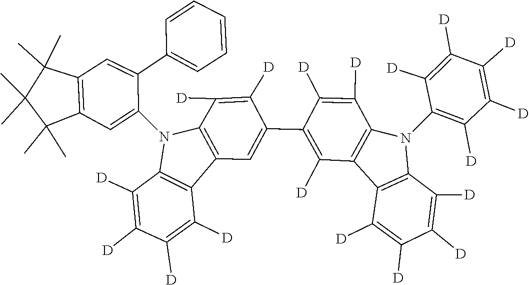 [SMILES: [2H]c1c([2H])c([2H])c(-n2c3c([2H])c([2H])c([2H])c([2H])c3c3c([2H])c(-c4cc5c6c([2H])c([2H])c([2H])c([2H])c6n(-c6cc7c(cc6-c6ccccc6)C(C)(C)C(C)(C)C7(C)C)c5c([2H])c4[2H])c([2H])c([2H])c32)c([2H])c1[2H]] | 49 |
| 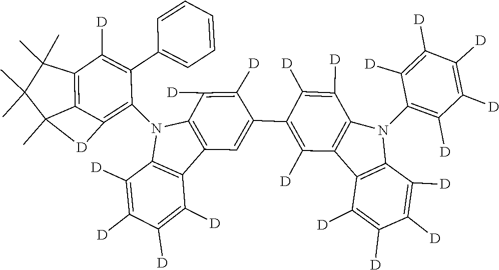 | 50 |
| 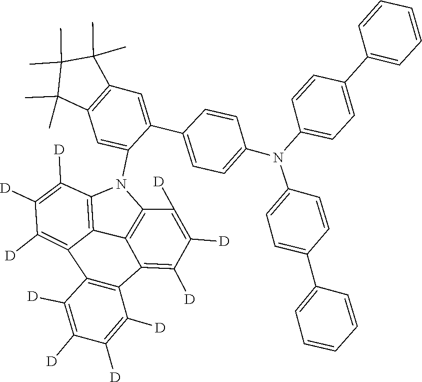 [SMILES: [2H]c1c([2H])c([2H])c2c(c1[2H])c1c([2H])c([2H])c([2H])c3c1c1c2c([2H])c([2H])c([2H])c1n3-c1cc2c(cc1-c1ccc(N(c3ccc(-c4ccccc4)cc3)c3ccc(-c4ccccc4)cc3)cc1)C(C)(C)C(C)(C)C2(C)C] | 51 |
| 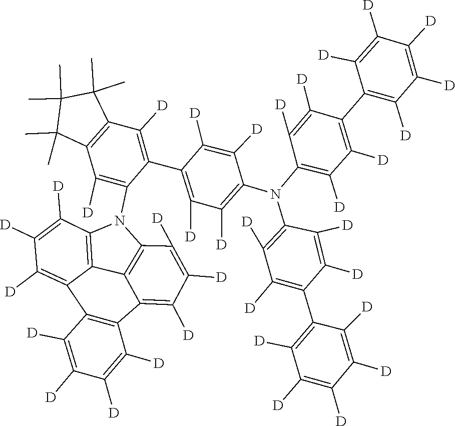 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c5c(c([2H])c4-n4c6c([2H])c([2H])c([2H])c7c8c([2H])c([2H])c([2H])c([2H])c8c8c([2H])c([2H])c([2H])c4c8c76)C(C)(C)C(C)(C)C5(C)C)c([2H])c3[2H])c([2H])c2[2H])c([2H])c1[2H]] | 52 |
| 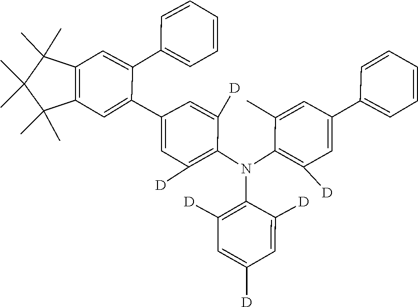 [SMILES: [2H]c1cc([2H])c(N(c2c([2H])cc(-c3cc4c(cc3-c3ccccc3)C(C)(C)C(C)(C)C4(C)C)cc2[2H])c2c([2H])cc(-c3ccccc3)cc2C)c([2H])c1] | 53 |
| 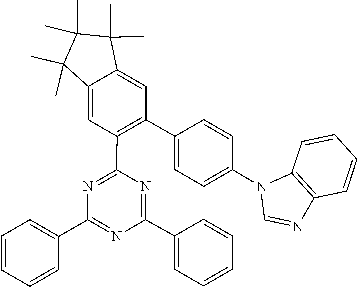 [SMILES: CC1(C)c2cc(-c3ccc(-n4cnc5ccccc54)cc3)c(-c3nc(-c4ccccc4)nc(-c4ccccc4)n3)cc2C(C)(C)C1(C)C] | 54 |
| 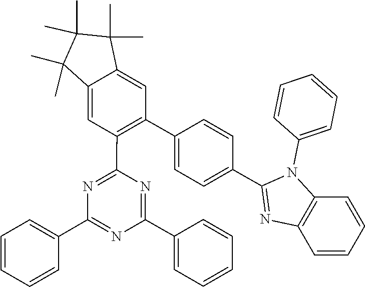 [SMILES: CC1(C)c2cc(-c3ccc(-c4nc5ccccc5n4-c4ccccc4)cc3)c(-c3nc(-c4ccccc4)nc(-c4ccccc4)n3)cc2C(C)(C)C1(C)C] | 55 |
| 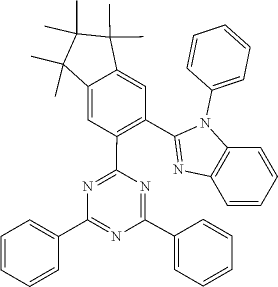 [SMILES: CC1(C)c2cc(-c3nc(-c4ccccc4)nc(-c4ccccc4)n3)c(-c3nc4ccccc4n3-c3ccccc3)cc2C(C)(C)C1(C)C] | 56 |
| 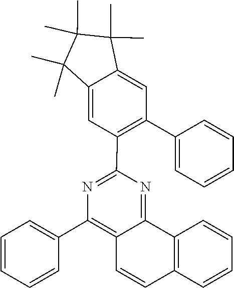 [SMILES: CC1(C)c2cc(-c3ccccc3)c(-c3nc(-c4ccccc4)c4ccc5ccccc5c4n3)cc2C(C)(C)C1(C)C] | 57 |
| 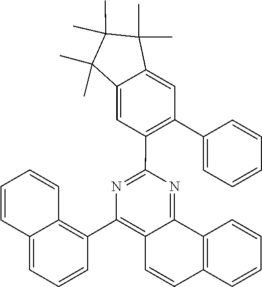 [SMILES: CC1(C)c2cc(-c3ccccc3)c(-c3nc(-c4cccc5ccccc45)c4ccc5ccccc5c4n3)cc2C(C)(C)C1(C)C] | 58 |
| 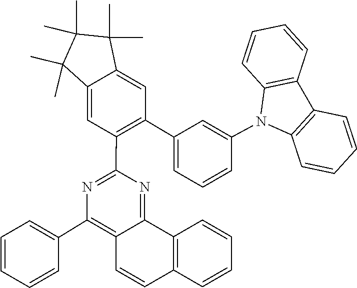 [SMILES: CC1(C)c2cc(-c3cccc(-n4c5ccccc5c5ccccc54)c3)c(-c3nc(-c4ccccc4)c4ccc5ccccc5c4n3)cc2C(C)(C)C1(C)C] | 59 |
| 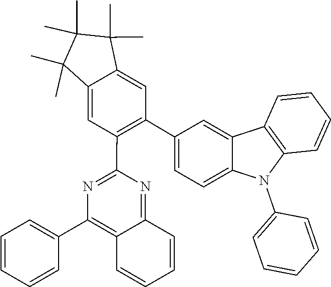 [SMILES: CC1(C)c2cc(-c3ccc4c(c3)c3ccccc3n4-c3ccccc3)c(-c3nc(-c4ccccc4)c4ccccc4n3)cc2C(C)(C)C1(C)C] | 60 |
| 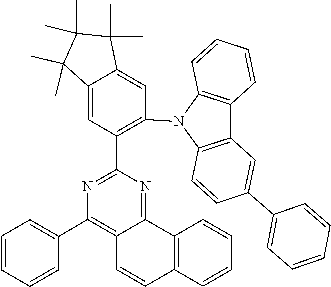 [SMILES: CC1(C)c2cc(-c3nc(-c4ccccc4)c4ccc5ccccc5c4n3)c(-n3c4ccccc4c4cc(-c5ccccc5)ccc43)cc2C(C)(C)C1(C)C] | 61 |
| 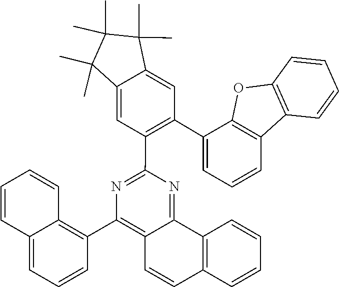 [SMILES: CC1(C)c2cc(-c3nc(-c4cccc5ccccc45)c4ccc5ccccc5c4n3)c(-c3cccc4c3oc3ccccc34)cc2C(C)(C)C1(C)C] | 62 |
| 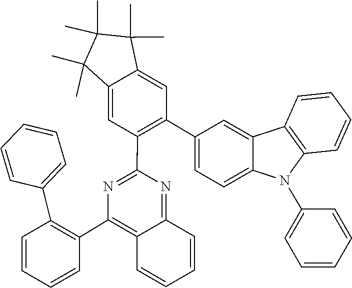 [SMILES: CC1(C)c2cc(-c3ccc4c(c3)c3ccccc3n4-c3ccccc3)c(-c3nc(-c4ccccc4-c4ccccc4)c4ccccc4n3)cc2C(C)(C)C1(C)C] | 63 |
| 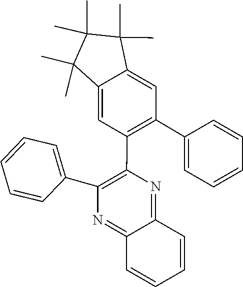 [SMILES: CC1(C)c2cc(-c3ccccc3)c(-c3nc4ccccc4nc3-c3ccccc3)cc2C(C)(C)C1(C)C] | 64 |
| 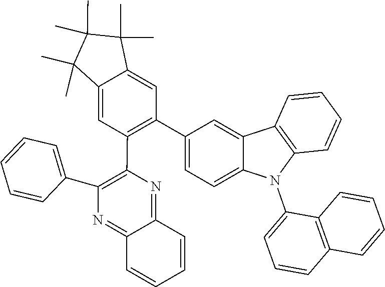 [SMILES: CC1(C)c2cc(-c3ccc4c(c3)c3ccccc3n4-c3cccc4ccccc34)c(-c3nc4ccccc4nc3-c3ccccc3)cc2C(C)(C)C1(C)C] | 65 |
| 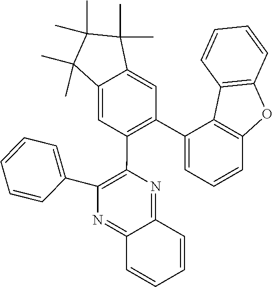 [SMILES: CC1(C)c2cc(-c3nc4ccccc4nc3-c3ccccc3)c(-c3cccc4oc5ccccc5c34)cc2C(C)(C)C1(C)C] | 66 |
| 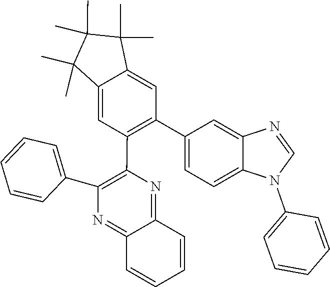 [SMILES: CC1(C)c2cc(-c3ccc4c(c3)ncn4-c3ccccc3)c(-c3nc4ccccc4nc3-c3ccccc3)cc2C(C)(C)C1(C)C] | 67 |

The base structure of the compounds of the invention can be prepared by the routes outlined in the schemes which follow. The individual synthesis steps here, for example coupling reactions that lead to C—C bond formation and/or C—N bond formation, are known in principle to the person skilled in the art. These include BUCHWALD, SUZUKI, YAMAMOTO, STILLE, HECK, NEGISHI, SONOGASHIRA and HIYAMA reactions. Further information relating to the synthesis of the compounds of the invention can be found in the synthesis examples.

The schemes that follow describe the preparation of the compounds of the invention by the use of 5,6-dibromo-2,3-dihydro-1,1,2,2,3,3-hexamethyl-1H-indene [1541101-19-2] or the partly or fully deuterated variants thereof. This use should be considered to be illustrative, and so further compounds of the invention can be obtained by similar synthesis routes that proceed from different base structures.

The synthesis of phenyl compounds to which a cyclopentyl group is fused is widely known in the specialist field. Many of these compounds, in particular 5,6-dibromo-2,3-dihydro-1,1,2,2,3,3-hexamethyl-1H-indene [1541101-19-2] or the partly or fully deuterated variants thereof, are commercially available. These include, for example, 5,6-dibromo-2,3-dihydro-1,1,2,2,3,3-hexamethyl-1H-indene-4,7-d2 [1541101-31-8] and 5,6-dibromo-2,3-dihydro-1,1,2,2,3,3-hexa(methyl-d₃)-1H-indene [1541101-25-0].

The compounds of the invention having amine groups, especially compounds comprising structures of formulae (I-2) to (I-6), can be obtained proceeding from 5,6-dibromo-2,3-dihydro-1,1,2,2,3,3-hexamethyl-1H-indene [1541101-19-2] or the partly or fully deuterated variants thereof by the following synthesis routes:
1) Suzuki couplings with an aryl/heteroarylboronic acid or ester ((HO)₂B-Ar), followed by a Buchwald-Hartwig or Ullmann coupling with introduction of a diarylamine radical NAr¹Ar²:

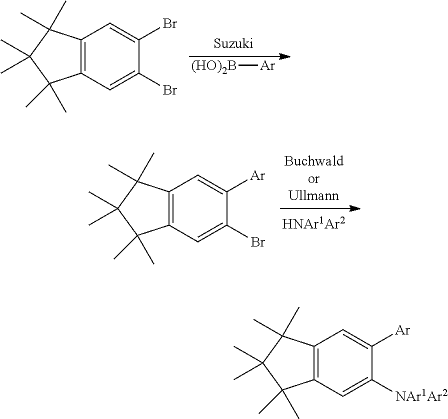

2) A first Suzuki coupling 1 with an aryl/heteroarylboronic acid or ester ((HO)₂B—Ar¹), followed by a second Suzuki coupling 2 with an aryl/heteroarylboronic acid or ester ((HO)₂B—Ar²), where at least one of the two aryl/heteroarylboronic acids or esters comprises an amine or carbazole group:

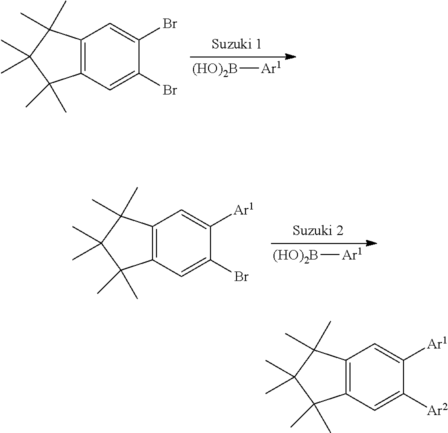

3) A first Buchwald-Hartwig or Ullmann coupling 1 with a diarylamine (HNAr¹Ar²), followed by a second Buchwald-Hartwig or Ullmann coupling 2 with a diarylamine (HNAr³Ar⁴):

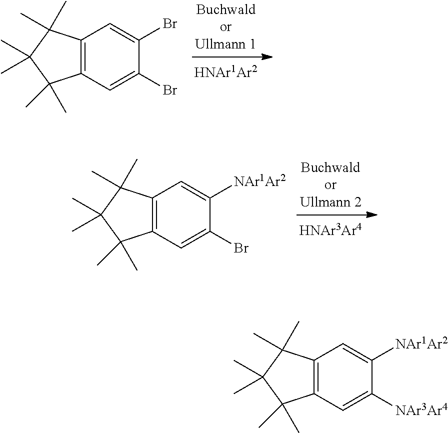

are shown.

The compounds of the invention bearing electron-deficient heterocycles, especially pyrimidines and triazines, for example compounds comprising structures of formula (I-1), can be obtained proceeding from 5,6-dibromo-2,3-dihydro-1, 1,2,2,3,3-hexamethyl-1H-indene [1541101-19-2] or the partly or fully deuterated variants thereof by the following synthesis routes:
1) Suzuki couplings with an aryl/heteroarylboronic acid or ester ((HO)₂B-Ar), followed by metallation (lithiation or Grignard formation) and reaction with a chlorodiarylpyrimidine or -triazine:

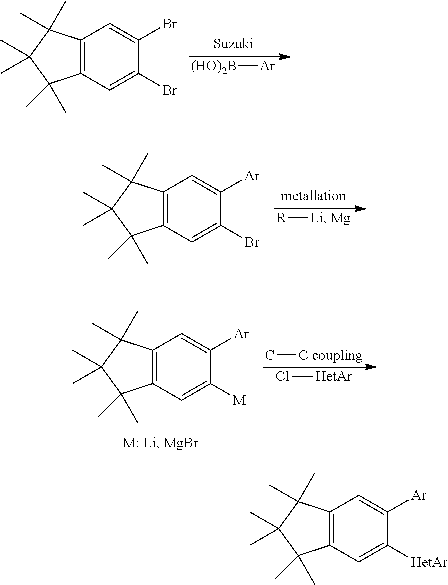

2) A first Suzuki coupling 1 with an aryl/heteroarylboronic acid or ester ((HO)₂B—Ar¹), followed by a second Suzuki coupling 2 with an aryl/heteroarylboronic acid or ester ((HO)₂B—Ar²), where at least one of the two aryl/heteroarylboronic acids or esters comprises a heteroaryl group of the invention:

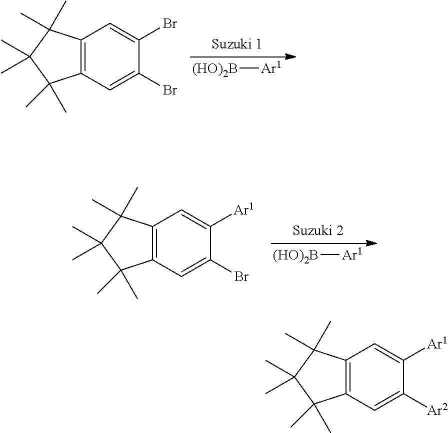

3) A bisborylation and subsequently a first Suzuki coupling 1 with a haloaromatic or -heteroaromatic (Hal-Ar¹), followed by a second Suzuki coupling 2 with a haloaromatic or -heteroaromatic (Hal-Ar²), where at least one of the two haloaromatics or -heteroaromatics (Hal-Ar¹) comprises a heteroaryl group of the invention, where it is thus preferably possible to join pyrimidines or triazines directly to the 2,3-dihydro-1,1,2,2,3,3-hexamethyl-1H-indene:

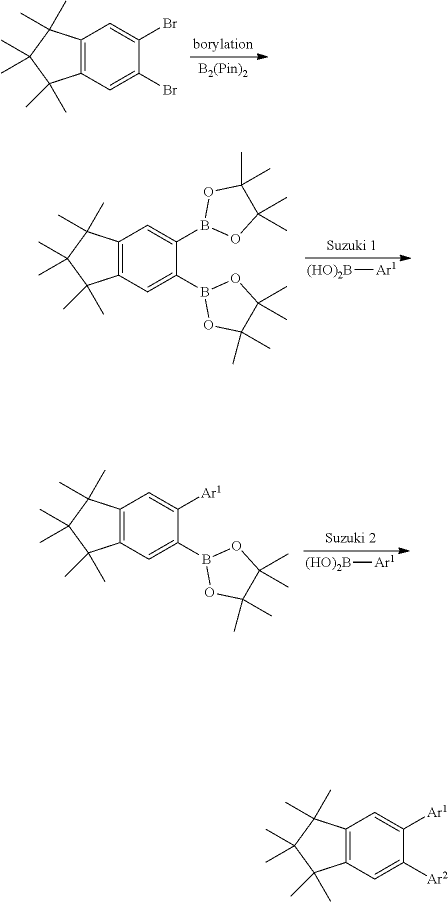

are shown.

The definition of the symbols used in the schemes set out above corresponds essentially to that which was defined for formula (I), dispensing with numbering and complete representation of all symbols for reasons of clarity.

The present invention therefore further provides a process for preparing a compound of the invention, wherein a phenyl compound to which a cyclopentyl group is fused is synthesized and at least one aromatic or heteroaromatic radical is introduced, preferably by a nucleophilic aromatic substitution reaction or a coupling reaction.

It is possible by these methods, if necessary followed by purification, for example recrystallization or sublimation, to obtain the compounds of the invention in high purity, preferably more than 99% (determined by means of ¹H NMR and/or HPLC).

The compounds of the invention may also be mixed with a polymer. It is likewise possible to incorporate these compounds covalently into a polymer. This is especially possible with compounds substituted by reactive leaving groups such as bromine, iodine, chlorine, boronic acid or boronic ester, or by reactive polymerizable groups such as olefins or oxetanes. These may find use as monomers for production of corresponding oligomers, dendrimers or polymers. The oligomerization or polymerization is preferably effected via the halogen functionality or the boronic acid functionality or via the polymerizable group. It is additionally possible to crosslink the polymers via groups of this kind. The compounds and polymers of the invention may be used in the form of a crosslinked or uncrosslinked layer.

The invention therefore further provides oligomers, polymers or dendrimers containing one or more of the above-detailed structures of the formula (I) and preferred embodiments of this formula or compounds of the invention, wherein one or more bonds of the compounds of the invention or of the structures of the formula (I) and preferred embodiments of that formula to the polymer, oligomer or dendrimer are present. According to the linkage of the structures of the formula (I) and preferred embodiments of this formula or of the compounds, these therefore form a side chain of the oligomer or polymer or are bonded within the main chain. The polymers, oligomers or dendrimers may be conjugated, partly conjugated or nonconjugated. The oligomers or polymers may be linear, branched or dendritic. For the repeat units of the compounds of the invention in oligomers, dendrimers and polymers, the same preferences apply as described above.

For preparation of the oligomers or polymers, the monomers of the invention are homopolymerized or copolymerized with further monomers. Preference is given to copolymers wherein the units of formula (I) or the preferred embodiments recited above and hereinafter are present to an extent of 0.01 to 99.9 mol %, preferably 5 to 90 mol %, more preferably 20 to 80 mol %. Suitable and preferred comonomers which form the polymer base skeleton are chosen from fluorenes (for example according to EP 842208 or WO 2000/022026), spirobifluorenes (for example according to EP 707020, EP 894107 or WO 2006/061181), paraphenylenes (for example according to WO 92/18552), carbazoles (for example according to WO 2004/070772 or WO 2004/113468), thiophenes (for example according to EP 1028136), dihydrophenanthrenes (for example according to WO 2005/014689), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers may contain still further units, for example hole transport units, especially those based on triarylamines, and/or electron transport units.

Additionally of particular interest are compounds of the invention which feature a high glass transition temperature. In this connection, preference is given especially to compounds of the invention comprising structures of the formula (I) or the preferred embodiments recited above and hereinafter which have a glass transition temperature of at least 70° C., more preferably of at least 110° C., even more preferably of at least 125° C. and especially preferably of at least 150° C., determined in accordance with DIN 51005 (2005-08 version).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitting compound and/or a further matrix material. It may preferably be the case that at least one further compound is selected from the group consisting of fluorescent emitters, phosphorescent emitters, emitters that exhibit TADF, host materials, electron transport materials, electron injection materials, hole conductor materials, hole injection materials, electron blocker materials and hole blocker materials, preferably host materials.

The present invention further provides for the use of a compound of the invention in an electronic device, especially in an organic electroluminescent device. It may preferably be the case that the compound of the invention is used in an electronic device as host material, electron transport material, electron injection material, hole conductor material, hole injection material, electron blocker material, hole blocker material.

The present invention still further provides an electronic device comprising at least one compound of the invention. An electronic device in the context of the present invention is a device comprising at least one layer comprising at least one organic compound. This component may also comprise inorganic materials or else layers formed entirely from inorganic materials.

The electronic device is more preferably selected from the group consisting of organic electroluminescent devices (OLEDs, SOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (O-laser), organic plasmon-emitting devices (D. M. Koller et al., Nature Photonics 2008, 1-4); organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, SOLED, PLEDs, LECs, etc.), more preferably organic light-emitting diodes (OLEDs), organic light-emitting diodes based on small molecules (SOLEDs), organic light-emitting diodes based on polymers (PLEDs), especially phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The compound of the invention may be used in different layers, according to the exact structure. Preference is given to an organic electroluminescent device comprising a compound of formula (I) or the above-recited preferred embodiments in an emitting layer as matrix material for phosphorescent emitters or for emitters that exhibit TADF (thermally activated delayed fluorescence), especially for phosphorescent emitters. In addition, the compound of the invention can also be used in an electron transport layer and/or in a hole transport layer and/or in an exciton blocker layer and/or in a hole blocker layer. More preferably, the compound of the invention is used as matrix material for phosphorescent emitters, especially for red-, orange-, green- or yellow-phosphorescing emitters, in an emitting layer, as electron transport or hole blocker material in an electron transport or hole blocker layer, or as a hole transport or electron blocker material in a hole transport or electron blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume, especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume, especially between 5% and 20% by volume of the emitter, based on the overall mixture of emitter and matrix material.

In one embodiment of the invention, the compound of the invention is used here as the sole matrix material (“single host”) for the phosphorescent emitter.

A further embodiment of the present invention is the use of the compound of the invention as matrix material for a phosphorescent emitter in combination with a further matrix material. Suitable matrix materials which can be used in combination with the compounds of the invention are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

It is likewise possible for a further phosphorescent emitter which emits at a shorter wavelength than the actual emitter to be present as co-host in the mixture. Particularly good results are achieved when the emitter used is a red-phosphorescing emitter and the co-host used in combination with the compound of the invention is a yellow-phosphorescing emitter.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680. In this context, it should be emphasized that compounds of the invention have advantageous properties without specific functional groups, for example hole transport groups and/or electron transport groups.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and WO 2018/011186. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

Examples of phosphorescent dopants are listed in the following table:

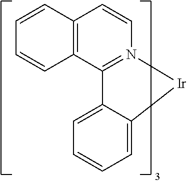

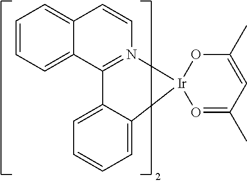

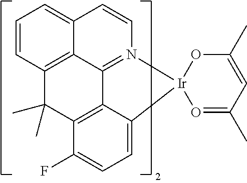

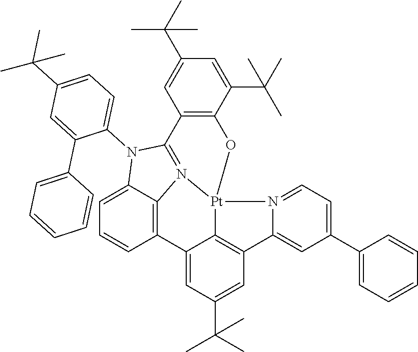

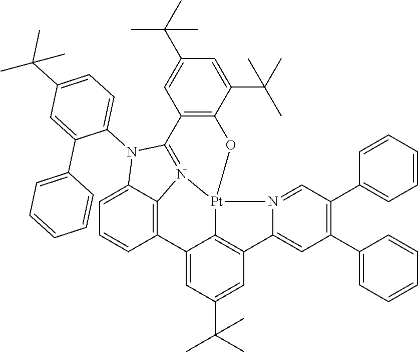

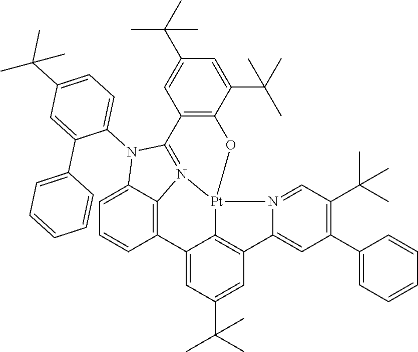

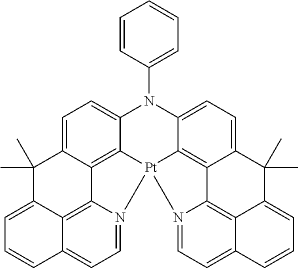

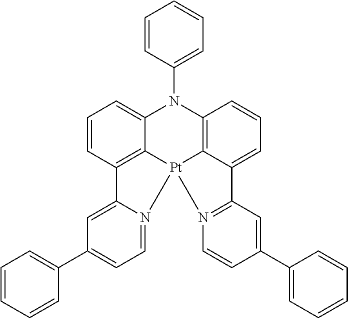

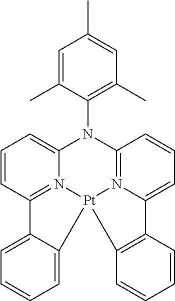

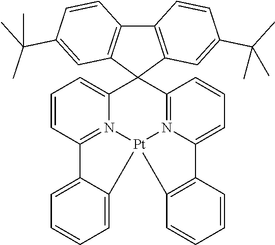

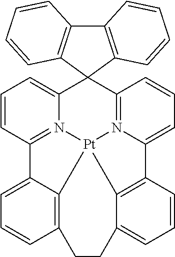

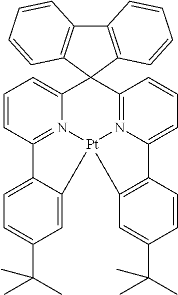

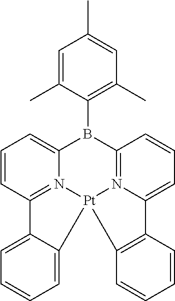

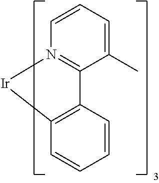

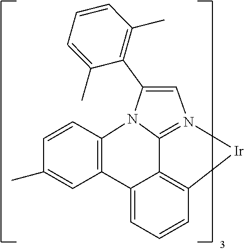

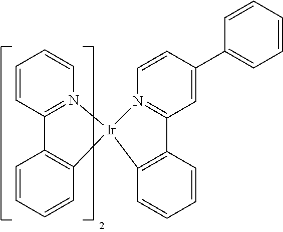

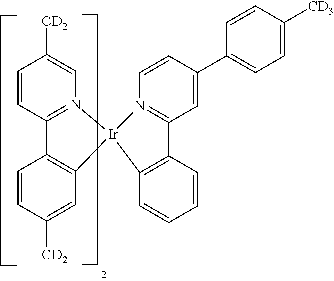

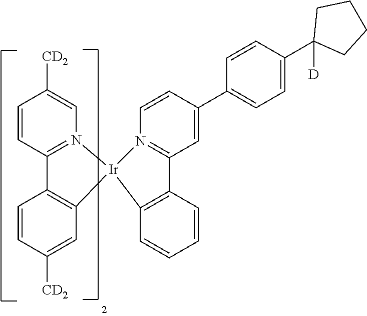

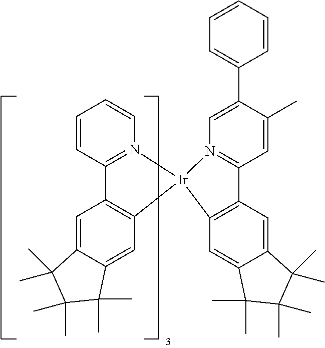

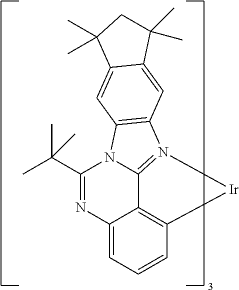

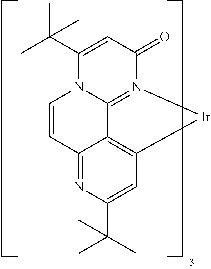

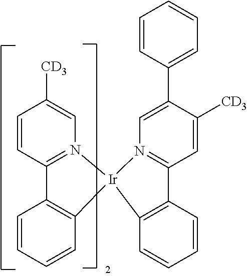

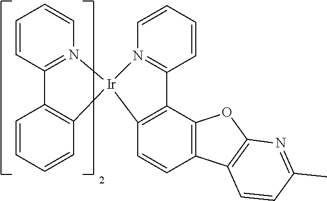

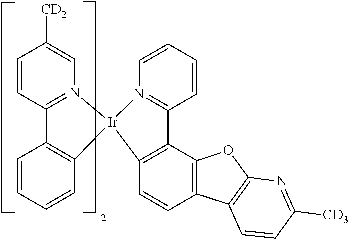

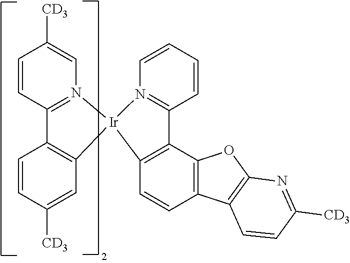

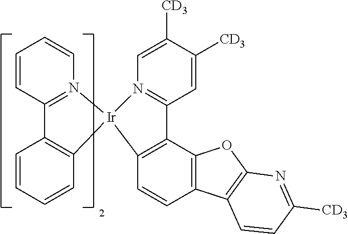

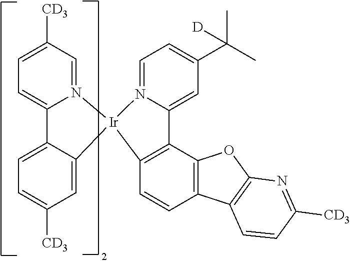

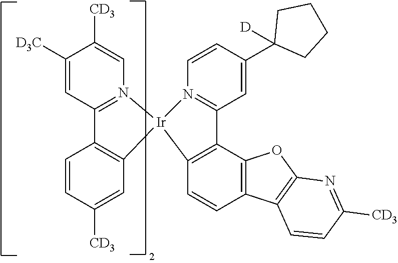

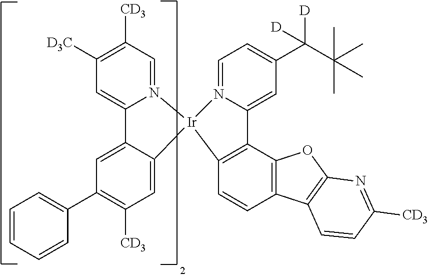

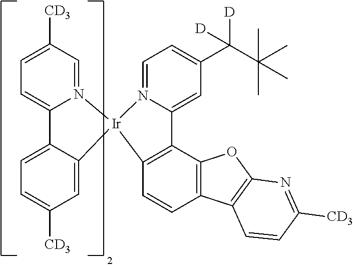

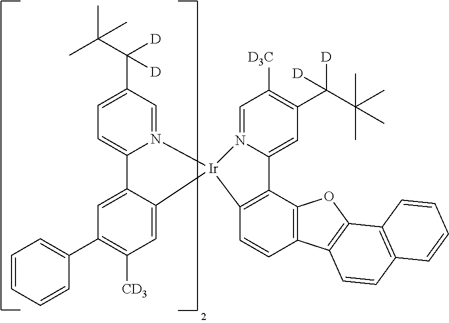

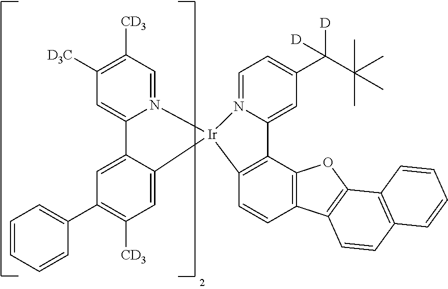

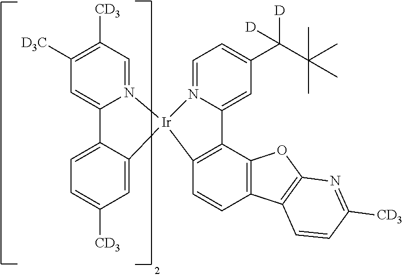

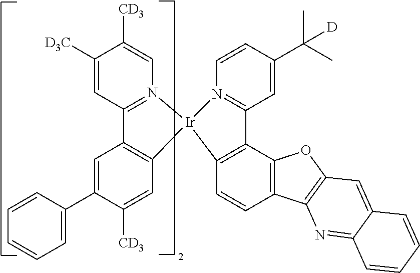

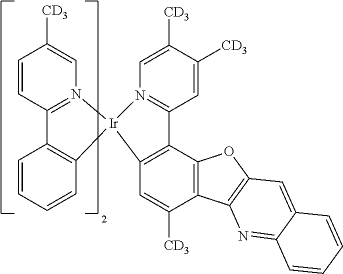

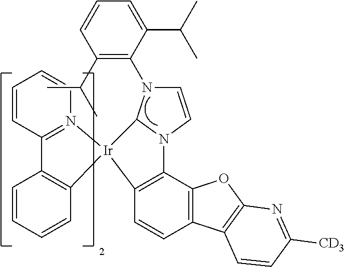

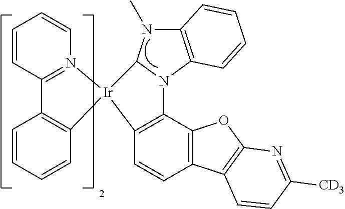

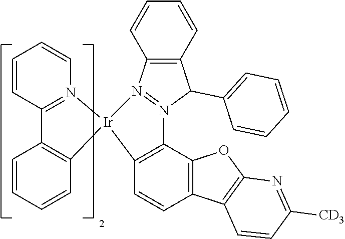

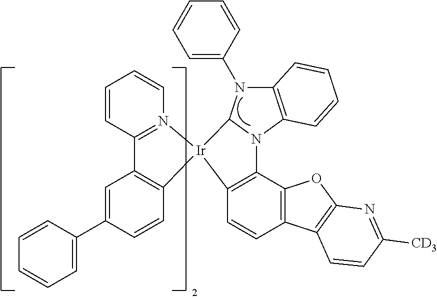

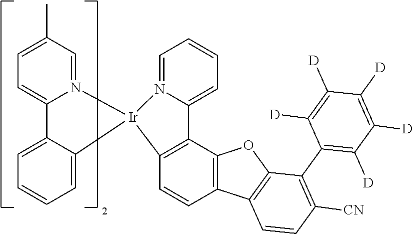

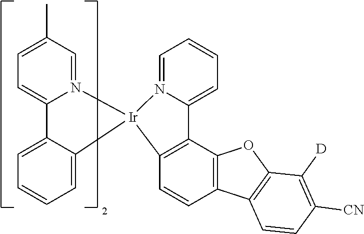

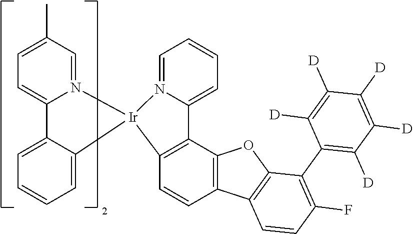

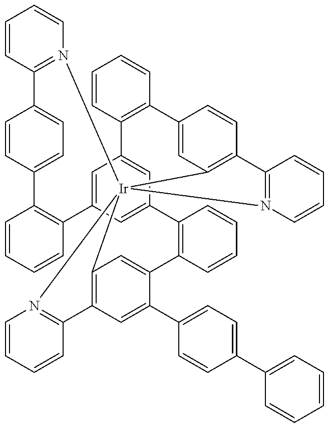

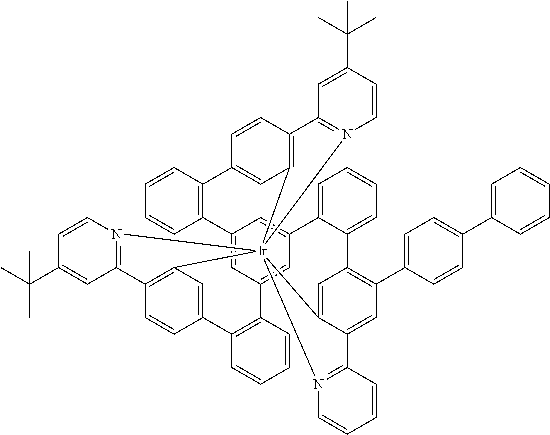

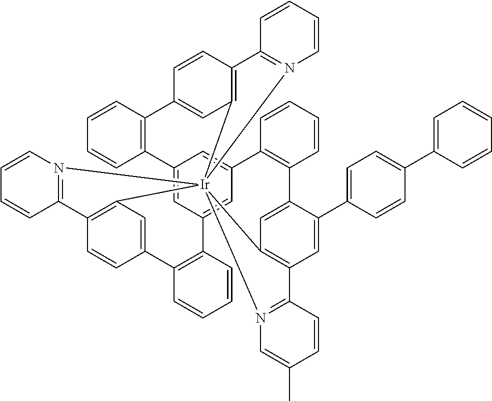

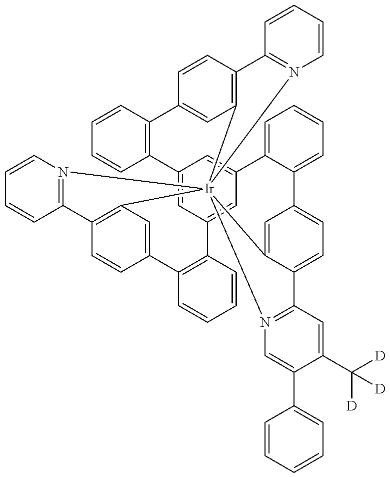

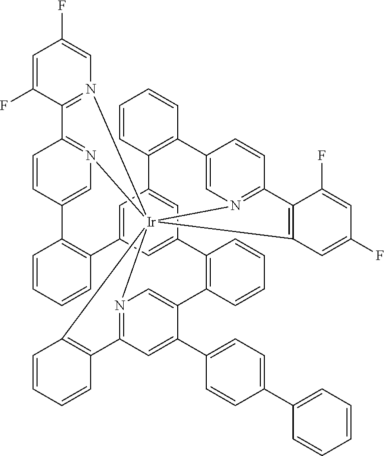

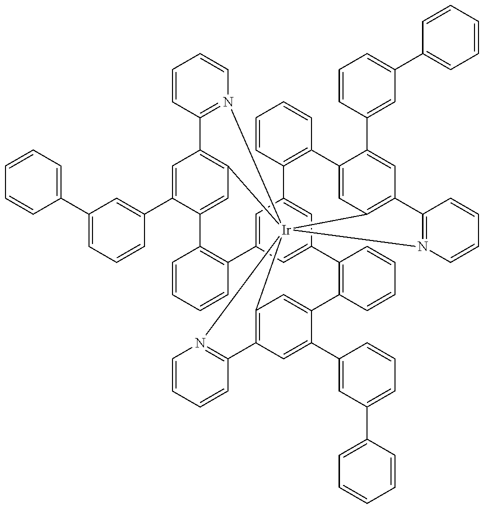

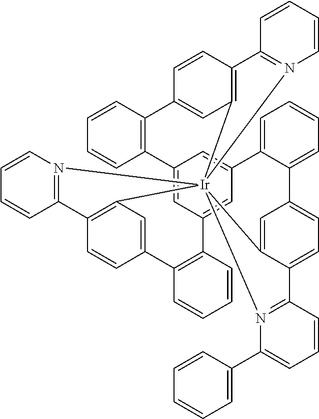

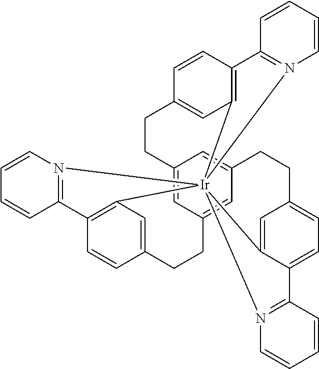

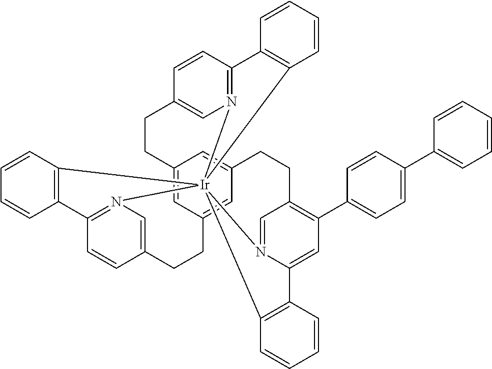

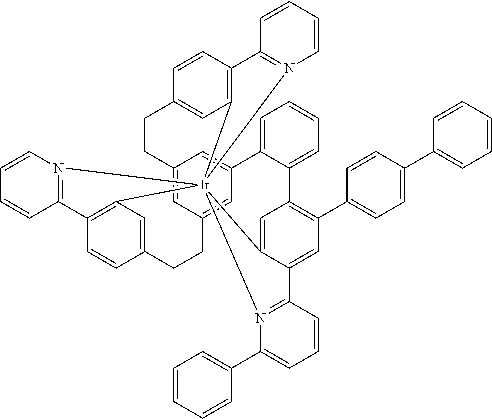

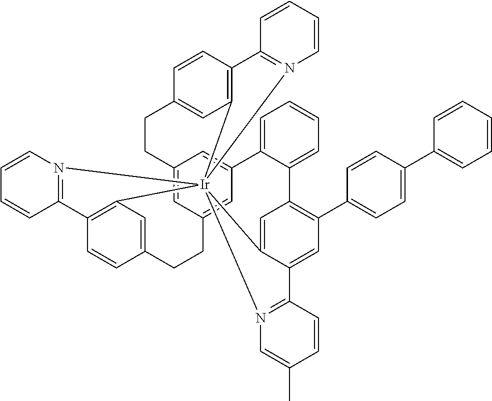

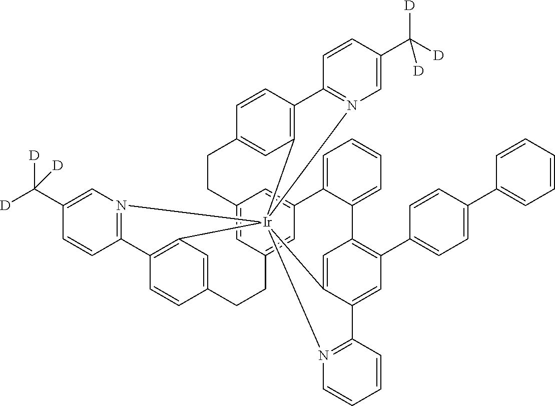

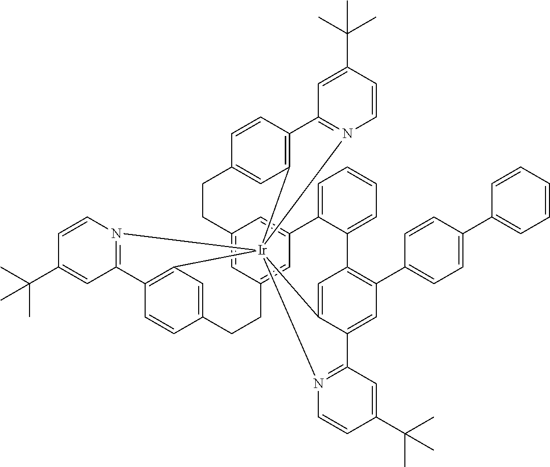

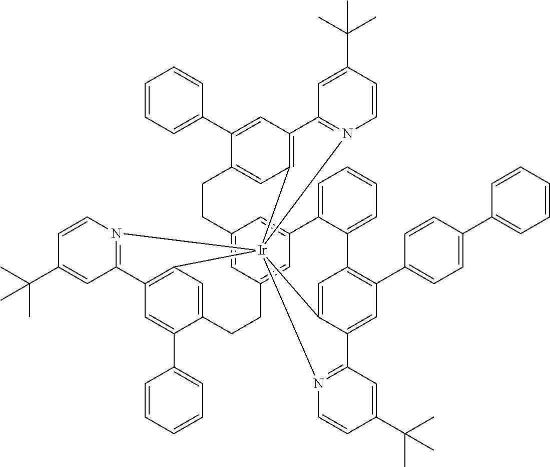

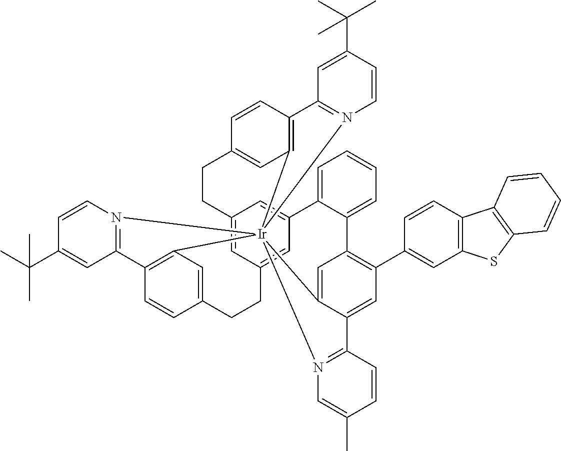

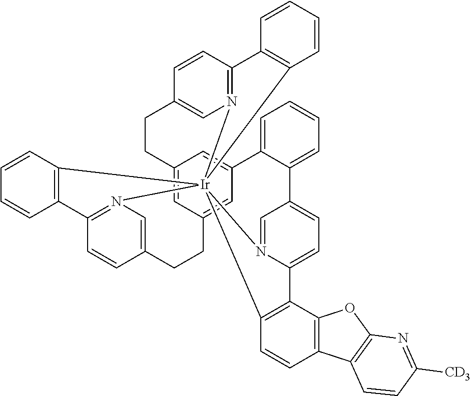

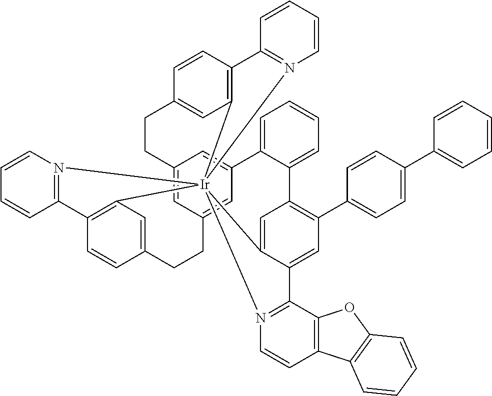

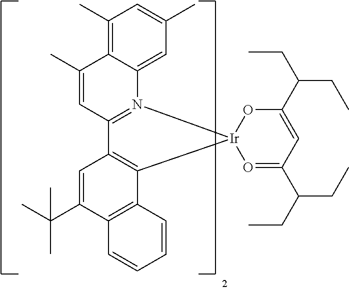

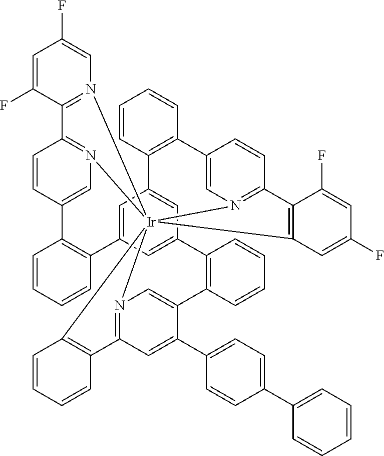

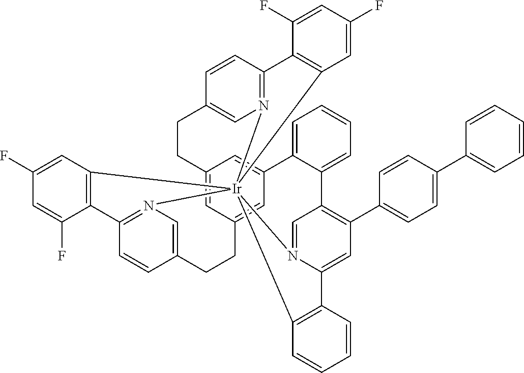

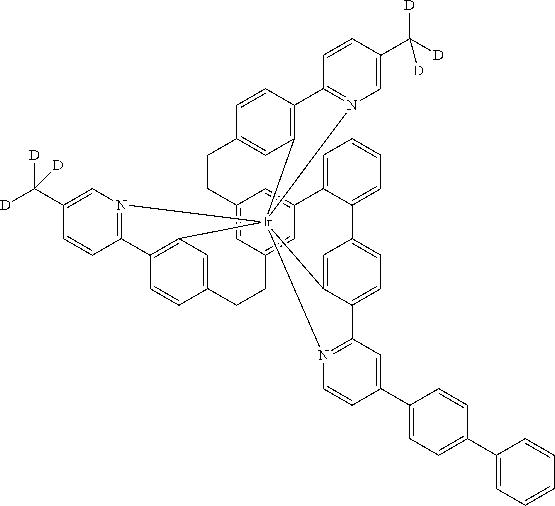

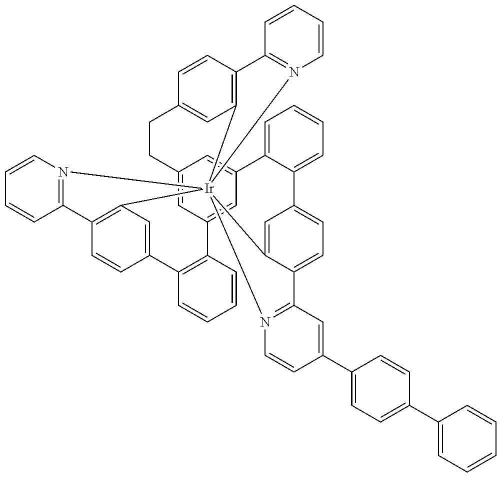

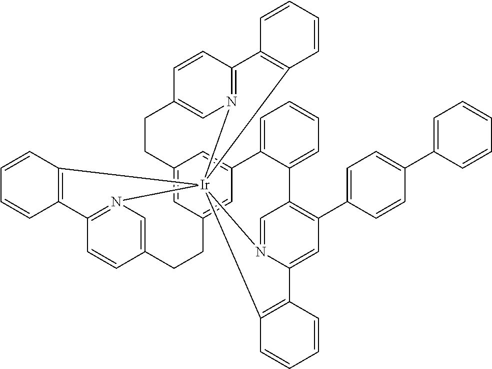

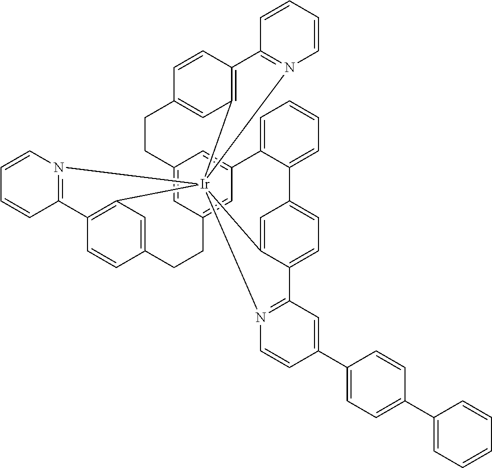

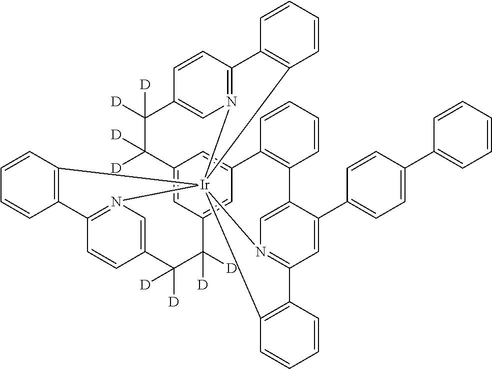

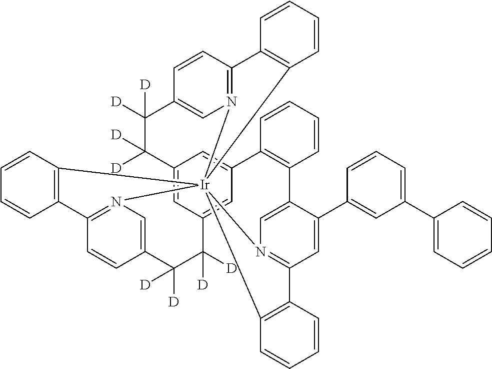

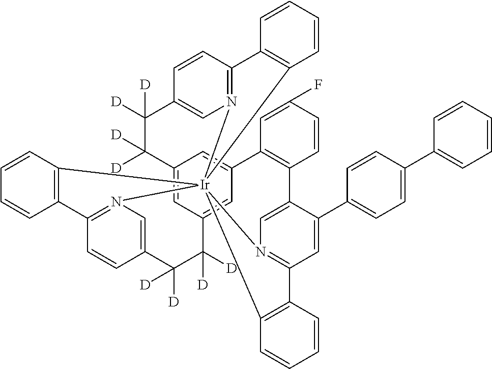

The compounds of the invention are especially also suitable as matrix materials for phosphorescent emitters in organic electroluminescent devices, as described, for example, in WO 98/24271, US 2011/0248247 and US 2012/0223633. In these multicolor display components, an additional blue emission layer is applied by vapor deposition over the full area to all pixels, including those having a color other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (I) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. It is alternatively possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for applying a compound of formula (I) or the preferred embodiments thereof detailed above are novel. The present invention therefore further provides formulations containing at least one solvent and a compound according to formula (I) or the preferred embodiments thereof detailed above.

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

The compounds of the invention and the organic electroluminescent devices of the invention are particularly notable with respect to the prior art for a low refractive index (RI). Furthermore, these compounds and the organic electroluminescent devices obtainable therefrom show an improved lifetime. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. Electronic devices, especially organic electroluminescent devices, comprising compounds of formula (I) or the preferred embodiments recited above and hereinafter, have excellent efficiency, especially as matrix material, as electron-conducting materials or as hole-conducting materials. In this context, compounds of the invention having structures of formula (I) or the preferred embodiments recited above and hereinafter bring about a low operating voltage when used in electronic devices.
2. Electronic devices, especially organic electroluminescent devices, comprising compounds of formula (I) or the preferred embodiments recited above and hereinafter, have a very good lifetime, especially as matrix material, as electron-conducting materials or as hole-conducting materials. In this context, these compounds especially bring about low roll-off, i.e. a small drop in power efficiency of the device at high luminances.
3. The inventive compounds of formula (I) or the preferred embodiments recited above and hereinafter exhibit very high stability and lifetime.
4. Electronic devices, especially organic electroluminescent devices, comprising compounds of formula (I) or the preferred embodiments recited above and hereinafter, have very low refractive indices, especially as matrix material, as electron-conducting materials or as hole-conducting materials.
5. With compounds of formula (I) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
6. Compounds of formula (I) or the preferred embodiments recited above and hereinafter have excellent glass film formation.
7. Compounds of formula (I) or the preferred embodiments recited above and hereinafter form very good films from solutions.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The metal complexes are additionally handled with exclusion of light or under yellow light. The solvents and reagents can be purchased, for example, from Sigma-ALDRICH or ABCR. The respective FIGURES in square brackets or the numbers quoted for individual compounds relate to the CAS numbers of the compounds known from the literature. In the case of compounds that can have multiple enantiomeric, diastereomeric or tautomeric forms, one form is shown in a representative manner.

Synthons LS Known from the Literature:

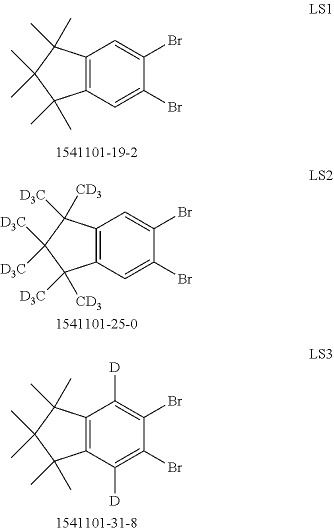 [SMILES: CC1(C)c2cc(Br)c(Br)cc2C(C)(C)C1(C)C ; [2H]C([2H])([2H])C1(C([2H])([2H])[2H])c2cc(Br)c(Br)cc2C(C([2H])([2H])[2H])(C([2H])([2H])[2H])C1(C([2H])([2H])[2H])C([2H])([2H])[2H] ; [2H]c1c(Br)c(Br)c([2H])c2c1C(C)(C)C(C)(C)C2(C)C]

### a) Synthesis of Synthons S:

### Example S1

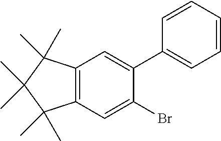 [SMILES: CC1(C)c2cc(Br)c(-c3ccccc3)cc2C(C)(C)C1(C)C]

Procedure analogous to C.-G-Dong et al, Synlett, 2009, No. 7, 1081, Table 2, Entry 3. Starting materials: 36.0 g (100 mmol) of LS1, 12.0 g (100 mmol) of phenylboronic acid, 3% Pd(PPh₃)₄+9% Ph₃P, toluene 80° C., 16 h. Yield: 28.7 g (80 mmol) 80%; purity: about 97% by 1H NMR.

The following compounds can be prepared analogously:

| Ex. | Reactants | Product | Yield |
|---|---|---|---|
| S2 | LS1 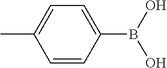 5720-05-8 | 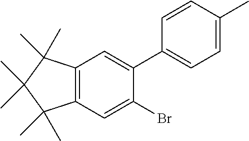 | 77% |
| S3 | LS1 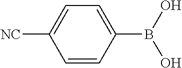 [SMILES: N#Cc1ccc(B(O)O)cc1] 12647-14-6 | 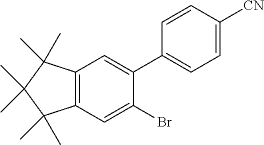 [SMILES: CC1(C)c2cc(Br)c(-c3ccc(C#N)cc3)cc2C(C)(C)C1(C)C] | 82% |
| S4 | LS1 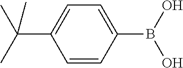 123324-71-0 | 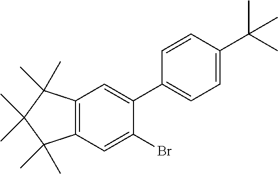 [SMILES: CC(C)(C)c1ccc(-c2cc3c(cc2Br)C(C)(C)C(C)(C)C3(C)C)cc1] | 80% |
| S5 | LS1 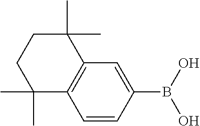 [SMILES: CC1(C)CCC(C)(C)c2cc(B(O)O)ccc21] 169126-63-0 | 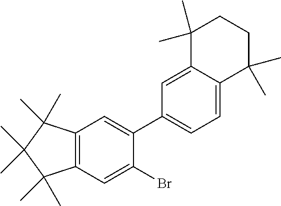 | 75% |
| S6 | LS1 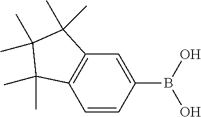 [SMILES: CC1(C)c2ccc(B(O)O)cc2C(C)(C)C1(C)C] 1562418-16-9 | 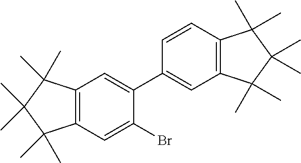 | 73% |
| S7 | LS1 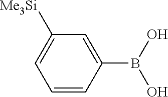 [SMILES: C[Si](C)(C)c1cccc(B(O)O)c1] 177171-16-3 | 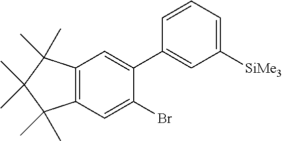 [SMILES: CC1(C)c2cc(Br)c(-c3cccc([Si](C)(C)C)c3)cc2C(C)(C)C1(C)C] | 48% |
| S8 | LS2 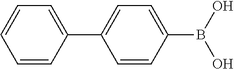 [SMILES: OB(O)c1ccc(-c2ccccc2)cc1] 5122-94-1 | 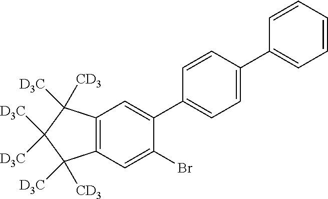 [SMILES: [2H]C([2H])([2H])C1(C([2H])([2H])[2H])c2cc(Br)c(-c3ccc(-c4ccccc4)cc3)cc2C(C([2H])([2H])[2H])(C([2H])([2H])[2H])C1(C([2H])([2H])[2H])C([2H])([2H])[2H]] | 83% |
| S9 | LS2 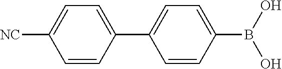 [SMILES: N#Cc1ccc(-c2ccc(B(O)O)cc2)cc1] 406482-73-3 | 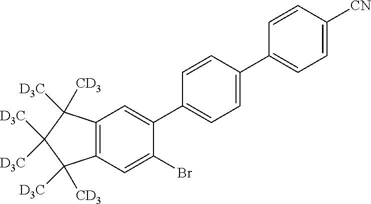 [SMILES: [2H]C([2H])([2H])C1(C([2H])([2H])[2H])c2cc(Br)c(-c3ccc(-c4ccc(C#N)cc4)cc3)cc2C(C([2H])([2H])[2H])(C([2H])([2H])[2H])C1(C([2H])([2H])[2H])C([2H])([2H])[2H]] | 80% |
| S10 | LS1 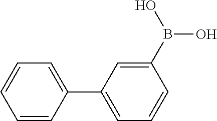 [SMILES: OB(O)c1cccc(-c2ccccc2)c1] 5122-95-2 | 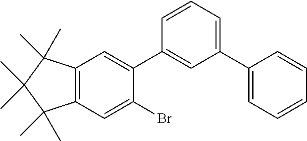 [SMILES: CC1(C)c2cc(Br)c(-c3cccc(-c4ccccc4)c3)cc2C(C)(C)C1(C)C] | 79% |
| S11 | LS3 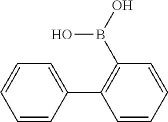 [SMILES: OB(O)c1ccccc1-c1ccccc1] 914675-52-8 | 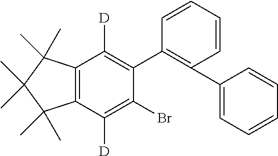 [SMILES: [2H]c1c(Br)c(-c2ccccc2-c2ccccc2)c([2H])c2c1C(C)(C)C(C)(C)C2(C)C] | 76% |
| S12 | LS1 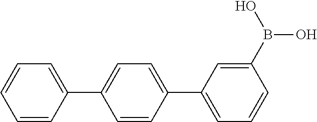 [SMILES: OB(O)c1cccc(-c2ccc(-c3ccccc3)cc2)c1] 881911-81-5 | 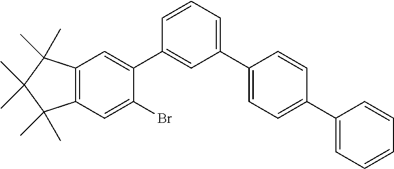 [SMILES: CC1(C)c2cc(Br)c(-c3cccc(-c4ccc(-c5ccccc5)cc4)c3)cc2C(C)(C)C1(C)C] | 82% |
| S13 | LS1 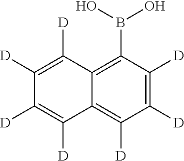 [SMILES: [2H]c1c([2H])c([2H])c2c(B(O)O)c([2H])c([2H])c([2H])c2c1[2H]] 1280709-91-2 | 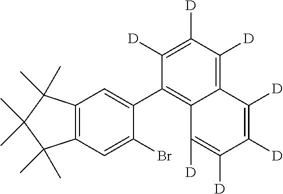 [SMILES: [2H]c1c([2H])c([2H])c2c(-c3cc4c(cc3Br)C(C)(C)C(C)(C)C4(C)C)c([2H])c([2H])c([2H])c2c1[2H]] | 78 |
| S14 | LS1 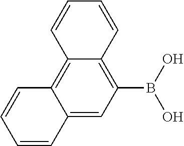 [SMILES: OB(O)c1cc2ccccc2c2ccccc12] 68572-87-2 | 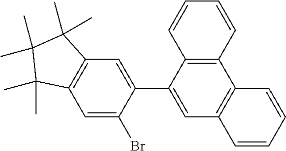 [SMILES: CC1(C)c2cc(Br)c(-c3cc4ccccc4c4ccccc34)cc2C(C)(C)C1(C)C] | 76% |
| S15 | LS1 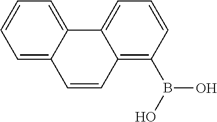 [SMILES: OB(O)c1cccc2c1ccc1ccccc12] 146746-63-6 | 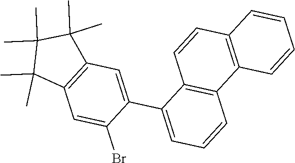 [SMILES: CC1(C)c2cc(Br)c(-c3cccc4c3ccc3ccccc34)cc2C(C)(C)C1(C)C] | 72% |
| S16 | LS1 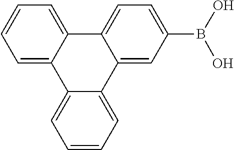 [SMILES: OB(O)c1ccc2c3ccccc3c3ccccc3c2c1] 654664-63-8 | 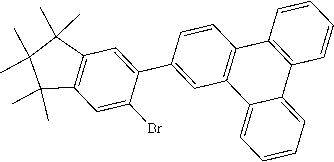 [SMILES: CC1(C)c2cc(Br)c(-c3ccc4c5ccccc5c5ccccc5c4c3)cc2C(C)(C)C1(C)C] | 84% |
| S17 | LS1 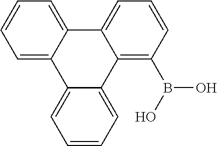 [SMILES: OB(O)c1cccc2c3ccccc3c3ccccc3c12] 1430392-46-3 | 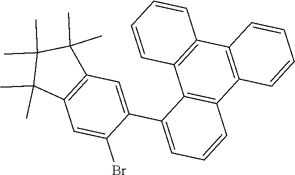 [SMILES: CC1(C)c2cc(Br)c(-c3cccc4c5ccccc5c5ccccc5c34)cc2C(C)(C)C1(C)C] | 69% |
| S18 | LS1 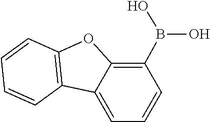 [SMILES: OB(O)c1cccc2c1oc1ccccc12] 100124-06-9 | 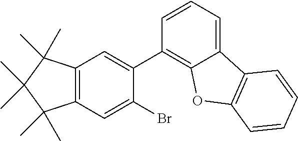 [SMILES: CC1(C)c2cc(Br)c(-c3cccc4c3oc3ccccc34)cc2C(C)(C)C1(C)C] | 76% |
| S19 | LS1 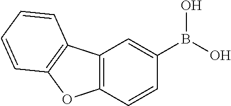 [SMILES: OB(O)c1ccc2oc3ccccc3c2c1] 402936-15-6 | 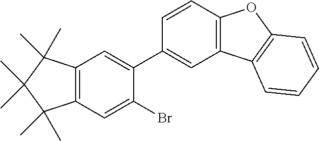 [SMILES: CC1(C)c2cc(Br)c(-c3ccc4oc5ccccc5c4c3)cc2C(C)(C)C1(C)C] | 81% |
| S20 | LS1 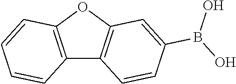 [SMILES: OB(O)c1ccc2c(c1)oc1ccccc12] 395087-89-5 | 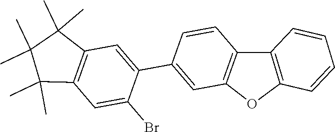 [SMILES: CC1(C)c2cc(Br)c(-c3ccc4c(c3)oc3ccccc34)cc2C(C)(C)C1(C)C] | 85% |
| S21 | LS1 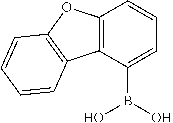 [SMILES: OB(O)c1cccc2oc3ccccc3c12] 162607-19-4 | 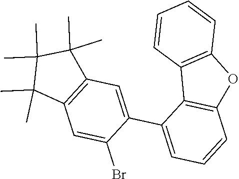 [SMILES: CC1(C)c2cc(Br)c(-c3cccc4oc5ccccc5c34)cc2C(C)(C)C1(C)C] | 68% |
| S22 | LS1 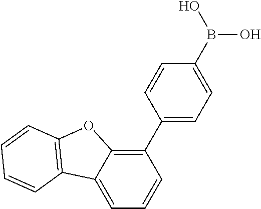 [SMILES: OB(O)c1ccc(-c2cccc3c2oc2ccccc23)cc1] 796071-96-0 | 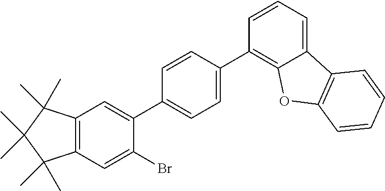 [SMILES: CC1(C)c2cc(Br)c(-c3ccc(-c4cccc5c4oc4ccccc45)cc3)cc2C(C)(C)C1(C)C] | 84% |
| S23 | LS1 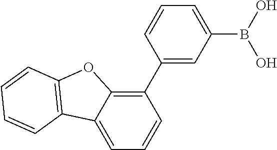 [SMILES: OB(O)c1cccc(-c2cccc3c2oc2ccccc23)c1] 1271726-52-3 | 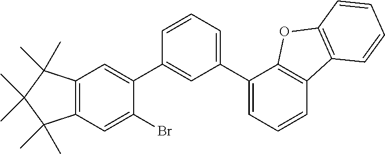 [SMILES: CC1(C)c2cc(Br)c(-c3cccc(-c4cccc5c4oc4ccccc45)c3)cc2C(C)(C)C1(C)C] | 80% |
| S24 | LS1 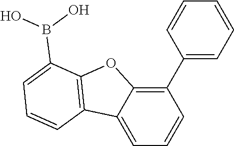 [SMILES: OB(O)c1cccc2c1oc1c(-c3ccccc3)cccc12] 1010068-85-5 | 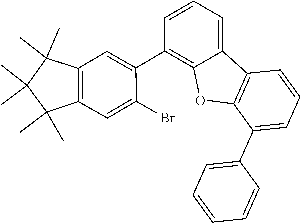 [SMILES: CC1(C)c2cc(Br)c(-c3cccc4c3oc3c(-c5ccccc5)cccc34)cc2C(C)(C)C1(C)C] | 75% |
| S25 | LS1 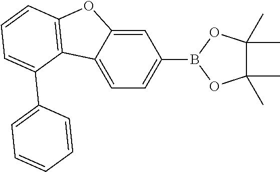 [SMILES: CC1(C)OB(c2ccc3c(c2)oc2cccc(-c4ccccc4)c23)OC1(C)C] 2360830-98-2 | 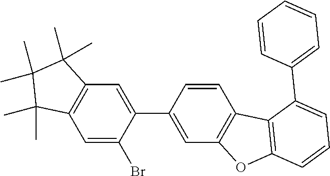 [SMILES: CC1(C)c2cc(Br)c(-c3ccc4c(c3)oc3cccc(-c5ccccc5)c34)cc2C(C)(C)C1(C)C] | 79% |
| S26 | LS1 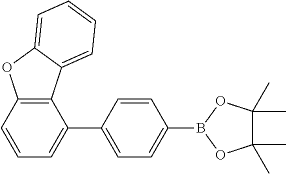 [SMILES: CC1(C)OB(c2ccc(-c3cccc4oc5ccccc5c34)cc2)OC1(C)C] 2186731-24-6 | 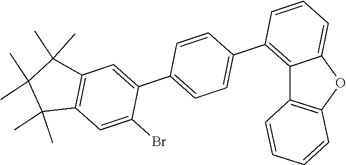 [SMILES: CC1(C)c2cc(Br)c(-c3ccc(-c4cccc5oc6ccccc6c45)cc3)cc2C(C)(C)C1(C)C] | 83% |
| S27 | LS1 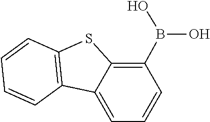 [SMILES: OB(O)c1cccc2c1sc1ccccc12] 108847-20-7 | 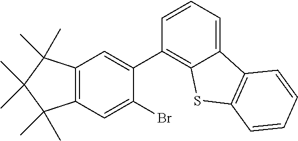 [SMILES: CC1(C)c2cc(Br)c(-c3cccc4c3sc3ccccc34)cc2C(C)(C)C1(C)C] | 70% |
| S28 | LS1 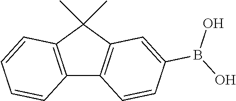 [SMILES: CC1(C)c2ccccc2-c2ccc(B(O)O)cc21] 333432-28-3 | 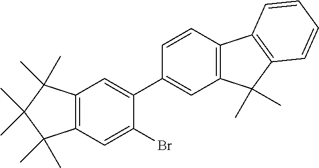 [SMILES: CC1(C)c2ccccc2-c2ccc(-c3cc4c(cc3Br)C(C)(C)C(C)(C)C4(C)C)cc21] | 79% |
| S29 | LS1 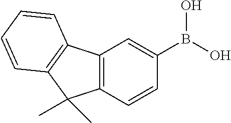 [SMILES: CC1(C)c2ccccc2-c2cc(B(O)O)ccc21] 1251773-34-8 | 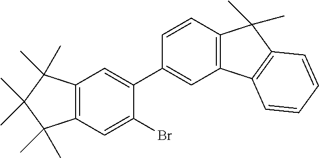 [SMILES: CC1(C)c2ccccc2-c2cc(-c3cc4c(cc3Br)C(C)(C)C(C)(C)C4(C)C)ccc21] | 80% |
| S30 | LS1 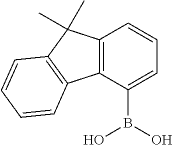 [SMILES: CC1(C)c2ccccc2-c2c(B(O)O)cccc21] 1246022-50-3 | 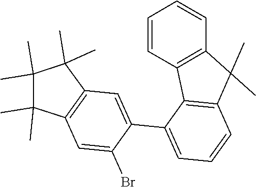 | 73% |
| S31 | LS1 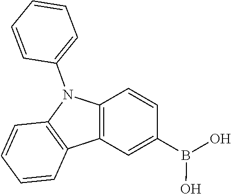 [SMILES: OB(O)c1ccc2c(c1)c1ccccc1n2-c1ccccc1] 854952-58-2 | 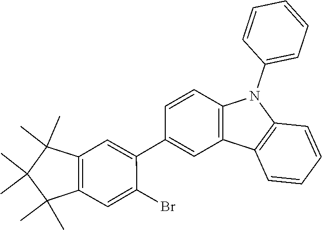 [SMILES: CC1(C)c2cc(Br)c(-c3ccc4c(c3)c3ccccc3n4-c3ccccc3)cc2C(C)(C)C1(C)C] | 79% |
| S32 | LS1 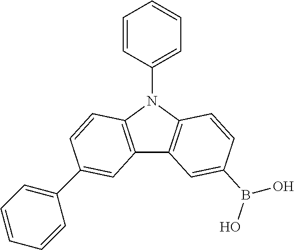 [SMILES: OB(O)c1ccc2c(c1)c1cc(-c3ccccc3)ccc1n2-c1ccccc1] 81359833-28-5 | 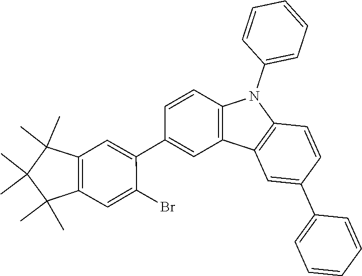 [SMILES: CC1(C)c2cc(Br)c(-c3ccc4c(c3)c3cc(-c5ccccc5)ccc3n4-c3ccccc3)cc2C(C)(C)C1(C)C] | 84% |
| S33 | LS1 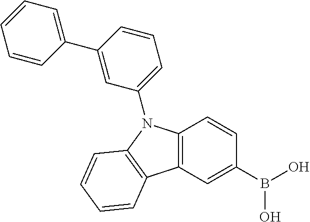 [SMILES: OB(O)c1ccc2c(c1)c1ccccc1n2-c1cccc(-c2ccccc2)c1] 1416814-68-0 | 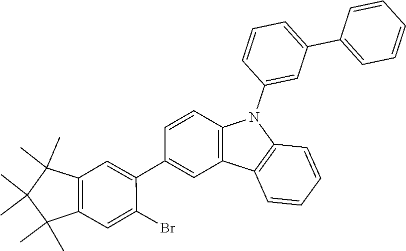 [SMILES: CC1(C)c2cc(Br)c(-c3ccc4c(c3)c3ccccc3n4-c3cccc(-c4ccccc4)c3)cc2C(C)(C)C1(C)C] | 80% |
| S34 | LS1 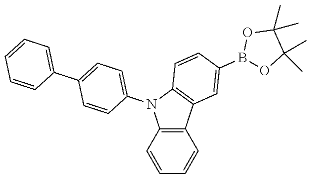 [SMILES: CC1(C)OB(c2ccc3c(c2)c2ccccc2n3-c2ccc(-c3ccccc3)cc2)OC1(C)C] 1391729-66-0 | 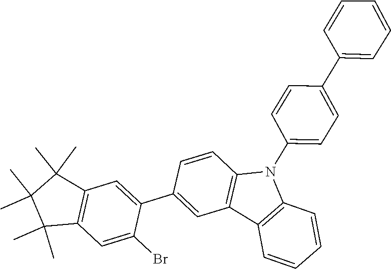 [SMILES: CC1(C)c2cc(Br)c(-c3ccc4c(c3)c3ccccc3n4-c3ccc(-c4ccccc4)cc3)cc2C(C)(C)C1(C)C] | 83% |
| S35 | LS1 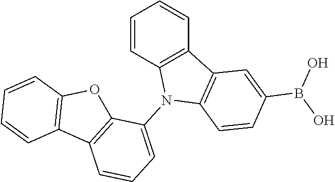 [SMILES: OB(O)c1ccc2c(c1)c1ccccc1n2-c1cccc2c1oc1ccccc12] 547397-15-8 | 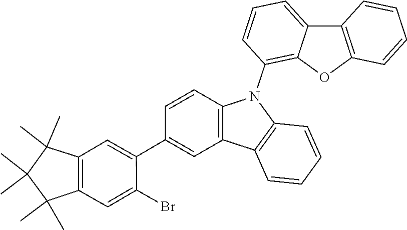 [SMILES: CC1(C)c2cc(Br)c(-c3ccc4c(c3)c3ccccc3n4-c3cccc4c3oc3ccccc34)cc2C(C)(C)C1(C)C] | 79% |
| S36 | LS1 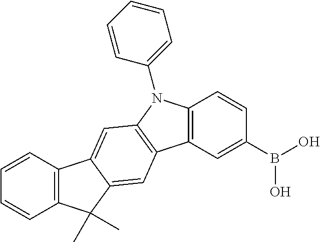 [SMILES: CC1(C)c2ccccc2-c2cc3c(cc21)c1cc(B(O)O)ccc1n3-c1ccccc1] 1373359-70-6 | 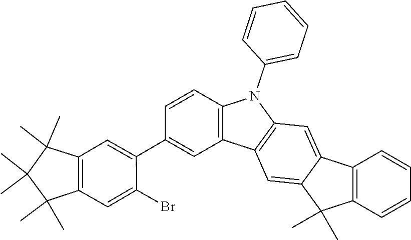 [SMILES: CC1(C)c2ccccc2-c2cc3c(cc21)c1cc(-c2cc4c(cc2Br)C(C)(C)C(C)(C)C4(C)C)ccc1n3-c1ccccc1] | 79% |
| S37 | LS1 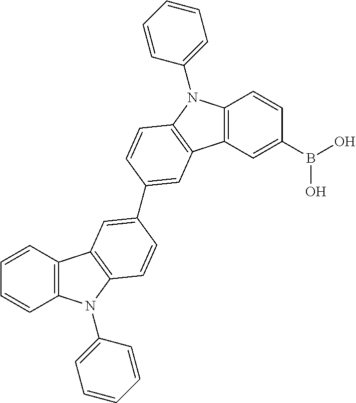 [SMILES: OB(O)c1ccc2c(c1)c1cc(-c3ccc4c(c3)c3ccccc3n4-c3ccccc3)ccc1n2-c1ccccc1] 918137-86-7 | 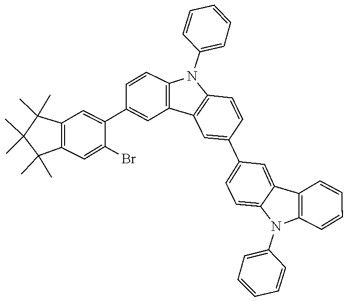 [SMILES: CC1(C)c2cc(Br)c(-c3ccc4c(c3)c3cc(-c5ccc6c(c5)c5ccccc5n6-c5ccccc5)ccc3n4-c3ccccc3)cc2C(C)(C)C1(C)C] | 82% |
| S38 | LS3 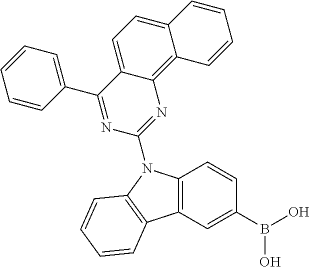 [SMILES: OB(O)c1ccc2c(c1)c1ccccc1n2-c1nc(-c2ccccc2)c2ccc3ccccc3c2n1] 1813537-15-3 | 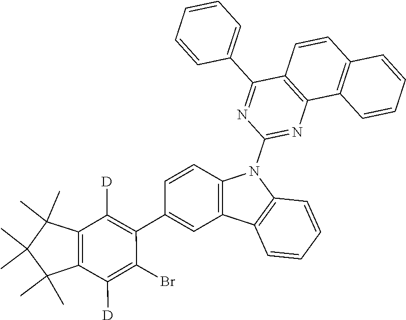 [SMILES: [2H]c1c(Br)c(-c2ccc3c(c2)c2ccccc2n3-c2nc(-c3ccccc3)c3ccc4ccccc4c3n2)c([2H])c2c1C(C)(C)C(C)(C)C2(C)C] | 80% |
| S39 | LS1 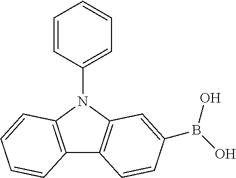 [SMILES: OB(O)c1ccc2c3ccccc3n(-c3ccccc3)c2c1] 1001911-63-2 | 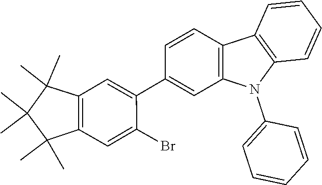 [SMILES: CC1(C)c2cc(Br)c(-c3ccc4c5ccccc5n(-c5ccccc5)c4c3)cc2C(C)(C)C1(C)C] | 82% |
| S40 | LS1 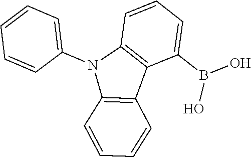 [SMILES: OB(O)c1cccc2c1c1ccccc1n2-c1ccccc1] 1370555-65-9 | 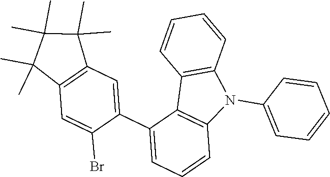 [SMILES: CC1(C)c2cc(Br)c(-c3cccc4c3c3ccccc3n4-c3ccccc3)cc2C(C)(C)C1(C)C] | 71% |
| S41 | LS1 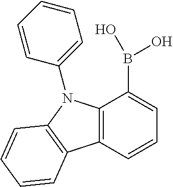 [SMILES: OB(O)c1cccc2c3ccccc3n(-c3ccccc3)c12] 1333002-41-7 | 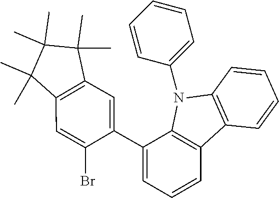 [SMILES: CC1(C)c2cc(Br)c(-c3cccc4c5ccccc5n(-c5ccccc5)c34)cc2C(C)(C)C1(C)C] | 55% |
| S42 | LS1 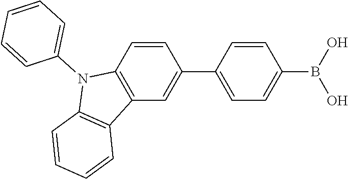 [SMILES: OB(O)c1ccc(-c2ccc3c(c2)c2ccccc2n3-c2ccccc2)cc1] 1240963-55-6 | 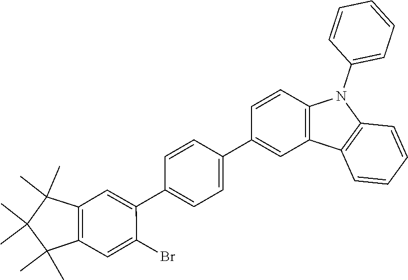 [SMILES: CC1(C)c2cc(Br)c(-c3ccc(-c4ccc5c(c4)c4ccccc4n5-c4ccccc4)cc3)cc2C(C)(C)C1(C)C] | 81% |
| S43 | LS1 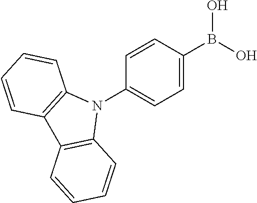 [SMILES: OB(O)c1ccc(-n2c3ccccc3c3ccccc32)cc1] 419536-33-7 | 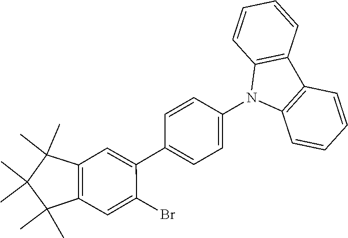 [SMILES: CC1(C)c2cc(Br)c(-c3ccc(-n4c5ccccc5c5ccccc54)cc3)cc2C(C)(C)C1(C)C] | 80% |
| S44 | LS1 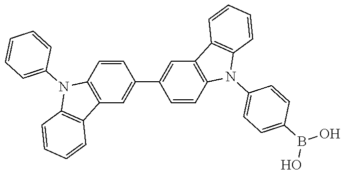 [SMILES: OB(O)c1ccc(-n2c3ccccc3c3cc(-c4ccc5c(c4)c4ccccc4n5-c4ccccc4)ccc32)cc1] 1398394-82-5 | 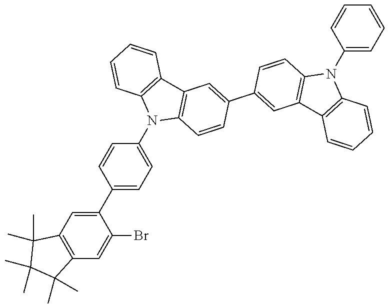 [SMILES: CC1(C)c2cc(Br)c(-c3ccc(-n4c5ccccc5c5cc(-c6ccc7c(c6)c6ccccc6n7-c6ccccc6)ccc54)cc3)cc2C(C)(C)C1(C)C] | 77% |
| S45 | LS1 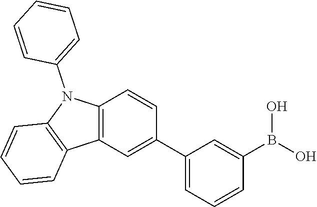 [SMILES: OB(O)c1cccc(-c2ccc3c(c2)c2ccccc2n3-c2ccccc2)c1] 854952-60-6 | 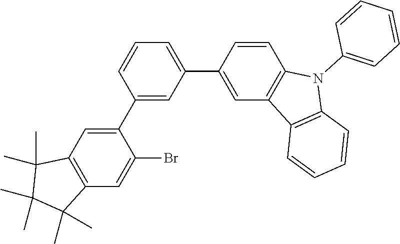 [SMILES: CC1(C)c2cc(Br)c(-c3cccc(-c4ccc5c(c4)c4ccccc4n5-c4ccccc4)c3)cc2C(C)(C)C1(C)C] | 78% |
| S46 | LS1 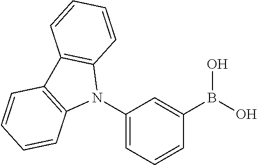 [SMILES: OB(O)c1cccc(-n2c3ccccc3c3ccccc32)c1] 864377-33-3 | 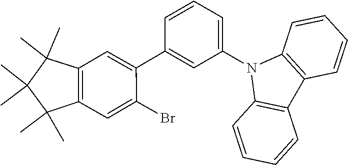 [SMILES: CC1(C)c2cc(Br)c(-c3cccc(-n4c5ccccc5c5ccccc54)c3)cc2C(C)(C)C1(C)C] | 78% |
| S47 | LS1 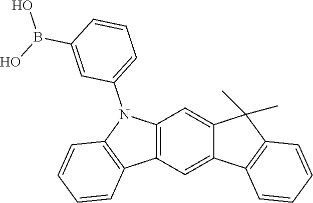 [SMILES: CC1(C)c2ccccc2-c2cc3c4ccccc4n(-c4cccc(B(O)O)c4)c3cc21] 1369587-64-3 | 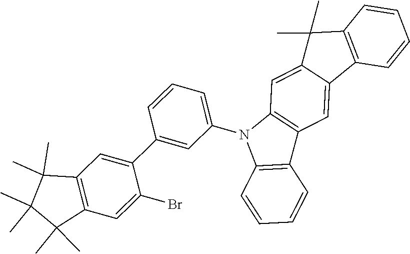 [SMILES: CC1(C)c2ccccc2-c2cc3c4ccccc4n(-c4cccc(-c5cc6c(cc5Br)C(C)(C)C(C)(C)C6(C)C)c4)c3cc21] | 79% |
| S48 | LS1 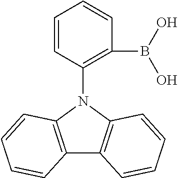 [SMILES: OB(O)c1ccccc1-n1c2ccccc2c2ccccc21] 1189047-28-6 | 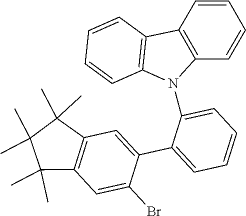 [SMILES: CC1(C)c2cc(Br)c(-c3ccccc3-n3c4ccccc4c4ccccc43)cc2C(C)(C)C1(C)C] | 68% |
| S49 | LS1 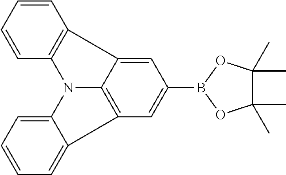 [SMILES: CC1(C)OB(c2cc3c4ccccc4n4c5ccccc5c(c2)c34)OC1(C)C] 13639369-44-7 | 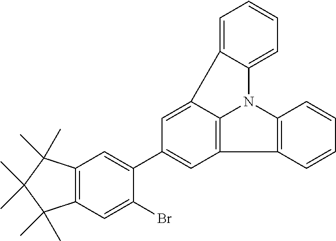 [SMILES: CC1(C)c2cc(Br)c(-c3cc4c5ccccc5n5c6ccccc6c(c3)c45)cc2C(C)(C)C1(C)C] | 84% |
| S50 | LS1 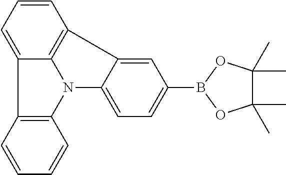 [SMILES: CC1(C)OB(c2ccc3c(c2)c2cccc4c5ccccc5n3c42)OC1(C)C] 1454807-26-1 | 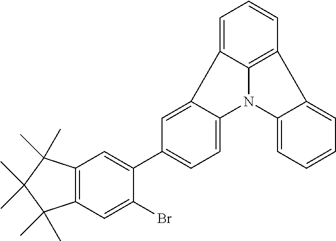 [SMILES: CC1(C)c2cc(Br)c(-c3ccc4c(c3)c3cccc5c6ccccc6n4c53)cc2C(C)(C)C1(C)C] | 81% |
| S51 | LS1 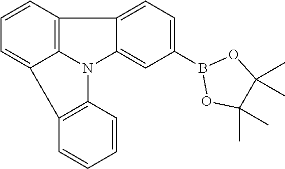 [SMILES: CC1(C)OB(c2ccc3c4cccc5c6ccccc6n(c3c2)c54)OC1(C)C] 2068731-68-8 | 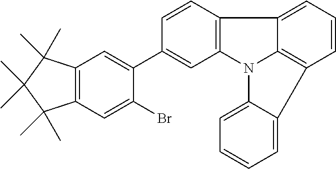 [SMILES: CC1(C)c2cc(Br)c(-c3ccc4c5cccc6c7ccccc7n(c4c3)c65)cc2C(C)(C)C1(C)C] | 83% |
| S52 | LS1 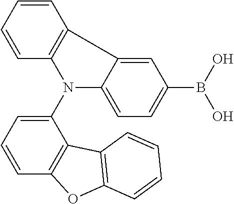 [SMILES: OB(O)c1ccc2c(c1)c1ccccc1n2-c1cccc2oc3ccccc3c12] 2413352-33-5 | 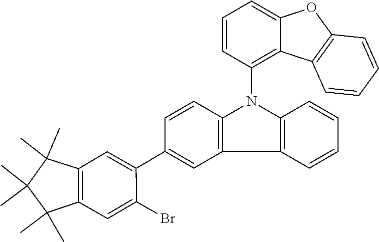 [SMILES: CC1(C)c2cc(Br)c(-c3ccc4c(c3)c3ccccc3n4-c3cccc4oc5ccccc5c34)cc2C(C)(C)C1(C)C] | 79% |
| S53 | LS1 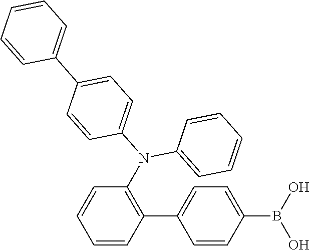 [SMILES: OB(O)c1ccc(-c2ccccc2N(c2ccccc2)c2ccc(-c3ccccc3)cc2)cc1] 1776937-60-0 | 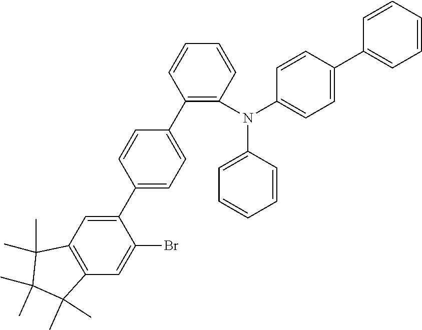 [SMILES: CC1(C)c2cc(Br)c(-c3ccc(-c4ccccc4N(c4ccccc4)c4ccc(-c5ccccc5)cc4)cc3)cc2C(C)(C)C1(C)C] | 74% |
| S54 | LS1 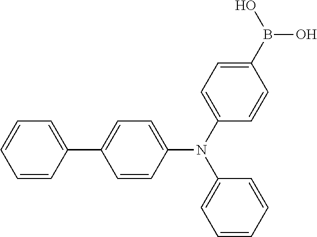 [SMILES: OB(O)c1ccc(N(c2ccccc2)c2ccc(-c3ccccc3)cc2)cc1] 1084334-86-0 | 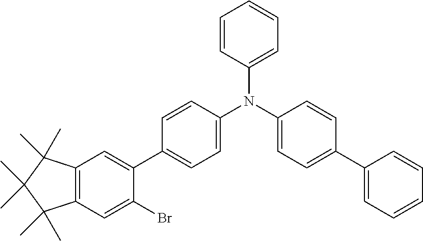 [SMILES: CC1(C)c2cc(Br)c(-c3ccc(N(c4ccccc4)c4ccc(-c5ccccc5)cc4)cc3)cc2C(C)(C)C1(C)C] | 81% |
| S55 | LS1 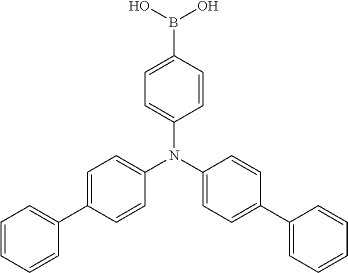 [SMILES: OB(O)c1ccc(N(c2ccc(-c3ccccc3)cc2)c2ccc(-c3ccccc3)cc2)cc1] 943836-24-6 | 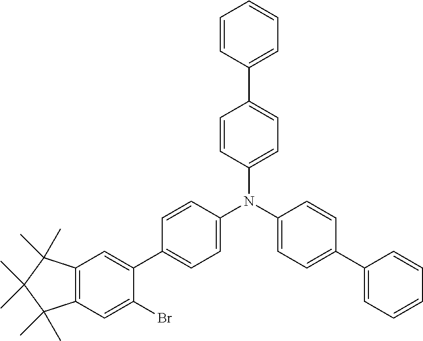 [SMILES: CC1(C)c2cc(Br)c(-c3ccc(N(c4ccc(-c5ccccc5)cc4)c4ccc(-c5ccccc5)cc4)cc3)cc2C(C)(C)C1(C)C] | 86% |
| S56 | LS1 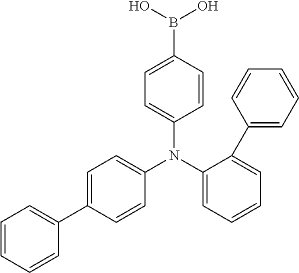 [SMILES: OB(O)c1ccc(N(c2ccc(-c3ccccc3)cc2)c2ccccc2-c2ccccc2)cc1] 16508462-54-9 | 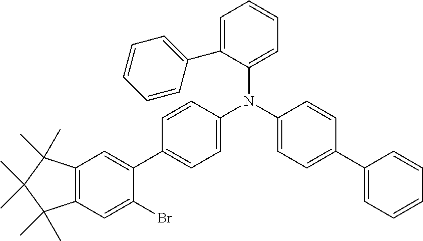 [SMILES: CC1(C)c2cc(Br)c(-c3ccc(N(c4ccc(-c5ccccc5)cc4)c4ccccc4-c4ccccc4)cc3)cc2C(C)(C)C1(C)C] | 81% |
| S57 | LS2 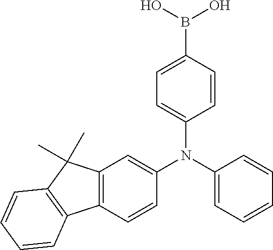 [SMILES: CC1(C)c2ccccc2-c2ccc(N(c3ccccc3)c3ccc(B(O)O)cc3)cc21] 950986-07-9 | 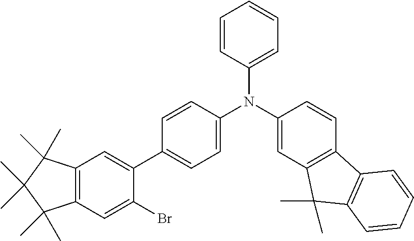 [SMILES: CC1(C)c2ccccc2-c2ccc(N(c3ccccc3)c3ccc(-c4cc5c(cc4Br)C(C)(C)C(C)(C)C5(C)C)cc3)cc21] | 82% |
| S58 | LS1 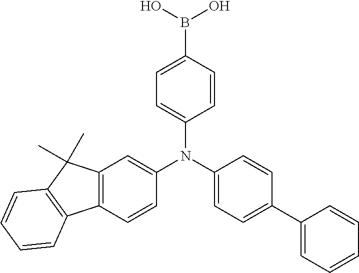 [SMILES: CC1(C)c2ccccc2-c2ccc(N(c3ccc(B(O)O)cc3)c3ccc(-c4ccccc4)cc3)cc21] 1265177-27-2 | 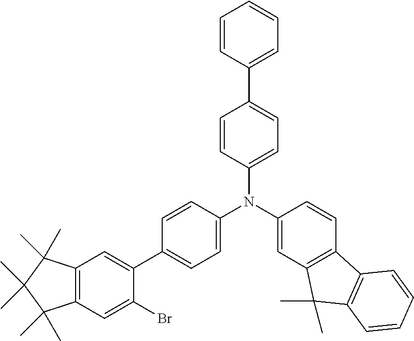 [SMILES: CC1(C)c2ccccc2-c2ccc(N(c3ccc(-c4ccccc4)cc3)c3ccc(-c4cc5c(cc4Br)C(C)(C)C(C)(C)C5(C)C)cc3)cc21] | 85% |
| S59 | LS1 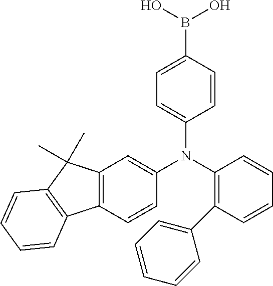 [SMILES: CC1(C)c2ccccc2-c2ccc(N(c3ccc(B(O)O)cc3)c3ccccc3-c3ccccc3)cc21] 1959599-90-6 | 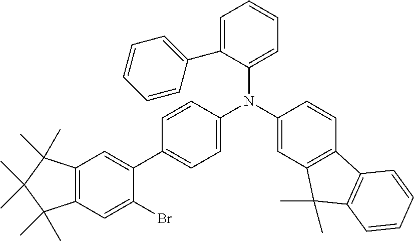 [SMILES: CC1(C)c2ccccc2-c2ccc(N(c3ccc(-c4cc5c(cc4Br)C(C)(C)C(C)(C)C5(C)C)cc3)c3ccccc3-c3ccccc3)cc21] | 80% |
| S60 | LS1 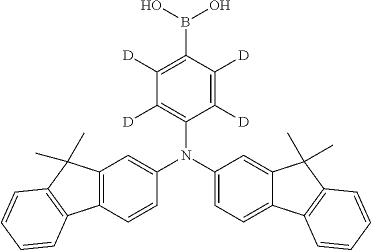 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1B(O)O] 1648570-88-0 | 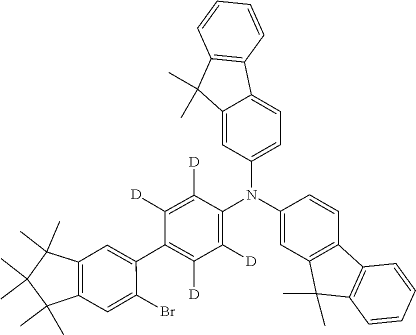 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1-c1cc2c(cc1Br)C(C)(C)C(C)(C)C2(C)C] | 80% |
| S61 | LS1 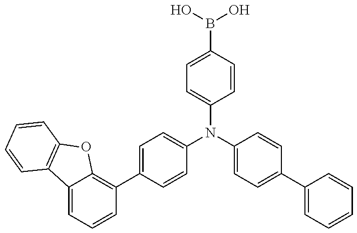 [SMILES: OB(O)c1ccc(N(c2ccc(-c3ccccc3)cc2)c2ccc(-c3cccc4c3oc3ccccc34)cc2)cc1] 2126887-02-1 | 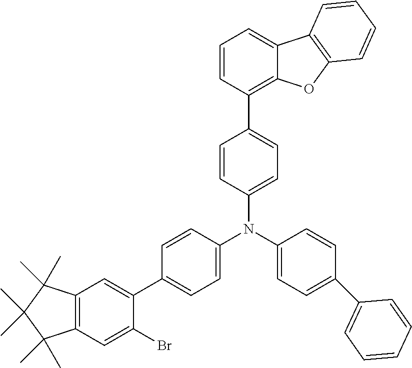 [SMILES: CC1(C)c2cc(Br)c(-c3ccc(N(c4ccc(-c5ccccc5)cc4)c4ccc(-c5cccc6c5oc5ccccc56)cc4)cc3)cc2C(C)(C)C1(C)C] | 83% |
| S62 | LS1 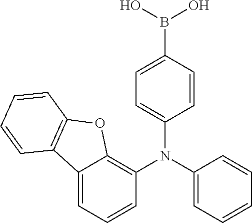 [SMILES: OB(O)c1ccc(N(c2ccccc2)c2cccc3c2oc2ccccc23)cc1] 1428329-78-5 | 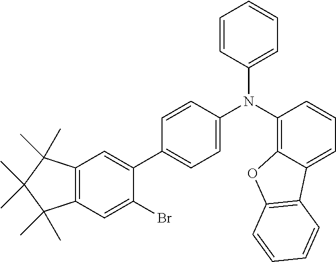 [SMILES: CC1(C)c2cc(Br)c(-c3ccc(N(c4ccccc4)c4cccc5c4oc4ccccc45)cc3)cc2C(C)(C)C1(C)C] | 80% |
| S63 | LS1 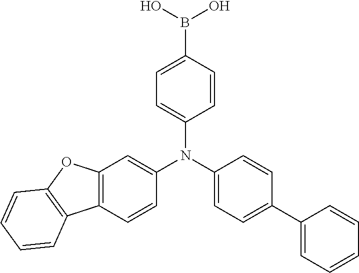 [SMILES: OB(O)c1ccc(N(c2ccc(-c3ccccc3)cc2)c2ccc3c(c2)oc2ccccc23)cc1] 1960443-69-9 | 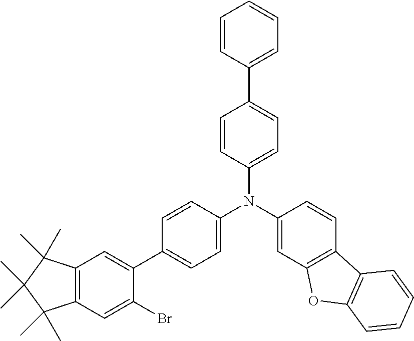 [SMILES: CC1(C)c2cc(Br)c(-c3ccc(N(c4ccc(-c5ccccc5)cc4)c4ccc5c(c4)oc4ccccc45)cc3)cc2C(C)(C)C1(C)C] | 84% |
| S64 | LS1 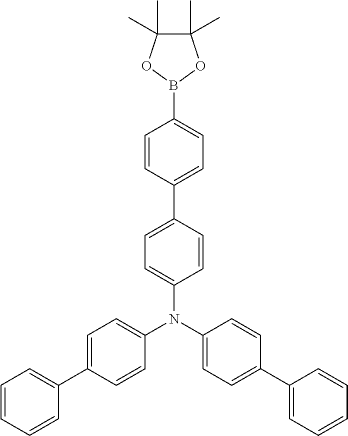 [SMILES: CC1(C)OB(c2ccc(-c3ccc(N(c4ccc(-c5ccccc5)cc4)c4ccc(-c5ccccc5)cc4)cc3)cc2)OC1(C)C] 1421701-43-0 | 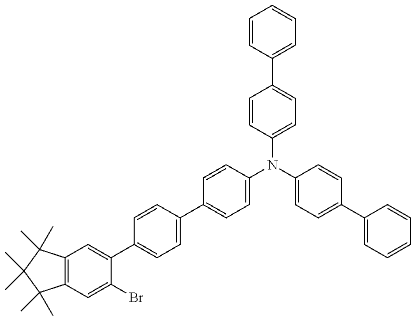 [SMILES: CC1(C)c2cc(Br)c(-c3ccc(-c4ccc(N(c5ccc(-c6ccccc6)cc5)c5ccc(-c6ccccc6)cc5)cc4)cc3)cc2C(C)(C)C1(C)C] | 86% |
| S65 | LS1 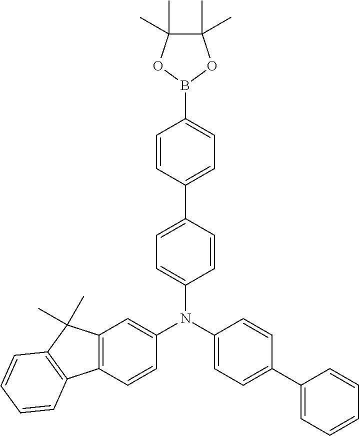 [SMILES: CC1(C)c2ccccc2-c2ccc(N(c3ccc(-c4ccccc4)cc3)c3ccc(-c4ccc(B5OC(C)(C)C(C)(C)O5)cc4)cc3)cc21] 1825336-99-9 | 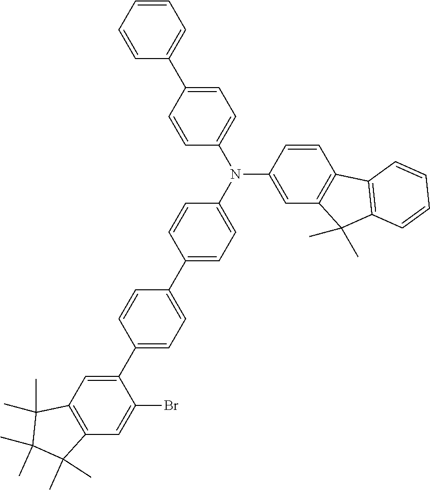 [SMILES: CC1(C)c2ccccc2-c2ccc(N(c3ccc(-c4ccccc4)cc3)c3ccc(-c4ccc(-c5cc6c(cc5Br)C(C)(C)C(C)(C)C6(C)C)cc4)cc3)cc21] | 83% |
| S66 | LS1 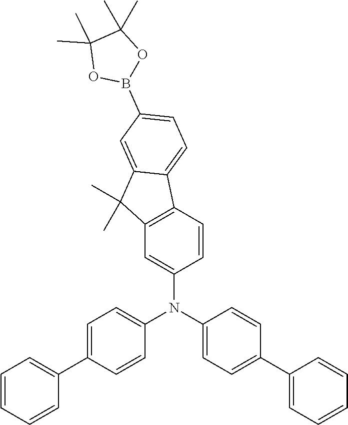 [SMILES: CC1(C)c2cc(B3OC(C)(C)C(C)(C)O3)ccc2-c2ccc(N(c3ccc(-c4ccccc4)cc3)c3ccc(-c4ccccc4)cc3)cc21] 1620895-07-9 | 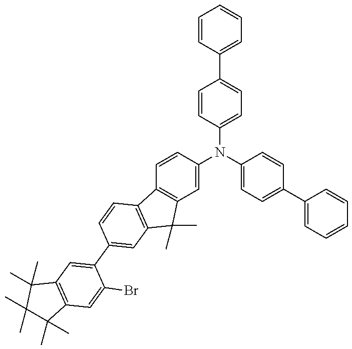 [SMILES: CC1(C)c2cc(-c3cc4c(cc3Br)C(C)(C)C(C)(C)C4(C)C)ccc2-c2ccc(N(c3ccc(-c4ccccc4)cc3)c3ccc(-c4ccccc4)cc3)cc21] | 81% |
| S67 | LS1 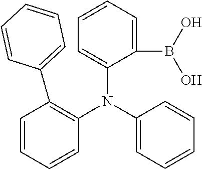 [SMILES: OB(O)c1ccccc1N(c1ccccc1)c1ccccc1-c1ccccc1] 1776935-49-9 | 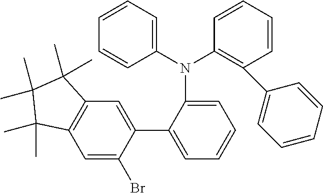 [SMILES: CC1(C)c2cc(Br)c(-c3ccccc3N(c3ccccc3)c3ccccc3-c3ccccc3)cc2C(C)(C)C1(C)C] | 64% |
| S68 | LS1 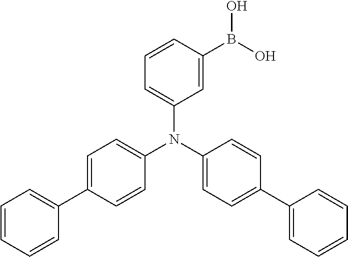 [SMILES: OB(O)c1cccc(N(c2ccc(-c3ccccc3)cc2)c2ccc(-c3ccccc3)cc2)c1] 1776936-65-2 | 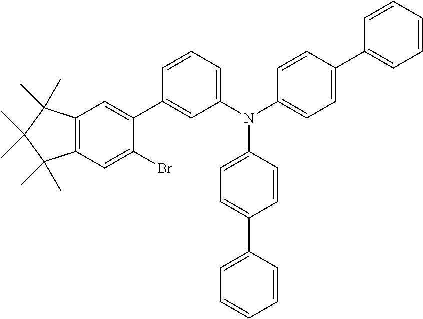 [SMILES: CC1(C)c2cc(Br)c(-c3cccc(N(c4ccc(-c5ccccc5)cc4)c4ccc(-c5ccccc5)cc4)c3)cc2C(C)(C)C1(C)C] | 80% |
| S69 | LS1 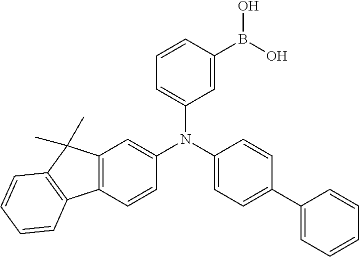 [SMILES: CC1(C)c2ccccc2-c2ccc(N(c3ccc(-c4ccccc4)cc3)c3cccc(B(O)O)c3)cc21] 1610950-84-9 | 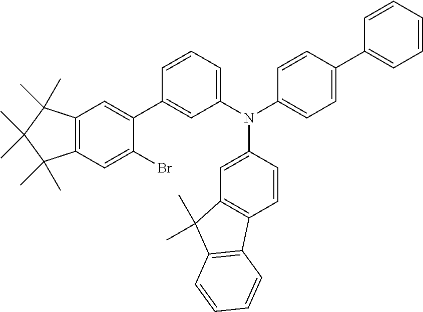 [SMILES: CC1(C)c2ccccc2-c2ccc(N(c3ccc(-c4ccccc4)cc3)c3cccc(-c4cc5c(cc4Br)C(C)(C)C(C)(C)C5(C)C)c3)cc21] | 79% |
| S70 | LS1 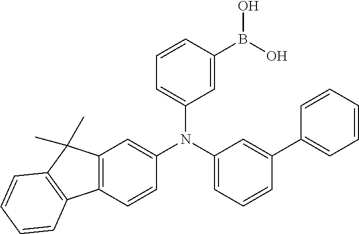 [SMILES: CC1(C)c2ccccc2-c2ccc(N(c3cccc(B(O)O)c3)c3cccc(-c4ccccc4)c3)cc21] 2334467-29-5 | 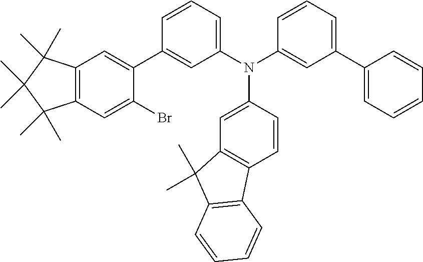 [SMILES: CC1(C)c2ccccc2-c2ccc(N(c3cccc(-c4ccccc4)c3)c3cccc(-c4cc5c(cc4Br)C(C)(C)C(C)(C)C5(C)C)c3)cc21] | 75% |
| S71 | LS1 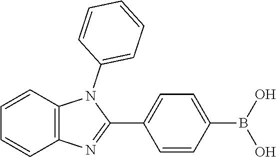 [SMILES: OB(O)c1ccc(-c2nc3ccccc3n2-c2ccccc2)cc1] 952514-79-3 | 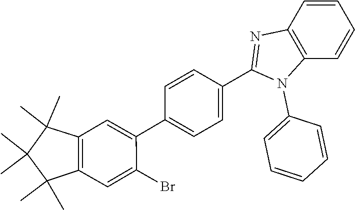 [SMILES: CC1(C)c2cc(Br)c(-c3ccc(-c4nc5ccccc5n4-c4ccccc4)cc3)cc2C(C)(C)C1(C)C] | 80% |
| S72 | LS1 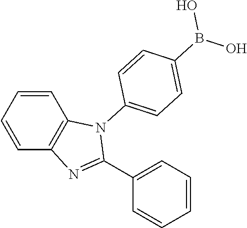 [SMILES: OB(O)c1ccc(-n2c(-c3ccccc3)nc3ccccc32)cc1] 867044-33-5 | 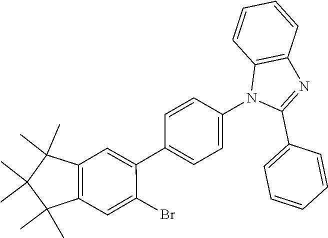 [SMILES: CC1(C)c2cc(Br)c(-c3ccc(-n4c(-c5ccccc5)nc5ccccc54)cc3)cc2C(C)(C)C1(C)C] | 83% |
| S73 | LS1 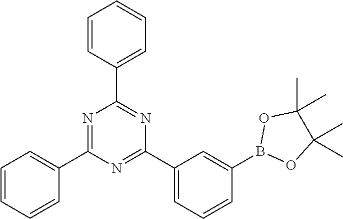 [SMILES: CC1(C)OB(c2cccc(-c3nc(-c4ccccc4)nc(-c4ccccc4)n3)c2)OC1(C)C] 1269508-31-7 | 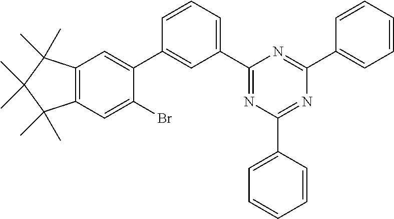 [SMILES: CC1(C)c2cc(Br)c(-c3cccc(-c4nc(-c5ccccc5)nc(-c5ccccc5)n4)c3)cc2C(C)(C)C1(C)C] | 77% |
| S74 | LS1 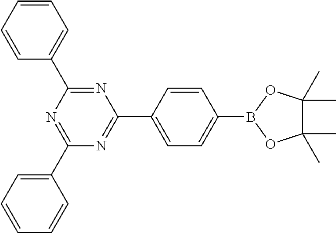 [SMILES: CC1(C)OB(c2ccc(-c3nc(-c4ccccc4)nc(-c4ccccc4)n3)cc2)OC1(C)C] 1219956-23-6 | 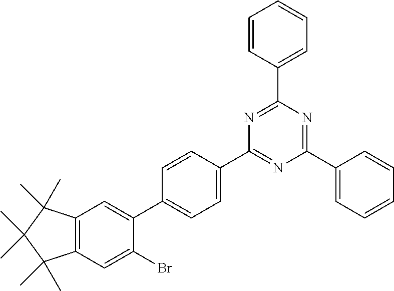 [SMILES: CC1(C)c2cc(Br)c(-c3ccc(-c4nc(-c5ccccc5)nc(-c5ccccc5)n4)cc3)cc2C(C)(C)C1(C)C] | 74% |
| S75 | LS1 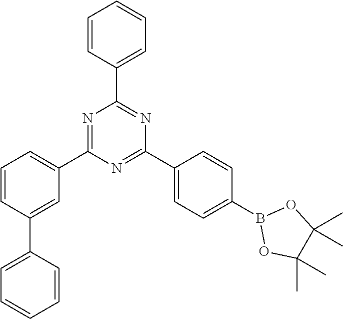 [SMILES: CC1(C)OB(c2ccc(-c3nc(-c4ccccc4)nc(-c4cccc(-c5ccccc5)c4)n3)cc2)OC1(C)C] 2168567-62-0 | 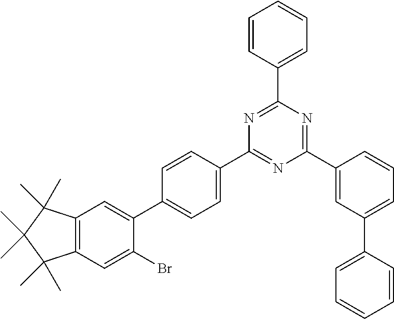 [SMILES: CC1(C)c2cc(Br)c(-c3ccc(-c4nc(-c5ccccc5)nc(-c5cccc(-c6ccccc6)c5)n4)cc3)cc2C(C)(C)C1(C)C] | 76% |
| S76 | LS1 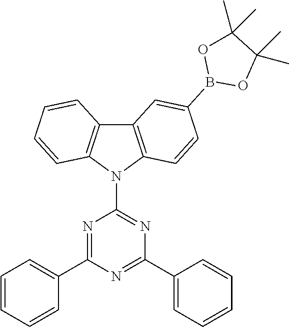 [SMILES: CC1(C)OB(c2ccc3c(c2)c2ccccc2n3-c2nc(-c3ccccc3)nc(-c3ccccc3)n2)OC1(C)C] 1361094-91-8 | 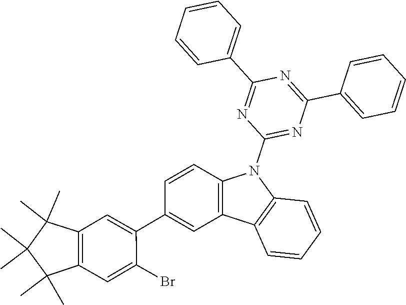 [SMILES: CC1(C)c2cc(Br)c(-c3ccc4c(c3)c3ccccc3n4-c3nc(-c4ccccc4)nc(-c4ccccc4)n3)cc2C(C)(C)C1(C)C] | 79% |
| S77 | LS1 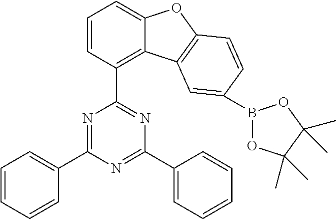 [SMILES: CC1(C)OB(c2ccc3oc4cccc(-c5nc(-c6ccccc6)nc(-c6ccccc6)n5)c4c3c2)OC1(C)C] 2138490-96-5 | 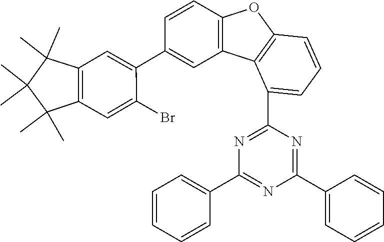 [SMILES: CC1(C)c2cc(Br)c(-c3ccc4oc5cccc(-c6nc(-c7ccccc7)nc(-c7ccccc7)n6)c5c4c3)cc2C(C)(C)C1(C)C] | 63% |

### B) Synthesis of the Compounds of the Invention

### Example A1

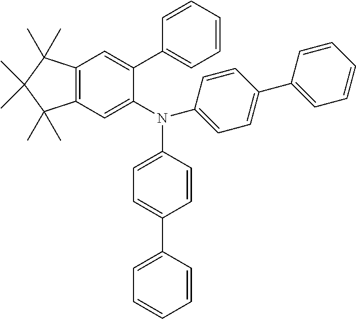 [SMILES: CC1(C)c2cc(-c3ccccc3)c(N(c3ccc(-c4ccccc4)cc3)c3ccc(-c4ccccc4)cc3)cc2C(C)(C)C1(C)C]

To a solution of 35.7 g (100 mmol) of S1 and 38.6 g (120 mmol) of bis-p-biphenylamine [102113-98-4] in 500 ml of toluene are added 4.0 ml (4.0 mmol) of a tri-tert-butylphosphine solution, 1.0 M in toluene, 449 mg (2 mmol) of palladium acetate and 15.4 g of sodium tert-butoxide (160 mmol), and the mixture was heated under reflux for 16 h. The reaction mixture is cooled down to room temperature, extended with toluene and filtered through a Celite bed. The filtrate is concentrated under reduced pressure and the residue is crystallized from ethyl acetate/n-heptane. The crude product is purified by hot-extraction crystallization (typical organic solvents, preferably acetonitrile or acetonitrile/dichloromethane mixtures 4:1 to 1:4 vv) or chromatography (Torrent automated column system from A. Semrau) and by zone sublimation twice under reduced pressure (p˜10⁻⁵ mbar, T˜280° C.). Yield: 44.8 g (75 mmol) 75%. Purity by HPLC >99.9%.

The following compounds can be prepared analogously:

| Ex. | Reactants | Product | Yield |
|---|---|---|---|
| A2 | S1 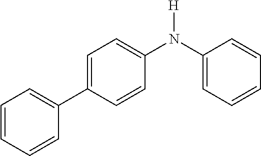 [SMILES: c1ccc(Nc2ccc(-c3ccccc3)cc2)cc1] 32228-99-2 | 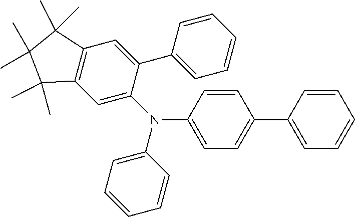 [SMILES: CC1(C)c2cc(-c3ccccc3)c(N(c3ccccc3)c3ccc(-c4ccccc4)cc3)cc2C(C)(C)C1(C)C] | 70% |
| A3 | S1 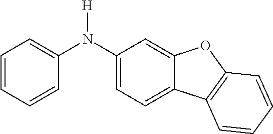 [SMILES: c1ccc(Nc2ccc3c(c2)oc2ccccc23)cc1] 406488-21-9 | 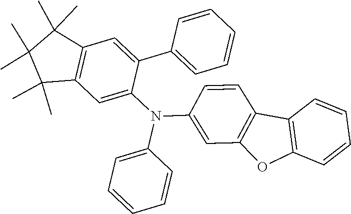 [SMILES: CC1(C)c2cc(-c3ccccc3)c(N(c3ccccc3)c3ccc4c(c3)oc3ccccc34)cc2C(C)(C)C1(C)C] | 72% |
| A4 | S1 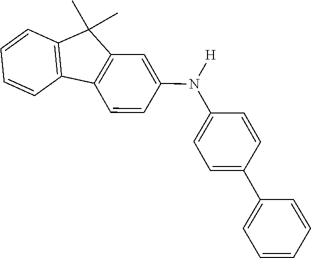 [SMILES: CC1(C)c2ccccc2-c2ccc(Nc3ccc(-c4ccccc4)cc3)cc21] 897671-69-1 | 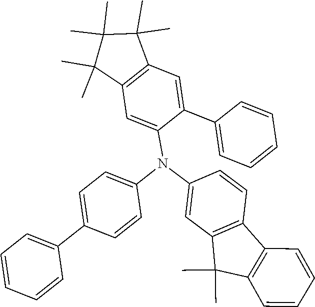 [SMILES: CC1(C)c2ccccc2-c2ccc(N(c3ccc(-c4ccccc4)cc3)c3cc4c(cc3-c3ccccc3)C(C)(C)C(C)(C)C4(C)C)cc21] | 69% |
| A5 | S2 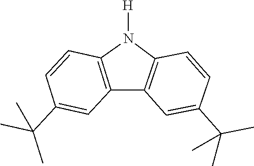 [SMILES: CC(C)(C)c1ccc2[nH]c3ccc(C(C)(C)C)cc3c2c1] 37500-95-1 | 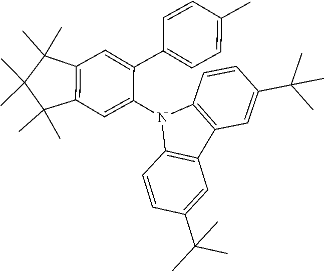 [SMILES: Cc1ccc(-c2cc3c(cc2-n2c4ccc(C(C)(C)C)cc4c4cc(C(C)(C)C)ccc42)C(C)(C)C(C)(C)C3(C)C)cc1] | 63% |
| A6 | S3 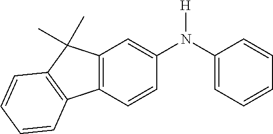 [SMILES: CC1(C)c2ccccc2-c2ccc(Nc3ccccc3)cc21] 355832-04-1 | 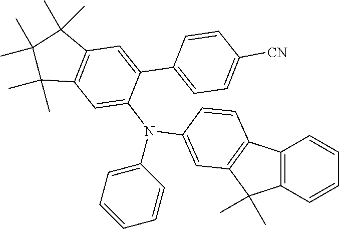 [SMILES: CC1(C)c2ccccc2-c2ccc(N(c3ccccc3)c3cc4c(cc3-c3ccc(C#N)cc3)C(C)(C)C(C)(C)C4(C)C)cc21] | 76% |
| A7 | S4 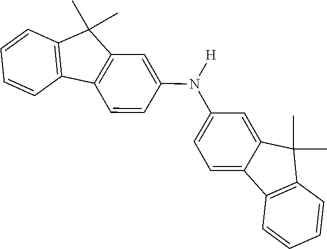 [SMILES: CC1(C)c2ccccc2-c2ccc(Nc3ccc4c(c3)C(C)(C)c3ccccc3-4)cc21] 500717-23-7 | 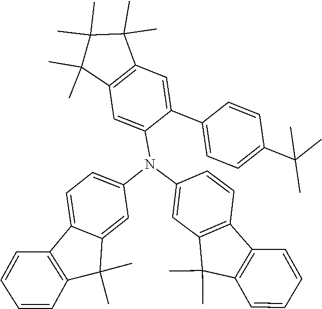 [SMILES: CC(C)(C)c1ccc(-c2cc3c(cc2N(c2ccc4c(c2)C(C)(C)c2ccccc2-4)c2ccc4c(c2)C(C)(C)c2ccccc2-4)C(C)(C)C(C)(C)C3(C)C)cc1] | 74% |
| A8 | S5 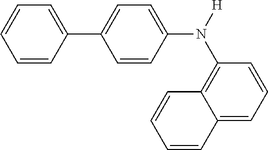 [SMILES: c1ccc(-c2ccc(Nc3cccc4ccccc34)cc2)cc1] 446242-37-1 | 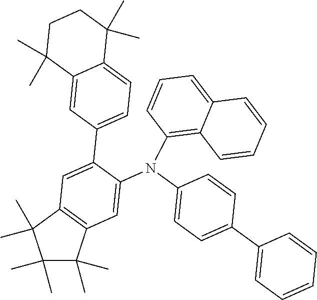 [SMILES: CC1(C)CCC(C)(C)c2cc(-c3cc4c(cc3N(c3ccc(-c5ccccc5)cc3)c3cccc5ccccc35)C(C)(C)C(C)(C)C4(C)C)ccc21] | 75% |
| A9 | S6 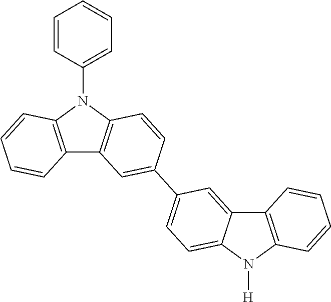 [SMILES: c1ccc(-n2c3ccccc3c3cc(-c4ccc5[nH]c6ccccc6c5c4)ccc32)cc1] | 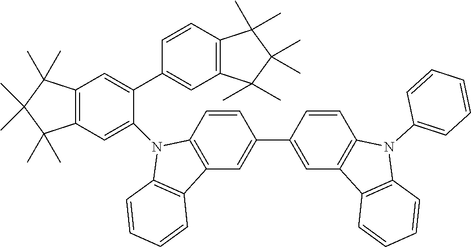 [SMILES: CC1(C)c2ccc(-c3cc4c(cc3-n3c5ccccc5c5cc(-c6ccc7c(c6)c6ccccc6n7-c6ccccc6)ccc53)C(C)(C)C(C)(C)C4(C)C)cc2C(C)(C)C1(C)C] | 73% |
| | 1060735-14-9 | | |
| A10 | S7 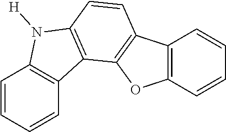 [SMILES: c1ccc2c(c1)[nH]c1ccc3c4ccccc4oc3c12] 1199616-66-4 | 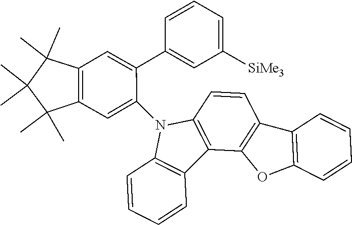 [SMILES: CC1(C)c2cc(-c3cccc([Si](C)(C)C)c3)c(-n3c4ccccc4c4c5oc6ccccc6c5ccc43)cc2C(C)(C)C1(C)C] | 37% |
| A11 | S8 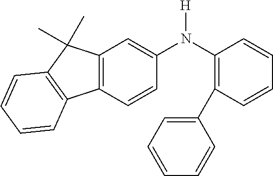 [SMILES: CC1(C)c2ccccc2-c2ccc(Nc3ccccc3-c3ccccc3)cc21] 1198395-24-2 | 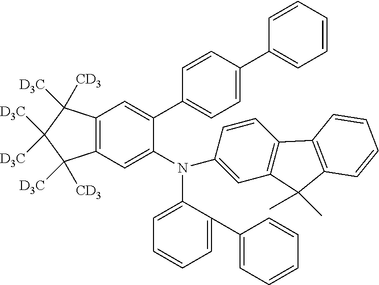 [SMILES: [2H]C([2H])([2H])C1(C([2H])([2H])[2H])c2cc(-c3ccc(-c4ccccc4)cc3)c(N(c3ccc4c(c3)C(C)(C)c3ccccc3-4)c3ccccc3-c3ccccc3)cc2C(C([2H])([2H])[2H])(C([2H])([2H])[2H])C1(C([2H])([2H])[2H])C([2H])([2H])[2H]] | 70% |
| A12 | S9 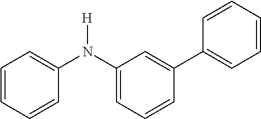 [SMILES: c1ccc(Nc2cccc(-c3ccccc3)c2)cc1] 198275-79-5 | 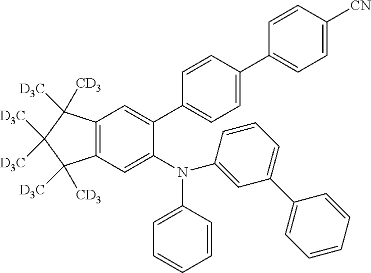 [SMILES: [2H]C([2H])([2H])C1(C([2H])([2H])[2H])c2cc(-c3ccc(-c4ccc(C#N)cc4)cc3)c(N(c3ccccc3)c3cccc(-c4ccccc4)c3)cc2C(C([2H])([2H])[2H])(C([2H])([2H])[2H])C1(C([2H])([2H])[2H])C([2H])([2H])[2H]] | 66% |
| A13 | S10 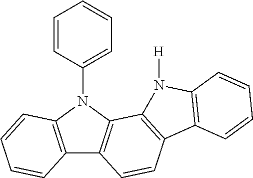 [SMILES: c1ccc(-n2c3ccccc3c3ccc4c5ccccc5[nH]c4c32)cc1] 1024598-06-8 | 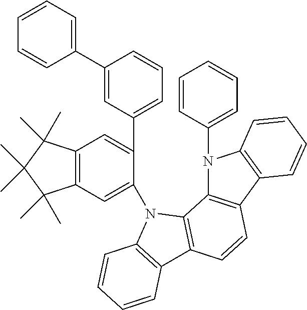 [SMILES: CC1(C)c2cc(-c3cccc(-c4ccccc4)c3)c(-n3c4ccccc4c4ccc5c6ccccc6n(-c6ccccc6)c5c43)cc2C(C)(C)C1(C)C] | 69% |
| A14 | S11 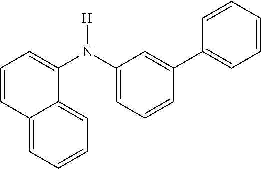 [SMILES: c1ccc(-c2cccc(Nc3cccc4ccccc34)c2)cc1] 59994-77-3 | 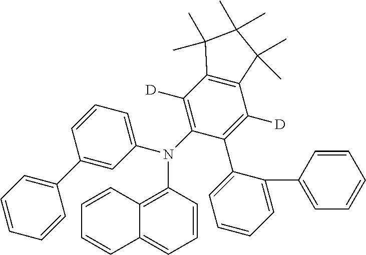 [SMILES: [2H]c1c(-c2ccccc2-c2ccccc2)c(N(c2cccc(-c3ccccc3)c2)c2cccc3ccccc23)c([2H])c2c1C(C)(C)C(C)(C)C2(C)C] | 70% |
| A15 | S12 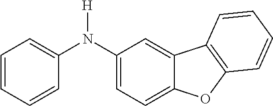 [SMILES: c1ccc(Nc2ccc3oc4ccccc4c3c2)cc1] 861317-95-5 | 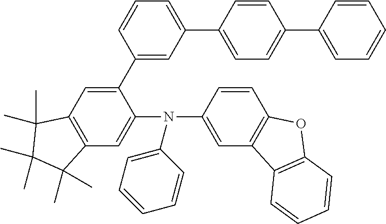 [SMILES: CC1(C)c2cc(-c3cccc(-c4ccc(-c5ccccc5)cc4)c3)c(N(c3ccccc3)c3ccc4oc5ccccc5c4c3)cc2C(C)(C)C1(C)C] | 74% |
| A16 | S13 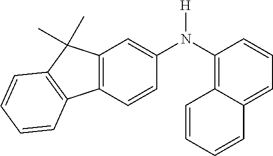 [SMILES: CC1(C)c2ccccc2-c2ccc(Nc3cccc4ccccc34)cc21] 672289-02-0 | 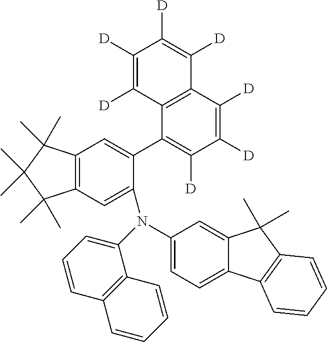 [SMILES: [2H]c1c([2H])c([2H])c2c(-c3cc4c(cc3N(c3ccc5c(c3)C(C)(C)c3ccccc3-5)c3cccc5ccccc35)C(C)(C)C(C)(C)C4(C)C)c([2H])c([2H])c([2H])c2c1[2H]] | 73% |
| A17 | S14 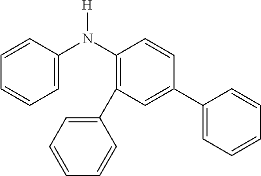 [SMILES: c1ccc(Nc2ccc(-c3ccccc3)cc2-c2ccccc2)cc1] 1417334-01-0 | 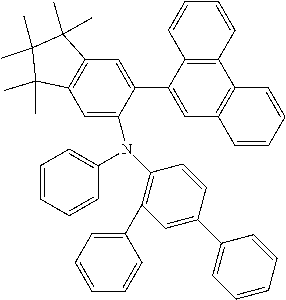 [SMILES: CC1(C)c2cc(-c3cc4ccccc4c4ccccc34)c(N(c3ccccc3)c3ccc(-c4ccccc4)cc3-c3ccccc3)cc2C(C)(C)C1(C)C] | 70% |
| A18 | S15 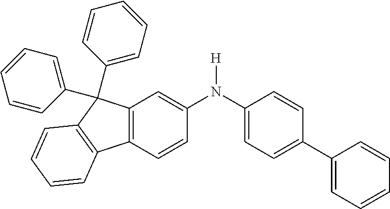 [SMILES: c1ccc(-c2ccc(Nc3ccc4c(c3)C(c3ccccc3)(c3ccccc3)c3ccccc3-4)cc2)cc1] 1268520-04-2 | 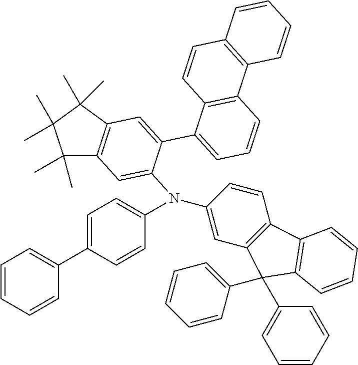 [SMILES: CC1(C)c2cc(-c3cccc4c3ccc3ccccc34)c(N(c3ccc(-c4ccccc4)cc3)c3ccc4c(c3)C(c3ccccc3)(c3ccccc3)c3ccccc3-4)cc2C(C)(C)C1(C)C] | 68% |
| A19 | S16 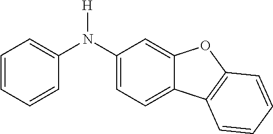 [SMILES: c1ccc(Nc2ccc3c(c2)oc2ccccc23)cc1] 406488-21-9 | 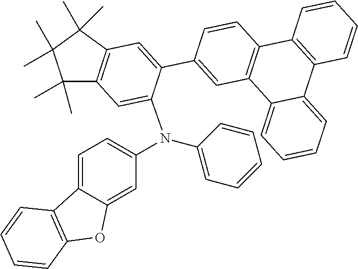 [SMILES: CC1(C)c2cc(-c3ccc4c5ccccc5c5ccccc5c4c3)c(N(c3ccccc3)c3ccc4c(c3)oc3ccccc34)cc2C(C)(C)C1(C)C] | 78% |
| A20 | S17 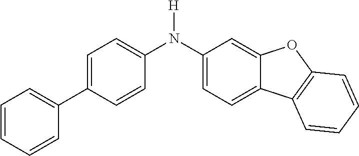 [SMILES: c1ccc(-c2ccc(Nc3ccc4c(c3)oc3ccccc34)cc2)cc1] 290039-85-8 | 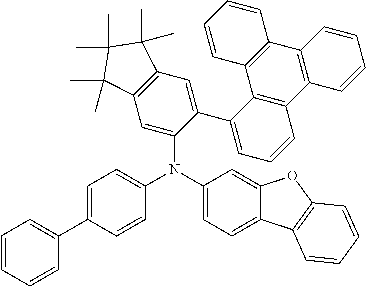 [SMILES: CC1(C)c2cc(-c3cccc4c5ccccc5c5ccccc5c34)c(N(c3ccc(-c4ccccc4)cc3)c3ccc4c(c3)oc3ccccc34)cc2C(C)(C)C1(C)C] | 71% |
| A21 | S18 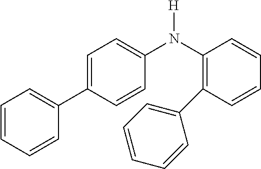 [SMILES: c1ccc(-c2ccc(Nc3ccccc3-c3ccccc3)cc2)cc1] 1372775-52-4 | 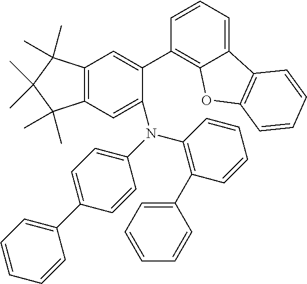 [SMILES: CC1(C)c2cc(-c3cccc4c3oc3ccccc34)c(N(c3ccc(-c4ccccc4)cc3)c3ccccc3-c3ccccc3)cc2C(C)(C)C1(C)C] | 69% |
| A22 | S19 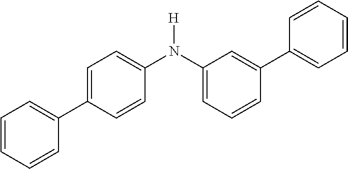 [SMILES: c1ccc(-c2ccc(Nc3cccc(-c4ccccc4)c3)cc2)cc1] 570391-47-8 | 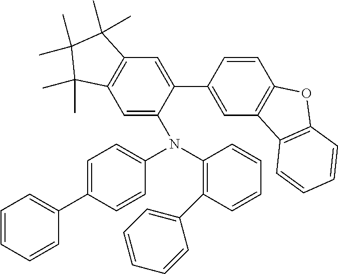 [SMILES: CC1(C)c2cc(-c3ccc4oc5ccccc5c4c3)c(N(c3ccc(-c4ccccc4)cc3)c3ccccc3-c3ccccc3)cc2C(C)(C)C1(C)C] | 74% |
| A23 | S20 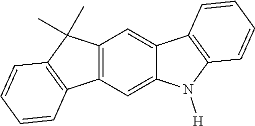 [SMILES: CC1(C)c2ccccc2-c2cc3[nH]c4ccccc4c3cc21] 1260228-95-2 | 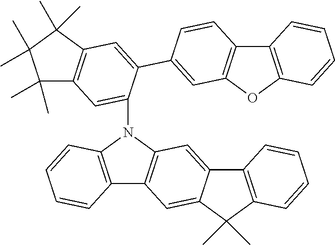 [SMILES: CC1(C)c2ccccc2-c2cc3c(cc21)c1ccccc1n3-c1cc2c(cc1-c1ccc3c(c1)oc1ccccc13)C(C)(C)C(C)(C)C2(C)C] | 74% |
| A24 | S21 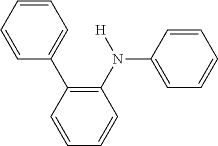 [SMILES: c1ccc(Nc2ccccc2-c2ccccc2)cc1] 35887-50-4 | 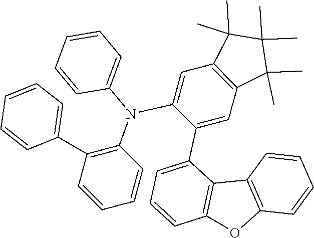 [SMILES: CC1(C)c2cc(-c3cccc4oc5ccccc5c34)c(N(c3ccccc3)c3ccccc3-c3ccccc3)cc2C(C)(C)C1(C)C] | 69% |
| A25 | S22 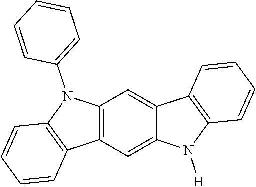 [SMILES: c1ccc(-n2c3ccccc3c3cc4[nH]c5ccccc5c4cc32)cc1] 1316311-27-9 | 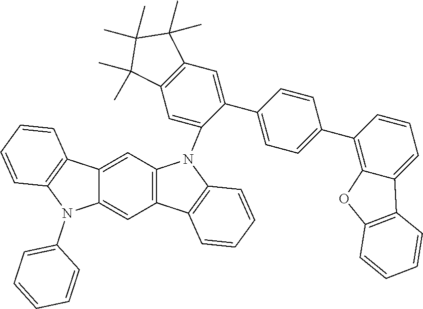 [SMILES: CC1(C)c2cc(-c3ccc(-c4cccc5c4oc4ccccc45)cc3)c(-n3c4ccccc4c4cc5c(cc43)c3ccccc3n5-c3ccccc3)cc2C(C)(C)C1(C)C] | 73% |
| A26 | S23 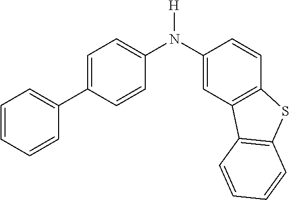 [SMILES: c1ccc(-c2ccc(Nc3ccc4sc5ccccc5c4c3)cc2)cc1] 1300028-93-6 | 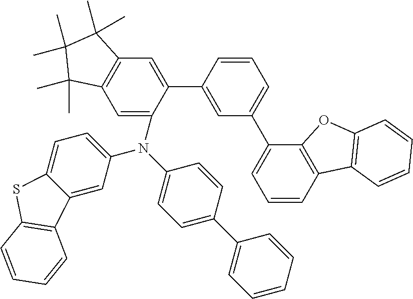 [SMILES: CC1(C)c2cc(-c3cccc(-c4cccc5c4oc4ccccc45)c3)c(N(c3ccc(-c4ccccc4)cc3)c3ccc4sc5ccccc5c4c3)cc2C(C)(C)C1(C)C] | 67% |
| A27 | S24 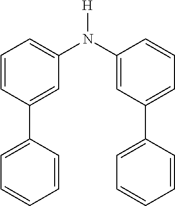 [SMILES: c1ccc(-c2cccc(Nc3cccc(-c4ccccc4)c3)c2)cc1] 169224-65-1 | 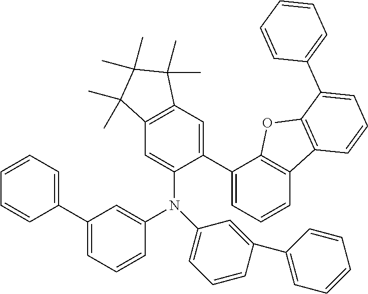 [SMILES: CC1(C)c2cc(-c3cccc4c3oc3c(-c5ccccc5)cccc34)c(N(c3cccc(-c4ccccc4)c3)c3cccc(-c4ccccc4)c3)cc2C(C)(C)C1(C)C] | 69% |
| A28 | S25 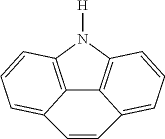 [SMILES: c1cc2ccc3cccc4[nH]c(c1)c2c34] 203-65-6 | 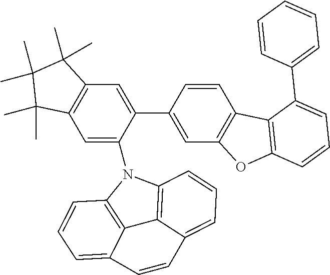 [SMILES: CC1(C)c2cc(-c3ccc4c(c3)oc3cccc(-c5ccccc5)c34)c(-n3c4cccc5ccc6cccc3c6c54)cc2C(C)(C)C1(C)C] | 76% |
| A29 | S26 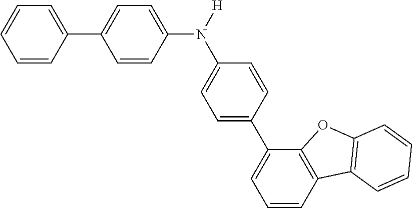 [SMILES: c1ccc(-c2ccc(Nc3ccc(-c4cccc5c4oc4ccccc45)cc3)cc2)cc1] 955959-89-4 | 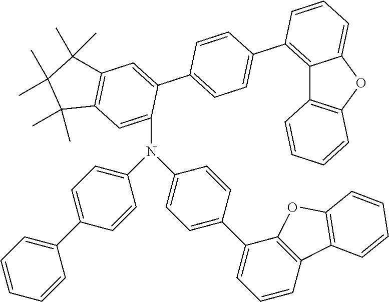 [SMILES: CC1(C)c2cc(-c3ccc(-c4cccc5oc6ccccc6c45)cc3)c(N(c3ccc(-c4ccccc4)cc3)c3ccc(-c4cccc5c4oc4ccccc45)cc3)cc2C(C)(C)C1(C)C] | 70% |
| A30 | S27 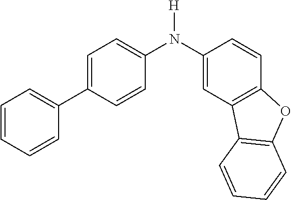 [SMILES: c1ccc(-c2ccc(Nc3ccc4oc5ccccc5c4c3)cc2)cc1] 1300028-94-7 | 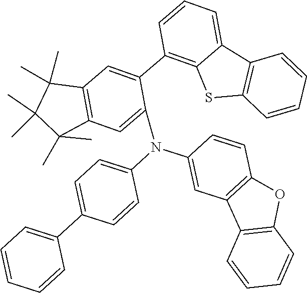 [SMILES: CC1(C)c2cc(-c3cccc4c3sc3ccccc34)c(N(c3ccc(-c4ccccc4)cc3)c3ccc4oc5ccccc5c4c3)cc2C(C)(C)C1(C)C] | 67% |
| A31 | S28 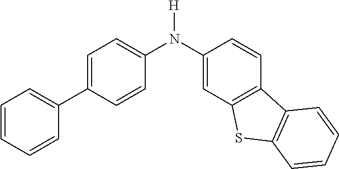 [SMILES: c1ccc(-c2ccc(Nc3ccc4c(c3)sc3ccccc34)cc2)cc1] 1290039-87-0 | 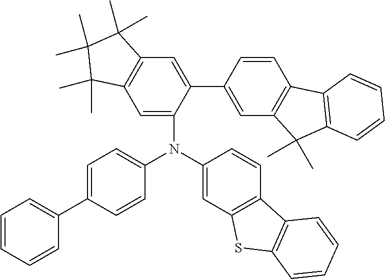 [SMILES: CC1(C)c2ccccc2-c2ccc(-c3cc4c(cc3N(c3ccc(-c5ccccc5)cc3)c3ccc5c(c3)sc3ccccc35)C(C)(C)C(C)(C)C4(C)C)cc21] | 63% |
| A32 | S29 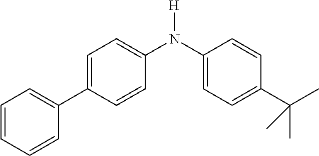 [SMILES: CC(C)(C)c1ccc(Nc2ccc(-c3ccccc3)cc2)cc1] 950917-84-7 | 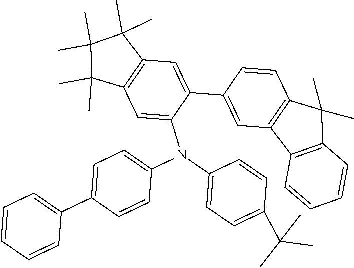 [SMILES: CC(C)(C)c1ccc(N(c2ccc(-c3ccccc3)cc2)c2cc3c(cc2-c2ccc4c(c2)-c2ccccc2C4(C)C)C(C)(C)C(C)(C)C3(C)C)cc1] | 69% |
| A33 | S30 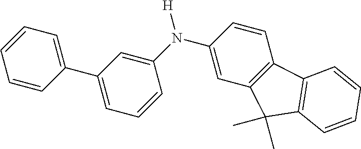 [SMILES: CC1(C)c2ccccc2-c2ccc(Nc3cccc(-c4ccccc4)c3)cc21] 1372778-66-9 | 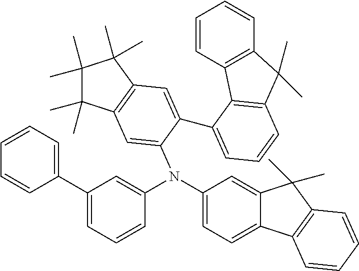 [SMILES: CC1(C)c2ccccc2-c2ccc(N(c3cccc(-c4ccccc4)c3)c3cc4c(cc3-c3cccc5c3-c3ccccc3C5(C)C)C(C)(C)C(C)(C)C4(C)C)cc21] | 68% |
| A34 | S31 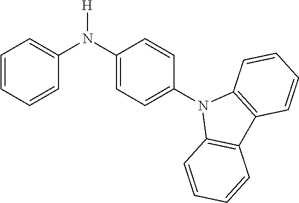 [SMILES: c1ccc(Nc2ccc(-n3c4ccccc4c4ccccc43)cc2)cc1] 858641-06-2 | 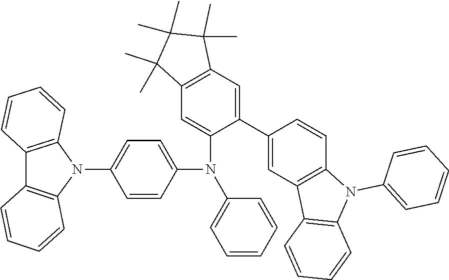 [SMILES: CC1(C)c2cc(-c3ccc4c(c3)c3ccccc3n4-c3ccccc3)c(N(c3ccccc3)c3ccc(-n4c5ccccc5c5ccccc54)cc3)cc2C(C)(C)C1(C)C] | 56% |
| A35 | S32 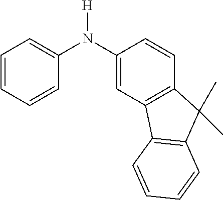 [SMILES: CC1(C)c2ccccc2-c2cc(Nc3ccccc3)ccc21] 850181-65-6 | 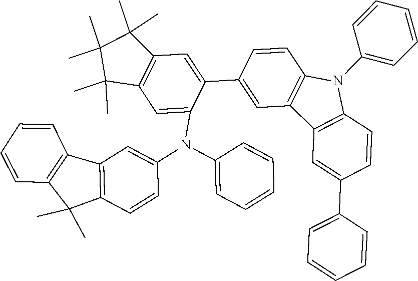 [SMILES: CC1(C)c2ccccc2-c2cc(N(c3ccccc3)c3cc4c(cc3-c3ccc5c(c3)c3cc(-c6ccccc6)ccc3n5-c3ccccc3)C(C)(C)C(C)(C)C4(C)C)ccc21] | 72% |
| A36 | S33 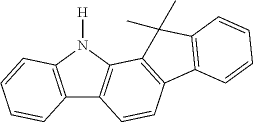 [SMILES: CC1(C)c2ccccc2-c2ccc3c([nH]c4ccccc43)c21] 1329054-41-2 | 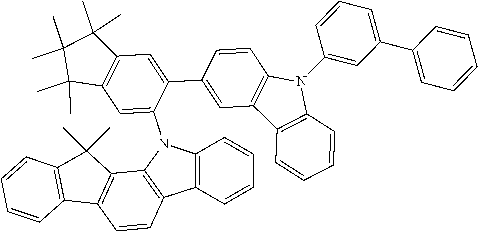 [SMILES: CC1(C)c2ccccc2-c2ccc3c4ccccc4n(-c4cc5c(cc4-c4ccc6c(c4)c4ccccc4n6-c4cccc(-c6ccccc6)c4)C(C)(C)C(C)(C)C5(C)C)c3c21] | 76% |
| A37 | S34 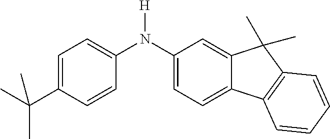 [SMILES: CC(C)(C)c1ccc(Nc2ccc3c(c2)C(C)(C)c2ccccc2-3)cc1] 944418-46-6 | 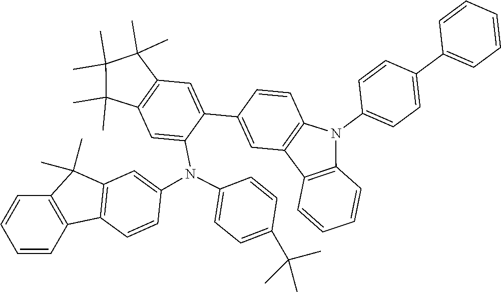 [SMILES: CC(C)(C)c1ccc(N(c2ccc3c(c2)C(C)(C)c2ccccc2-3)c2cc3c(cc2-c2ccc4c(c2)c2ccccc2n4-c2ccc(-c4ccccc4)cc2)C(C)(C)C(C)(C)C3(C)C)cc1] | 75% |
| A38 | S35 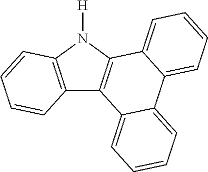 [SMILES: c1ccc2c(c1)[nH]c1c3ccccc3c3ccccc3c21] 201-67-2 | 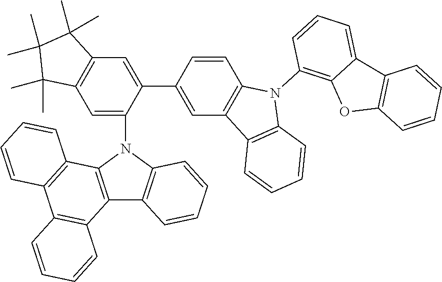 [SMILES: CC1(C)c2cc(-c3ccc4c(c3)c3ccccc3n4-c3cccc4c3oc3ccccc34)c(-n3c4ccccc4c4c5ccccc5c5ccccc5c43)cc2C(C)(C)C1(C)C] | 77% |
| A39 | S36 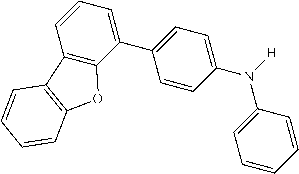 [SMILES: c1ccc(Nc2ccc(-c3cccc4c3oc3ccccc34)cc2)cc1] 955959-87-2 | 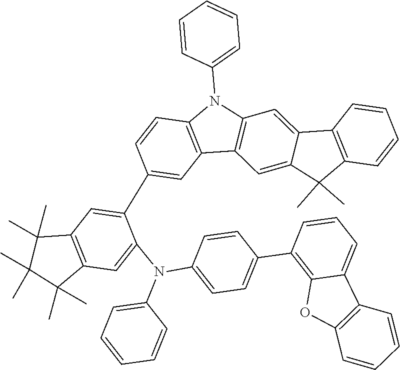 [SMILES: CC1(C)c2ccccc2-c2cc3c(cc21)c1cc(-c2cc4c(cc2N(c2ccccc2)c2ccc(-c5cccc6c5oc5ccccc56)cc2)C(C)(C)C(C)(C)C4(C)C)ccc1n3-c1ccccc1] | 69% |
| A40 | S37 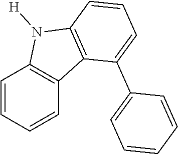 [SMILES: c1ccc(-c2cccc3[nH]c4ccccc4c23)cc1] 1201561-34-3 | 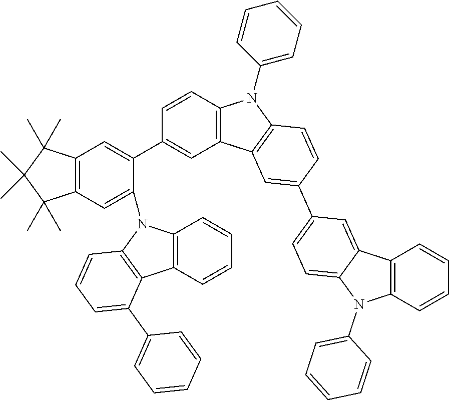 [SMILES: CC1(C)c2cc(-c3ccc4c(c3)c3cc(-c5ccc6c(c5)c5ccccc5n6-c5ccccc5)ccc3n4-c3ccccc3)c(-n3c4ccccc4c4c(-c5ccccc5)cccc43)cc2C(C)(C)C1(C)C] | 80% |
| A41 | S38 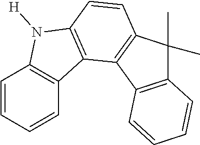 [SMILES: CC1(C)c2ccccc2-c2c1ccc1[nH]c3ccccc3c21] 1623813-70-6 | 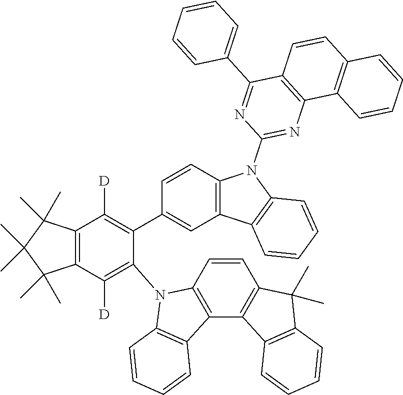 [SMILES: [2H]c1c(-c2ccc3c(c2)c2ccccc2n3-c2nc(-c3ccccc3)c3ccc4ccccc4c3n2)c(-n2c3ccccc3c3c4c(ccc32)C(C)(C)c2ccccc2-4)c([2H])c2c1C(C)(C)C(C)(C)C2(C)C] | 78% |
| A42 | S39 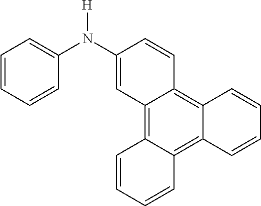 [SMILES: c1ccc(Nc2ccc3c4ccccc4c4ccccc4c3c2)cc1] 1203922-52-4 | 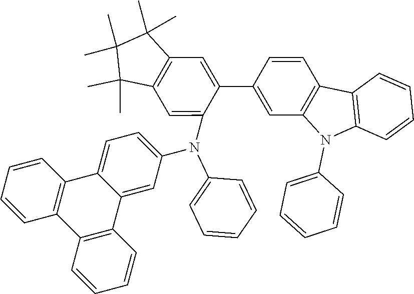 [SMILES: CC1(C)c2cc(-c3ccc4c5ccccc5n(-c5ccccc5)c4c3)c(N(c3ccccc3)c3ccc4c5ccccc5c5ccccc5c4c3)cc2C(C)(C)C1(C)C] | 74% |
| A43 | S40 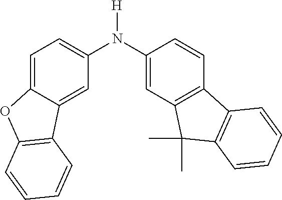 [SMILES: CC1(C)c2ccccc2-c2ccc(Nc3ccc4oc5ccccc5c4c3)cc21] 1359833-89-8 | 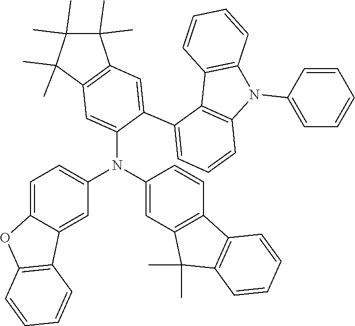 [SMILES: CC1(C)c2ccccc2-c2ccc(N(c3ccc4oc5ccccc5c4c3)c3cc4c(cc3-c3cccc5c3c3ccccc3n5-c3ccccc3)C(C)(C)C(C)(C)C4(C)C)cc21] | 70% |
| A44 | S41 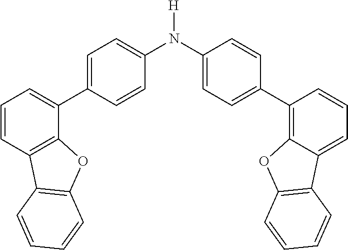 [SMILES: c1ccc2c(c1)oc1c(-c3ccc(Nc4ccc(-c5cccc6c5oc5ccccc56)cc4)cc3)cccc12] 955959-91-8 | 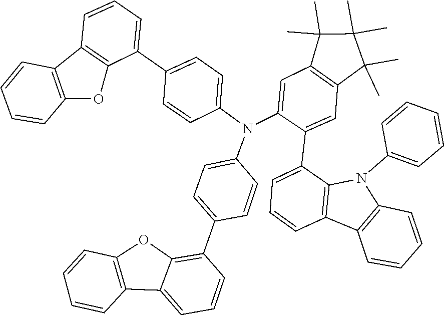 [SMILES: CC1(C)c2cc(-c3cccc4c5ccccc5n(-c5ccccc5)c34)c(N(c3ccc(-c4cccc5c4oc4ccccc45)cc3)c3ccc(-c4cccc5c4oc4ccccc45)cc3)cc2C(C)(C)C1(C)C] | 68% |
| A45 | S42 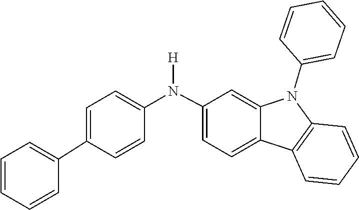 [SMILES: c1ccc(-c2ccc(Nc3ccc4c5ccccc5n(-c5ccccc5)c4c3)cc2)cc1] 1427316-58-2 | 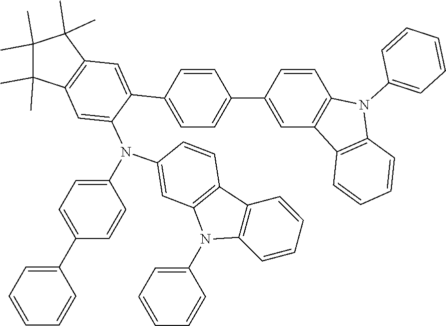 [SMILES: CC1(C)c2cc(-c3ccc(-c4ccc5c(c4)c4ccccc4n5-c4ccccc4)cc3)c(N(c3ccc(-c4ccccc4)cc3)c3ccc4c5ccccc5n(-c5ccccc5)c4c3)cc2C(C)(C)C1(C)C] | 74% |
| A46 | S43 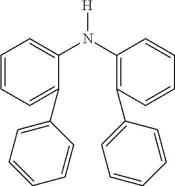 [SMILES: c1ccc(-c2ccccc2Nc2ccccc2-c2ccccc2)cc1] 91923-32-9 | 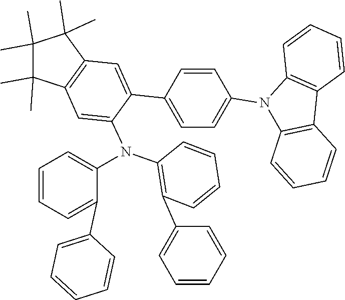 [SMILES: CC1(C)c2cc(-c3ccc(-n4c5ccccc5c5ccccc54)cc3)c(N(c3ccccc3-c3ccccc3)c3ccccc3-c3ccccc3)cc2C(C)(C)C1(C)C] | 67% |
| A47 | S44 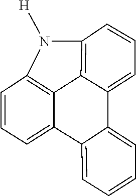 [SMILES: c1ccc2c(c1)c1cccc3[nH]c4cccc2c4c31] 109606-75-9 | 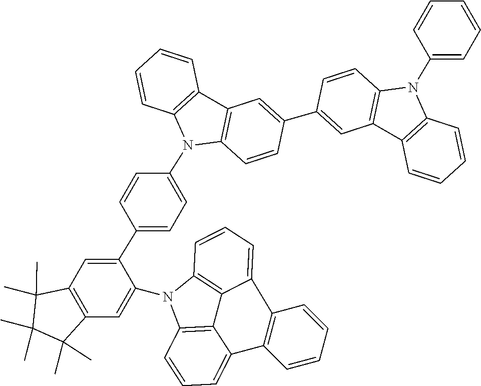 [SMILES: CC1(C)c2cc(-c3ccc(-n4c5ccccc5c5cc(-c6ccc7c(c6)c6ccccc6n7-c6ccccc6)ccc54)cc3)c(-n3c4cccc5c6ccccc6c6cccc3c6c54)cc2C(C)(C)C1(C)C] | 78% |
| A48 | S45 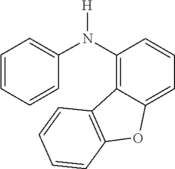 [SMILES: c1ccc(Nc2cccc3oc4ccccc4c23)cc1] 1325195-27-4 | 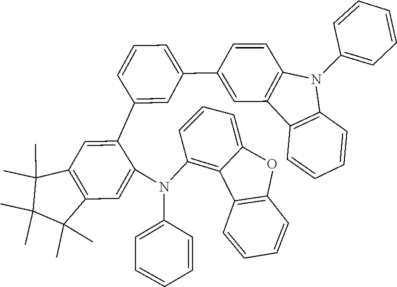 [SMILES: CC1(C)c2cc(-c3cccc(-c4ccc5c(c4)c4ccccc4n5-c4ccccc4)c3)c(N(c3ccccc3)c3cccc4oc5ccccc5c34)cc2C(C)(C)C1(C)C] | 67% |
| A49 | S46 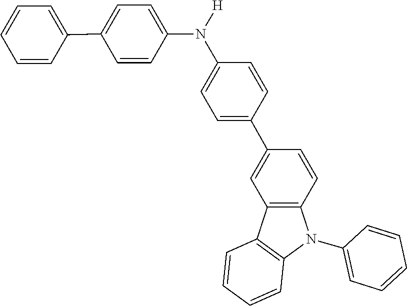 [SMILES: c1ccc(-c2ccc(Nc3ccc(-c4ccc5c(c4)c4ccccc4n5-c4ccccc4)cc3)cc2)cc1] 1160294-96-1 | 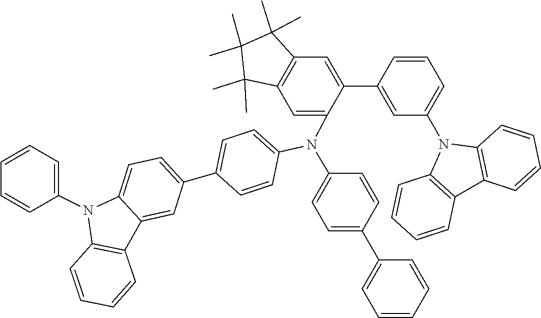 [SMILES: CC1(C)c2cc(-c3cccc(-n4c5ccccc5c5ccccc54)c3)c(N(c3ccc(-c4ccccc4)cc3)c3ccc(-c4ccc5c(c4)c4ccccc4n5-c4ccccc4)cc3)cc2C(C)(C)C1(C)C] | 70% |
| A50 | S47 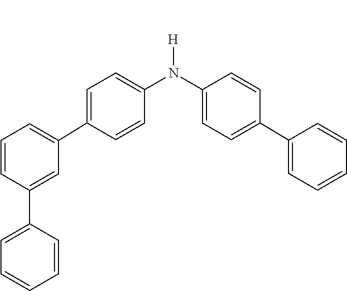 1222633-96-6 | 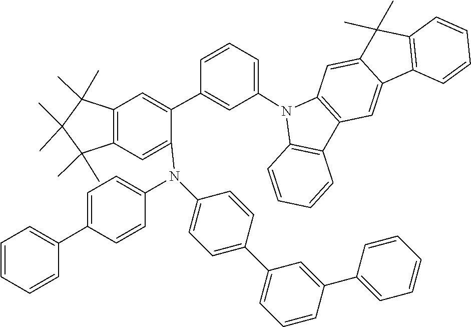 [SMILES: CC1(C)c2ccccc2-c2cc3c4ccccc4n(-c4cccc(-c5cc6c(cc5N(c5ccc(-c7ccccc7)cc5)c5ccc(-c7cccc(-c8ccccc8)c7)cc5)C(C)(C)C(C)(C)C6(C)C)c4)c3cc21] | 75% |
| A51 | S48 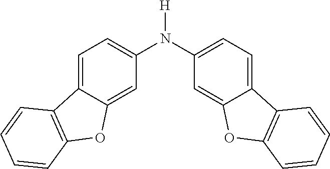 [SMILES: c1ccc2c(c1)oc1cc(Nc3ccc4c(c3)oc3ccccc34)ccc12] 1705595-86-3 | 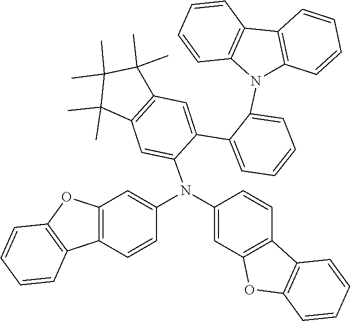 [SMILES: CC1(C)c2cc(-c3ccccc3-n3c4ccccc4c4ccccc43)c(N(c3ccc4c(c3)oc3ccccc34)c3ccc4c(c3)oc3ccccc34)cc2C(C)(C)C1(C)C] | 72% |
| A52 | S49 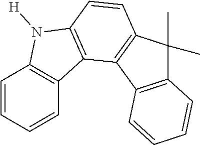 [SMILES: CC1(C)c2ccccc2-c2c1ccc1[nH]c3ccccc3c21] 1623813-70-6 | 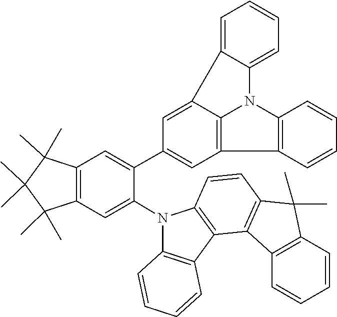 [SMILES: CC1(C)c2ccccc2-c2c1ccc1c2c2ccccc2n1-c1cc2c(cc1-c1cc3c4ccccc4n4c5ccccc5c(c1)c34)C(C)(C)C(C)(C)C2(C)C] | 76% |
| A53 | S50 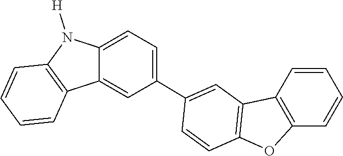 [SMILES: c1ccc2c(c1)[nH]c1ccc(-c3ccc4oc5ccccc5c4c3)cc12] 953805-18-0 | 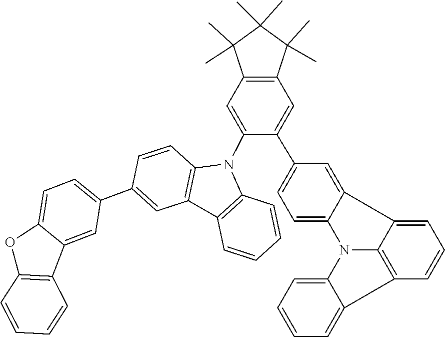 [SMILES: CC1(C)c2cc(-c3ccc4c(c3)c3cccc5c6ccccc6n4c53)c(-n3c4ccccc4c4cc(-c5ccc6oc7ccccc7c6c5)ccc43)cc2C(C)(C)C1(C)C] | 75% |
| A54 | S51 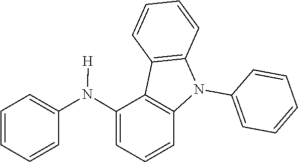 [SMILES: c1ccc(Nc2cccc3c2c2ccccc2n3-c2ccccc2)cc1] 1607445-46-4 | 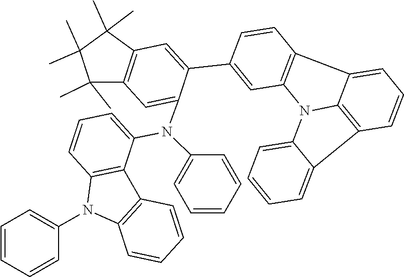 [SMILES: CC1(C)c2cc(-c3ccc4c5cccc6c7ccccc7n(c4c3)c65)c(N(c3ccccc3)c3cccc4c3c3ccccc3n4-c3ccccc3)cc2C(C)(C)C1(C)C] | 68% |
| A55 | S52 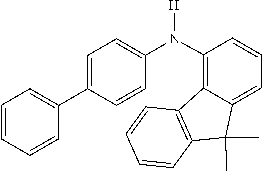 [SMILES: CC1(C)c2ccccc2-c2c(Nc3ccc(-c4ccccc4)cc3)cccc21] 1421789-16-3 | 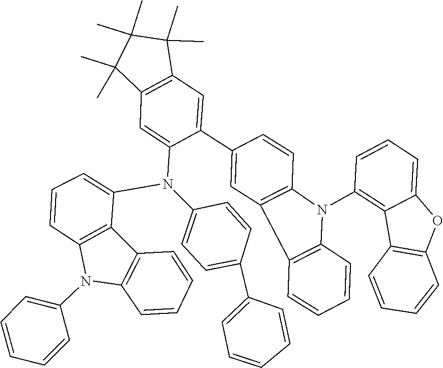 [SMILES: CC1(C)c2cc(-c3ccc4c(c3)c3ccccc3n4-c3cccc4oc5ccccc5c34)c(N(c3ccc(-c4ccccc4)cc3)c3cccc4c3c3ccccc3n4-c3ccccc3)cc2C(C)(C)C1(C)C] | 66% |
| A56 | S53 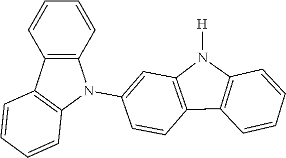 [SMILES: c1ccc2c(c1)[nH]c1cc(-n3c4ccccc4c4ccccc43)ccc12] 1226810-15-6 | 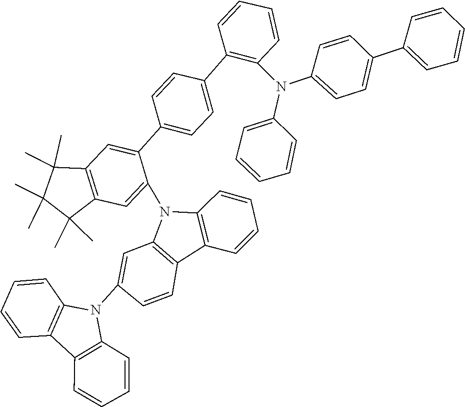 [SMILES: CC1(C)c2cc(-c3ccc(-c4ccccc4N(c4ccccc4)c4ccc(-c5ccccc5)cc4)cc3)c(-n3c4ccccc4c4ccc(-n5c6ccccc6c6ccccc65)cc43)cc2C(C)(C)C1(C)C] | 68% |
| A57 | S54 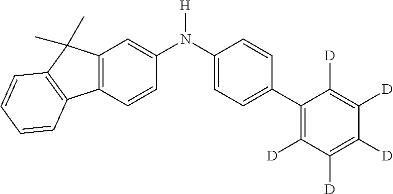 [SMILES: [2H]c1c([2H])c([2H])c(-c2ccc(Nc3ccc4c(c3)C(C)(C)c3ccccc3-4)cc2)c([2H])c1[2H]] 1359833-90-1 | 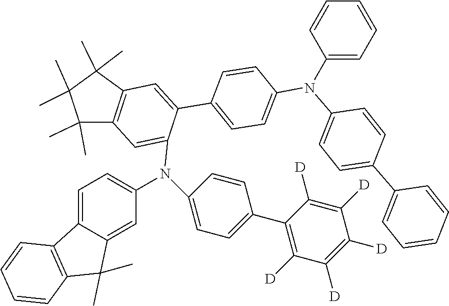 [SMILES: [2H]c1c([2H])c([2H])c(-c2ccc(N(c3ccc4c(c3)C(C)(C)c3ccccc3-4)c3cc4c(cc3-c3ccc(N(c5ccccc5)c5ccc(-c6ccccc6)cc5)cc3)C(C)(C)C(C)(C)C4(C)C)cc2)c([2H])c1[2H]] | 70% |
| A58 | S55 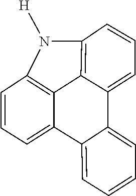 [SMILES: c1ccc2c(c1)c1cccc3[nH]c4cccc2c4c31] 109606-75-9 | 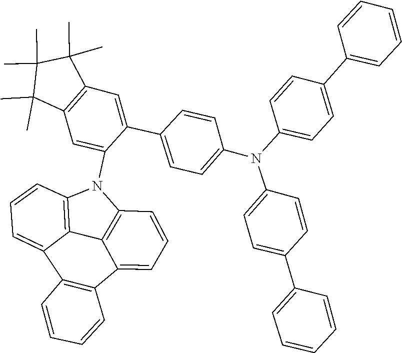 [SMILES: CC1(C)c2cc(-c3ccc(N(c4ccc(-c5ccccc5)cc4)c4ccc(-c5ccccc5)cc4)cc3)c(-n3c4cccc5c6ccccc6c6cccc3c6c54)cc2C(C)(C)C1(C)C] | 74% |
| A59 | S56 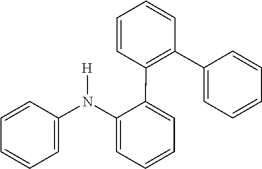 [SMILES: c1ccc(Nc2ccccc2-c2ccccc2-c2ccccc2)cc1] 1776936-11-8 | 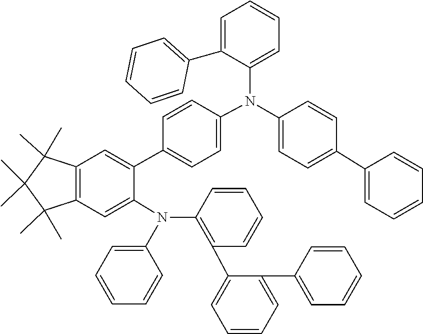 [SMILES: CC1(C)c2cc(-c3ccc(N(c4ccc(-c5ccccc5)cc4)c4ccccc4-c4ccccc4)cc3)c(N(c3ccccc3)c3ccccc3-c3ccccc3-c3ccccc3)cc2C(C)(C)C1(C)C] | 68% |
| A60 | S57 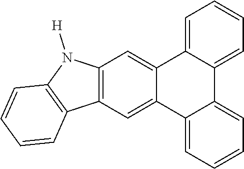 [SMILES: c1ccc2c(c1)[nH]c1cc3c4ccccc4c4ccccc4c3cc12] 1374446-05-5 | 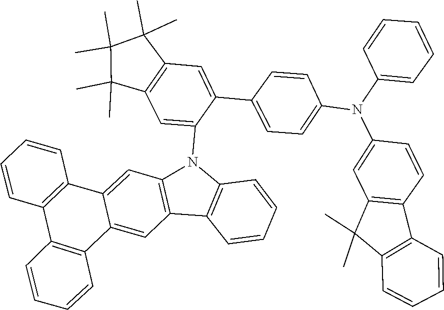 [SMILES: CC1(C)c2ccccc2-c2ccc(N(c3ccccc3)c3ccc(-c4cc5c(cc4-n4c6ccccc6c6cc7c8ccccc8c8ccccc8c7cc64)C(C)(C)C(C)(C)C5(C)C)cc3)cc21] | 71% |
| A61 | S58 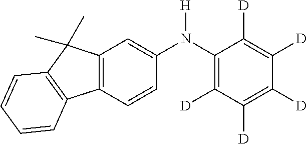 [SMILES: [2H]c1c([2H])c([2H])c(Nc2ccc3c(c2)C(C)(C)c2ccccc2-3)c([2H])c1[2H]] 1359833-31-0 | 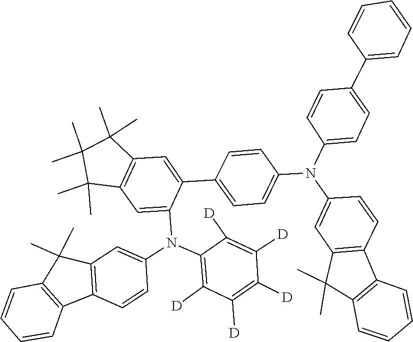 [SMILES: [2H]c1c([2H])c([2H])c(N(c2ccc3c(c2)C(C)(C)c2ccccc2-3)c2cc3c(cc2-c2ccc(N(c4ccc(-c5ccccc5)cc4)c4ccc5c(c4)C(C)(C)c4ccccc4-5)cc2)C(C)(C)C(C)(C)C3(C)C)c([2H])c1[2H]] | 69% |
| A62 | S59 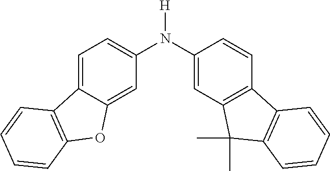 [SMILES: CC1(C)c2ccccc2-c2ccc(Nc3ccc4c(c3)oc3ccccc34)cc21] 1427556-50-0 | 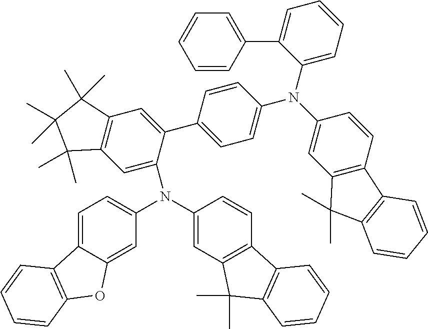 [SMILES: CC1(C)c2ccccc2-c2ccc(N(c3ccc(-c4cc5c(cc4N(c4ccc6c(c4)C(C)(C)c4ccccc4-6)c4ccc6c(c4)oc4ccccc46)C(C)(C)C(C)(C)C5(C)C)cc3)c3ccccc3-c3ccccc3)cc21] | 69% |
| A63 | S60 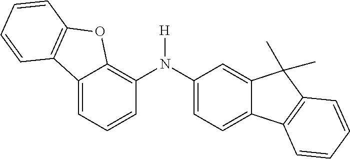 [SMILES: CC1(C)c2ccccc2-c2ccc(Nc3cccc4c3oc3ccccc34)cc21] 1426933-82-5 | 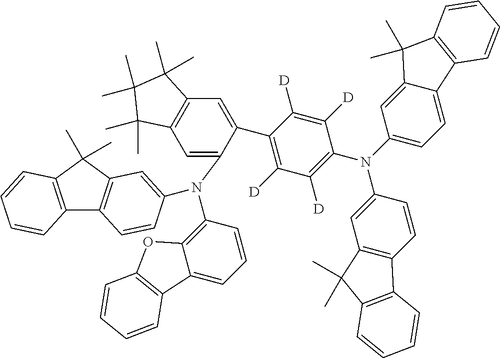 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1-c1cc2c(cc1N(c1ccc3c(c1)C(C)(C)c1ccccc1-3)c1cccc3c1oc1ccccc13)C(C)(C)C(C)(C)C2(C)C] | 70% |
| A64 | S61 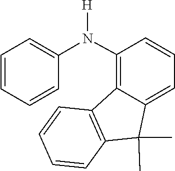 [SMILES: CC1(C)c2ccccc2-c2c(Nc3ccccc3)cccc21] 1776969-70-0 | 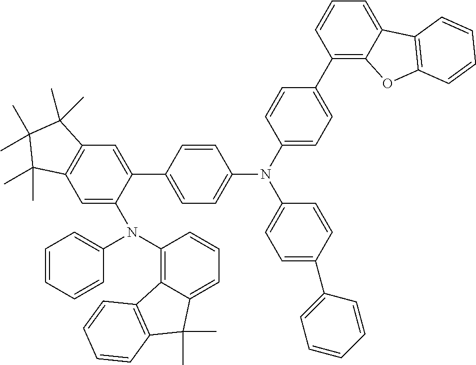 [SMILES: CC1(C)c2ccccc2-c2c(N(c3ccccc3)c3cc4c(cc3-c3ccc(N(c5ccc(-c6ccccc6)cc5)c5ccc(-c6cccc7c6oc6ccccc67)cc5)cc3)C(C)(C)C(C)(C)C4(C)C)cccc21] | 72% |
| A65 | S62 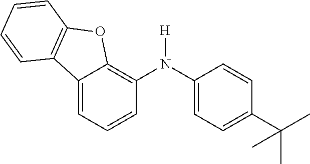 [SMILES: CC(C)(C)c1ccc(Nc2cccc3c2oc2ccccc23)cc1] 1438401-13-8 | 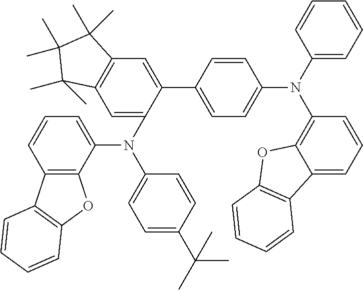 [SMILES: CC(C)(C)c1ccc(N(c2cc3c(cc2-c2ccc(N(c4ccccc4)c4cccc5c4oc4ccccc45)cc2)C(C)(C)C(C)(C)C3(C)C)c2cccc3c2oc2ccccc23)cc1] | 66% |
| A66 | S63 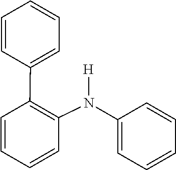 [SMILES: c1ccc(Nc2ccccc2-c2ccccc2)cc1] 35887-50-4 | 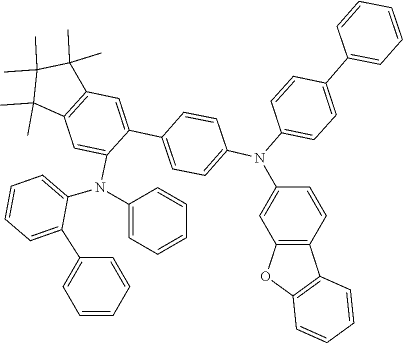 [SMILES: CC1(C)c2cc(-c3ccc(N(c4ccc(-c5ccccc5)cc4)c4ccc5c(c4)oc4ccccc45)cc3)c(N(c3ccccc3)c3ccccc3-c3ccccc3)cc2C(C)(C)C1(C)C] | 72% |
| A67 | S64 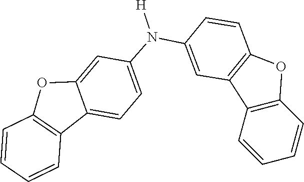 [SMILES: c1ccc2c(c1)oc1cc(Nc3ccc4oc5ccccc5c4c3)ccc12] 1923735-83-4 | 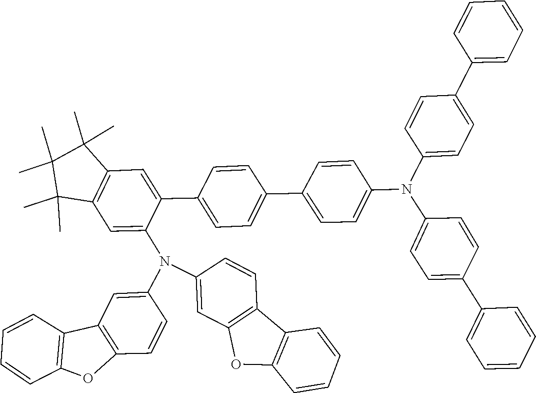 [SMILES: CC1(C)c2cc(-c3ccc(-c4ccc(N(c5ccc(-c6ccccc6)cc5)c5ccc(-c6ccccc6)cc5)cc4)cc3)c(N(c3ccc4c(c3)oc3ccccc34)c3ccc4oc5ccccc5c4c3)cc2C(C)(C)C1(C)C] | 69% |
| A68 | S65 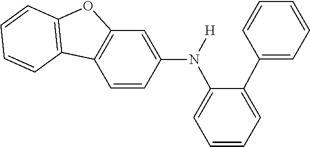 [SMILES: c1ccc(-c2ccccc2Nc2ccc3c(c2)oc2ccccc23)cc1] 1427556-44-2 | 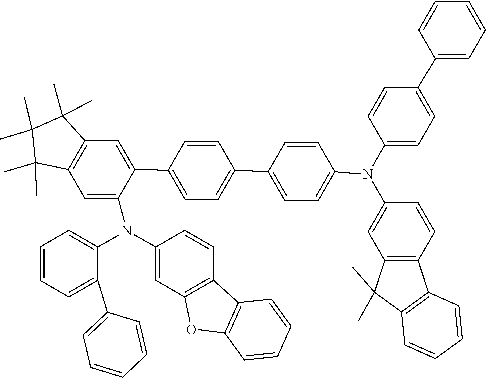 [SMILES: CC1(C)c2ccccc2-c2ccc(N(c3ccc(-c4ccccc4)cc3)c3ccc(-c4ccc(-c5cc6c(cc5N(c5ccc7c(c5)oc5ccccc57)c5ccccc5-c5ccccc5)C(C)(C)C(C)(C)C6(C)C)cc4)cc3)cc21] | 70% |
| A69 | S66 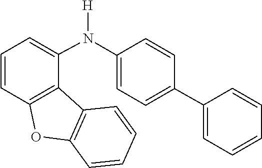 [SMILES: c1ccc(-c2ccc(Nc3cccc4oc5ccccc5c34)cc2)cc1] 1776057-10-3 | 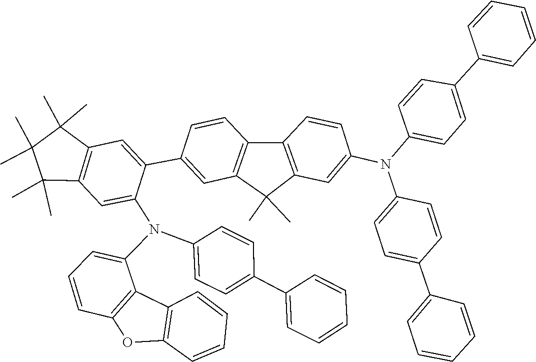 [SMILES: CC1(C)c2cc(-c3cc4c(cc3N(c3ccc(-c5ccccc5)cc3)c3cccc5oc6ccccc6c35)C(C)(C)C(C)(C)C4(C)C)ccc2-c2ccc(N(c3ccc(-c4ccccc4)cc3)c3ccc(-c4ccccc4)cc3)cc21] | 68% |
| A70 | S67 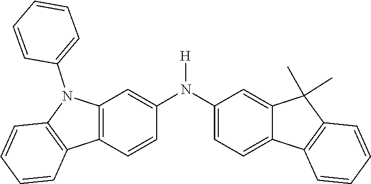 [SMILES: CC1(C)c2ccccc2-c2ccc(Nc3ccc4c5ccccc5n(-c5ccccc5)c4c3)cc21] 1456702-57-0 | 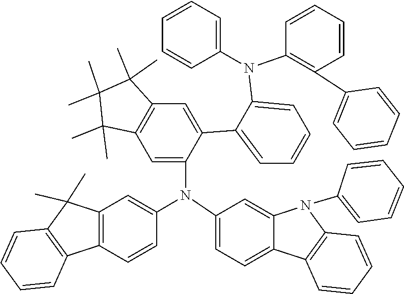 [SMILES: CC1(C)c2ccccc2-c2ccc(N(c3ccc4c5ccccc5n(-c5ccccc5)c4c3)c3cc4c(cc3-c3ccccc3N(c3ccccc3)c3ccccc3-c3ccccc3)C(C)(C)C(C)(C)C4(C)C)cc21] | 67 & |
| A71 | S68 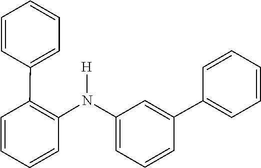 [SMILES: c1ccc(-c2cccc(Nc3ccccc3-c3ccccc3)c2)cc1] 1258515-01-3 | 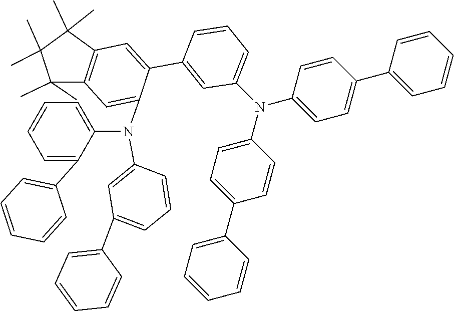 [SMILES: CC1(C)c2cc(-c3cccc(N(c4ccc(-c5ccccc5)cc4)c4ccc(-c5ccccc5)cc4)c3)c(N(c3cccc(-c4ccccc4)c3)c3ccccc3-c3ccccc3)cc2C(C)(C)C1(C)C] | 68% |
| A72 | S69 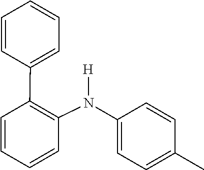 [SMILES: Cc1ccc(Nc2ccccc2-c2ccccc2)cc1] 118987-69-2 | 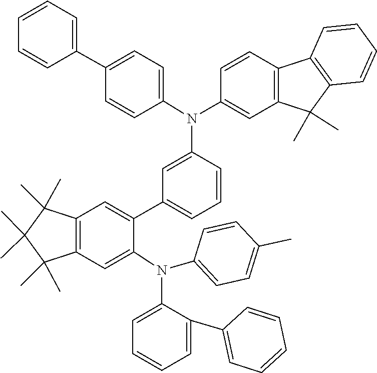 [SMILES: Cc1ccc(N(c2ccccc2-c2ccccc2)c2cc3c(cc2-c2cccc(N(c4ccc(-c5ccccc5)cc4)c4ccc5c(c4)C(C)(C)c4ccccc4-5)c2)C(C)(C)C(C)(C)C3(C)C)cc1] | 70% |
| A73 | S70 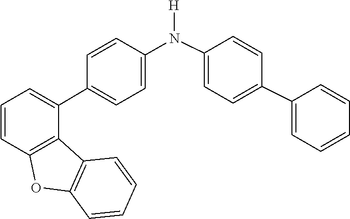 [SMILES: c1ccc(-c2ccc(Nc3ccc(-c4cccc5oc6ccccc6c45)cc3)cc2)cc1] 1922919-50-3 | 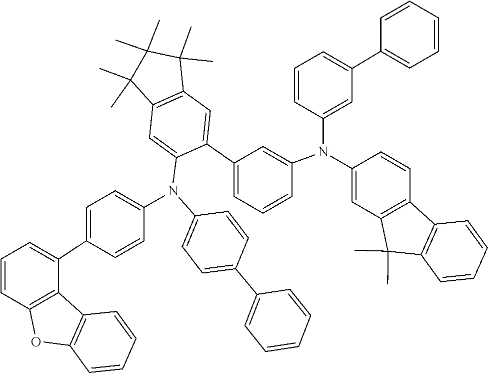 [SMILES: CC1(C)c2ccccc2-c2ccc(N(c3cccc(-c4ccccc4)c3)c3cccc(-c4cc5c(cc4N(c4ccc(-c6ccccc6)cc4)c4ccc(-c6cccc7oc8ccccc8c67)cc4)C(C)(C)C(C)(C)C5(C)C)c3)cc21] | 70% |
| A74 | S71 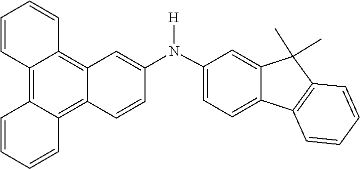 [SMILES: CC1(C)c2ccccc2-c2ccc(Nc3ccc4c5ccccc5c5ccccc5c4c3)cc21] 1430393-63-7 | 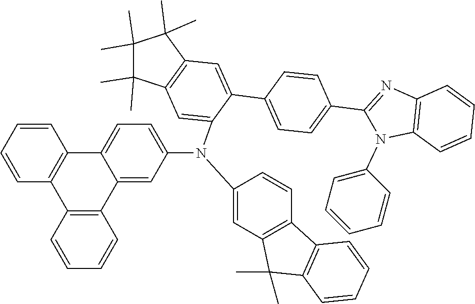 [SMILES: CC1(C)c2ccccc2-c2ccc(N(c3ccc4c5ccccc5c5ccccc5c4c3)c3cc4c(cc3-c3ccc(-c5nc6ccccc6n5-c5ccccc5)cc3)C(C)(C)C(C)(C)C4(C)C)cc21] | 74% |
| A75 | S72 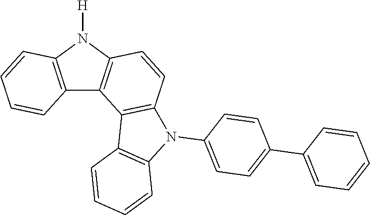 [SMILES: c1ccc(-c2ccc(-n3c4ccccc4c4c5c(ccc43)[nH]c3ccccc35)cc2)cc1] 2071630-78-7 | 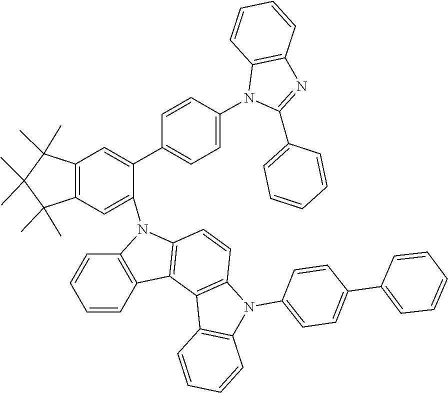 [SMILES: CC1(C)c2cc(-c3ccc(-n4c(-c5ccccc5)nc5ccccc54)cc3)c(-n3c4ccccc4c4c5c6ccccc6n(-c6ccc(-c7ccccc7)cc6)c5ccc43)cc2C(C)(C)C1(C)C] | 75% |
| A76 | S73 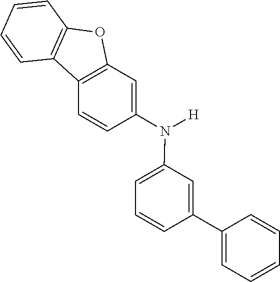 [SMILES: c1ccc(-c2cccc(Nc3ccc4c(c3)oc3ccccc34)c2)cc1] 1427556-45-3 | 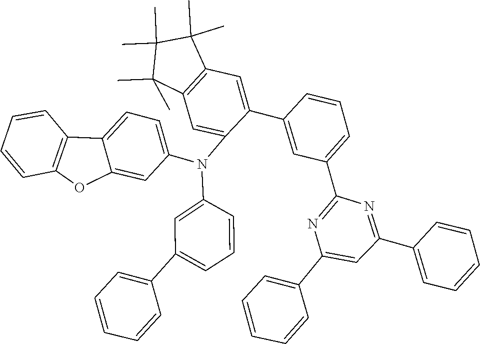 [SMILES: CC1(C)c2cc(-c3cccc(-c4nc(-c5ccccc5)cc(-c5ccccc5)n4)c3)c(N(c3cccc(-c4ccccc4)c3)c3ccc4c(c3)oc3ccccc34)cc2C(C)(C)C1(C)C] | 70% |
| A77 | S74 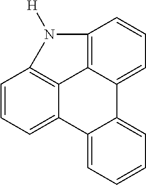 [SMILES: c1ccc2c(c1)c1cccc3[nH]c4cccc2c4c31] 109606-75-9 | 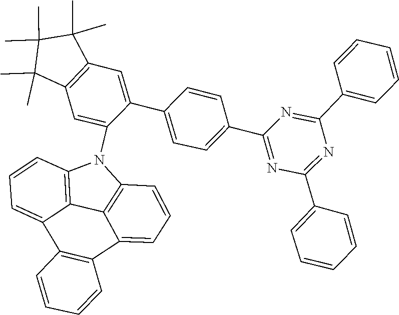 [SMILES: CC1(C)c2cc(-c3ccc(-c4nc(-c5ccccc5)nc(-c5ccccc5)n4)cc3)c(-n3c4cccc5c6ccccc6c6cccc3c6c54)cc2C(C)(C)C1(C)C] | 78% |
| A78 | S75 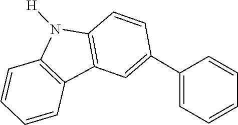 [SMILES: c1ccc(-c2ccc3[nH]c4ccccc4c3c2)cc1] 103012-26-6 | 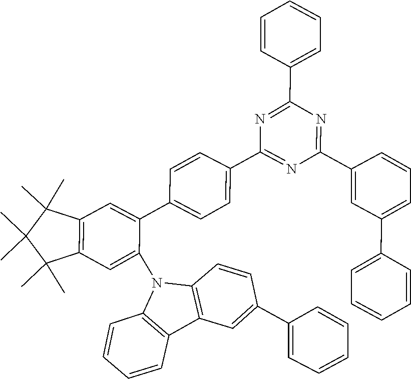 [SMILES: CC1(C)c2cc(-c3ccc(-c4nc(-c5ccccc5)nc(-c5cccc(-c6ccccc6)c5)n4)cc3)c(-n3c4ccccc4c4cc(-c5ccccc5)ccc43)cc2C(C)(C)C1(C)C] | 75% |
| A79 | S76 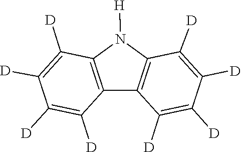 [SMILES: [2H]c1c([2H])c([2H])c2c([nH]c3c([2H])c([2H])c([2H])c([2H])c32)c1[2H]] | 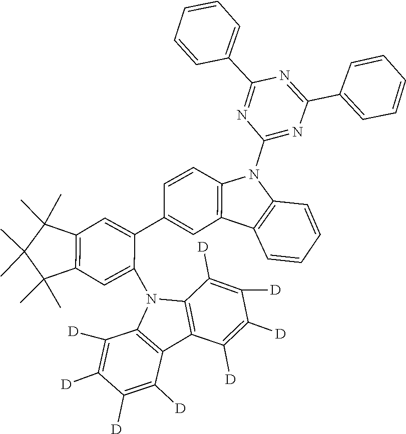 [SMILES: [2H]c1c([2H])c([2H])c2c(c1[2H])c1c([2H])c([2H])c([2H])c([2H])c1n2-c1cc2c(cc1-c1ccc3c(c1)c1ccccc1n3-c1nc(-c3ccccc3)nc(-c3ccccc3)n1)C(C)(C)C(C)(C)C2(C)C] | 68% |
| A80 | S77 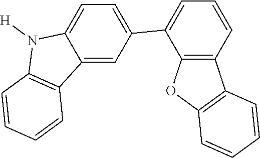 [SMILES: c1ccc2c(c1)[nH]c1ccc(-c3cccc4c3oc3ccccc34)cc12] 1346669-46-2 | 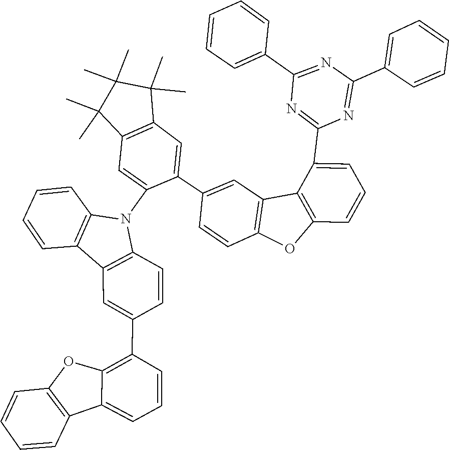 [SMILES: CC1(C)c2cc(-c3ccc4oc5cccc(-c6nc(-c7ccccc7)nc(-c7ccccc7)n6)c5c4c3)c(-n3c4ccccc4c4cc(-c5cccc6c5oc5ccccc56)ccc43)cc2C(C)(C)C1(C)C] | 65% |

### Example A100

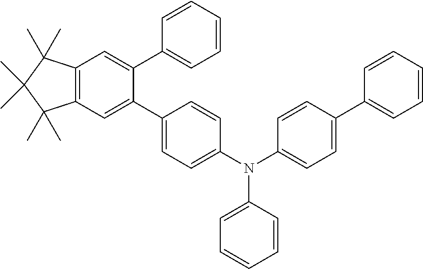 [SMILES: CC1(C)c2cc(-c3ccccc3)c(-c3ccc(N(c4ccccc4)c4ccc(-c5ccccc5)cc4)cc3)cc2C(C)(C)C1(C)C]

Procedure analogous to J. L. Bolliger et al., Chem. Eur. J. 2010, 16, 4075, Table 1, Entry 28. Starting materials: 36.0 g (100 mmol) of LS1, 40.2 g (110 mmol) of B-[4-([1,1′-biphenyl]-4-ylphenylamino)phenyl]boronic acid [1084334-86-0]. The crude product is purified by chromatography and repeated hot-extraction crystallization (customary organic solvents or the combinations thereof, preferably acetonitrile-DCM, 1:3 to 3:1 vv) and fractional sublimation or heat treatment under high vacuum. Yield: 45.6 g (76 mmol), 76%; purity: about 99.9% by HPLC.

The following compounds can be prepared analogously:

| Ex. | Reactants | Product | Yield |
|---|---|---|---|
| A101 | S2 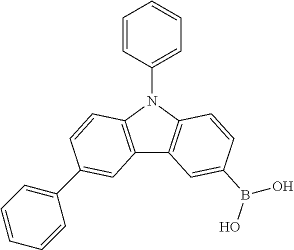 | 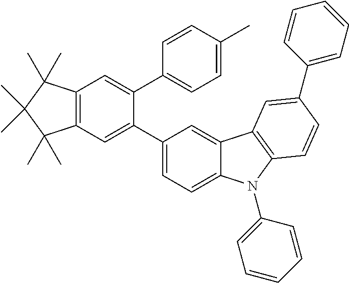 [SMILES: Cc1ccc(-c2cc3c(cc2-c2ccc4c(c2)c2cc(-c5ccccc5)ccc2n4-c2ccccc2)C(C)(C)C(C)(C)C3(C)C)cc1] | 75% |
| | 81359833-28-5 | | |
| A102 | S3 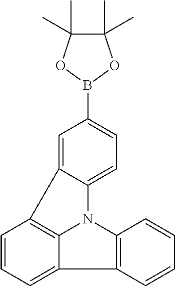 | 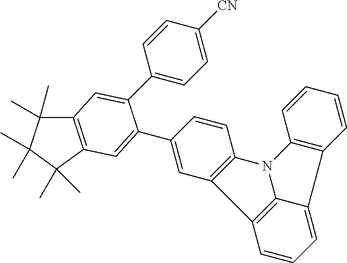 [SMILES: CC1(C)c2cc(-c3ccc(C#N)cc3)c(-c3ccc4c(c3)c3cccc5c6ccccc6n4c53)cc2C(C)(C)C1(C)C] | 81% |
| | 1454807-26-1 | | |
| A103 | S4 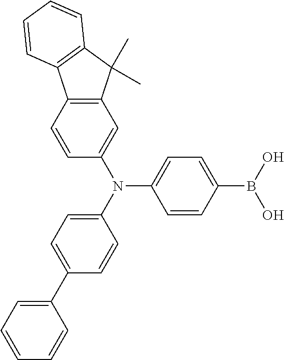 | 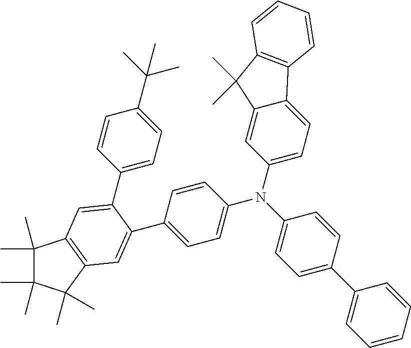 [SMILES: CC(C)(C)c1ccc(-c2cc3c(cc2-c2ccc(N(c4ccc(-c5ccccc5)cc4)c4ccc5c(c4)C(C)(C)c4ccccc4-5)cc2)C(C)(C)C(C)(C)C3(C)C)cc1] | 79% |
| | 1265177-27-2 | | |
| A104 | S5 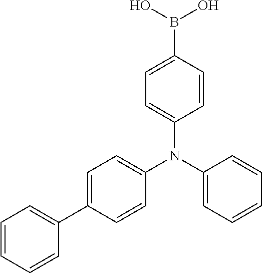 1084334-86-0 | 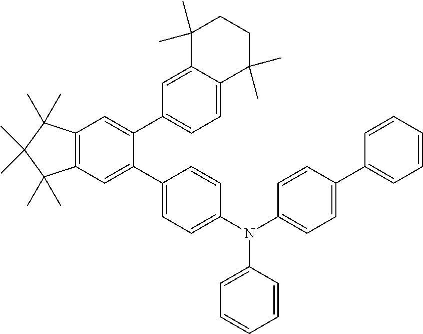 [SMILES: CC1(C)CCC(C)(C)c2cc(-c3cc4c(cc3-c3ccc(N(c5ccccc5)c5ccc(-c6ccccc6)cc5)cc3)C(C)(C)C(C)(C)C4(C)C)ccc21] | 77% |
| A105 | S6 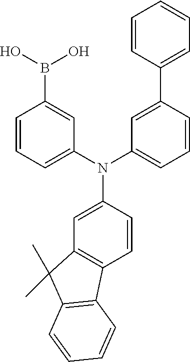 | 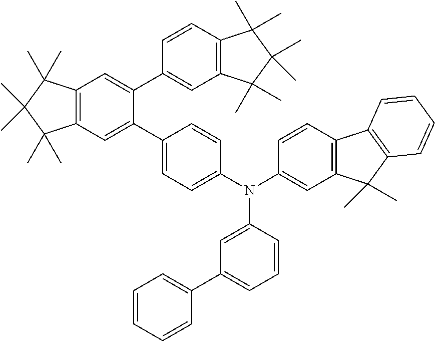 [SMILES: CC1(C)c2ccccc2-c2ccc(N(c3ccc(-c4cc5c(cc4-c4ccc6c(c4)C(C)(C)C(C)(C)C6(C)C)C(C)(C)C(C)(C)C5(C)C)cc3)c3cccc(-c4ccccc4)c3)cc21] | 73% |
| | 2334467-29-5 | | |
| A106 | S7 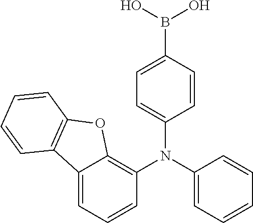 | 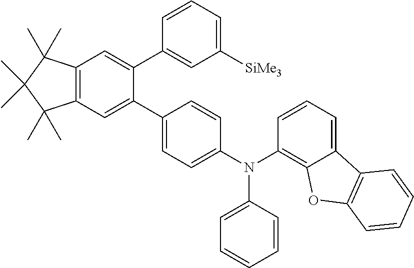 [SMILES: CC1(C)c2cc(-c3ccc(N(c4ccccc4)c4cccc5c4oc4ccccc45)cc3)c(-c3cccc([Si](C)(C)C)c3)cc2C(C)(C)C1(C)C] | 55% |
| | 1428329-78-5 | | |
| A107 | S8 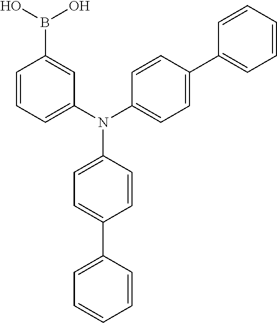 | 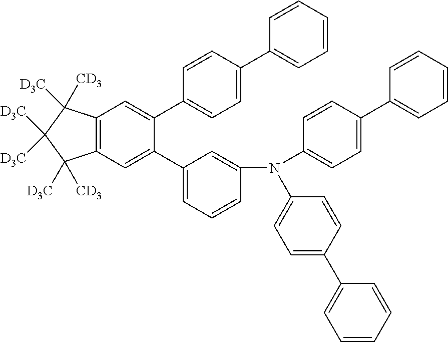 [SMILES: [2H]C([2H])([2H])C1(C([2H])([2H])[2H])c2cc(-c3ccc(-c4ccccc4)cc3)c(-c3cccc(N(c4ccc(-c5ccccc5)cc4)c4ccc(-c5ccccc5)cc4)c3)cc2C(C([2H])([2H])[2H])(C([2H])([2H])[2H])C1(C([2H])([2H])[2H])C([2H])([2H])[2H]] | 76% |
| | 1776936-65-2 | | |
| A108 | S9 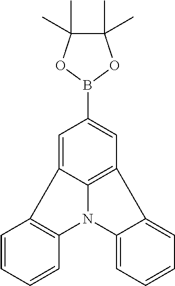 | 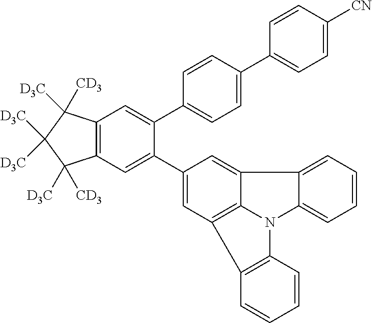 [SMILES: [2H]C([2H])([2H])C1(C([2H])([2H])[2H])c2cc(-c3ccc(-c4ccc(C#N)cc4)cc3)c(-c3cc4c5ccccc5n5c6ccccc6c(c3)c45)cc2C(C([2H])([2H])[2H])(C([2H])([2H])[2H])C1(C([2H])([2H])[2H])C([2H])([2H])[2H]] | 79% |
| | 1369369-44-7 | | |
| A109 | S10 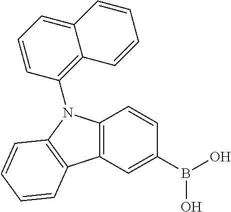 | 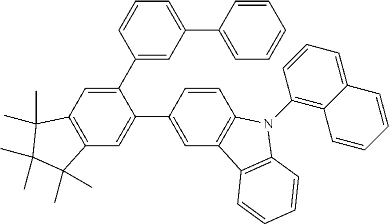 [SMILES: CC1(C)c2cc(-c3cccc(-c4ccccc4)c3)c(-c3ccc4c(c3)c3ccccc3n4-c3cccc4ccccc34)cc2C(C)(C)C1(C)C] | 69% |
| | 1133057-97-2 | | |
| A110 | S11 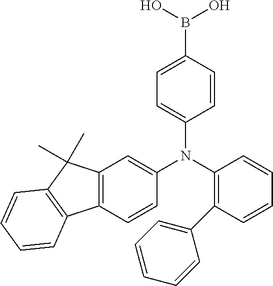 | 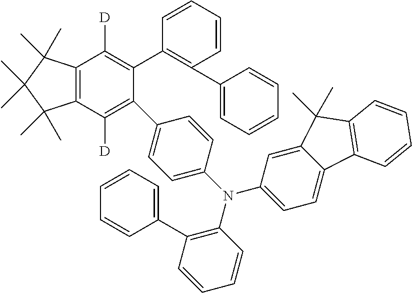 [SMILES: [2H]c1c(-c2ccc(N(c3ccc4c(c3)C(C)(C)c3ccccc3-4)c3ccccc3-c3ccccc3)cc2)c(-c2ccccc2-c2ccccc2)c([2H])c2c1C(C)(C)C(C)(C)C2(C)C] | 67% |
| | 1959599-90-6 | | |
| A111 | S12 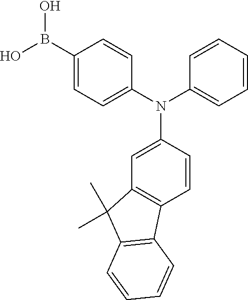 | 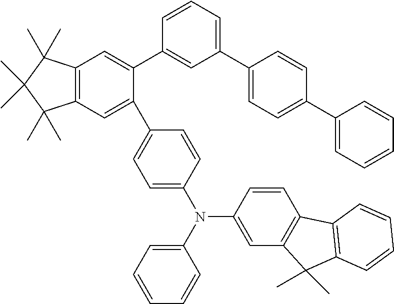 [SMILES: CC1(C)c2ccccc2-c2ccc(N(c3ccccc3)c3ccc(-c4cc5c(cc4-c4cccc(-c6ccc(-c7ccccc7)cc6)c4)C(C)(C)C(C)(C)C5(C)C)cc3)cc21] | 69% |
| | 950986-07-9 | | |
| A112 | S13 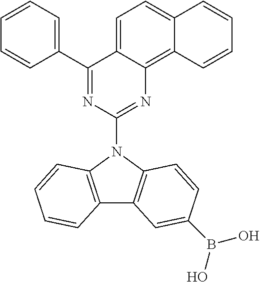 | 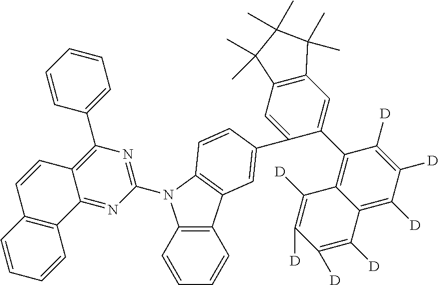 [SMILES: [2H]c1c([2H])c([2H])c2c(-c3cc4c(cc3-c3ccc5c(c3)c3ccccc3n5-c3nc(-c5ccccc5)c5ccc6ccccc6c5n3)C(C)(C)C(C)(C)C4(C)C)c([2H])c([2H])c([2H])c2c1[2H]] | 64% |
| | 1813537-15-3 | | |
| A113 | S14 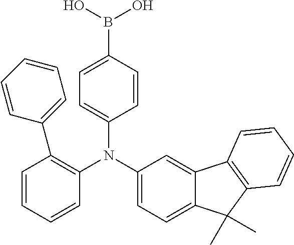 2609773-06-8 | 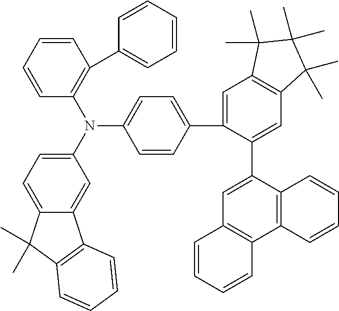 [SMILES: CC1(C)c2ccccc2-c2cc(N(c3ccc(-c4cc5c(cc4-c4cc6ccccc6c6ccccc46)C(C)(C)C(C)(C)C5(C)C)cc3)c3ccccc3-c3ccccc3)ccc21] | 68% |
| A114 | S15 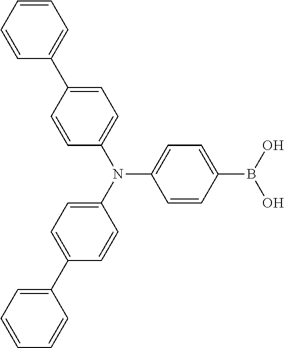 | 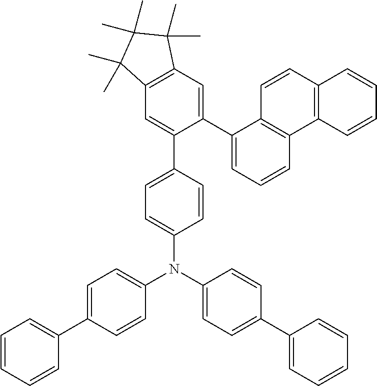 [SMILES: CC1(C)c2cc(-c3ccc(N(c4ccc(-c5ccccc5)cc4)c4ccc(-c5ccccc5)cc4)cc3)c(-c3cccc4c3ccc3ccccc34)cc2C(C)(C)C1(C)C] | 81% |
| | 943836-24-6 | | |
| A115 | S16 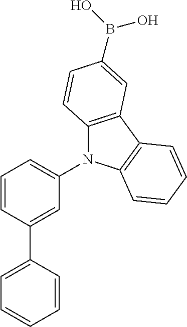 | 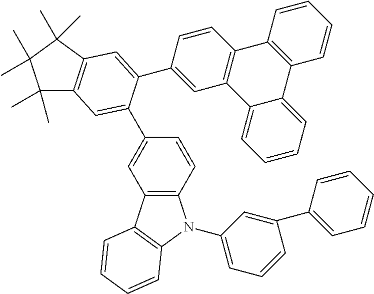 [SMILES: CC1(C)c2cc(-c3ccc4c5ccccc5c5ccccc5c4c3)c(-c3ccc4c(c3)c3ccccc3n4-c3cccc(-c4ccccc4)c3)cc2C(C)(C)C1(C)C] | 78% |
| | 1416814-68-0 | | |
| A116 | S17 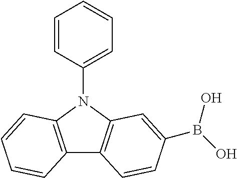 1001911-63-2 | 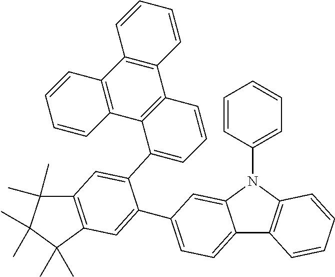 [SMILES: CC1(C)c2cc(-c3ccc4c5ccccc5n(-c5ccccc5)c4c3)c(-c3cccc4c5ccccc5c5ccccc5c34)cc2C(C)(C)C1(C)C] | 71% |
| A117 | S18 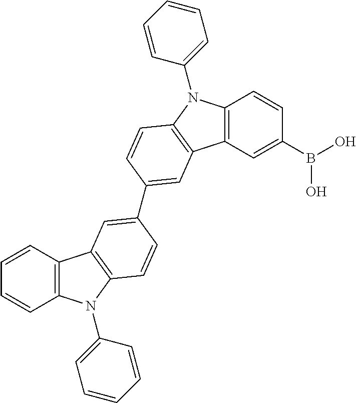 | 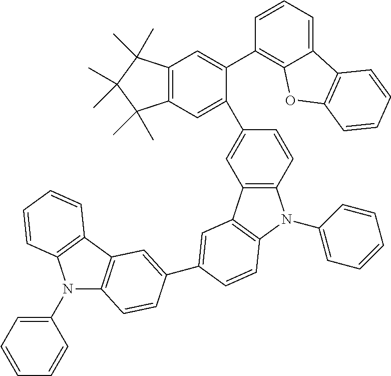 [SMILES: CC1(C)c2cc(-c3ccc4c(c3)c3cc(-c5ccc6c(c5)c5ccccc5n6-c5ccccc5)ccc3n4-c3ccccc3)c(-c3cccc4c3oc3ccccc34)cc2C(C)(C)C1(C)C] | 67% |
| | 918137-86-7 | | |
| A118 | S19 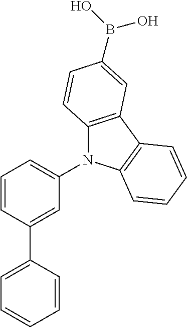 | 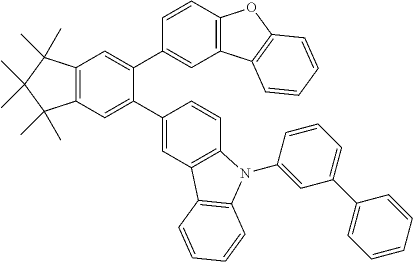 [SMILES: CC1(C)c2cc(-c3ccc4oc5ccccc5c4c3)c(-c3ccc4c(c3)c3ccccc3n4-c3cccc(-c4ccccc4)c3)cc2C(C)(C)C1(C)C] | 70% |
| | 1416814-68-0 | | |
| A119 | S20 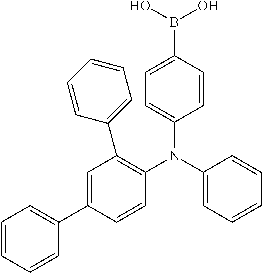 | 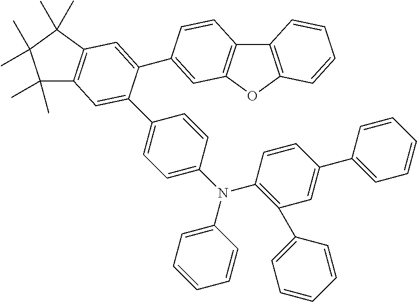 [SMILES: CC1(C)c2cc(-c3ccc(N(c4ccccc4)c4ccc(-c5ccccc5)cc4-c4ccccc4)cc3)c(-c3ccc4c(c3)oc3ccccc34)cc2C(C)(C)C1(C)C] | 76% |
| | 1417334-02-1 | | |
| A120 | S21 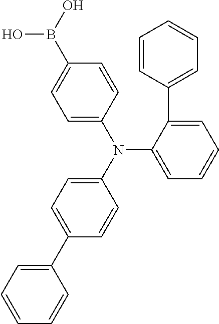 | 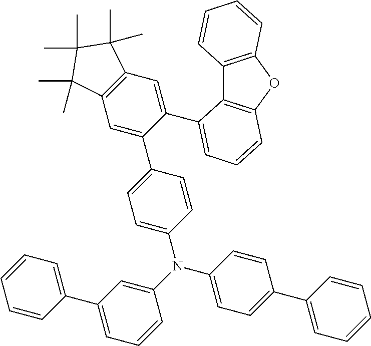 [SMILES: CC1(C)c2cc(-c3ccc(N(c4ccc(-c5ccccc5)cc4)c4cccc(-c5ccccc5)c4)cc3)c(-c3cccc4oc5ccccc5c34)cc2C(C)(C)C1(C)C] | 70% |
| | 1608462-54-9 | | |
| A121 | S22 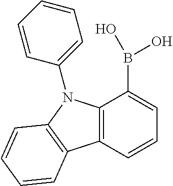 1333002-41-7 | 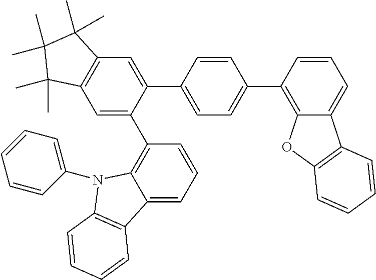 [SMILES: CC1(C)c2cc(-c3ccc(-c4cccc5c4oc4ccccc45)cc3)c(-c3cccc4c5ccccc5n(-c5ccccc5)c34)cc2C(C)(C)C1(C)C] | 65% |
| A122 | S23 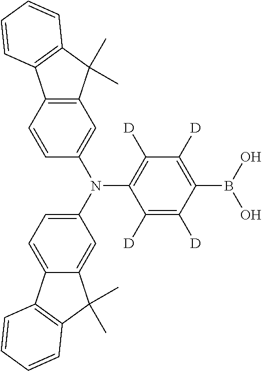 | 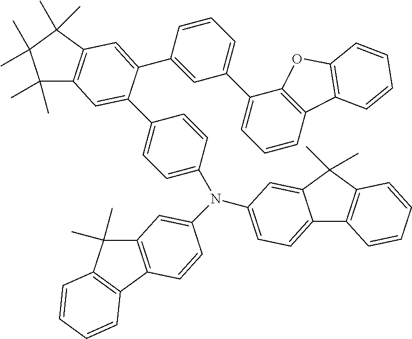 [SMILES: CC1(C)c2ccccc2-c2ccc(N(c3ccc(-c4cc5c(cc4-c4cccc(-c6cccc7c6oc6ccccc67)c4)C(C)(C)C(C)(C)C5(C)C)cc3)c3ccc4c(c3)C(C)(C)c3ccccc3-4)cc21] | 68% |
| | 1648570-88-0 | | |
| A123 | S24 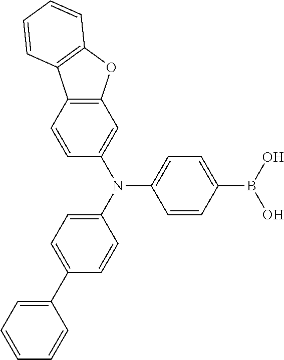 | 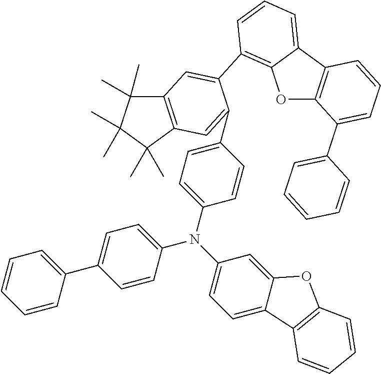 [SMILES: CC1(C)c2cc(-c3ccc(N(c4ccc(-c5ccccc5)cc4)c4ccc5c(c4)oc4ccccc45)cc3)c(-c3cccc4c3oc3c(-c5ccccc5)cccc34)cc2C(C)(C)C1(C)C] | 70% |
| | 1960443-69-9 | | |
| A124 | S25 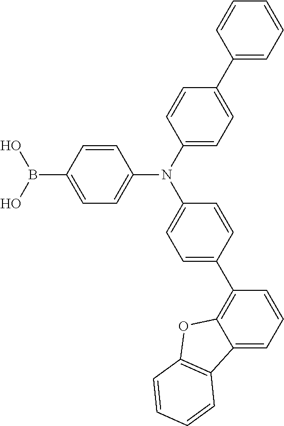 | 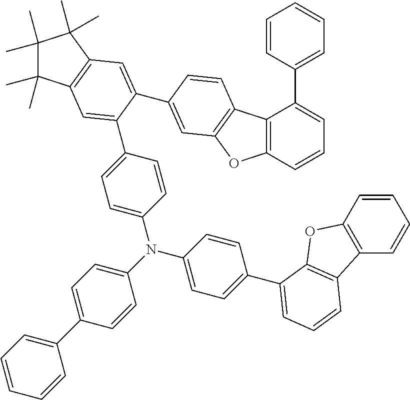 [SMILES: CC1(C)c2cc(-c3ccc(N(c4ccc(-c5ccccc5)cc4)c4ccc(-c5cccc6c5oc5ccccc56)cc4)cc3)c(-c3ccc4c(c3)oc3cccc(-c5ccccc5)c34)cc2C(C)(C)C1(C)C] | 69% |
| | 2126887-02- | | |
| A125 | S26 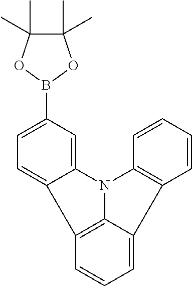 | 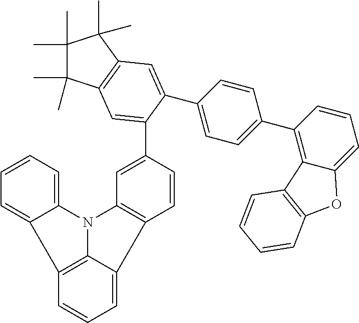 [SMILES: CC1(C)c2cc(-c3ccc(-c4cccc5oc6ccccc6c45)cc3)c(-c3ccc4c5cccc6c7ccccc7n(c4c3)c65)cc2C(C)(C)C1(C)C] | 79% |
| | 2068731-68-8 | | |
| A126 | S27 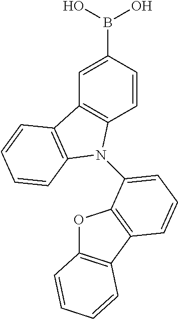 | 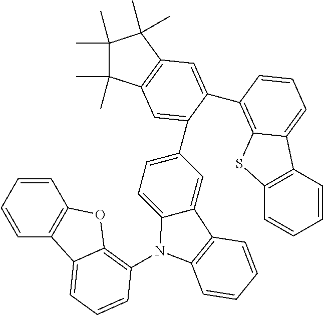 [SMILES: CC1(C)c2cc(-c3ccc4c(c3)c3ccccc3n4-c3cccc4c3oc3ccccc34)c(-c3cccc4c3sc3ccccc34)cc2C(C)(C)C1(C)C] | 76% |
| | 1547397-15-8 | | |
| A127 | S28 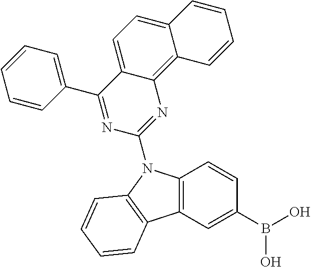 1813537-15-3 | 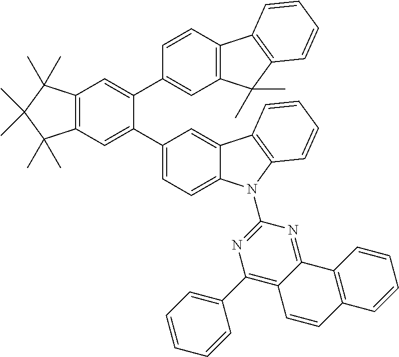 [SMILES: CC1(C)c2ccccc2-c2ccc(-c3cc4c(cc3-c3ccc5c(c3)c3ccccc3n5-c3nc(-c5ccccc5)c5ccc6ccccc6c5n3)C(C)(C)C(C)(C)C4(C)C)cc21] | 77% |
| A128 | S29 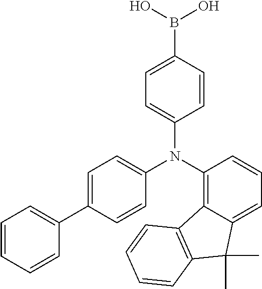 | 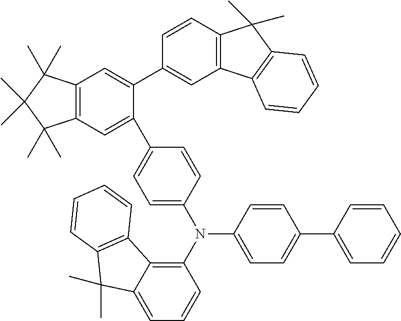 [SMILES: CC1(C)c2ccccc2-c2cc(-c3cc4c(cc3-c3ccc(N(c5ccc(-c6ccccc6)cc5)c5cccc6c5-c5ccccc5C6(C)C)cc3)C(C)(C)C(C)(C)C4(C)C)ccc21] | 69% |
| | 2410401-87-3 | | |
| A129 | S30 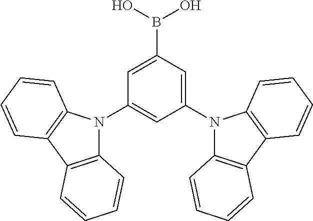 854952-51-5 | 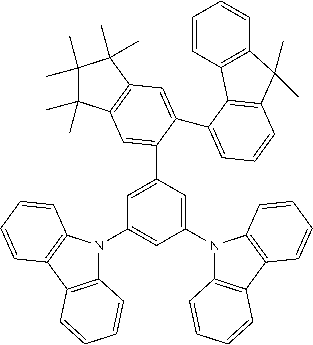 [SMILES: CC1(C)c2ccccc2-c2c(-c3cc4c(cc3-c3cc(-n5c6ccccc6c6ccccc65)cc(-n5c6ccccc6c6ccccc65)c3)C(C)(C)C(C)(C)C4(C)C)cccc21] | 73% |
| A130 | S31 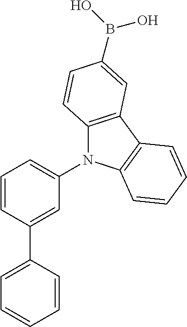 | 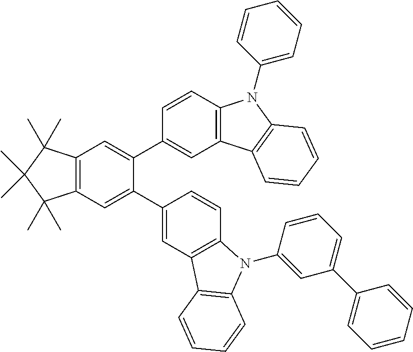 [SMILES: CC1(C)c2cc(-c3ccc4c(c3)c3ccccc3n4-c3ccccc3)c(-c3ccc4c(c3)c3ccccc3n4-c3cccc(-c4ccccc4)c3)cc2C(C)(C)C1(C)C] | 70% |
| | 1416814-68-0 | | |
| A131 | S32 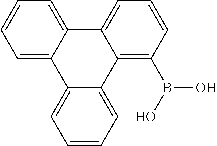 1430392-46-3 | 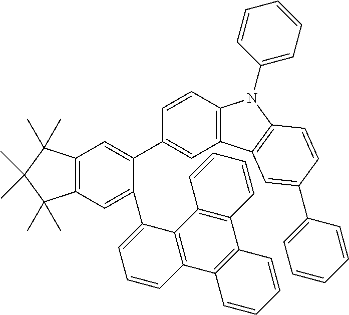 [SMILES: CC1(C)c2cc(-c3ccc4c(c3)c3cc(-c5ccccc5)ccc3n4-c3ccccc3)c(-c3cccc4c5ccccc5c5ccccc5c34)cc2C(C)(C)C1(C)C] | 66% |
| A132 | S33 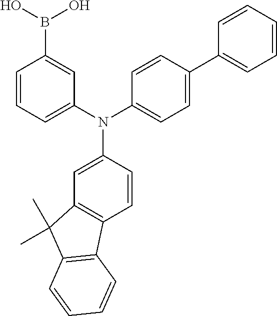 | 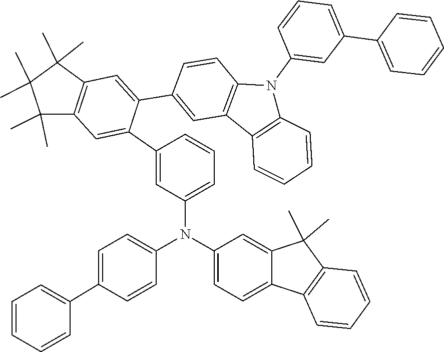 [SMILES: CC1(C)c2ccccc2-c2ccc(N(c3ccc(-c4ccccc4)cc3)c3cccc(-c4cc5c(cc4-c4ccc6c(c4)c4ccccc4n6-c4cccc(-c6ccccc6)c4)C(C)(C)C(C)(C)C5(C)C)c3)cc21] | 68% |
| | 1610950-84-9 | | |
| A133 | S34 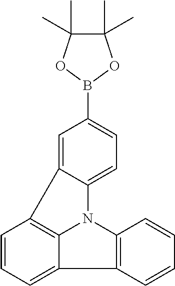 | 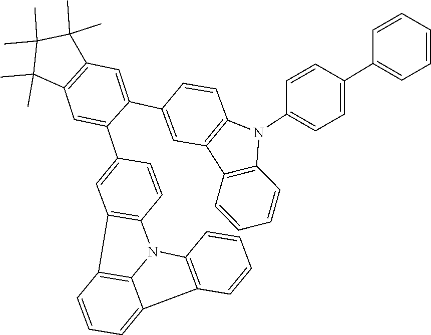 [SMILES: CC1(C)c2cc(-c3ccc4c(c3)c3ccccc3n4-c3ccc(-c4ccccc4)cc3)c(-c3ccc4c(c3)c3cccc5c6ccccc6n4c53)cc2C(C)(C)C1(C)C] | 74% |
| | 1454807-26-1 | | |
| A134 | S35 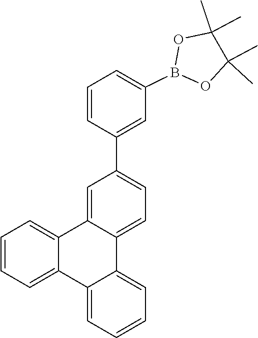 | 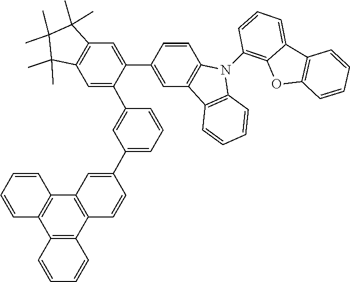 [SMILES: CC1(C)c2cc(-c3cccc(-c4ccc5c6ccccc6c6ccccc6c5c4)c3)c(-c3ccc4c(c3)c3ccccc3n4-c3cccc4c3oc3ccccc34)cc2C(C)(C)C1(C)C] | 74% |
| | 1115639-92-3 | | |
| A135 | S36 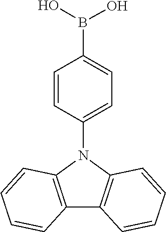 419536-33-7 | 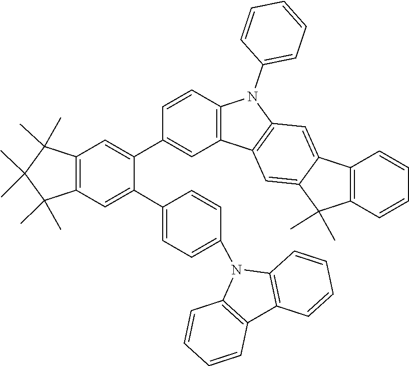 [SMILES: CC1(C)c2ccccc2-c2cc3c(cc21)c1cc(-c2cc4c(cc2-c2ccc(-n5c6ccccc6c6ccccc65)cc2)C(C)(C)C(C)(C)C4(C)C)ccc1n3-c1ccccc1] | 70% |
| A136 | S37 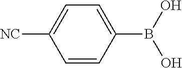 126747-14-6 | 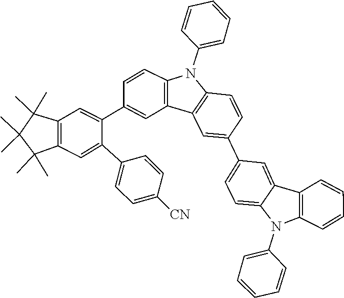 [SMILES: CC1(C)c2cc(-c3ccc(C#N)cc3)c(-c3ccc4c(c3)c3cc(-c5ccc6c(c5)c5ccccc5n6-c5ccccc5)ccc3n4-c3ccccc3)cc2C(C)(C)C1(C)C] | 72% |
| A137 | S38 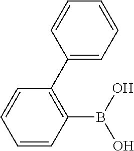 914675-52-8 | 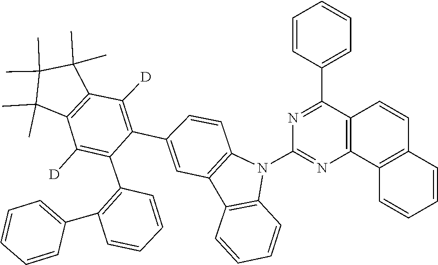 [SMILES: [2H]c1c(-c2ccc3c(c2)c2ccccc2n3-c2nc(-c3ccccc3)c3ccc4ccccc4c3n2)c(-c2ccccc2-c2ccccc2)c([2H])c2c1C(C)(C)C(C)(C)C2(C)C] | 75% |
| A138 | S39 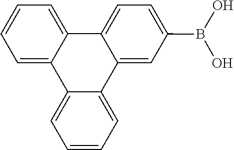 654664-63-8 | 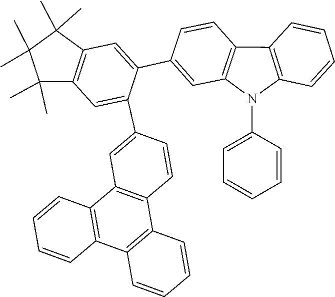 [SMILES: CC1(C)c2cc(-c3ccc4c5ccccc5c5ccccc5c4c3)c(-c3ccc4c5ccccc5n(-c5ccccc5)c4c3)cc2C(C)(C)C1(C)C] | 78% |
| A139 | S40 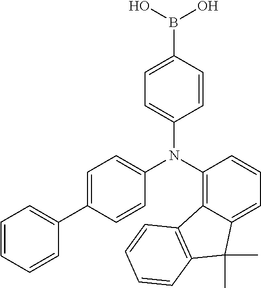 2410401-87-3 | 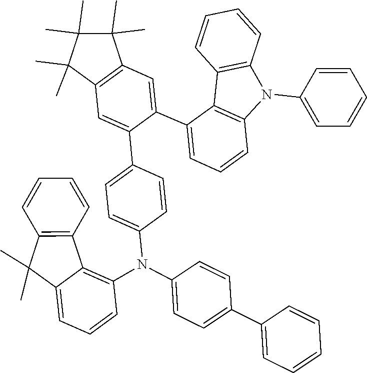 [SMILES: CC1(C)c2ccccc2-c2c(N(c3ccc(-c4ccccc4)cc3)c3ccc(-c4cc5c(cc4-c4cccc6c4c4ccccc4n6-c4ccccc4)C(C)(C)C(C)(C)C5(C)C)cc3)cccc21] | 71% |
| A140 | S41 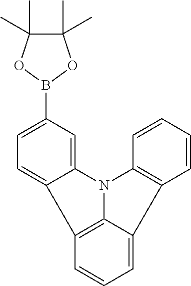 | 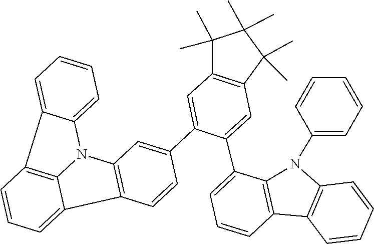 [SMILES: CC1(C)c2cc(-c3ccc4c5cccc6c7ccccc7n(c4c3)c65)c(-c3cccc4c5ccccc5n(-c5ccccc5)c34)cc2C(C)(C)C1(C)C] | 74% |
| | 2068731-68-8 | | |
| A141 | S42 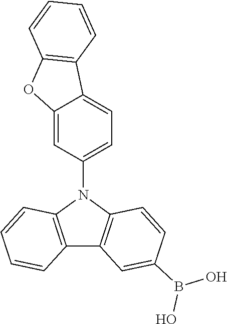 1637323-05-7 | 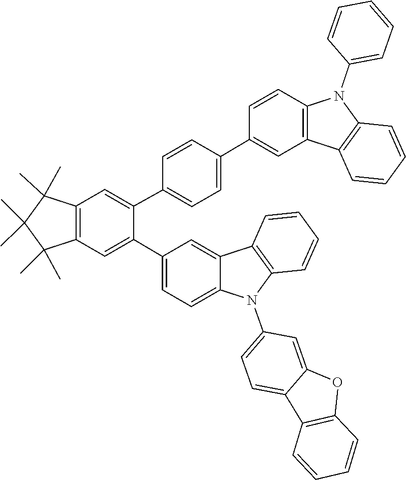 [SMILES: CC1(C)c2cc(-c3ccc(-c4ccc5c(c4)c4ccccc4n5-c4ccccc4)cc3)c(-c3ccc4c(c3)c3ccccc3n4-c3ccc4c(c3)oc3ccccc34)cc2C(C)(C)C1(C)C] | 70% |
| A142 | S43 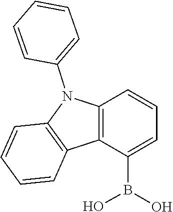 1370555-65-9 | 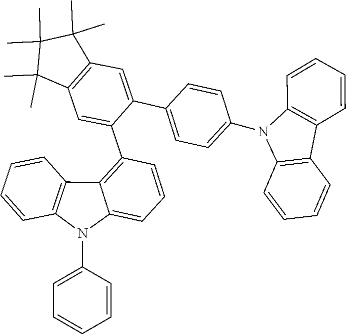 [SMILES: CC1(C)c2cc(-c3ccc(-n4c5ccccc5c5ccccc54)cc3)c(-c3cccc4c3c3ccccc3n4-c3ccccc3)cc2C(C)(C)C1(C)C] | 78% |
| A143 | S44 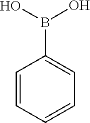 98-80-60 | 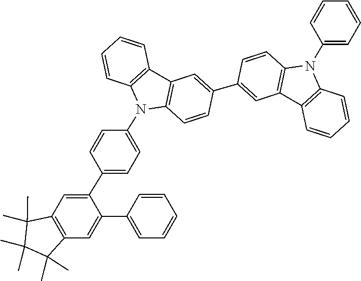 [SMILES: CC1(C)c2cc(-c3ccccc3)c(-c3ccc(-n4c5ccccc5c5cc(-c6ccc7c(c6)c6ccccc6n7-c6ccccc6)ccc54)cc3)cc2C(C)(C)C1(C)C] | 77% |
| A144 | S45 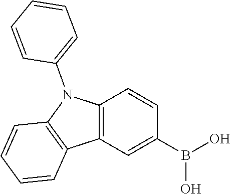 854952-58-2 | 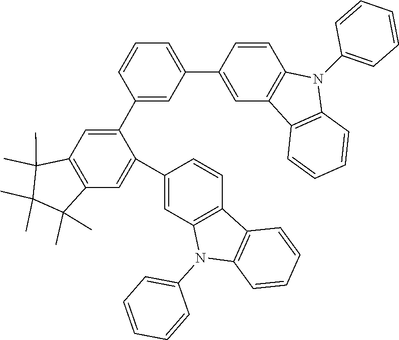 [SMILES: CC1(C)c2cc(-c3cccc(-c4ccc5c(c4)c4ccccc4n5-c4ccccc4)c3)c(-c3ccc4c5ccccc5n(-c5ccccc5)c4c3)cc2C(C)(C)C1(C)C] | 78% |
| A145 | S46 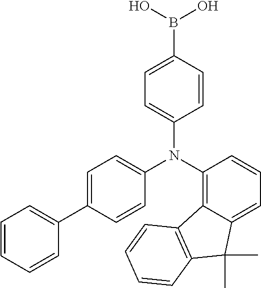 2410401-87-3 | 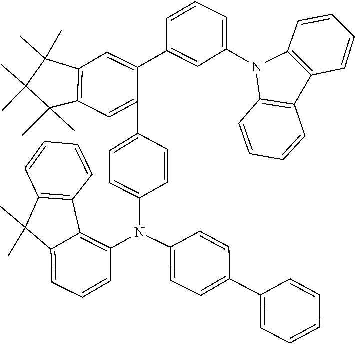 [SMILES: CC1(C)c2ccccc2-c2c(N(c3ccc(-c4ccccc4)cc3)c3ccc(-c4cc5c(cc4-c4cccc(-n6c7ccccc7c7ccccc76)c4)C(C)(C)C(C)(C)C5(C)C)cc3)cccc21] | 73% |
| A146 | S47 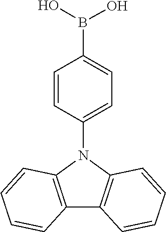 419536-33-7 | 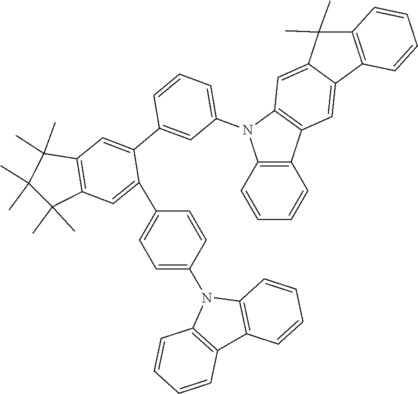 [SMILES: CC1(C)c2ccccc2-c2cc3c4ccccc4n(-c4cccc(-c5cc6c(cc5-c5ccc(-n7c8ccccc8c8ccccc87)cc5)C(C)(C)C(C)(C)C6(C)C)c4)c3cc21] | 71% |
| A147 | S48 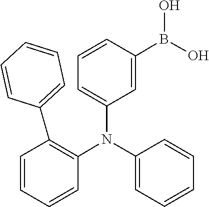 1776936-42-5 | 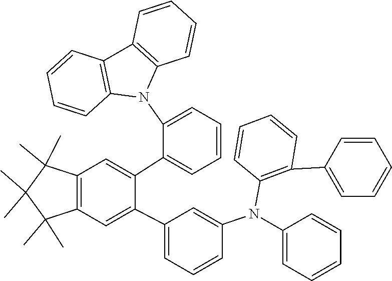 [SMILES: CC1(C)c2cc(-c3cccc(N(c4ccccc4)c4ccccc4-c4ccccc4)c3)c(-c3ccccc3-n3c4ccccc4c4ccccc43)cc2C(C)(C)C1(C)C] | 67% |
| A148 | S49 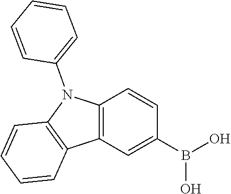 854952-58-2 | 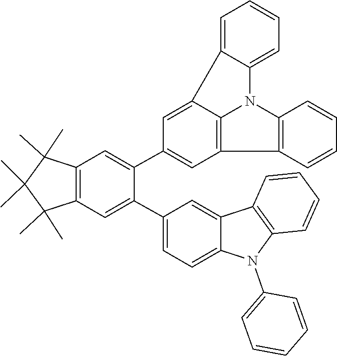 [SMILES: CC1(C)c2cc(-c3ccc4c(c3)c3ccccc3n4-c3ccccc3)c(-c3cc4c5ccccc5n5c6ccccc6c(c3)c45)cc2C(C)(C)C1(C)C] | 79% |
| A149 | S50 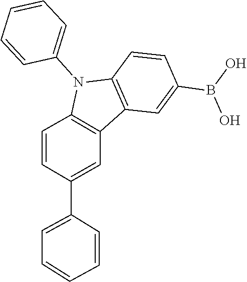 81359833-28-5 | 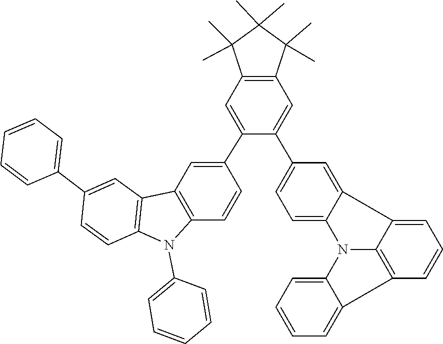 [SMILES: CC1(C)c2cc(-c3ccc4c(c3)c3cc(-c5ccccc5)ccc3n4-c3ccccc3)c(-c3ccc4c(c3)c3cccc5c6ccccc6n4c53)cc2C(C)(C)C1(C)C] | 80% |
| A150 | S51 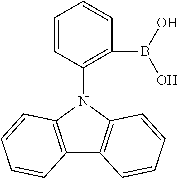 1189047-28-6 | 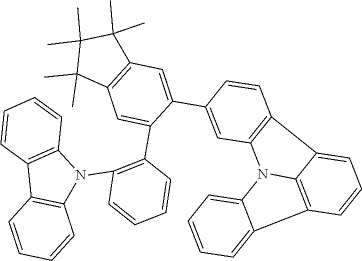 [SMILES: CC1(C)c2cc(-c3ccc4c5cccc6c7ccccc7n(c4c3)c65)c(-c3ccccc3-n3c4ccccc4c4ccccc43)cc2C(C)(C)C1(C)C] | 70% |
| A151 | S52 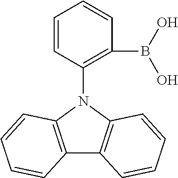 1189047-28-6 | 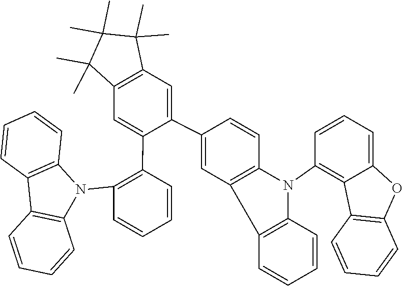 [SMILES: CC1(C)c2cc(-c3ccc4c(c3)c3ccccc3n4-c3cccc4oc5ccccc5c34)c(-c3ccccc3-n3c4ccccc4c4ccccc43)cc2C(C)(C)C1(C)C] | 68% |
| A152 | S53 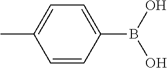 5720-05-8 | 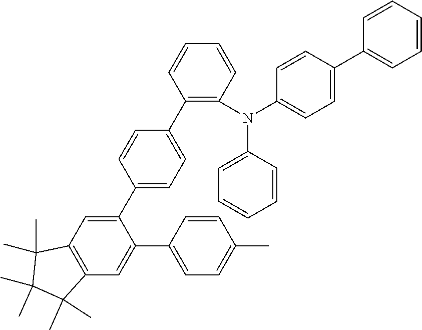 [SMILES: Cc1ccc(-c2cc3c(cc2-c2ccc(-c4ccccc4N(c4ccccc4)c4ccc(-c5ccccc5)cc4)cc2)C(C)(C)C(C)(C)C3(C)C)cc1] | 74% |
| A153 | S54 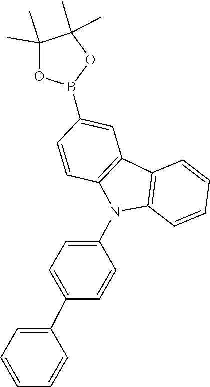 | 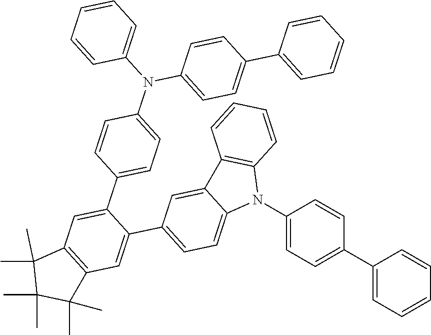 [SMILES: CC1(C)c2cc(-c3ccc(N(c4ccccc4)c4ccc(-c5ccccc5)cc4)cc3)c(-c3ccc4c(c3)c3ccccc3n4-c3ccc(-c4ccccc4)cc3)cc2C(C)(C)C1(C)C] | 76% |
| | 1391729-66-0 | | |
| A154 | S55 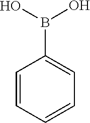 98-80-6 | 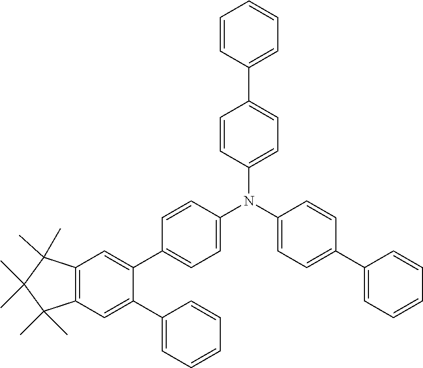 [SMILES: CC1(C)c2cc(-c3ccccc3)c(-c3ccc(N(c4ccc(-c5ccccc5)cc4)c4ccc(-c5ccccc5)cc4)cc3)cc2C(C)(C)C1(C)C] | 79% |
| A155 | S56 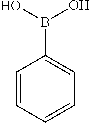 98-80-6 | 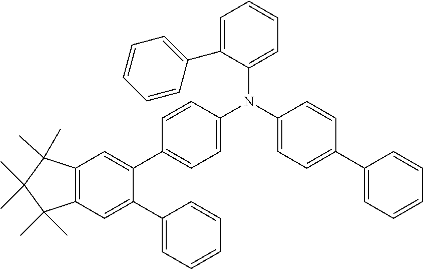 [SMILES: CC1(C)c2cc(-c3ccccc3)c(-c3ccc(N(c4ccc(-c5ccccc5)cc4)c4ccccc4-c4ccccc4)cc3)cc2C(C)(C)C1(C)C] | 70% |
| A156 | S57 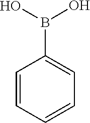 98-80-6 | 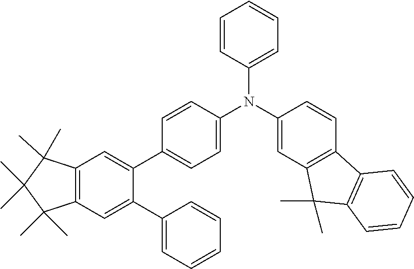 [SMILES: CC1(C)c2ccccc2-c2ccc(N(c3ccccc3)c3ccc(-c4cc5c(cc4-c4ccccc4)C(C)(C)C(C)(C)C5(C)C)cc3)cc21] | 75% |
| A157 | S58 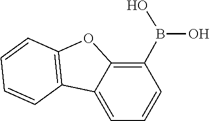 100124-06-9 | 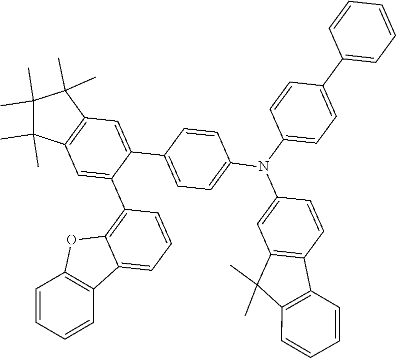 [SMILES: CC1(C)c2ccccc2-c2ccc(N(c3ccc(-c4ccccc4)cc3)c3ccc(-c4cc5c(cc4-c4cccc6c4oc4ccccc46)C(C)(C)C(C)(C)C5(C)C)cc3)cc21] | 77% |
| A158 | S59 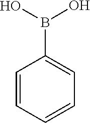 98-80-6 | 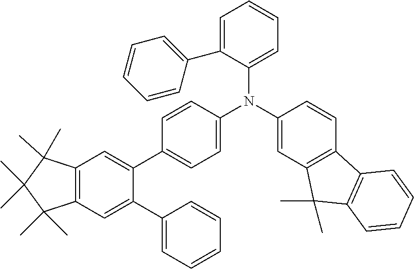 [SMILES: CC1(C)c2ccccc2-c2ccc(N(c3ccc(-c4cc5c(cc4-c4ccccc4)C(C)(C)C(C)(C)C5(C)C)cc3)c3ccccc3-c3ccccc3)cc21] | 70% |
| A159 | S60 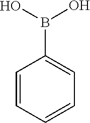 98-80-6 | 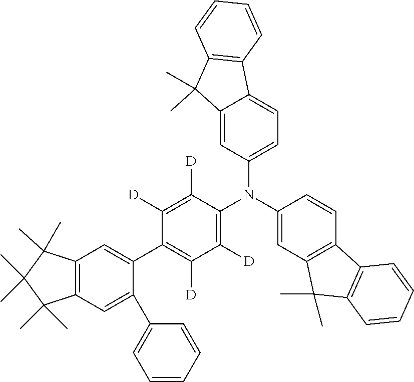 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1-c1cc2c(cc1-c1ccccc1)C(C)(C)C(C)(C)C2(C)C] | 73% |
| A160 | S61 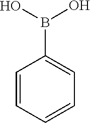 98-80-6 | 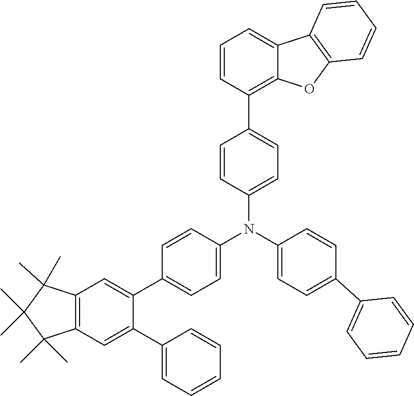 [SMILES: CC1(C)c2cc(-c3ccccc3)c(-c3ccc(N(c4ccc(-c5ccccc5)cc4)c4ccc(-c5cccc6c5oc5ccccc56)cc4)cc3)cc2C(C)(C)C1(C)C] | 72% |
| A161 | S62 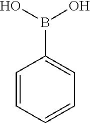 98-80-6 | 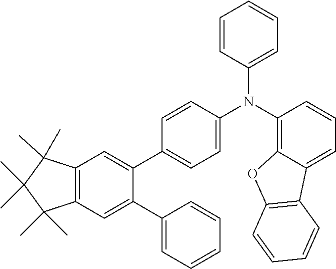 [SMILES: CC1(C)c2cc(-c3ccccc3)c(-c3ccc(N(c4ccccc4)c4cccc5c4oc4ccccc45)cc3)cc2C(C)(C)C1(C)C] | 76% |
| A162 | S63 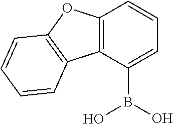 162607-19-4 | 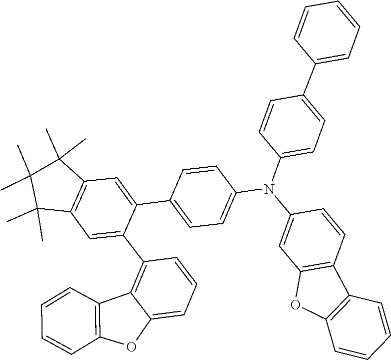 [SMILES: CC1(C)c2cc(-c3ccc(N(c4ccc(-c5ccccc5)cc4)c4ccc5c(c4)oc4ccccc45)cc3)c(-c3cccc4oc5ccccc5c34)cc2C(C)(C)C1(C)C] | 77% |
| A163 | S64 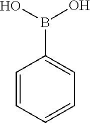 98-80-6 | 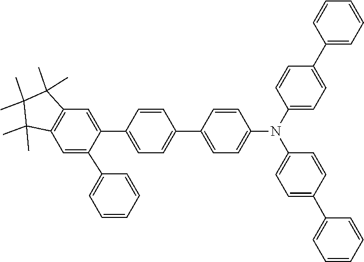 [SMILES: CC1(C)c2cc(-c3ccccc3)c(-c3ccc(-c4ccc(N(c5ccc(-c6ccccc6)cc5)c5ccc(-c6ccccc6)cc5)cc4)cc3)cc2C(C)(C)C1(C)C] | 79% |
| A164 | S65 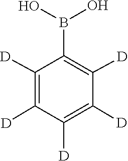 215527-70-1 | 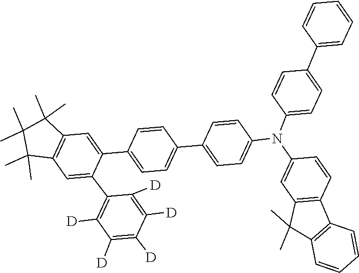 [SMILES: [2H]c1c([2H])c([2H])c(-c2cc3c(cc2-c2ccc(-c4ccc(N(c5ccc(-c6ccccc6)cc5)c5ccc6c(c5)C(C)(C)c5ccccc5-6)cc4)cc2)C(C)(C)C(C)(C)C3(C)C)c([2H])c1[2H]] | 76% |
| A165 | S66 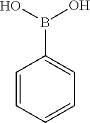 98-80-6 | 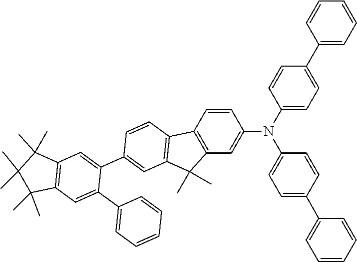 [SMILES: CC1(C)c2cc(-c3cc4c(cc3-c3ccccc3)C(C)(C)C(C)(C)C4(C)C)ccc2-c2ccc(N(c3ccc(-c4ccccc4)cc3)c3ccc(-c4ccccc4)cc3)cc21] | 75% |
| A166 | S67 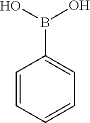 98-80-6 | 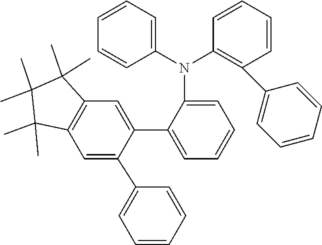 [SMILES: CC1(C)c2cc(-c3ccccc3)c(-c3ccccc3N(c3ccccc3)c3ccccc3-c3ccccc3)cc2C(C)(C)C1(C)C] | 64% |
| A167 | S68 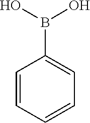 98-80-6 | 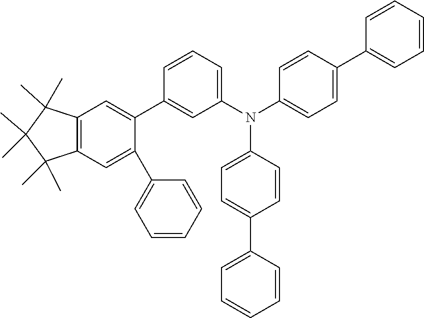 [SMILES: CC1(C)c2cc(-c3ccccc3)c(-c3cccc(N(c4ccc(-c5ccccc5)cc4)c4ccc(-c5ccccc5)cc4)c3)cc2C(C)(C)C1(C)C] | 68% |
| A168 | S69 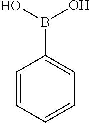 98-80-6 | 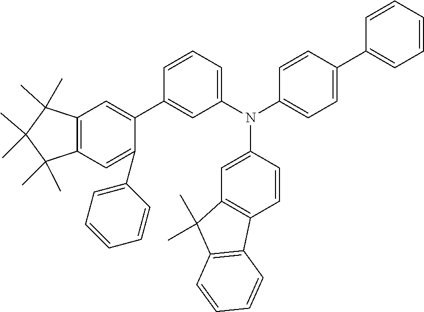 [SMILES: CC1(C)c2ccccc2-c2ccc(N(c3ccc(-c4ccccc4)cc3)c3cccc(-c4cc5c(cc4-c4ccccc4)C(C)(C)C(C)(C)C5(C)C)c3)cc21] | 69% |
| A169 | S70 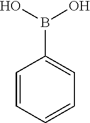 98-80-6 | 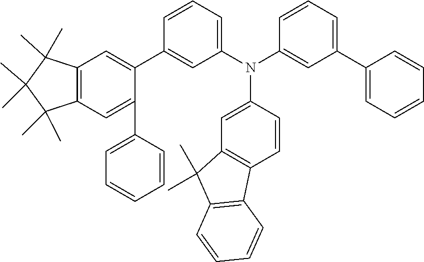 [SMILES: CC1(C)c2ccccc2-c2ccc(N(c3cccc(-c4ccccc4)c3)c3cccc(-c4cc5c(cc4-c4ccccc4)C(C)(C)C(C)(C)C5(C)C)c3)cc21] | 67% |
| A170 | S71 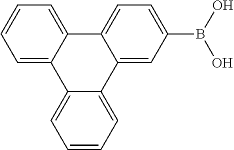 654664-63-8 | 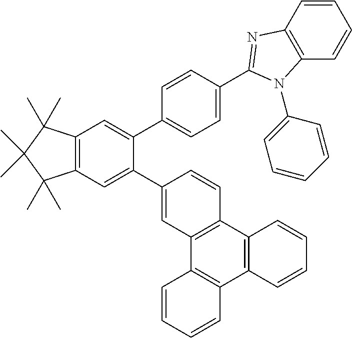 [SMILES: CC1(C)c2cc(-c3ccc(-c4nc5ccccc5n4-c4ccccc4)cc3)c(-c3ccc4c5ccccc5c5ccccc5c4c3)cc2C(C)(C)C1(C)C] | 82% |
| A171 | S72 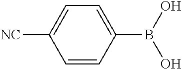 126747-14-6 | 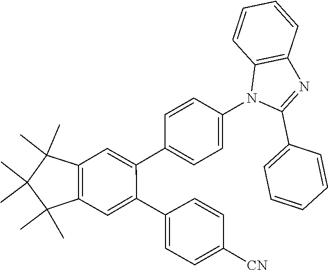 [SMILES: CC1(C)c2cc(-c3ccc(C#N)cc3)c(-c3ccc(-n4c(-c5ccccc5)nc5ccccc54)cc3)cc2C(C)(C)C1(C)C] | 78% |
| A172 | S73 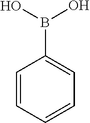 98-80-6 | 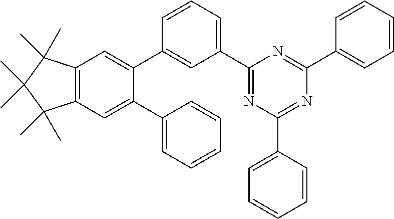 [SMILES: CC1(C)c2cc(-c3ccccc3)c(-c3cccc(-c4nc(-c5ccccc5)nc(-c5ccccc5)n4)c3)cc2C(C)(C)C1(C)C] | 76% |
| A173 | S74 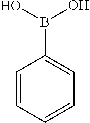 98-80-6 | 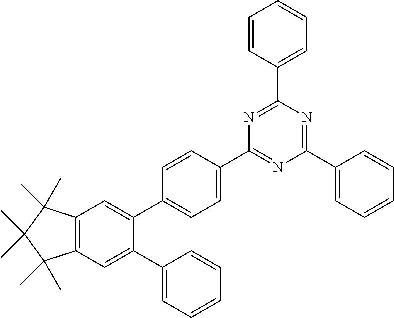 [SMILES: CC1(C)c2cc(-c3ccccc3)c(-c3ccc(-c4nc(-c5ccccc5)nc(-c5ccccc5)n4)cc3)cc2C(C)(C)C1(C)C] | 78% |
| A174 | S75 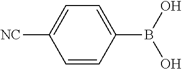 126747-14-6 | 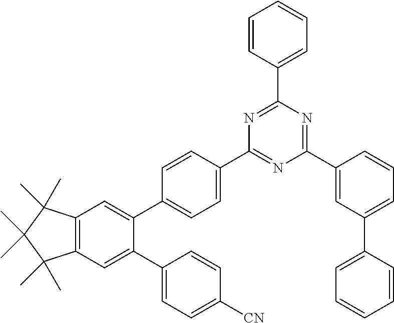 [SMILES: CC1(C)c2cc(-c3ccc(C#N)cc3)c(-c3ccc(-c4nc(-c5ccccc5)nc(-c5cccc(-c6ccccc6)c5)n4)cc3)cc2C(C)(C)C1(C)C] | 79% |
| A175 | S76 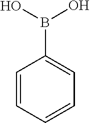 98-80-6 | 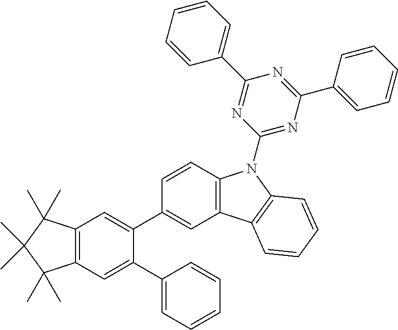 [SMILES: CC1(C)c2cc(-c3ccccc3)c(-c3ccc4c(c3)c3ccccc3n4-c3nc(-c4ccccc4)nc(-c4ccccc4)n3)cc2C(C)(C)C1(C)C] | 75% |
| A176 | S77 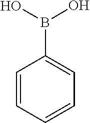 98-80-6 | 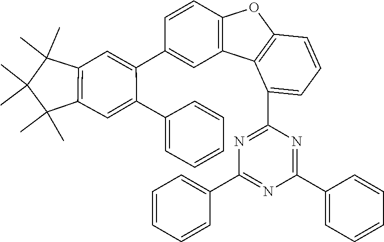 [SMILES: CC1(C)c2cc(-c3ccccc3)c(-c3ccc4oc5cccc(-c6nc(-c7ccccc7)nc(-c7ccccc7)n6)c5c4c3)cc2C(C)(C)C1(C)C] | 64% |
| A177 | S74 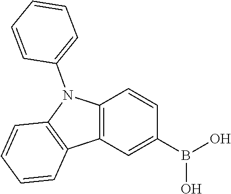 854952-58-2 | 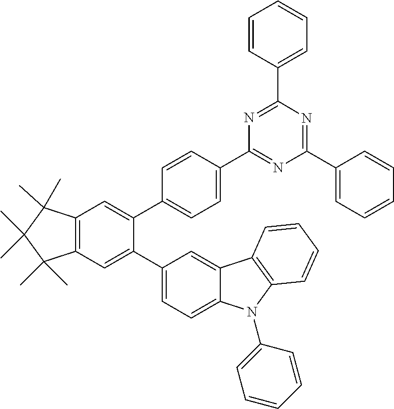 [SMILES: CC1(C)c2cc(-c3ccc(-c4nc(-c5ccccc5)nc(-c5ccccc5)n4)cc3)c(-c3ccc4c(c3)c3ccccc3n4-c3ccccc3)cc2C(C)(C)C1(C)C] | 78% |
| A178 | S74 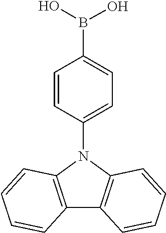 419536-33-7 | 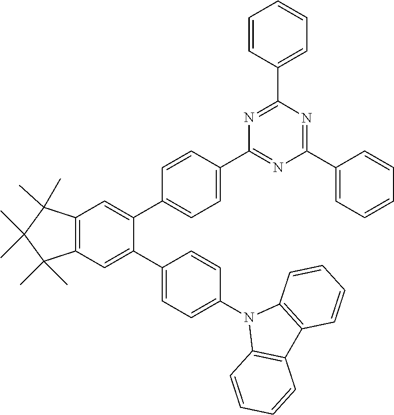 [SMILES: CC1(C)c2cc(-c3ccc(-c4nc(-c5ccccc5)nc(-c5ccccc5)n4)cc3)c(-c3ccc(-n4c5ccccc5c5ccccc54)cc3)cc2C(C)(C)C1(C)C] | 78% |
| A179 | S74 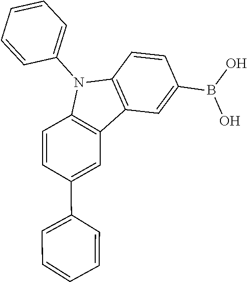 81359833-28-5 | 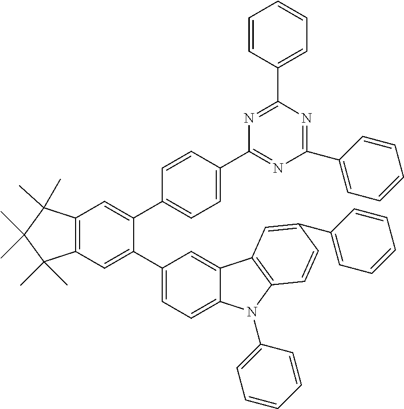 [SMILES: CC1(C)c2cc(-c3ccc(-c4nc(-c5ccccc5)nc(-c5ccccc5)n4)cc3)c(-c3ccc4c(c3)c3cc(-c5ccccc5)ccc3n4-c3ccccc3)cc2C(C)(C)C1(C)C] | 67% |
| A180 | S74 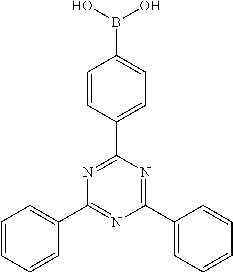 1313018-07-3 | 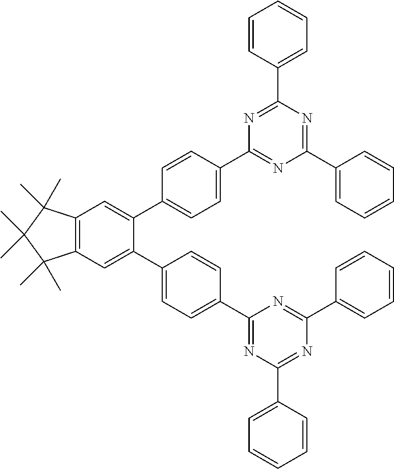 [SMILES: CC1(C)c2cc(-c3ccc(-c4nc(-c5ccccc5)nc(-c5ccccc5)n4)cc3)c(-c3ccc(-c4nc(-c5ccccc5)nc(-c5ccccc5)n4)cc3)cc2C(C)(C)C1(C)C] | 80% |
| A181 | S74 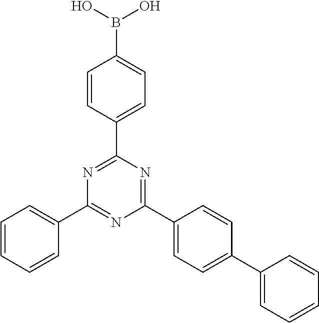 | 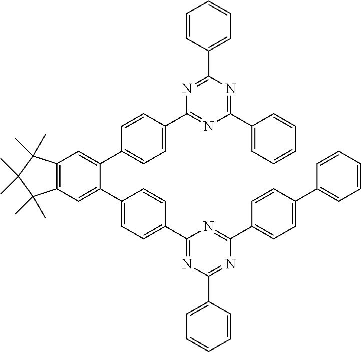 [SMILES: CC1(C)c2cc(-c3ccc(-c4nc(-c5ccccc5)nc(-c5ccccc5)n4)cc3)c(-c3ccc(-c4nc(-c5ccccc5)nc(-c5ccc(-c6ccccc6)cc5)n4)cc3)cc2C(C)(C)C1(C)C] | 79% |
| | 1987895-22-6 | | |
| A182 | S74 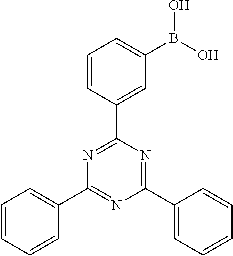 1269508-31-7 | 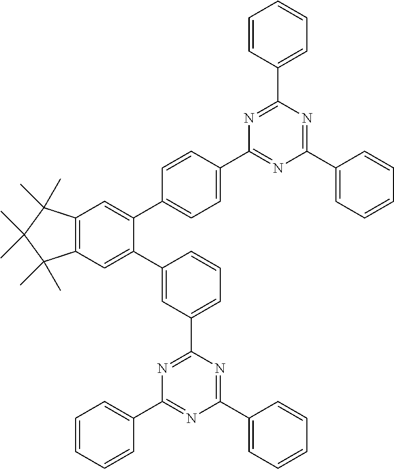 [SMILES: CC1(C)c2cc(-c3ccc(-c4nc(-c5ccccc5)nc(-c5ccccc5)n4)cc3)c(-c3cccc(-c4nc(-c5ccccc5)nc(-c5ccccc5)n4)c3)cc2C(C)(C)C1(C)C] | 76% |
| A183 | S74 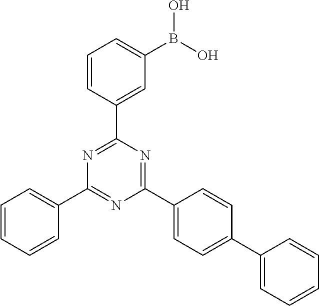 1696425-30-5 | 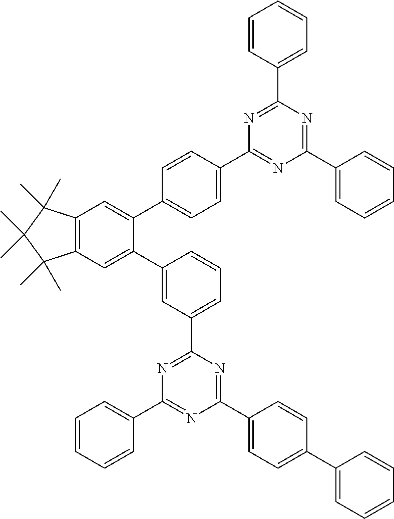 [SMILES: CC1(C)c2cc(-c3ccc(-c4nc(-c5ccccc5)nc(-c5ccccc5)n4)cc3)c(-c3cccc(-c4nc(-c5ccccc5)nc(-c5ccc(-c6ccccc6)cc5)n4)c3)cc2C(C)(C)C1(C)C] | 73% |
| A184 | S1 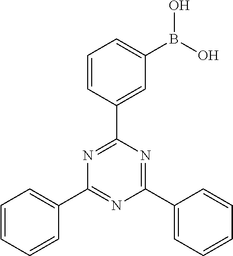 1269508-31-7 | 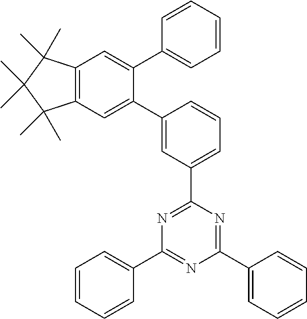 [SMILES: CC1(C)c2cc(-c3ccccc3)c(-c3cccc(-c4nc(-c5ccccc5)nc(-c5ccccc5)n4)c3)cc2C(C)(C)C1(C)C] | 70% |
| A185 | S1 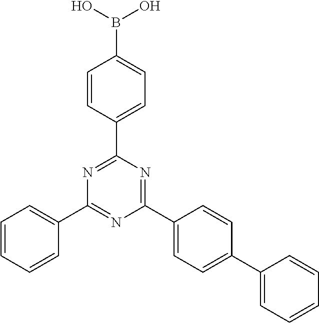 1987895-22-6 | 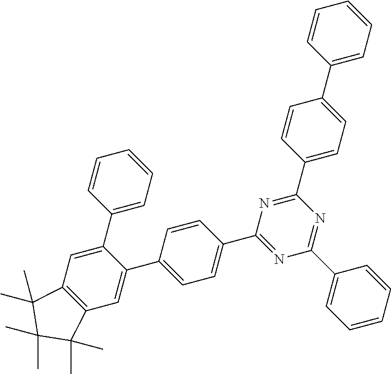 [SMILES: CC1(C)c2cc(-c3ccccc3)c(-c3ccc(-c4nc(-c5ccccc5)nc(-c5ccc(-c6ccccc6)cc5)n4)cc3)cc2C(C)(C)C1(C)C] | 76% |
| A186 | S2 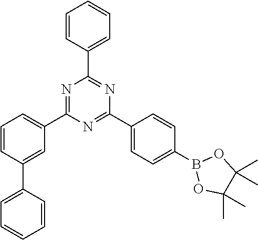 2168567-62-0 | 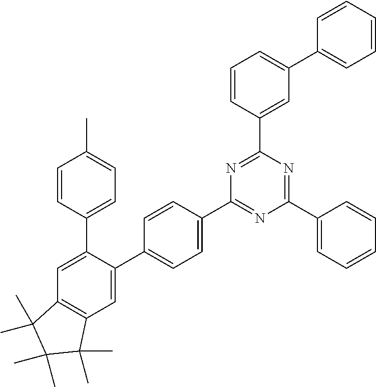 [SMILES: Cc1ccc(-c2cc3c(cc2-c2ccc(-c4nc(-c5ccccc5)nc(-c5cccc(-c6ccccc6)c5)n4)cc2)C(C)(C)C(C)(C)C3(C)C)cc1] | 74% |
| A187 | S3 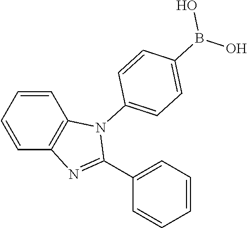 | 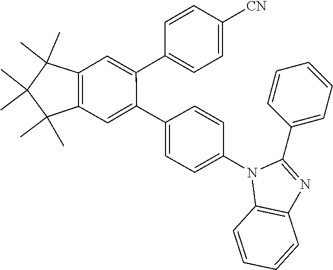 [SMILES: CC1(C)c2cc(-c3ccc(C#N)cc3)c(-c3ccc(-n4c(-c5ccccc5)nc5ccccc54)cc3)cc2C(C)(C)C1(C)C] | 75% |
| | 867044-33-5 | | |
| A188 | S4 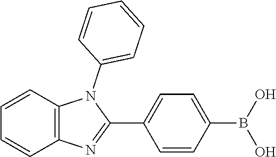 952514-79-3 | 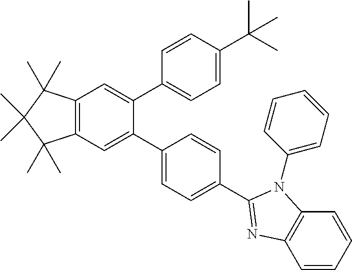 [SMILES: CC(C)(C)c1ccc(-c2cc3c(cc2-c2ccc(-c4nc5ccccc5n4-c4ccccc4)cc2)C(C)(C)C(C)(C)C3(C)C)cc1] | 77% |
| A189 | S8 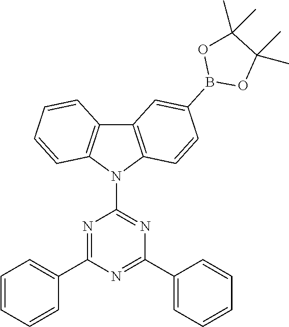 | 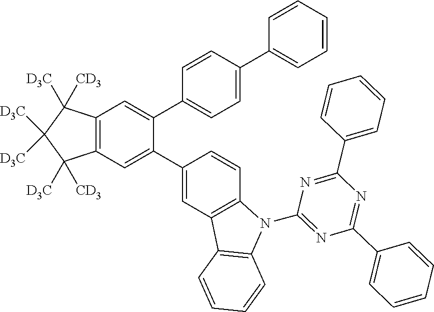 [SMILES: [2H]C([2H])([2H])C1(C([2H])([2H])[2H])c2cc(-c3ccc(-c4ccccc4)cc3)c(-c3ccc4c(c3)c3ccccc3n4-c3nc(-c4ccccc4)nc(-c4ccccc4)n3)cc2C(C([2H])([2H])[2H])(C([2H])([2H])[2H])C1(C([2H])([2H])[2H])C([2H])([2H])[2H]] | 75% |
| | 1361094-91-8 | | |
| A190 | S9 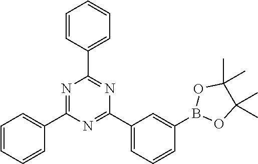 1269508-31-7 | 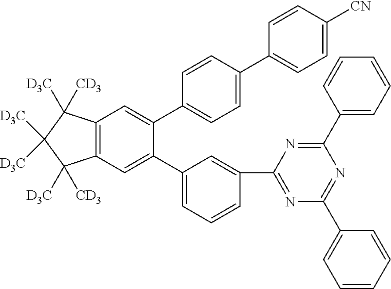 [SMILES: [2H]C([2H])([2H])C1(C([2H])([2H])[2H])c2cc(-c3ccc(-c4ccc(C#N)cc4)cc3)c(-c3cccc(-c4nc(-c5ccccc5)nc(-c5ccccc5)n4)c3)cc2C(C([2H])([2H])[2H])(C([2H])([2H])[2H])C1(C([2H])([2H])[2H])C([2H])([2H])[2H]] | 78% |
| A191 | S10 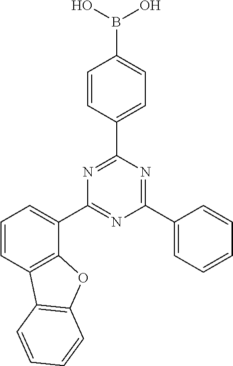 | 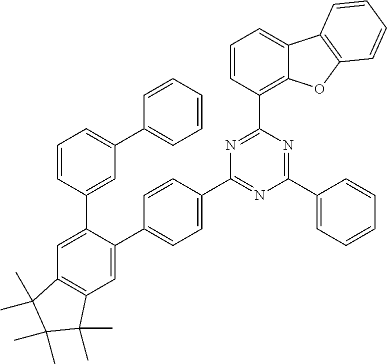 [SMILES: CC1(C)c2cc(-c3ccc(-c4nc(-c5ccccc5)nc(-c5cccc6c5oc5ccccc56)n4)cc3)c(-c3cccc(-c4ccccc4)c3)cc2C(C)(C)C1(C)C] | 72% |
| | 2378846-11-6 | | |
| A192 | S16 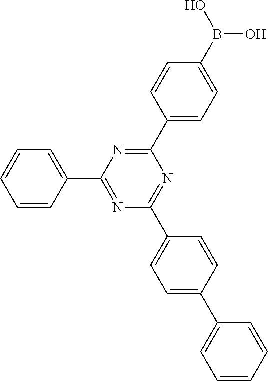 | 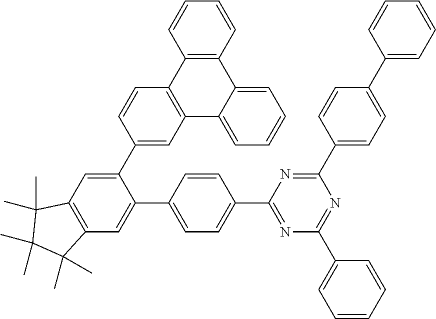 [SMILES: CC1(C)c2cc(-c3ccc(-c4nc(-c5ccccc5)nc(-c5ccc(-c6ccccc6)cc5)n4)cc3)c(-c3ccc4c5ccccc5c5ccccc5c4c3)cc2C(C)(C)C1(C)C] | 79% |
| | 1987895-22-6 | | |
| A193 | S17 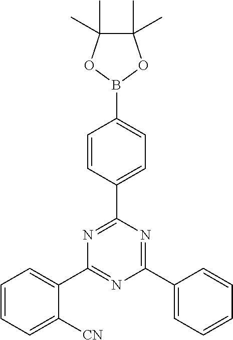 2226916-97-6 | 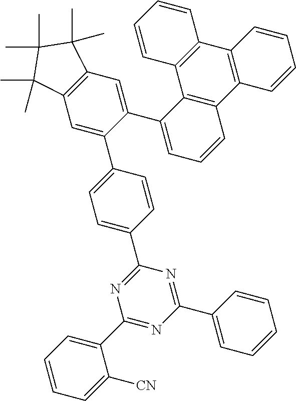 [SMILES: CC1(C)c2cc(-c3ccc(-c4nc(-c5ccccc5)nc(-c5ccccc5C#N)n4)cc3)c(-c3cccc4c5ccccc5c5ccccc5c34)cc2C(C)(C)C1(C)C] | 75% |
| A194 | S18 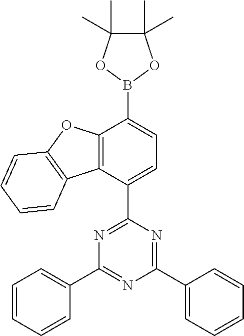 | 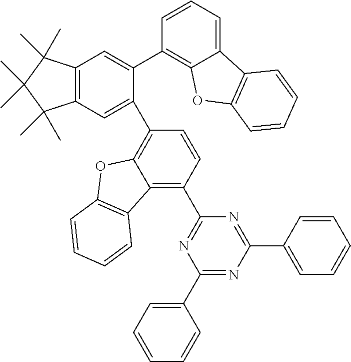 [SMILES: CC1(C)c2cc(-c3cccc4c3oc3ccccc34)c(-c3ccc(-c4nc(-c5ccccc5)nc(-c5ccccc5)n4)c4c3oc3ccccc34)cc2C(C)(C)C1(C)C] | 75% |
| | 2265924-57-8 | | |
| A195 | S21 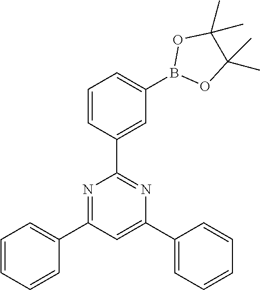 1381862-91-4 | 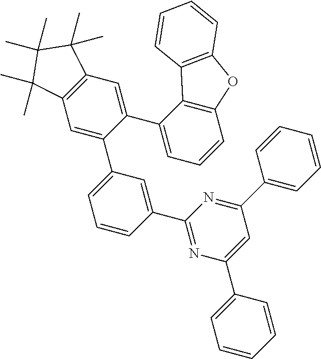 [SMILES: CC1(C)c2cc(-c3cccc(-c4nc(-c5ccccc5)cc(-c5ccccc5)n4)c3)c(-c3cccc4oc5ccccc5c34)cc2C(C)(C)C1(C)C] | 70% |
| A196 | S22 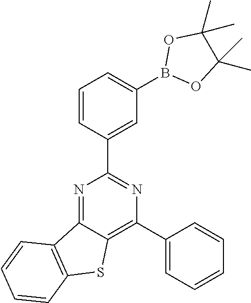 1801325-73-4 | 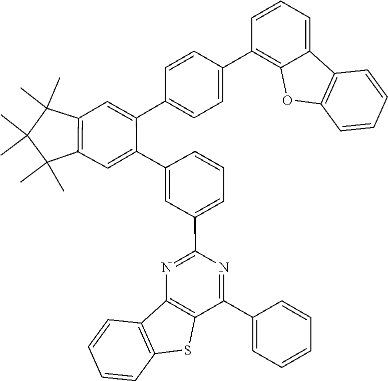 [SMILES: CC1(C)c2cc(-c3ccc(-c4cccc5c4oc4ccccc45)cc3)c(-c3cccc(-c4nc(-c5ccccc5)c5sc6ccccc6c5n4)c3)cc2C(C)(C)C1(C)C] | 63% |
| A197 | S26 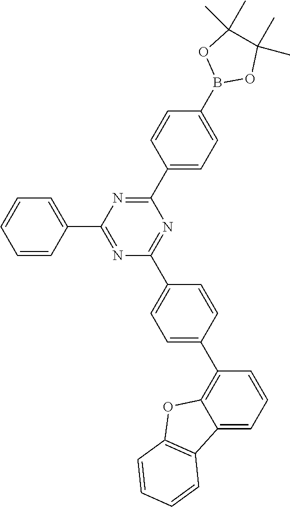 | 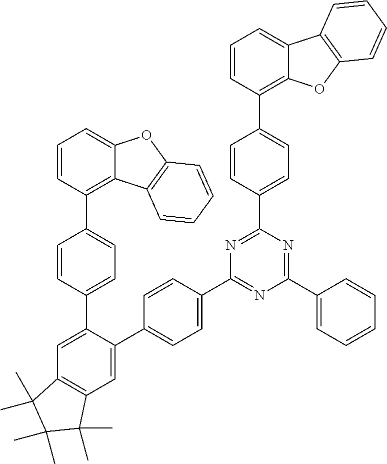 [SMILES: CC1(C)c2cc(-c3ccc(-c4nc(-c5ccccc5)nc(-c5ccc(-c6cccc7c6oc6ccccc67)cc5)n4)cc3)c(-c3ccc(-c4cccc5oc6ccccc6c45)cc3)cc2C(C)(C)C1(C)C] | 71% |
| | 2287210-68-6 | | |
| A198 | S31 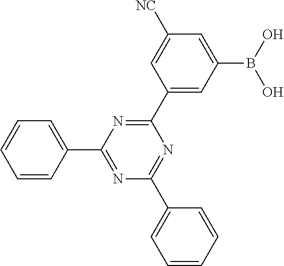 2140928-48-7 | 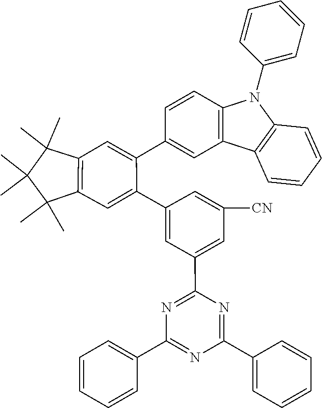 [SMILES: CC1(C)c2cc(-c3cc(C#N)cc(-c4nc(-c5ccccc5)nc(-c5ccccc5)n4)c3)c(-c3ccc4c(c3)c3ccccc3n4-c3ccccc3)cc2C(C)(C)C1(C)C] | 69% |
| A199 | S32 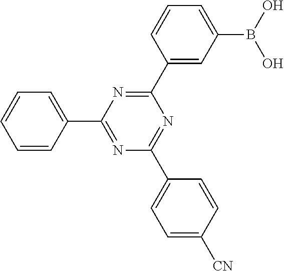 2259756-07-3 | 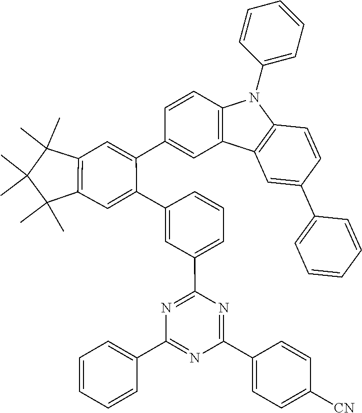 [SMILES: CC1(C)c2cc(-c3cccc(-c4nc(-c5ccccc5)nc(-c5ccc(C#N)cc5)n4)c3)c(-c3ccc4c(c3)c3cc(-c5ccccc5)ccc3n4-c3ccccc3)cc2C(C)(C)C1(C)C] | 69% |
| A200 | S33 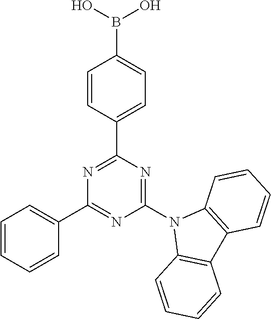 2378846-09-2 | 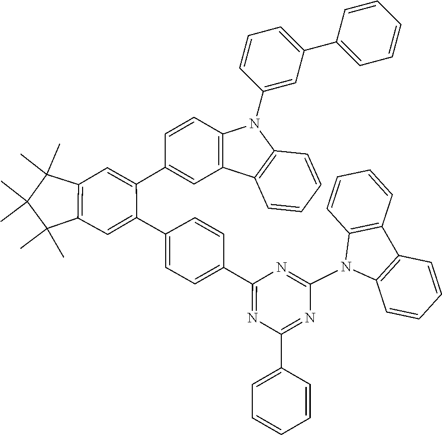 [SMILES: CC1(C)c2cc(-c3ccc(-c4nc(-c5ccccc5)nc(-n5c6ccccc6c6ccccc65)n4)cc3)c(-c3ccc4c(c3)c3ccccc3n4-c3cccc(-c4ccccc4)c3)cc2C(C)(C)C1(C)C] | 61% |
| A201 | S34 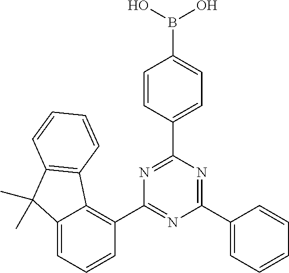 2308565-18-4 | 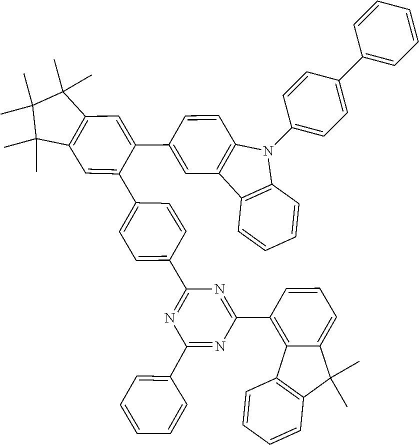 [SMILES: CC1(C)c2ccccc2-c2c(-c3nc(-c4ccccc4)nc(-c4ccc(-c5cc6c(cc5-c5ccc7c(c5)c5ccccc5n7-c5ccc(-c7ccccc7)cc5)C(C)(C)C(C)(C)C6(C)C)cc4)n3)cccc21] | 75% |
| A202 | S35 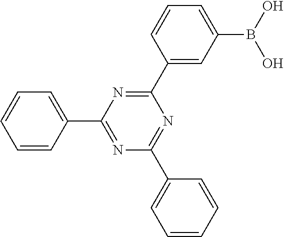 1612243-82-9 | 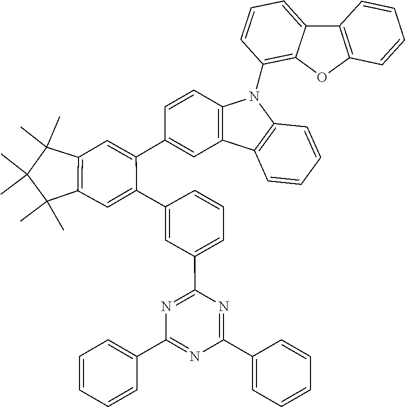 [SMILES: CC1(C)c2cc(-c3cccc(-c4nc(-c5ccccc5)nc(-c5ccccc5)n4)c3)c(-c3ccc4c(c3)c3ccccc3n4-c3cccc4c3oc3ccccc34)cc2C(C)(C)C1(C)C] | 76% |
| A203 | S39 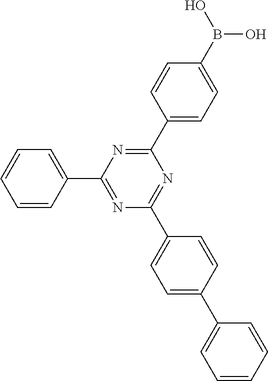 1987895-22-6 | 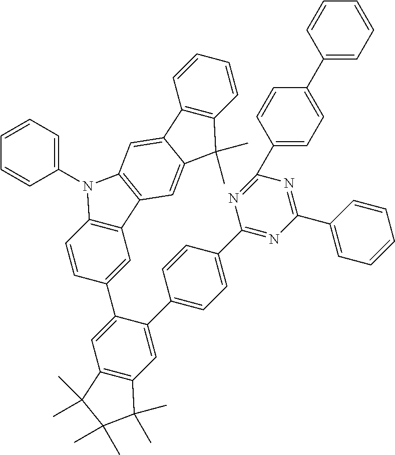 [SMILES: CC1(C)c2ccccc2-c2cc3c(cc21)c1cc(-c2cc4c(cc2-c2ccc(-c5nc(-c6ccccc6)nc(-c6ccc(-c7ccccc7)cc6)n5)cc2)C(C)(C)C(C)(C)C4(C)C)ccc1n3-c1ccccc1] | 67% |
| A204 | S39 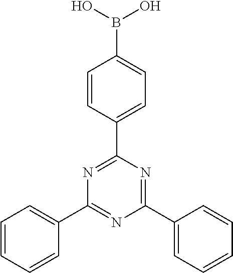 1313018-07-3 | 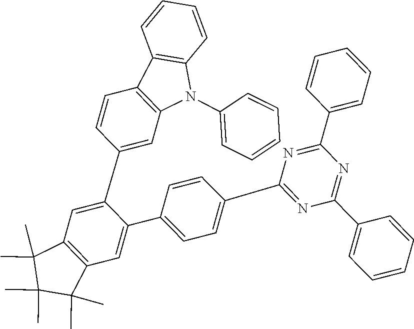 [SMILES: CC1(C)c2cc(-c3ccc(-c4nc(-c5ccccc5)nc(-c5ccccc5)n4)cc3)c(-c3ccc4c5ccccc5n(-c5ccccc5)c4c3)cc2C(C)(C)C1(C)C] | 73% |
| A205 | S40 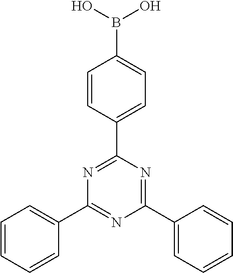 1313018-07-3 | 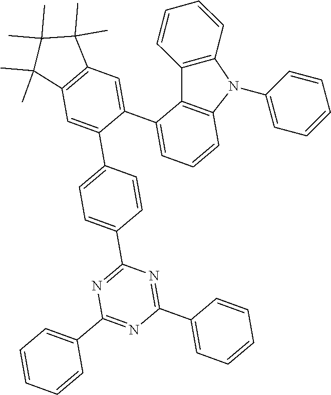 [SMILES: CC1(C)c2cc(-c3ccc(-c4nc(-c5ccccc5)nc(-c5ccccc5)n4)cc3)c(-c3cccc4c3c3ccccc3n4-c3ccccc3)cc2C(C)(C)C1(C)C] | 74% |
| A206 | S49 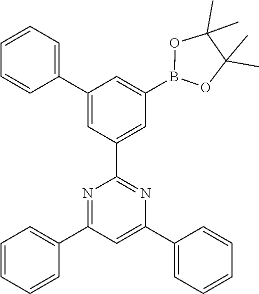 1835206-58-0 | 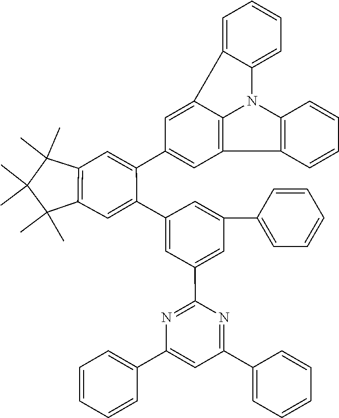 [SMILES: CC1(C)c2cc(-c3cc(-c4ccccc4)cc(-c4nc(-c5ccccc5)cc(-c5ccccc5)n4)c3)c(-c3cc4c5ccccc5n5c6ccccc6c(c3)c45)cc2C(C)(C)C1(C)C] | 73% |
| A207 | S50 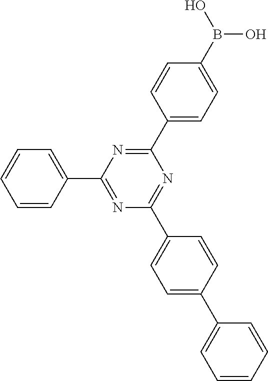 | 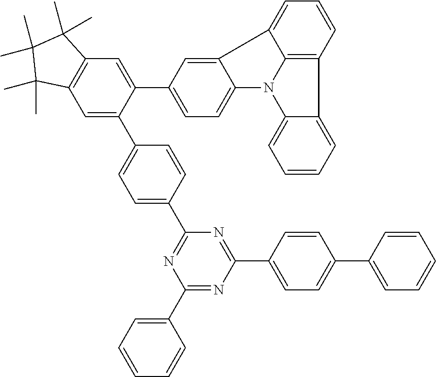 [SMILES: CC1(C)c2cc(-c3ccc(-c4nc(-c5ccccc5)nc(-c5ccc(-c6ccccc6)cc5)n4)cc3)c(-c3ccc4c(c3)c3cccc5c6ccccc6n4c53)cc2C(C)(C)C1(C)C] | 79% |
| | 1987895-22-6 | | |

### Example A300

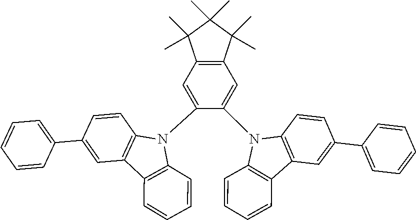 [SMILES: CC1(C)c2cc(-n3c4ccccc4c4cc(-c5ccccc5)ccc43)c(-n3c4ccccc4c4cc(-c5ccccc5)ccc43)cc2C(C)(C)C1(C)C]

Procedure analogous to T. Taisei et al., Chem. Lett., 2019, 48, 1160. Synthesis of 3c. Starting materials: 18.0 g (50 mmol), 26.7 g (110 mmol) of 3-phenyl-9H-carbazole [103012-26-6]. The crude product is purified by chromatography and repeated hot-extraction crystallization (customary organic solvents or the combinations thereof, preferably acetonitrile-DCM, 1:3 to 3:1 vv) and fractional sublimation or heat treatment under high vacuum. Yield: 13.7 g (20 mmol), 40%; purity: about 99.9% by HPLC. If first 25 mmol of a first amine/carbazole and then, after a reaction time of 6 h, 25 mmol of a second amine/carbazole are added, it is possible to obtain unsymmetrically substituted compounds.

The following compounds can be prepared analogously:

| Ex. | Reactants | Product | Yield |
|---|---|---|---|
| A301 | LS1 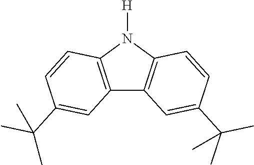 [SMILES: CC(C)(C)c1ccc2[nH]c3ccc(C(C)(C)C)cc3c2c1] 37500-95-1 | 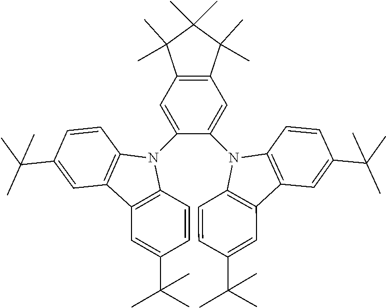 [SMILES: CC(C)(C)c1ccc2c(c1)c1cc(C(C)(C)C)ccc1n2-c1cc2c(cc1-n1c3ccc(C(C)(C)C)cc3c3cc(C(C)(C)C)ccc31)C(C)(C)C(C)(C)C2(C)C] | 42% |
| A302 | LS2 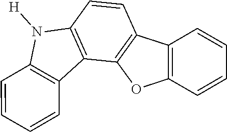 [SMILES: c1ccc2c(c1)[nH]c1ccc3c4ccccc4oc3c12] 1199616-66-4 | 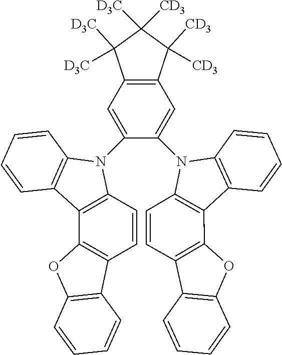 [SMILES: [2H]C([2H])([2H])C1(C([2H])([2H])[2H])c2cc(-n3c4ccccc4c4c5oc6ccccc6c5ccc43)c(-n3c4ccccc4c4c5oc6ccccc6c5ccc43)cc2C(C([2H])([2H])[2H])(C([2H])([2H])[2H])C1(C([2H])([2H])[2H])C([2H])([2H])[2H]] | 38% |
| A303 | LS3 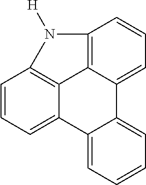 [SMILES: c1ccc2c(c1)c1cccc3[nH]c4cccc2c4c31] 109606-75-9 | 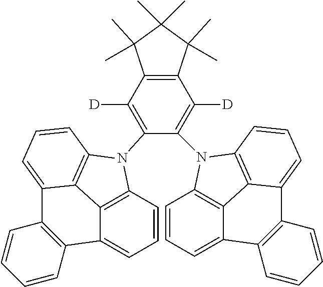 [SMILES: [2H]c1c(-n2c3cccc4c5ccccc5c5cccc2c5c43)c(-n2c3cccc4c5ccccc5c5cccc2c5c43)c([2H])c2c1C(C)(C)C(C)(C)C2(C)C] | 41% |
| A304 | LS1 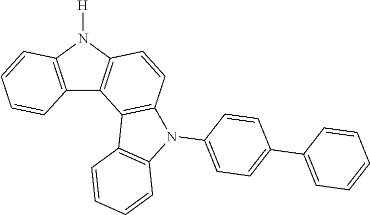 [SMILES: c1ccc(-c2ccc(-n3c4ccccc4c4c5c(ccc43)[nH]c3ccccc35)cc2)cc1] 2071630-78-7 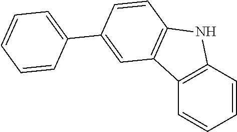 [SMILES: c1ccc(-c2ccc3[nH]c4ccccc4c3c2)cc1] 103012-26-6 | 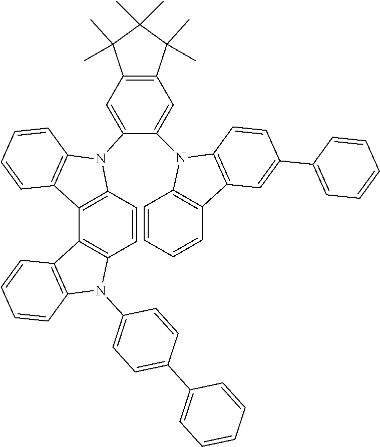 [SMILES: CC1(C)c2cc(-n3c4ccccc4c4cc(-c5ccccc5)ccc43)c(-n3c4ccccc4c4c5c6ccccc6n(-c6ccc(-c7ccccc7)cc6)c5ccc43)cc2C(C)(C)C1(C)C] | 26% |
| A305 | LS1 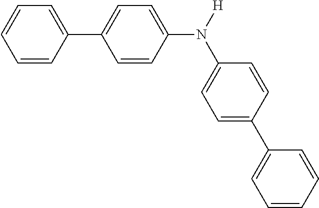 [SMILES: c1ccc(-c2ccc(Nc3ccc(-c4ccccc4)cc3)cc2)cc1] 102113-98-4 | 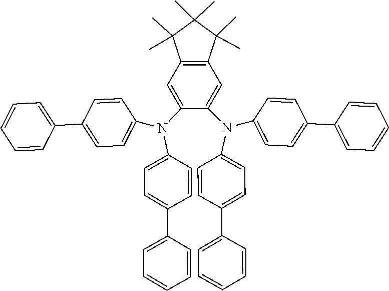 [SMILES: CC1(C)c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2C(C)(C)C1(C)C] | 31% |
| A306 | LS1 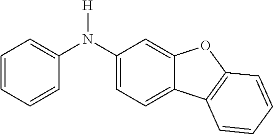 [SMILES: c1ccc(Nc2ccc3c(c2)oc2ccccc23)cc1] 406488-21-9 | 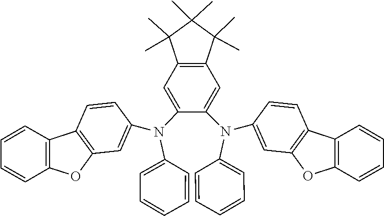 [SMILES: CC1(C)c2cc(N(c3ccccc3)c3ccc4c(c3)oc3ccccc34)c(N(c3ccccc3)c3ccc4c(c3)oc3ccccc34)cc2C(C)(C)C1(C)C] | 29% |
| A307 | LS1 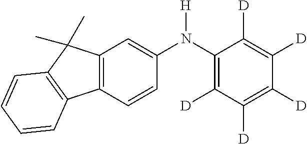 [SMILES: [2H]c1c([2H])c([2H])c(Nc2ccc3c(c2)C(C)(C)c2ccccc2-3)c([2H])c1[2H]] 1359833-31-0 | 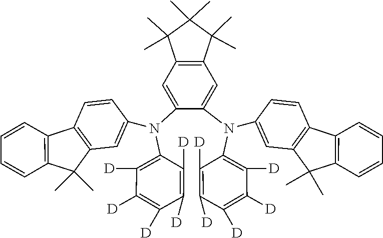 [SMILES: [2H]c1c([2H])c([2H])c(N(c2ccc3c(c2)C(C)(C)c2ccccc2-3)c2cc3c(cc2N(c2ccc4c(c2)C(C)(C)c2ccccc2-4)c2c([2H])c([2H])c([2H])c([2H])c2[2H])C(C)(C)C(C)(C)C3(C)C)c([2H])c1[2H]] | 33% |
| A308 | LS1 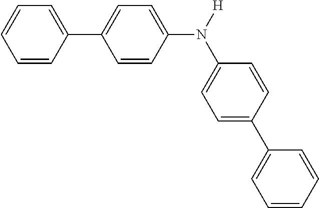 [SMILES: c1ccc(-c2ccc(Nc3ccc(-c4ccccc4)cc3)cc2)cc1] 102113-98-4 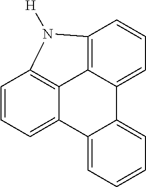 [SMILES: c1ccc2c(c1)c1cccc3[nH]c4cccc2c4c31] 109606-75-9 | 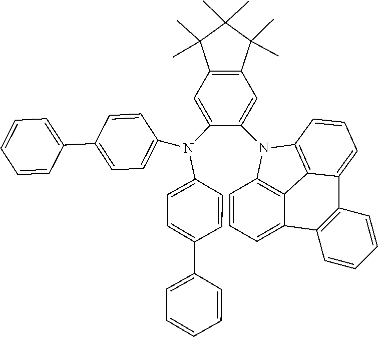 [SMILES: CC1(C)c2cc(N(c3ccc(-c4ccccc4)cc3)c3ccc(-c4ccccc4)cc3)c(-n3c4cccc5c6ccccc6c6cccc3c6c54)cc2C(C)(C)C1(C)C] | 24% |
| A309 | LS1 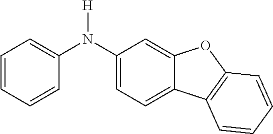 [SMILES: c1ccc(Nc2ccc3c(c2)oc2ccccc23)cc1] 406488-21-9 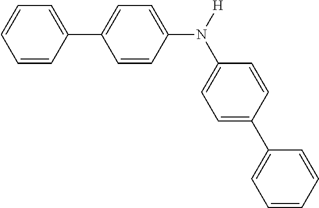 [SMILES: c1ccc(-c2ccc(Nc3ccc(-c4ccccc4)cc3)cc2)cc1] 102113-98-4 | 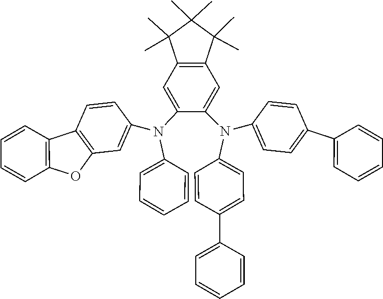 [SMILES: CC1(C)c2cc(N(c3ccc(-c4ccccc4)cc3)c3ccc(-c4ccccc4)cc3)c(N(c3ccccc3)c3ccc4c(c3)oc3ccccc34)cc2C(C)(C)C1(C)C] | 21% |

In addition, the following synthons are synthesized by the method set out for preparation of A300:

| | | | |
|---|---|---|---|
| S100 | LS1 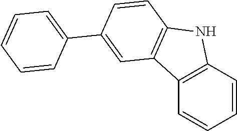 [SMILES: c1ccc(-c2ccc3[nH]c4ccccc4c3c2)cc1] 103012-26-6 25 mmol | 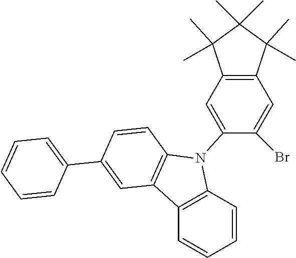 [SMILES: CC1(C)c2cc(Br)c(-n3c4ccccc4c4cc(-c5ccccc5)ccc43)cc2C(C)(C)C1(C)C] | 57% |
| S101 | LS1 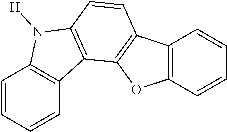 [SMILES: c1ccc2c(c1)[nH]c1ccc3c4ccccc4oc3c12] 1199616-66-4 25 mmol | 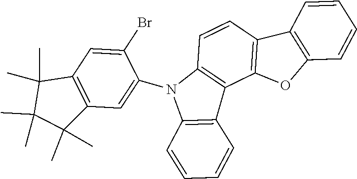 [SMILES: CC1(C)c2cc(Br)c(-n3c4ccccc4c4c5oc6ccccc6c5ccc43)cc2C(C)(C)C1(C)C] | 54% |
| S102 | LS1 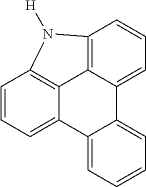 [SMILES: c1ccc2c(c1)c1cccc3[nH]c4cccc2c4c31] 109606-75-9 | 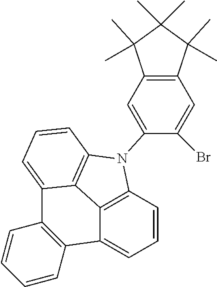 [SMILES: CC1(C)c2cc(Br)c(-n3c4cccc5c6ccccc6c6cccc3c6c54)cc2C(C)(C)C1(C)C] | 64% |
| S103 | LS1 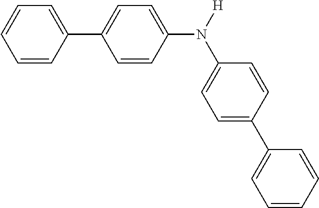 [SMILES: c1ccc(-c2ccc(Nc3ccc(-c4ccccc4)cc3)cc2)cc1] | 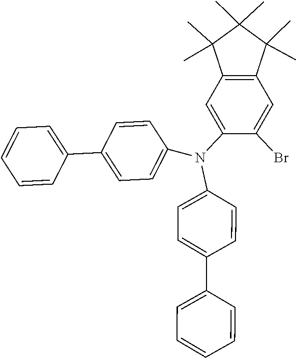 [SMILES: CC1(C)c2cc(Br)c(N(c3ccc(-c4ccccc4)cc3)c3ccc(-c4ccccc4)cc3)cc2C(C)(C)C1(C)C] | 46% |

Among other compounds, the synthons detailed above are used to prepare compounds of the invention A400 to A409.

### Example A400

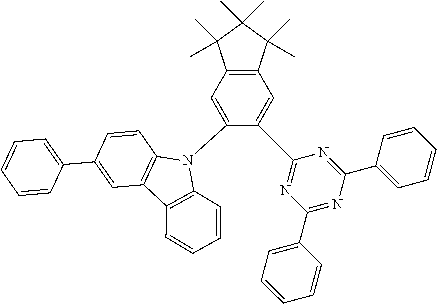 [SMILES: CC1(C)c2cc(-c3nc(-c4ccccc4)nc(-c4ccccc4)n3)c(-n3c4ccccc4c4cc(-c5ccccc5)ccc43)cc2C(C)(C)C1(C)C]

To a well-stirred solution, cooled to 0° C., of 26.1 g (50 mmol) of S100 in 500 ml THF is added dropwise 42.3 ml (55 mmol) of isopropylmagnesium chloride-lithium chloride complex solution, 1.3 M in THF. The mixture is stirred for 1 h, and then a solution of g (60 mmol) of 2-chloro-4,6-diphenyl-1,3,5-triazine [3842-55-5] in 200 ml is added dropwise, and the mixture is allowed to warm up to RT and stirred at 60° C. for 5 h. The reaction is quenched cautiously by adding 50 ml of methanol, the solvent is largely removed under reduced pressure, the residue is taken up in 500 ml of dichloromethane (DCM), and the mixture is washed three times with 200 ml each time of water and once with 200 ml of saturated sodium chloride solution and dried over magnesium sulfate. The desiccant is filtered off and the filtrate is concentrated to about 200 ml under reduced pressure, with continuous replacement of the DCM distilled off by methanol. The crystallized product is filtered off with suction, washed twice with 50 ml each time of methanol and dried under reduced pressure. The crude product is purified by chromatography and repeated hot-extraction crystallization (customary organic solvents or the combinations thereof, preferably acetonitrile-DCM, 1:3 to 3:1 vv) and fractional sublimation or heat treatment under high vacuum. Yield: 26.5 g (39 mmol), 78%; purity: about 99.9% by HPLC.

The following compounds can be prepared analogously:

| Ex. | Reactants | Product | Yield |
|---|---|---|---|
| A401 | S100 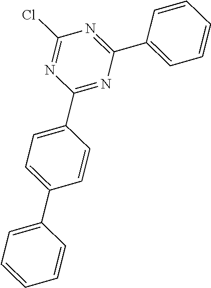 [SMILES: Clc1nc(-c2ccccc2)nc(-c2ccc(-c3ccccc3)cc2)n1] 1472062-94-4 | 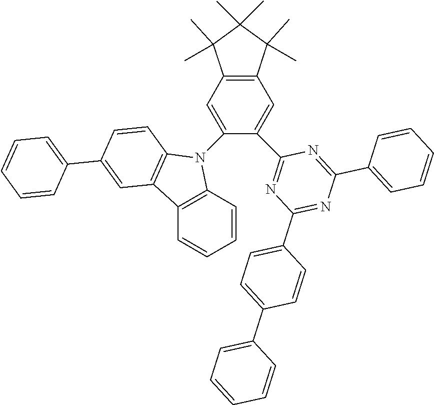 [SMILES: CC1(C)c2cc(-c3nc(-c4ccccc4)nc(-c4ccc(-c5ccccc5)cc4)n3)c(-n3c4ccccc4c4cc(-c5ccccc5)ccc43)cc2C(C)(C)C1(C)C] | 73% |
| A402 | S100 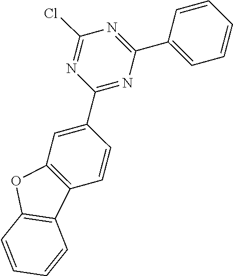 [SMILES: Clc1nc(-c2ccccc2)nc(-c2ccc3c(c2)oc2ccccc23)n1] 2142681-84-1 | 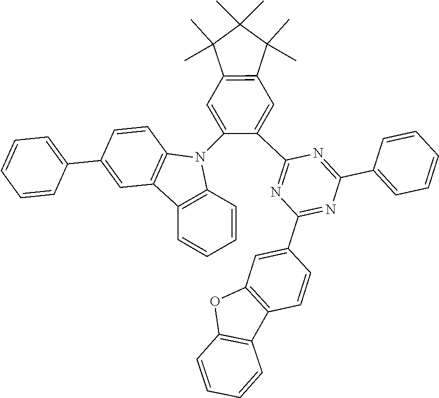 [SMILES: CC1(C)c2cc(-c3nc(-c4ccccc4)nc(-c4ccc5c(c4)oc4ccccc45)n3)c(-n3c4ccccc4c4cc(-c5ccccc5)ccc43)cc2C(C)(C)C1(C)C] | 75% |
| A403 | S101 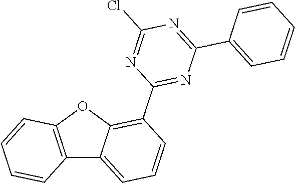 [SMILES: Clc1nc(-c2ccccc2)nc(-c2cccc3c2oc2ccccc23)n1] 1472729-25-1 | 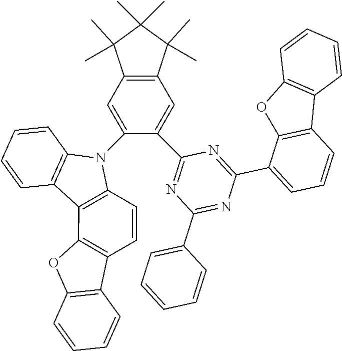 [SMILES: CC1(C)c2cc(-c3nc(-c4ccccc4)nc(-c4cccc5c4oc4ccccc45)n3)c(-n3c4ccccc4c4c5oc6ccccc6c5ccc43)cc2C(C)(C)C1(C)C] | 71% |
| A404 | S101 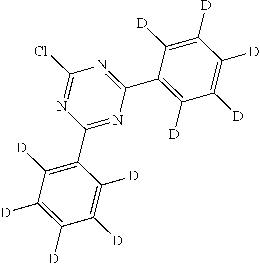 [SMILES: [2H]c1c([2H])c([2H])c(-c2nc(Cl)nc(-c3c([2H])c([2H])c([2H])c([2H])c3[2H])n2)c([2H])c1[2H]] 1300115-09-6 | 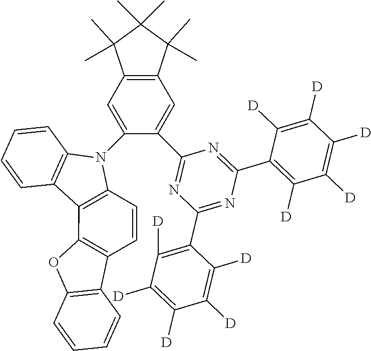 [SMILES: [2H]c1c([2H])c([2H])c(-c2nc(-c3cc4c(cc3-n3c5ccccc5c5c6oc7ccccc7c6ccc53)C(C)(C)C(C)(C)C4(C)C)nc(-c3c([2H])c([2H])c([2H])c([2H])c3[2H])n2)c([2H])c1[2H]] | 77% |
| A405 | S102 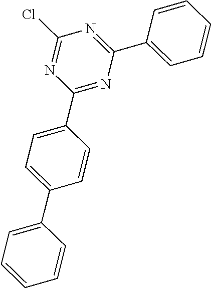 [SMILES: Clc1nc(-c2ccccc2)nc(-c2ccc(-c3ccccc3)cc2)n1] 1472062-94-4 | 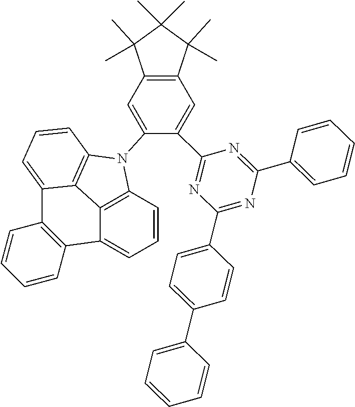 [SMILES: CC1(C)c2cc(-c3nc(-c4ccccc4)nc(-c4ccc(-c5ccccc5)cc4)n3)c(-n3c4cccc5c6ccccc6c6cccc3c6c54)cc2C(C)(C)C1(C)C] | 78% |
| A406 | S102 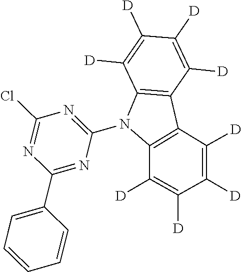 [SMILES: [2H]c1c([2H])c([2H])c2c(c1[2H])c1c([2H])c([2H])c([2H])c([2H])c1n2-c1nc(Cl)nc(-c2ccccc2)n1] 2574571-56-3 | 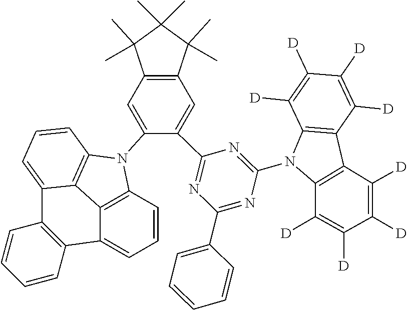 [SMILES: [2H]c1c([2H])c([2H])c2c(c1[2H])c1c([2H])c([2H])c([2H])c([2H])c1n2-c1nc(-c2ccccc2)nc(-c2cc3c(cc2-n2c4cccc5c6ccccc6c6cccc2c6c54)C(C)(C)C(C)(C)C3(C)C)n1] | 76% |
| A407 | S102 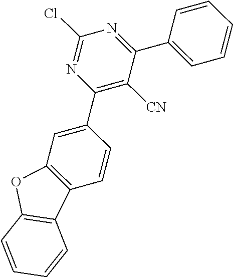 [SMILES: N#Cc1c(-c2ccccc2)nc(Cl)nc1-c1ccc2c(c1)oc1ccccc12] | 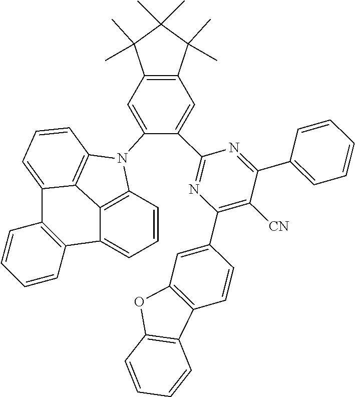 [SMILES: CC1(C)c2cc(-c3nc(-c4ccccc4)c(C#N)c(-c4ccc5c(c4)oc4ccccc45)n3)c(-n3c4cccc5c6ccccc6c6cccc3c6c54)cc2C(C)(C)C1(C)C] | 64% |
| A408 | S103 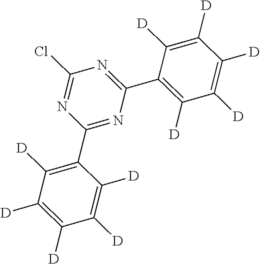 [SMILES: [2H]c1c([2H])c([2H])c(-c2nc(Cl)nc(-c3c([2H])c([2H])c([2H])c([2H])c3[2H])n2)c([2H])c1[2H]] 1300115-09-6 | 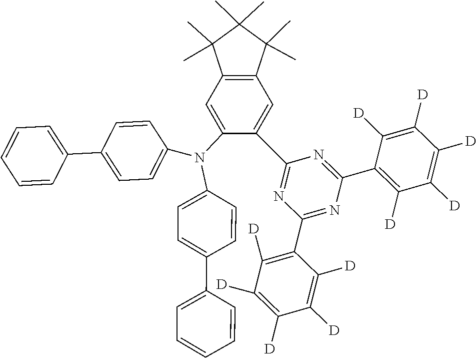 [SMILES: [2H]c1c([2H])c([2H])c(-c2nc(-c3cc4c(cc3N(c3ccc(-c5ccccc5)cc3)c3ccc(-c5ccccc5)cc3)C(C)(C)C(C)(C)C4(C)C)nc(-c3c([2H])c([2H])c([2H])c([2H])c3[2H])n2)c([2H])c1[2H]] | 63% |
| A409 | S103 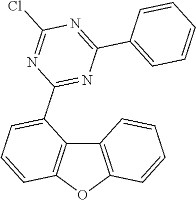 [SMILES: Clc1nc(-c2ccccc2)nc(-c2cccc3oc4ccccc4c23)n1] 1883265-32-4 | 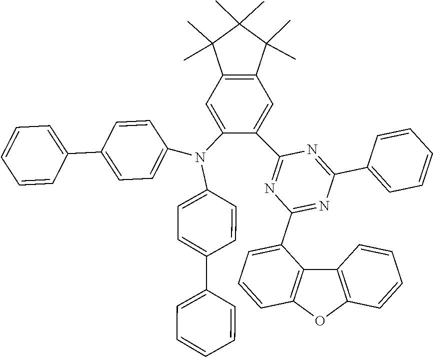 [SMILES: CC1(C)c2cc(-c3nc(-c4ccccc4)nc(-c4cccc5oc6ccccc6c45)n3)c(N(c3ccc(-c4ccccc4)cc3)c3ccc(-c4ccccc4)cc3)cc2C(C)(C)C1(C)C] | 61% |

### Example: Production of the OLEDs

### 1) Vacuum-Processed Devices:

OLEDs of the invention and OLEDs according to the prior art are produced by a general method according to WO 2004/058911, which is adapted to the circumstances described here (variation in layer thickness, materials used).

In the examples which follow, the results for various OLEDs are presented. Cleaned glass plates (cleaning in Miele laboratory glass washer, Merck Extran detergent) coated with structured ITO (indium tin oxide) of thickness 50 nm are pretreated with UV ozone for 25 minutes (UVP PR-100 UV ozone generator). These coated glass plates form the substrates to which the OLEDs are applied.

### 1a) Blue Fluorescent OLED Components—BF:

The compounds of the invention can be used in the hole injection layer (HIL), hole transport layer (HTL), the electron blocker layer (EBL) and the electron transport layer (ETL). All materials are applied by thermal vapor deposition in a vacuum chamber. The emission layer (EML) here always consists of at least one matrix material (host material) SMB (see table 1) and an emitting dopant (dopant, emitter) D, which is added to the matrix material(s) in a particular proportion by volume by co-evaporation. Details given in such a form as SMB:D (97:3%) mean here that the material SMB is present in the layer in a proportion by volume of 97% and the dopant D in a proportion of 3%. Analogously, the electron transport layer may also consist of a mixture of two materials; see table 1. The materials used to produce the OLEDs are shown in table 5 or relate to the synthesis examples detailed above.

The OLEDs are characterized in a standard manner. For this purpose, the electroluminescence spectra, current efficiency (measured in cd/A), power efficiency (measured in Im/W) and external quantum efficiency (EQE, measured in percent) as a function of luminance, calculated from current-voltage-luminance characteristics (IUL characteristics) assuming Lambertian emission characteristics, and also lifetime are determined. EQE in (%) and voltage in (V) are reported at a luminance of 1000 cd/m². Lifetime is determined at a starting luminance of 10 000 cd/m². The measured period of time within which the brightness of the reference has dropped to 80% of initial brightness is set at 100%. The lifetime of the OLED components containing the compounds of the invention is reported in percent relative to the reference.

The OLEDs have the Following Layer Structure:

### Substrate

Hole injection layer (HIL) composed of HTM1 doped with 5% NDP-9 (commercially available from Novaled), 20 nm
Hole transport layer (HTL), see table 1
Electron blocker layer (EBL), see table 1
Emission layer (EML), see table 1
Electron transport layer (ETL), see table 1
Electron injection layer (EIL) composed of ETM2, 1 nm
Cathode composed of aluminum, 100 nm

TABLE 1
Structure of blue fluorescent OLED components

| | HTL | EBL | EML | ETL |
|---|---|---|---|---|
| Ex. | thickness | thickness | thickness | thickness |
| BF- | Ref-HTM1 | EBM1 | SMB1:Ref-D1 | ETM1:ETM2 |
| Ref1 | 180 nm | 10 nm | (95%:5%) | (50%:50%) |
| | | | 20 nm | 30 nm |
| BF- | HTM1 | Ref-EBM1 | SMB1:Ref-D1 | ETM1:ETM2 |
| Ref2 | 180 nm | 10 nm | (95%:5%) | (50%:50%) |
| | | | 20 nm | 30 nm |
| BF- | Ref-HTM1 | Ref-EBM1 | SMB1:Ref-D1 | ETM1:ETM2 |
| Ref3 | 180 nm | 10 nm | (95%:5%) | (50%:50%) |
| | | | 20 nm | 30 nm |
| BF1 | A4 | EBM1 | SMB1:Ref-D1 | ETM1:ETM2 |
| | 180 nm | 10 nm | (95%:5%) | (50%:50%) |
| | | | 20 nm | 30 nm |
| BF2 | HTM1 | A1 | SMB1:Ref-D1 | ETM1:ETM2 |
| | 180 nm | 10 nm | (95%:5%) | (50%:50%) |
| | | | 20 nm | 30 nm |
| BF3 | A4 | A1 | SMB1:Ref-D1 | ETM1:ETM2 |
| | 180 nm | 10 nm | (95%:5%) | (50%:50%) |
| | | | 20 nm | 30 nm |
| BF4 | A7 | EBM1 | SMB1:Ref-D1 | ETM1:ETM2 |
| | 180 nm | 10 nm | (95%:5%) | (50%:50%) |
| | | | 20 nm | 30 nm |
| BF5 | HTM1 | A11 | SMB2:Ref-D1 | ETM1:ETM2 |
| | 180 nm | 10 nm | (95%:5%) | (50%:50%) |
| | | | 20 nm | 30 nm |
| BF6 | A103 | EBM1 | SMB3:Ref-D1 | ETM1:ETM2 |
| | 180 nm | 10 nm | (95%:5%) | (50%:50%) |
| | | | 20 nm | 30 nm |
| BF7 | HTM1 | EBM1 | SMB1:Ref-D1 | A173:ETM2 |
| | 180 nm | 10 nm | (95%:5%) | (50%:50%) |
| | | | 20 nm | 30 nm |

TABLE 2
Results for blue fluorescence OLED components

| | EQE (%) | Voltage (V) | LT80 [%] |
|---|---|---|---|
| Ex. | 1000 cd/m² | 1000 cd/m² | 10 000 cd/m² |
| BF-Ref1 | 7.7 | 4.0 | 100 |
| BF-Ref2 | 7.9 | 4.1 | 100 |
| BF-Ref3 | 7.5 | 4.0 | 100 |
| BF1 | 8.2 | 3.8 | 125 |
| BF2 | 8.4 | 3.9 | 140 |
| BF3 | 8.5 | 3.8 | 180 |
| BF4 | 8.3 | 3.7 | 130 |
| BF5 | 8.6 | 4.0 | 140 |
| BF6 | 8.5 | 3.9 | 155 |
| BF7 | 8.3 | 4.1 | 130 |

### 1b) Phosphorescent OLED Components:

The compounds of the invention A can be used in the hole injection layer (HIL), the hole transport layer (HTL), the electron blocker layer (EBL) and in the emission layer (EML) as matrix material (host material) M (see table 5) or A (see materials of the invention). For this purpose, all the materials are applied by thermal vapor deposition in a vacuum chamber. The emission layer here always consists of at least one or more than one matrix material M and a phosphorescent dopant Ir which is added to the matrix material(s) in a particular proportion by volume by co-evaporation. Details given in such a form as M1:M2:Ir (55%:35%:10%) mean here that the material M1 is present in the layer in a proportion by volume of 55%, M2 in a proportion by volume of 35% and Ir in a proportion by volume of 10%. Analogously, the electron transport layer may also consist of a mixture of two materials. The exact structure of the OLEDs can be found in table 3. The materials used to produce the OLEDs are shown in table 5 or relate to the synthesis examples detailed above.

The OLEDs are characterized in a standard manner. For this purpose, the electroluminescence spectra, current efficiency (measured in cd/A), power efficiency (measured in Im/W) and external quantum efficiency (EQE, measured in percent) as a function of luminance, calculated from current-voltage-luminance characteristics (IUL characteristics) assuming Lambertian emission characteristics, and also lifetime are determined. EQE in (%) and voltage in (V) are reported at a luminance of 1000 cd/m². Lifetime is determined at a starting luminance of 1000 cd/m² for blue and red, and 10 000 cd/m² for green and yellow. The measured period of time within which the brightness of the reference has dropped to 80% of initial brightness is set at 100%. The lifetime of the OLED components containing the compounds of the invention is reported in percent relative to the respective reference.

The OLEDs have the Following Layer Structure:

### Substrate

Hole injection layer (HIL) composed of HTM1 doped with 5% NDP-9 (commercially available from Novaled), 20 nm
Hole transport layer (HTL), see table 3
Electron blocker layer (EBL), see table 3
Emission layer (EML), see table 3
Hole blocker layer (HBL), see table 3
Electron transport layer (ETL), composed of ETM1:ETM2 (50%:50%), 30 nm
Electron injection layer (EIL) composed of ETM2, 1 nm
Cathode composed of aluminum, 100 nm

TABLE 3
Structure of phosphorescence OLED components

| | | | | |
|---|---|---|---|---|
| | HTL | EBL | EML | HBL |
| Ex. | thickness | thickness | thickness | thickness |

Blue

| | | | | |
|---|---|---|---|---|
| Ref-BP1 | HTM1 | EBM2 | M3:Ref-M1:IrB1 | HBM2 |
| | 180 nm | 20 nm | (30%:65%:5%) | 5 nm |
| | | | 25 nm | |
| BP1 | HTM1 | EBM2 | M3:A301:IrB1 | HBM2 |
| | 180 nm | 20 nm | (30%:65%:5%) | 5 nm |
| | | | 25 nm | |

Green

| | | | | |
|---|---|---|---|---|
| GP-Ref1 | Ref-HTM1 | EBM1 | M1:M2:IrG1 | HBM1 |
| | 50 nm | 20 nm | (30%:60%:10%) | 5 nm |
| | | | 40 nm | |
| GP-Ref2 | HTM1 | Ref-EBM1 | M1:M2:IrG1 | HBM1 |
| | 50 nm | 20 nm | (30%:60%:10%) | 5 nm |
| | | | 40 nm | |
| GP-Ref3 | HTM1 | EBM1 | M1:Ref-M2:IrG1 | HBM1 |
| | 50 nm | 20 nm | (30%:60%:10%) | 5 nm |
| | | | 40 nm | |
| GP1 | A4 | EBM1 | M1:M2:IrG1 | HBM1 |
| | 50 nm | 20 nm | (30%:60%:10%) | 5 nm |
| | | | 40 nm | |
| GP2 | HTM1 | A1 | M1:M2:IrG1 | HBM1 |
| | 50 nm | 20 nm | (30%:60%:10%) | 5 nm |
| | | | 40 nm | |
| GP3 | HTM1 | A21 | M1:M2:IrG1 | HBM1 |
| | 50 nm | 20 nm | (30%:60%:10%) | 5 nm |
| | | | 40 nm | |
| GP4 | HTM1 | A120 | M1:M2:IrG1 | HBM1 |
| | 50 nm | 20 nm | (30%:60%:10%) | 5 nm |
| | | | 40 nm | |
| GP5 | HTM1 | A155 | M1:M2:IrG1 | HBM1 |
| | 50 nm | 20 nm | (30%:60%:10%) | 5 nm |
| | | | 40 nm | |
| GP6 | A7 | EBM1 | M1:M2:IrG1 | A185 |
| | 50 nm | 20 nm | (30%:60%:10%) | 5 nm |
| | | | 40 nm | |
| GP7 | A168 | EBM1 | M1:A143:IrG1 | HBM1 |
| | 50 nm | 20 nm | (30%:60%:10%) | 5 nm |
| | | | 40 nm | |
| GP8 | HTM1 | EBM1 | A194:M2:IrG1 | HBM1 |
| | 50 nm | 20 nm | (40%:50%:10%) | 5 nm |
| | | | 40 nm | |
| GP9 | HTM1 | EBM1 | A189:IrG1 | HBM1 |
| | 50 nm | 20 nm | (84%:16%) | 5 nm |
| | | | 40 nm | |

Yellow

| | | | | |
|---|---|---|---|---|
| GP- | Ref-HTM1 | EBM1 | M1:M2:IrG2 | HBM1 |
| Ref50 | 50 nm | 20 nm | (30%:70%:10%) | 5 nm |
| | | | 40 nm | |
| GP- | HTM1 | Ref-EBM1 | M1:M2:IrG2 | HBM1 |
| Ref51 | 50 nm | 20 nm | (30%:70%:10%) | 5 nm |
| | | | 40 nm | |
| GP- | HTM1 | EBM1 | M1:Ref-M2:IrG1 | HBM1 |
| Ref52 | 50 nm | 20 nm | (30%:60%:10%) | 5 nm |
| | | | 40 nm | |
| GP50 | A4 | EBM1 | M1:M2:IrG2 | HBM1 |
| | 50 nm | 20 nm | (30%:70%:10%) | 5 nm |
| | | | 40 nm | |
| GP51 | HTM1 | A1 | M1:M2:IrG2 | HBM1 |
| | 50 nm | 20 nm | (30%:70%:10%) | 5 nm |
| | | | 40 nm | |
| GP52 | A7 | EBM1 | M1:M2:IrG2 | A172 |
| | 50 nm | 20 nm | (30%:60%:10%) | 5 nm |
| | | | 40 nm | |
| GP53 | A7 | EBM1 | M1:M2:IrG2 | A174 |
| | 50 nm | 20 nm | (30%:60%:10%) | 5 nm |
| | | | 40 nm | |
| GP54 | A7 | EBM1 | M1:M2:IrG2 | A191 |
| | 50 nm | 20 nm | (30%:60%:10%) | 5 nm |
| | | | 40 nm | |
| GP55 | A7 | EBM1 | M1:M2:IrG2 | A193 |
| | 50 nm | 20 nm | (30%:60%:10%) | 5 nm |
| | | | 40 nm | |
| GP56 | A7 | EBM1 | M1:M2:IrG2 | A195 |
| | 50 nm | 20 nm | (30%:60%:10%) | 5 nm |
| | | | 40 nm | |
| GP57 | A169 | EBM1 | M1:A143:IrG2 | HBM1 |
| | 50 nm | 20 nm | (30%:60%:10%) | 5 nm |
| | | | 40 nm | |
| GP58 | A169 | EBM1 | M1:A36:IrG2 | HBM1 |
| | 50 nm | 20 nm | (30%:60%:10%) | 5 nm |
| | | | 40 nm | |
| GP59 | A169 | EBM1 | M1:A52:IrG2 | HBM1 |
| | 50 nm | 20 nm | (30%:60%:10%) | 5 nm |
| | | | 40 nm | |
| GP60 | A169 | EBM1 | M1:A136:IrG2 | HBM1 |
| | 50 nm | 20 nm | (30%:60%:10%) | 5 nm |
| | | | 40 nm | |

Red

| | | | | |
|---|---|---|---|---|
| RP-Ref1 | Ref-HTM1 | EBM1 | M5:IrR1 | HBM1 |
| | 50 nm | 20 nm | (95%:5%) | 10 nm |
| | | | 35 nm | |
| RP-Ref2 | HTM1 | Ref-EBM1 | M5:IrR1 | HBM1 |
| | 50 nm | 20 nm | (95%:5%) | 10 nm |
| | | | 35 nm | |
| RP1 | A4 | EBM1 | M5:IrR1 | HBM1 |
| | 50 nm | 20 nm | (95%:5%) | 10 nm |
| | | | 35 nm | |
| RP2 | A156 | EBM1 | M5:IrR1 | HBM1 |
| | 50 nm | 20 nm | (95%:5%) | 10 nm |
| | | | 35 nm | |
| RP3 | A158 | EBM1 | M5:IrR1 | HBM1 |
| | 50 nm | 20 nm | (95%:5%) | 10 nm |
| | | | 35 nm | |
| RP4 | HTM1 | A1 | M5:IrR1 | HBM1 |
| | 50 nm | 20 nm | (95%:5%) | 10 nm |
| | | | 35 nm | |
| RP5 | HTM1 | A161 | M5:IrR1 | HBM1 |
| | 50 nm | 20 nm | (95%:5%) | 10 nm |
| | | | 35 nm | |
| RP6 | HTM1 | A162 | M5:IrR1 | HBM1 |
| | 50 nm | 20 nm | (95%:5%) | 10 nm |
| | | | 35 nm | |
| RP7 | HTM1 | A167 | M5:IrR1 | HBM1 |
| | 50 nm | 20 nm | (95%:5%) | 10 nm |
| | | | 35 nm | |
| RP8 | A4 | A1 | A41:IrR1 | HBM1 |
| | 50 nm | 20 nm | (95%:5%) | 10 nm |
| | | | 35 nm | |
| RP9 | A4 | A1 | A75:IrR1 | HBM1 |
| | 50 nm | 20 nm | (95%:5%) | 10 nm |
| | | | 35 nm | |
| RP10 | A4 | A1 | A77:IrR1 | HBM1 |
| | 50 nm | 20 nm | (94%:6%) | 10 nm |
| | | | 35 nm | |
| RP11 | A4 | A1 | A112:IrR1 | HBM1 |
| | 50 nm | 20 nm | (95%:5%) | 10 nm |
| | | | 35 nm | |
| RP12 | A4 | A1 | A137:IrR1 | HBM1 |
| | 50 nm | 20 nm | (95%:5%) | 10 nm |
| | | | 35 nm | |
| RP13 | A4 | A1 | A401:IrR1 | HBM1 |
| | 50 nm | 20 nm | (95%:5%) | 10 nm |
| | | | 35 nm | |
| RP14 | A4 | A1 | A406:IrR1 | HBM1 |
| | 50 nm | 20 nm | (95%:5%) | 10 nm |
| | | | 35 nm | |
| RP15 | A4 | A1 | A204:IrR1 | HBM1 |
| | 50 nm | 20 nm | (95%:5%) | 10 nm |
| | | | 35 nm | |

TABLE 4
Results for phosphorescence OLED components
Blue

| | EQE (%) | Voltage (V) | LT80 (%) |
|---|---|---|---|
| Ex. | 1000 cd/m² | 1000 cd/m² | 1000 cd/m² |
| Ref-BP1 | 21.0 | 4.5 | 100 |
| BP1 | 21.9 | 4.2 | 350 |

| | | | |
|---|---|---|---|
| | EQE (%) | Voltage (V) | LT80 (%) |
| Ex. | 1000 cd/m² | 1000 cd/m² | 10 000 cd/m² |

Green

| | | | |
|---|---|---|---|
| GP-Ref1 | 22.3 | 3.5 | 100 |
| GP-Ref2 | 22.7 | 3.4 | 100 |
| GP-Ref3 | 22.2 | 3.3 | 100 |
| GP1 | 22.4 | 3.2 | 120 |
| GP2 | 22.7 | 3.1 | 160 |
| GP3 | 22.6 | 3.2 | 150 |
| GP4 | 23.0 | 3.1 | 120 |
| GP5 | 23.2 | 3.1 | 170 |
| GP6 | 23.0 | 3.0 | 115 |
| GP7 | 22.7 | 3.2 | 135 |
| GP8 | 23.1 | 3.3 | 120 |
| GP9 | 23.5 | 3.2 | 70 |

Yellow

| | | | |
|---|---|---|---|
| GP-Ref50 | 29.0 | 3.3 | 100 |
| GP-Ref51 | 29.6 | 3.1 | 100 |
| GP-Ref52 | 30.1 | 3.2 | 100 |
| GP50 | 30.2 | 3.0 | 135 |
| GP51 | 30.3 | 2.9 | 160 |
| GP52 | 29.8 | 3.0 | 120 |
| GP53 | 30.3 | 3.1 | 110 |
| GP54 | 30.5 | 2.9 | 130 |
| GP55 | 31.0 | 3.0 | 125 |
| GP56 | 30.7 | 2.9 | 120 |
| GP57 | 30.4 | 3.0 | 140 |
| GP58 | 29.8 | 3.0 | 155 |
| GP59 | 30.5 | 2.9 | 135 |
| GP60 | 30.7 | 3.1 | 125 |

Red

| | EQE (%) | Voltage (V) | LT80 (%) |
|---|---|---|---|
| Ex. | 1000 cd/m² | 1000 cd/m² | 1000 cd/m² |
| RP-Ref1 | 16.2 | 3.6 | 100 |
| RP-Ref2 | 16.4 | 3.4 | 100 |
| RP1 | 17.0 | 3.3 | 140 |
| RP2 | 16.9 | 3.3 | 130 |
| RP3 | 17.2 | 3.2 | 135 |
| RP4 | 17.1 | 3.2 | 170 |
| RP5 | 17.5 | 3.1 | 190 |
| RP6 | 17.2 | 3.3 | 180 |
| RP7 | 17.1 | 3.2 | 145 |
| RP8 | 17.5 | 3.3 | 160 |
| RP9 | 17.7 | 3.2 | 150 |
| RP10 | 17.4 | 3.1 | 180 |
| RP11 | 17.4 | 3.3 | 155 |
| RP12 | 17.1 | 3.2 | 130 |
| RP13 | 17.6 | 3.2 | 190 |
| RP14 | 17.5 | 3.2 | 160 |
| RP15 | 17.3 | 3.1 | 145 |

TABLE 5
Structural formulae of the materials used

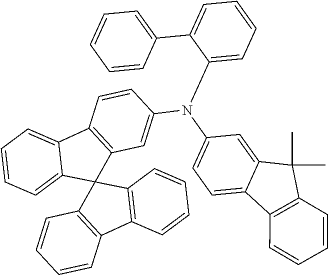 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

HTM1
136463-07-5

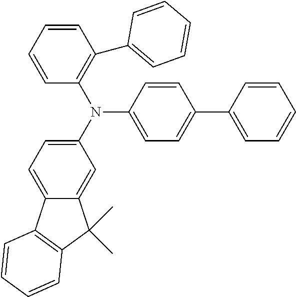 [SMILES: CC1(C)c2ccccc2-c2ccc(N(c3ccc(-c4ccccc4)cc3)c3ccccc3-c3ccccc3)cc21]

Ref-HTM1
1549792-41-7

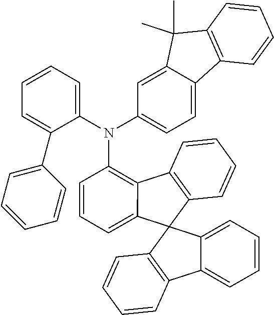 [SMILES: CC1(C)c2ccccc2-c2ccc(N(c3ccccc3-c3ccccc3)c3cccc4c3-c3ccccc3C43c4ccccc4-c4ccccc43)cc21]

EBM1
1450933-44-4

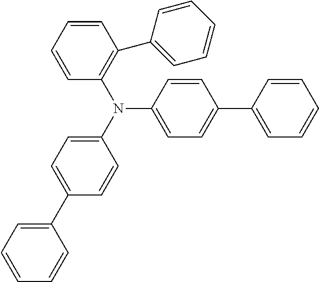 [SMILES: c1ccc(-c2ccc(N(c3ccc(-c4ccccc4)cc3)c3ccccc3-c3ccccc3)cc2)cc1]

Ref-EBM1
1443540-42-8

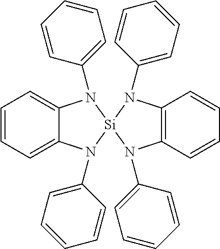 [SMILES: c1ccc(N2c3ccccc3N(c3ccccc3)[Si]23N(c2ccccc2)c2ccccc2N3c2ccccc2)cc1]

EBM2
1206465-62-4

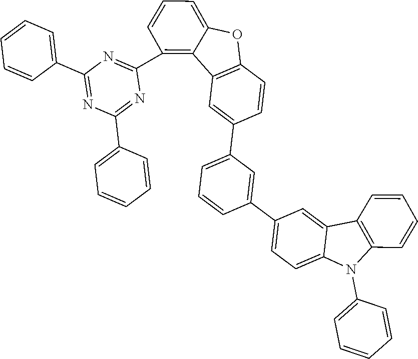 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

M1
1822310-86-0

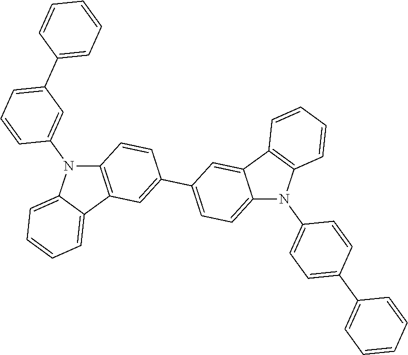 [SMILES: c1ccc(-c2ccc(-n3c4ccccc4c4cc(-c5ccc6c(c5)c5ccccc5n6-c5cccc(-c6ccccc6)c5)ccc43)cc2)cc1]

M2
1643479-47-3

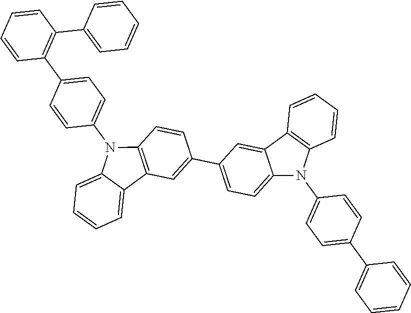 [SMILES: c1ccc(-c2ccc(-n3c4ccccc4c4cc(-c5ccc6c(c5)c5ccccc5n6-c5ccc(-c6ccccc6-c6ccccc6)cc5)ccc43)cc2)cc1]

Ref-M2
1548581-37-8

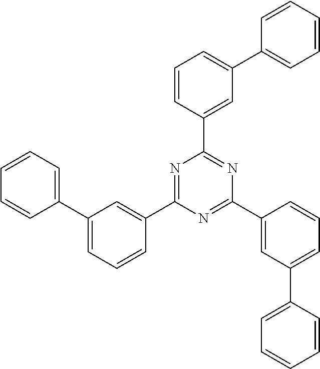 [SMILES: c1ccc(-c2cccc(-c3nc(-c4cccc(-c5ccccc5)c4)nc(-c4cccc(-c5ccccc5)c4)n3)c2)cc1]

M3 = HBM2
1201800-83-0

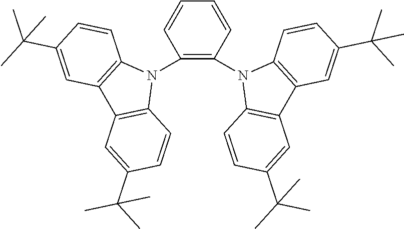 [SMILES: CC(C)(C)c1ccc2c(c1)c1cc(C(C)(C)C)ccc1n2-c1ccccc1-n1c2ccc(C(C)(C)C)cc2c2cc(C(C)(C)C)ccc21]

Ref-M1
2378004-55-6

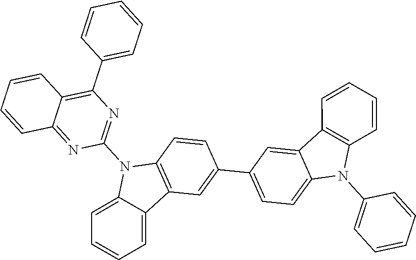 [SMILES: c1ccc(-c2nc(-n3c4ccccc4c4cc(-c5ccc6c(c5)c5ccccc5n6-c5ccccc5)ccc43)nc3ccccc23)cc1]

M5
1398395-92-0

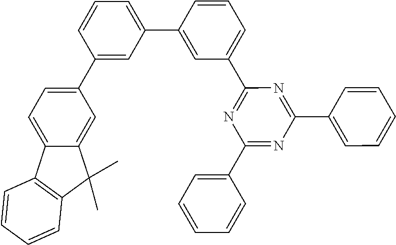 [SMILES: CC1(C)c2ccccc2-c2ccc(-c3cccc(-c4cccc(-c5nc(-c6ccccc6)nc(-c6ccccc6)n5)c4)c3)cc21]

HBM1
1955543-57-3

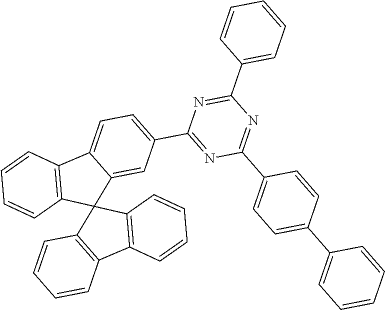 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

ETM1
1819335-36-8

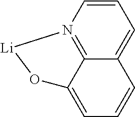

ETM2
25387-93-3

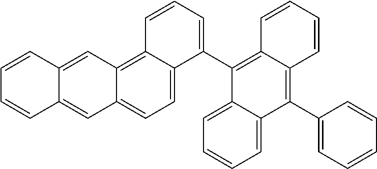 [SMILES: c1ccc(-c2c3ccccc3c(-c3cccc4c3ccc3cc5ccccc5cc34)c3ccccc23)cc1]

SMB1
1087346-88-0

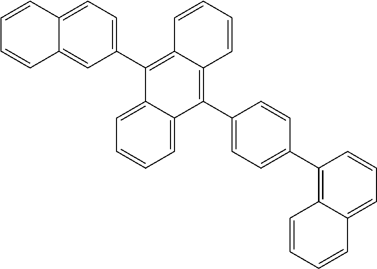 [SMILES: c1ccc2cc(-c3c4ccccc4c(-c4ccc(-c5cccc6ccccc56)cc4)c4ccccc34)ccc2c1]

SMB2
667940-34-3

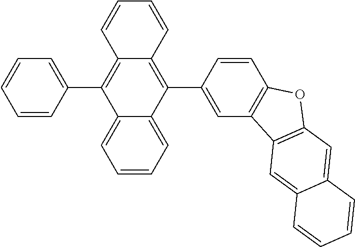 [SMILES: c1ccc(-c2c3ccccc3c(-c3ccc4oc5cc6ccccc6cc5c4c3)c3ccccc23)cc1]

SMB3
1627916-48-6
Fluorescent blue

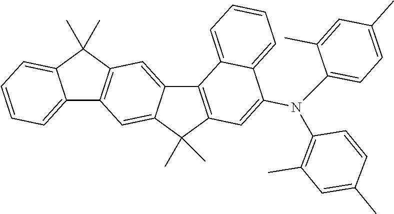 [SMILES: Cc1ccc(N(c2ccc(C)cc2C)c2cc3c(c4ccccc24)-c2cc4c(cc2C3(C)C)-c2ccccc2C4(C)C)c(C)c1]

Ref-D1
1182175-27-4
Phosphorescent blue

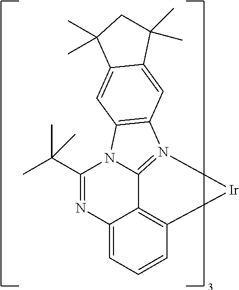

IrB1
1541114-98-0
Phosphorescent green

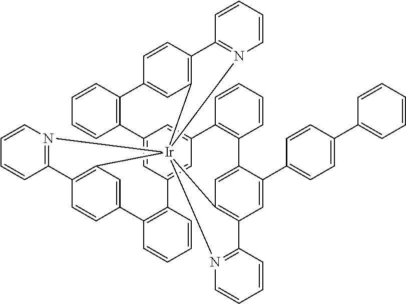

IrG1
2245866-06-0
Phosphorescent yellow

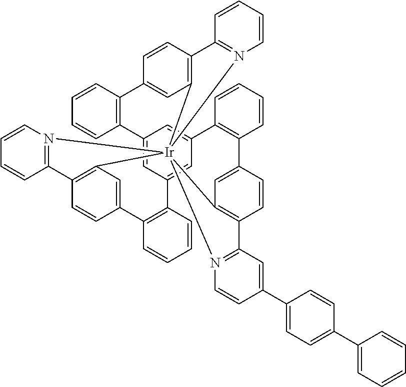

IrG2
2245945-28-0
Phosphorescent deep red

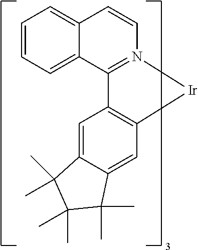

IrR1
1562420-79-4

1.-19. (canceled)

20. A compound comprising at least one structure of the formula (I): 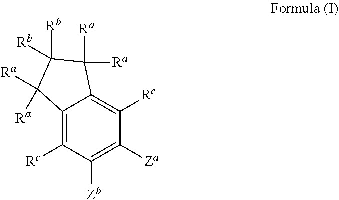
where the symbols are as follows:
Z^{a} is the same or different at each instance and is Ar^{a}, N(Ar^{c})₂ or (Ar)N(Ar^{c})₂;
Z^{b} is the same or different at each instance and is Ar^{b}, N(Ar^{d})₂ or (Ar)N(Ar^{d})₂;
R^{a} is the same or different at each instance and is a straight-chain alkyl, alkoxy or thioalkoxy group having 1 to 10 carbon atoms or a branched or cyclic alkyl, alkoxy or thioalkoxy group having 3 to 10 carbon atoms, each of which may be substituted by one or more R² radicals;
R^{b} is the same or different at each instance and is a straight-chain alkyl, alkoxy or thioalkoxy group having 1 to 10 carbon atoms or a branched or cyclic alkyl, alkoxy or thioalkoxy group having 3 to 10 carbon atoms, each of which may be substituted by one or more R² radicals;
R^{c} is the same or different at each instance and is H, D, a straight-chain alkyl, alkoxy or thioalkoxy group having 1 to 10 carbon atoms, or a branched or cyclic alkyl, alkoxy or thioalkoxy group having 3 to 10 carbon atoms, each of which may be substituted by one or more R² radicals;
Ar^{a}, Ar^{b}, Ar^{c}, Ar^{d} is the same or different at each instance and is an aromatic or heteroaromatic ring system which has 6 to 60 aromatic ring atoms and may be substituted by one or more R radicals; at the same time, two Ar^{c}, Ar^{d} radicals bonded to the same nitrogen atom may also be bridged to one another by a single bond or a bridge selected from B(R), C(R)₂, Si(R)₂, C═O, C═NR, C═C(R)₂, RC═CR, O, S, S═O, SO₂, N(R), P(R), P(═O)R and an ortho-linked phenylene group which may be substituted by one or more R radicals;
Ar is the same or different at each instance and is a connecting aromatic or heteroaromatic ring system which has 5 to 60 aromatic ring atoms and may be substituted by one or more R radicals; at the same time, one Ar radical together with one or both of the Ar^{c}, Ar^{d} radicals bonded to the same nitrogen atom may also be bridged to one another by a single bond or a bridge selected from B(R), C(R)₂, Si(R)₂, C—O, C═NR, C═C(R)₂, RC═CR, O, S, S═O, SO₂, N(R), P(R), P(═O)R and an ortho-linked phenylene group which may be substituted by one or more R radicals;
R is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals may also form a ring system together or with a further group;
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be bridged to one another by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more, adjacent R¹ radicals together may form a ring system; at the same time, one or more R¹ radicals may form a ring system with a further part of the compound;
Ar″ is the same or different at each instance and is an aromatic or heteroaromatic ring system which has 5 to 30 aromatic ring atoms and may be substituted by one or more R² radicals; at the same time, it is possible for two Ar″ radicals bonded to the same carbon atom, silicon atom, nitrogen atom, phosphorus atom or boron atom also to be bridged to one another by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, adjacent substituents R² together may form a ring system;
where the Z^{a} and Z^{b} groups do not form a ring system.

21. The compound as claimed in claim 20, comprising at least one structure of the formulae (I-1) to (I-6): 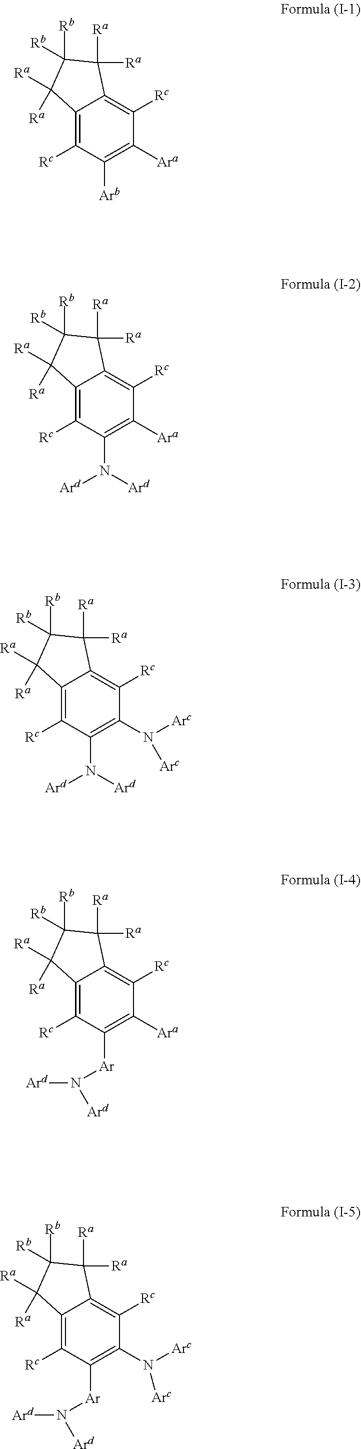 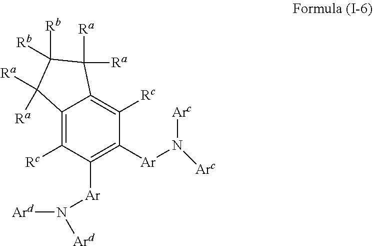
where the symbols Ar, Ar^{a}, Ar^{b}, Ar^{c}, Ar^{d}, R^{a}, R^{b} and R^{c} have the definitions given in claim 20.

22. The compound as claimed in claim 20, wherein the Ar^{a}, Ar^{b}, Ar^{c} and/or Ar^{d} group is the same or different at each instance and is selected from structures of the formulae (Ar^{a}-1) to (Ar^{a}-29): 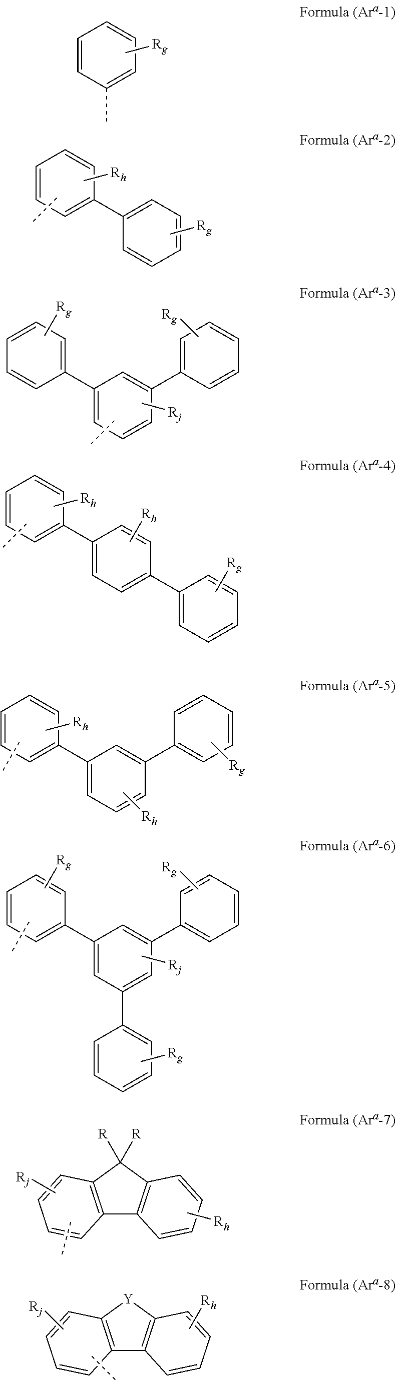 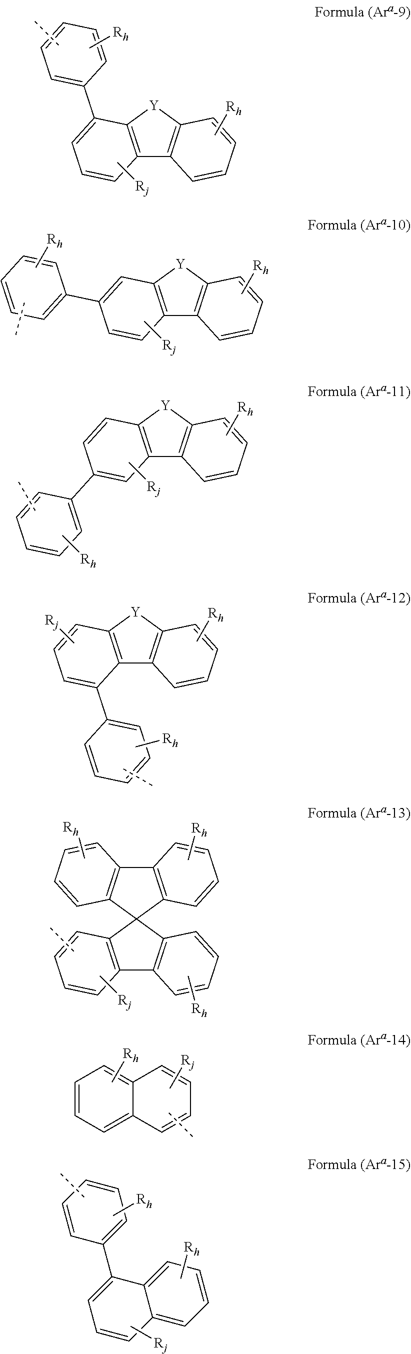 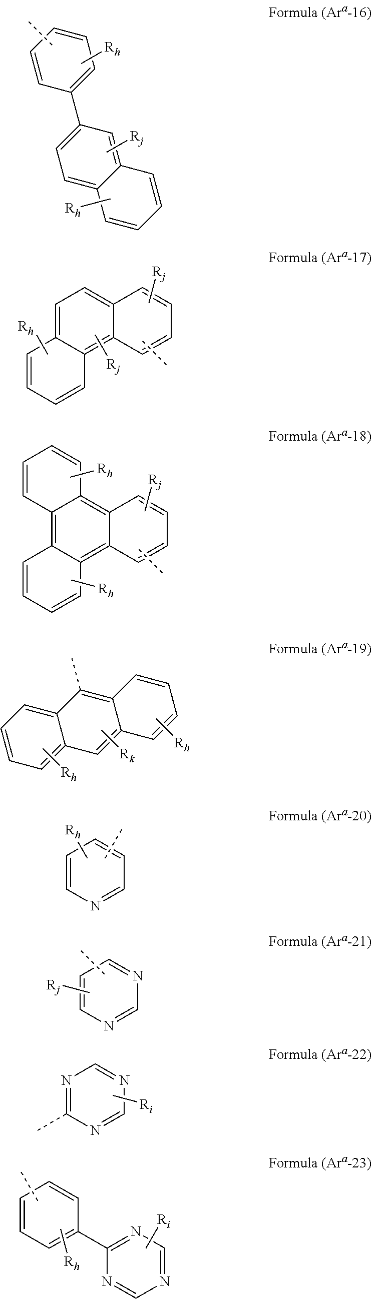 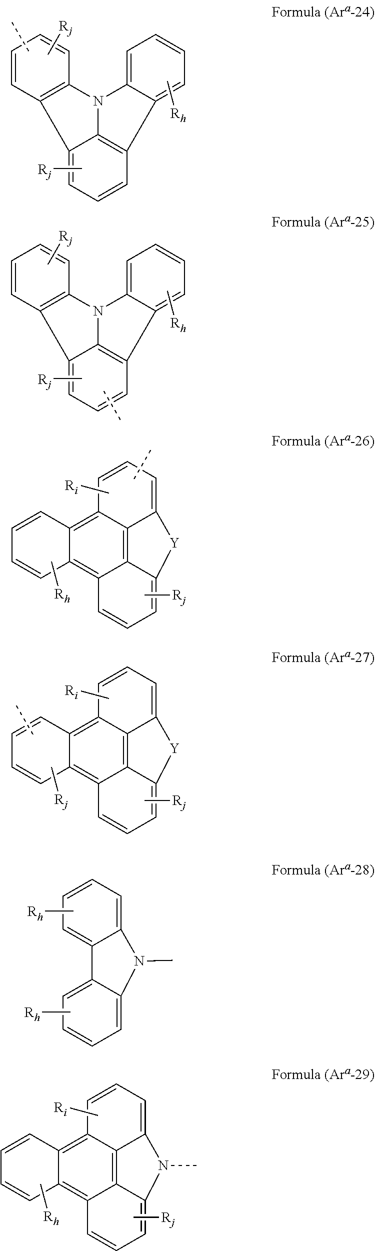
where the symbols used are as follows:
Y is O, S or NR;
k at each instance is independently 0 or 1;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5;
R has the definition given above, especially for claim 20, and the dotted bond marks the position of attachment.

23. The compound as claimed in claim 20, wherein the Ar^{a} and/or Ar^{b} group is a structure of the formula —Ar^{e}-Q, where Ar^{e} is the same or different at each instance and is a connecting aromatic or heteroaromatic ring system which has 5 to 40 aromatic ring atoms and may be substituted by one or more R radicals; and Q is an electron transport group.

24. The compound as claimed in claim 20, wherein the Ar and/or Ar^{e} group is the same or different at each instance and is selected from structures of the formulae (Ar^{e}-1) to (Ar^{e}-9): 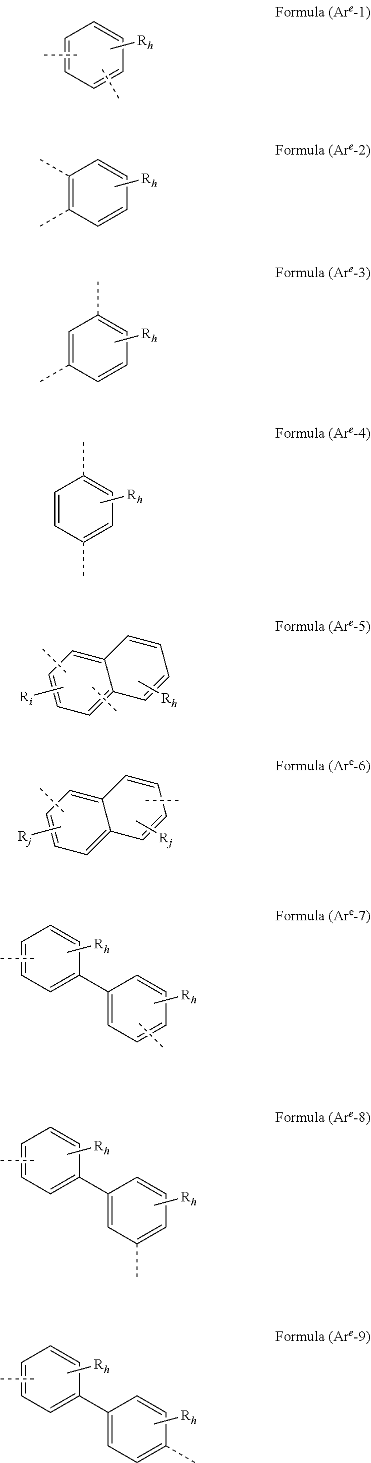
where the symbols used are as follows:
j at each instance is independently 0, 1, 2 or 3;
h at each instance is independently 0, 1, 2, 3 or 4;
R has the definition given above, especially for claim 20, and the dotted bonds mark the positions of attachment.

25. The compound as claimed in claim 20, wherein the compound comprises at least one structure of formula (I-2), where the Ar^{a} group is selected from structures of the formulae (Ar^{a}-1) to (Ar^{a}-18), where, in structures of the formulae (Ar^{a}-8) to (Ar^{a}-12), the Y group is O or NR, and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, where the Ar^{d} groups are the same or different at each instance and are selected from structures of the formulae (Ar^{a}-1), (Ar^{a}-2) and (Ar^{a}-7) to (Ar^{a}-18), and/or the compound comprises at least one structure of formula (I-4), where the Ar group is selected from structures of the formulae (Ar^{e}-1) to (Ar^{e}-4), the Ar^{a} group is selected from structures of the formulae (Ar^{a}-1) to (Ar^{a}-18), where, in structures of the formulae (Ar^{a}-8) to (Ar^{a}-12), the Y group is O or NR, and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, where the Ar^{d} groups are the same or different at each instance and are selected from structures of the formulae (Ar^{a}-1), (Ar^{a}-2), (Ar^{a}-7) to (Ar^{a}-18).

26. The compound as claimed in claim 20, wherein the compound comprises at least one structure of formula (I-3), where the Ar^{c} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, the Ar^{e} groups are the same or different at each instance and are selected from structures of the formulae (Ar^{a}-1), (Ar^{a}-2), (Ar^{a}-7) to (Ar^{a}-18); and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, where the Ar^{d} groups are the same or different at each instance and are selected from structures of the formulae (Ar^{a}-1), (Ar^{a}-2), (Ar^{a}-7) to (Ar^{a}-18), and/or the compound comprises at least one structure of formula (I-5) and/or formula (I-6), where the Ar group is the same or different at each instance and is selected from structures of the formulae (Ar^{e}-1) to (Ar^{e}-4), the Ar^{c} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, the Ar^{e} groups are the same or different at each instance and are selected from structures of the formulae (Ar^{a}-1), (Ar^{a}-2), (Ar^{a}-7) to (Ar^{a}-18); and the Ar^{d} groups are the same or different at each instance and are selected from structures of the formulae (Ar^{a}-1) to (Ar^{a}-18), where, in structures of the formulae (Ar^{a}-8) to (Ar^{a}-12), the Y group is O or NR, where the Ar^{d} groups are the same or different at each instance and are selected from structures of the formulae (Ar^{a}-1), (Ar^{a}-2), (Ar^{a}-7) to (Ar^{a}-18).

27. The compound as claimed in claim 23, wherein the compound comprises at least one structure of formula (I-2), where the Ar^{a} group is a structure of the formula Ar^{e}-Q, where the symbols Ar^{e} is the same or different at each instance and is selected from structures of the formulae (Ar^{e}-1) to (Ar^{e}-9): 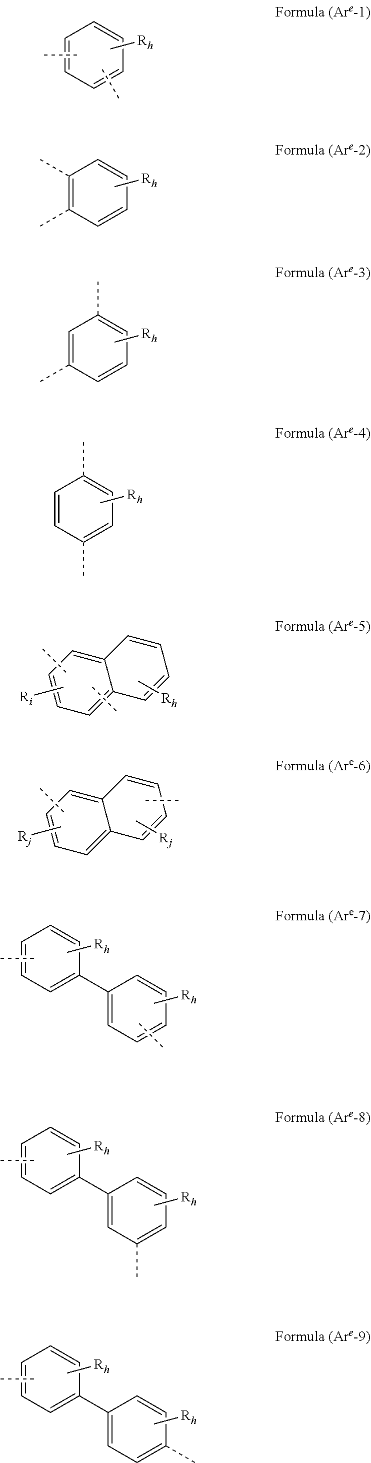
where the symbols used are as follows:
j at each instance is independently 0, 1, 2 or 3;
h at each instance is independently 0, 1, 2, 3 or 4,
and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, where the Ar^{d} groups are the same or different at each instance and are selected from structures of the formulae (Ar^{a}-1), (Ar^{a}-2), (Ar^{a}-7) to (Ar^{a}-18) and/or the compound comprises at least one structure of formula (I-4), where the Ar group is selected from structures of the formulae (Ar^{e}-1) to (Ar^{e}-4), the Ar^{a} group is a structure of the formula Ar^{e}-Q, where the symbols Ar^{e} is the same or different at each instance and is selected from structures of the formulae (Ar^{e}-1) to (Ar^{e}-9): 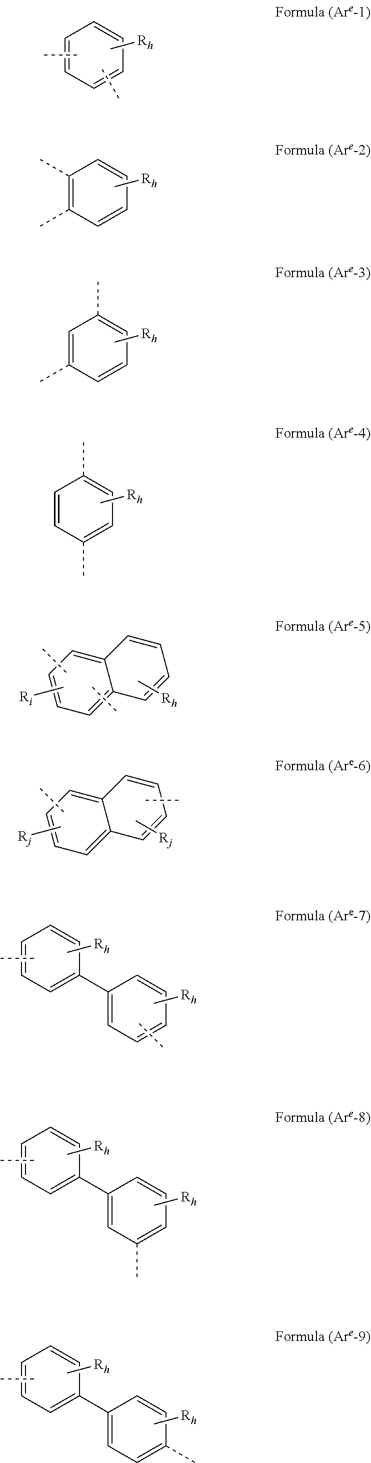
where the symbols used are as follows:
j at each instance is independently 0, 1, 2 or 3;
h at each instance is independently 0, 1, 2, 3 or 4;
and the Ar^{d} groups are the same or different at each instance and are selected from structures of the formulae (Ar^{a}-1) to (Ar^{a}-23), where, in structures of the formulae (Ar^{a}-8) to (Ar^{a}-12), the Y group is O or NR, where the Ar^{d} groups are the same or different at each instance and are selected from structures of the formulae (Ar^{a}-1), (Ar^{a}-2), (Ar^{a}-7) to (Ar^{a}-18).

28. The compound as claimed in claim 20, wherein the compound comprises at least one structure of formula (I-1) and/or formula (I-2), where the Ar^{a} group comprises an electron transport group or is an electron transport group, optionally substituted by one or more R radicals.

29. The compound as claimed in claim 28, wherein the compound comprises at least one structure of formula (I-1), where the Ar^{b} group comprises an electron transport group or is an electron transport group, optionally substituted by one or more R radicals.

30. The compound as claimed in claim 28, wherein the compound comprises at least one structure of formula (I-1), where the Ar^{b} group is selected from structures of the formulae (Ar^{a}-1) to (Ar^{a}-18), where, in structures of the formulae (Ar^{a}-8) to (Ar^{a}-12), the Y group is O or NR; where the Ar^{a} group is a structure of the formula Ar^{e}-Q, where the Ar^{e} group is the same or different at each instance and is selected from structures of the formulae (Ar^{e}-1) to (Ar^{e}-9): 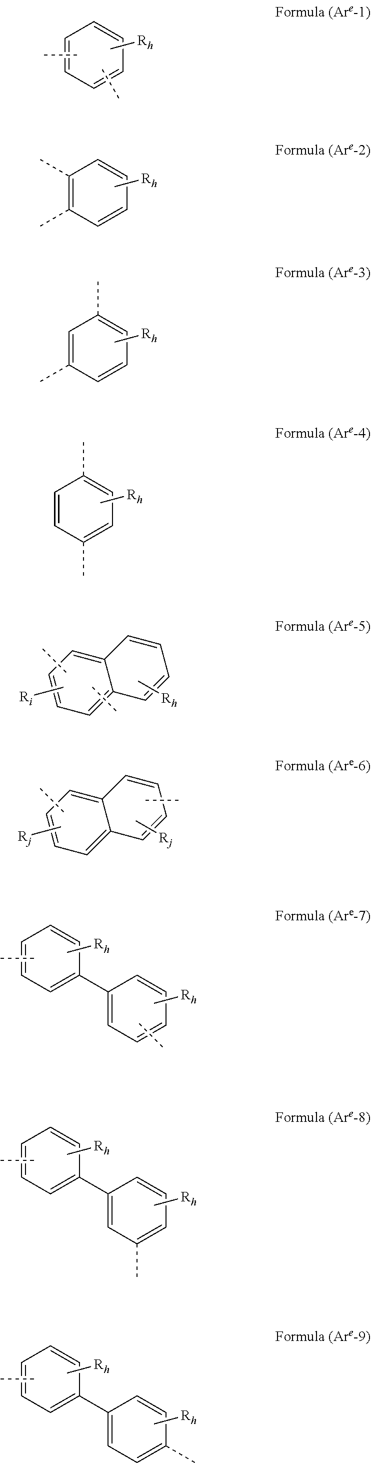
where the symbols used are as follows:
j at each instance is independently 0, 1, 2 or 3;
h at each instance is independently 0, 1, 2, 3 or 4;
R has the definition given above, especially for claim 20, and the dotted bonds mark the positions of attachment.

31. The compound as claimed in claim 20, comprising at least one structure of the formulae (II-1) to (II-154): 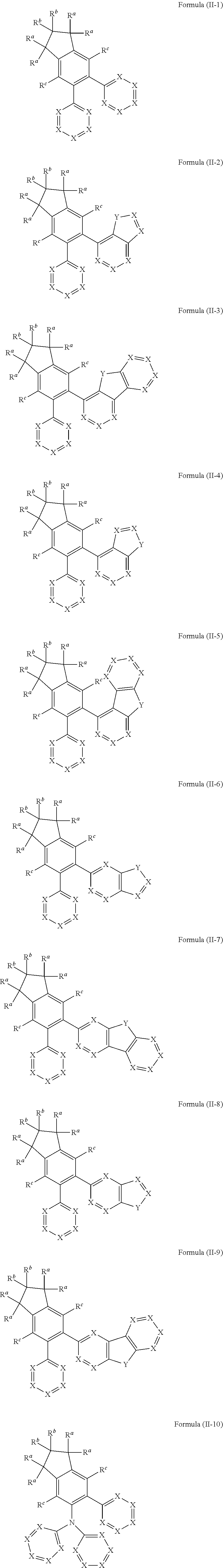 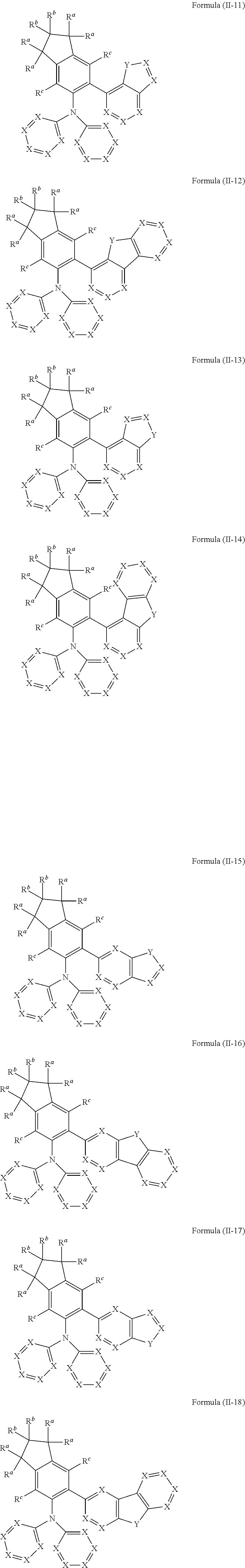 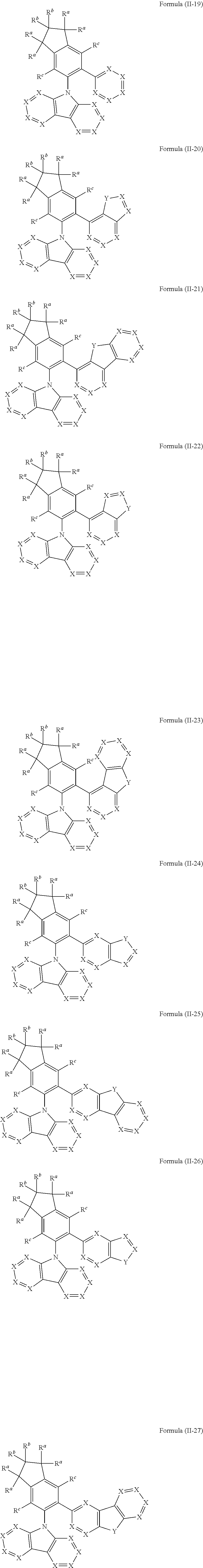 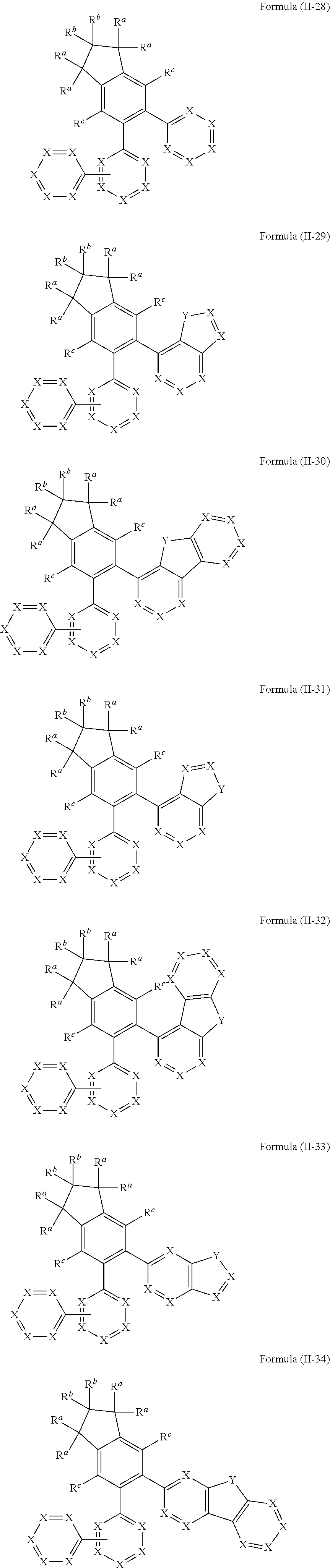 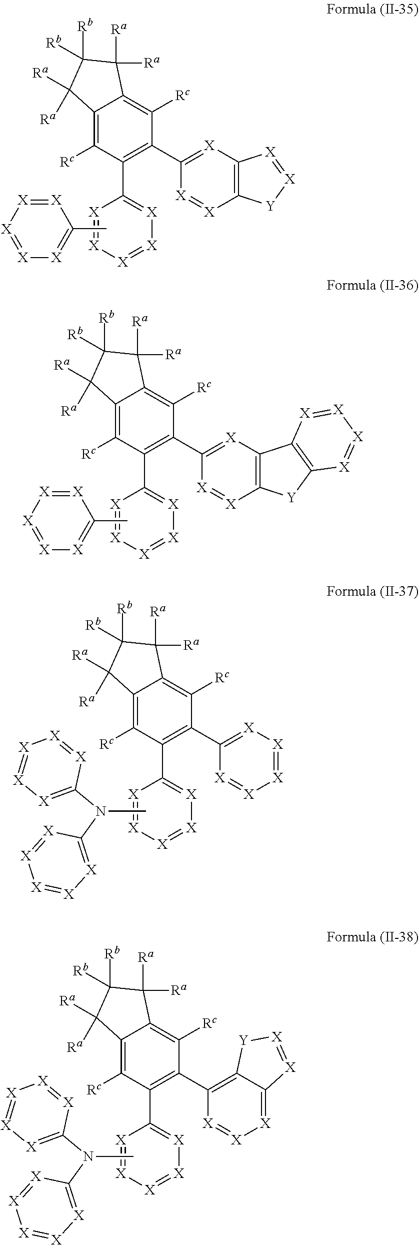 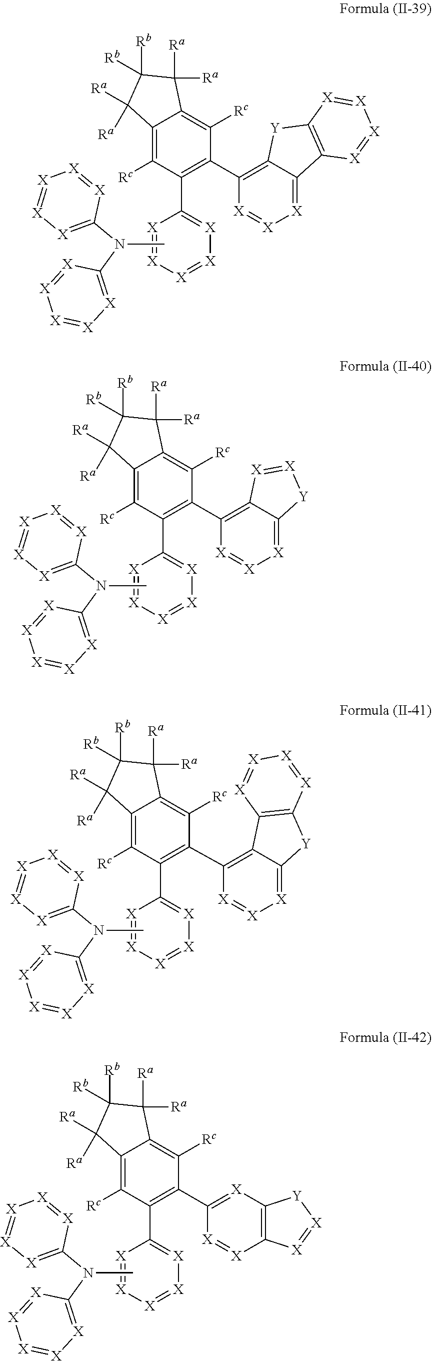 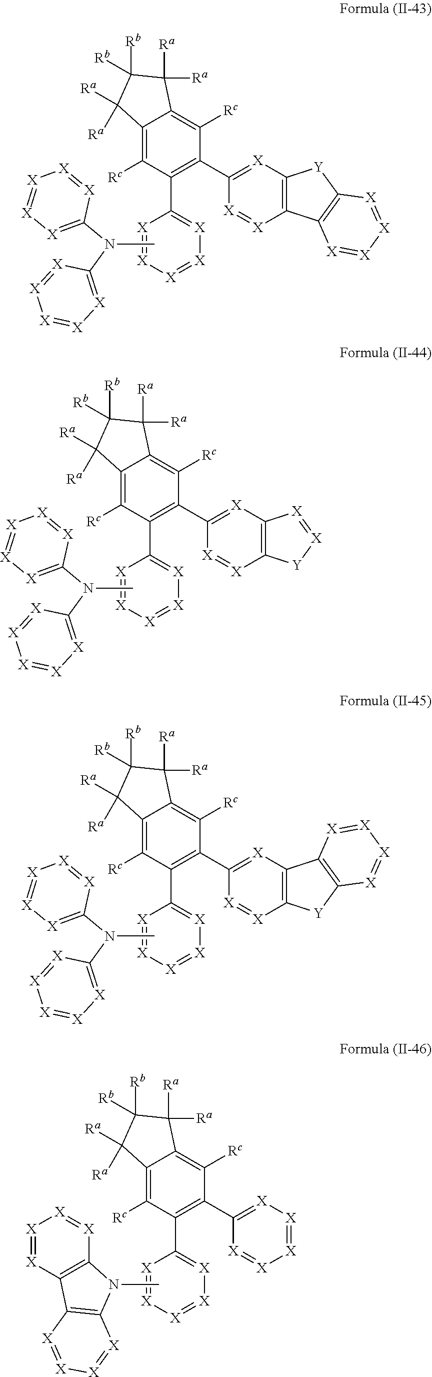 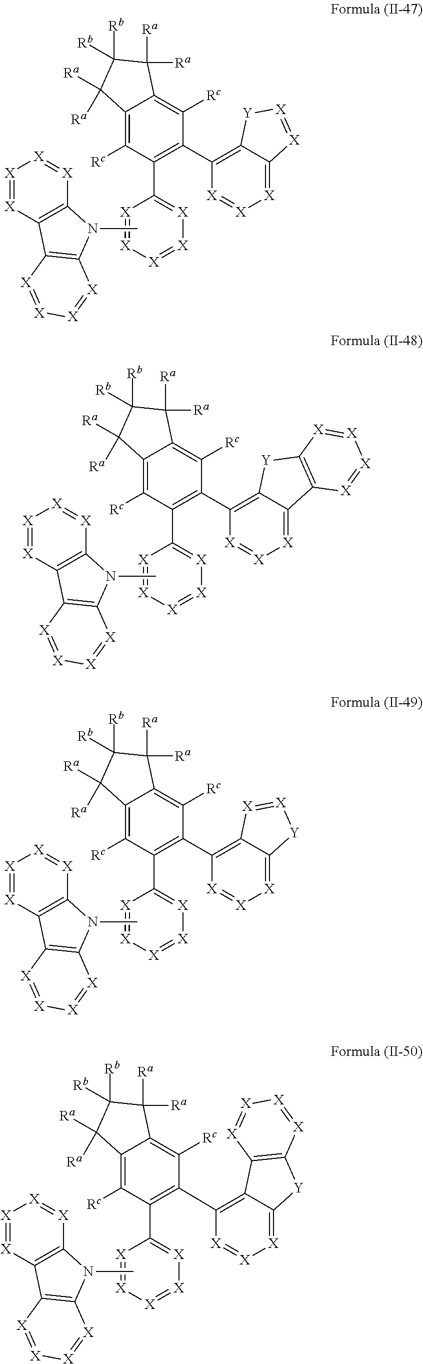 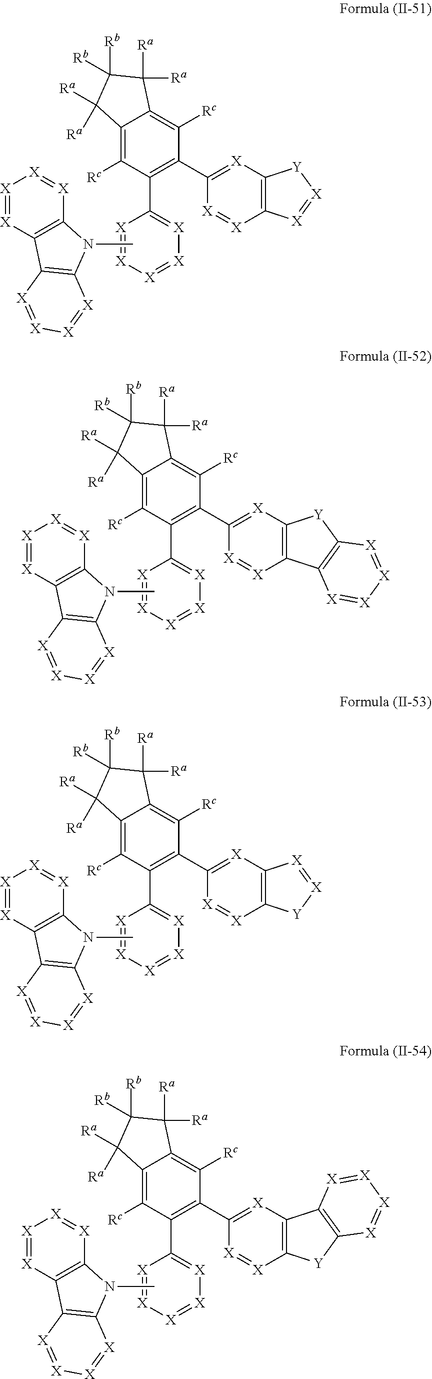 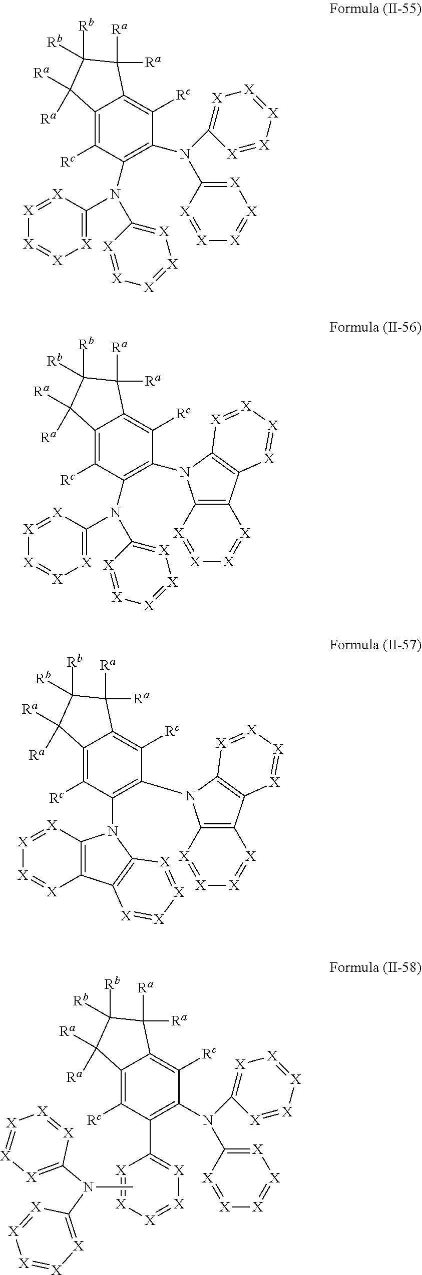 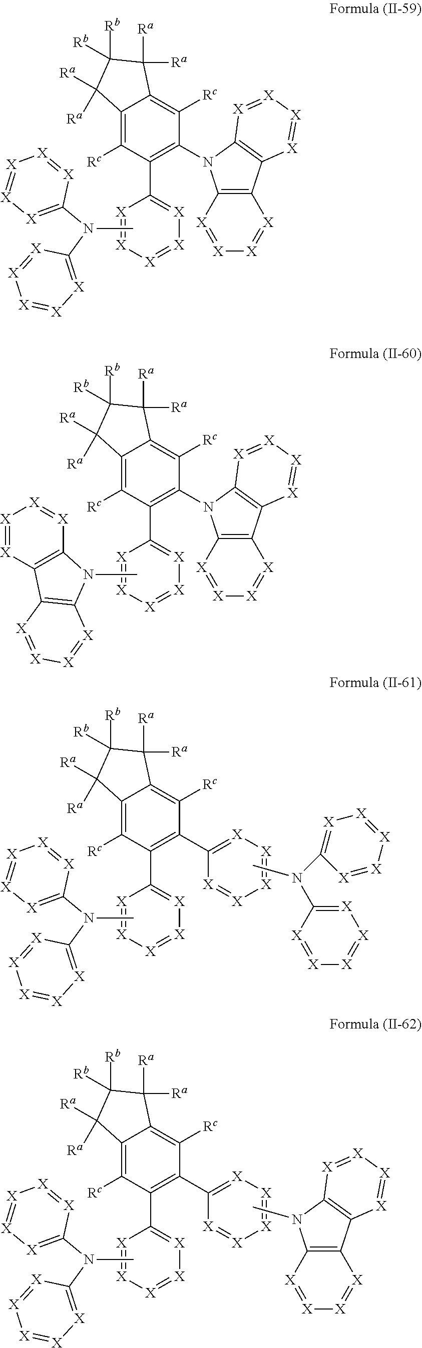 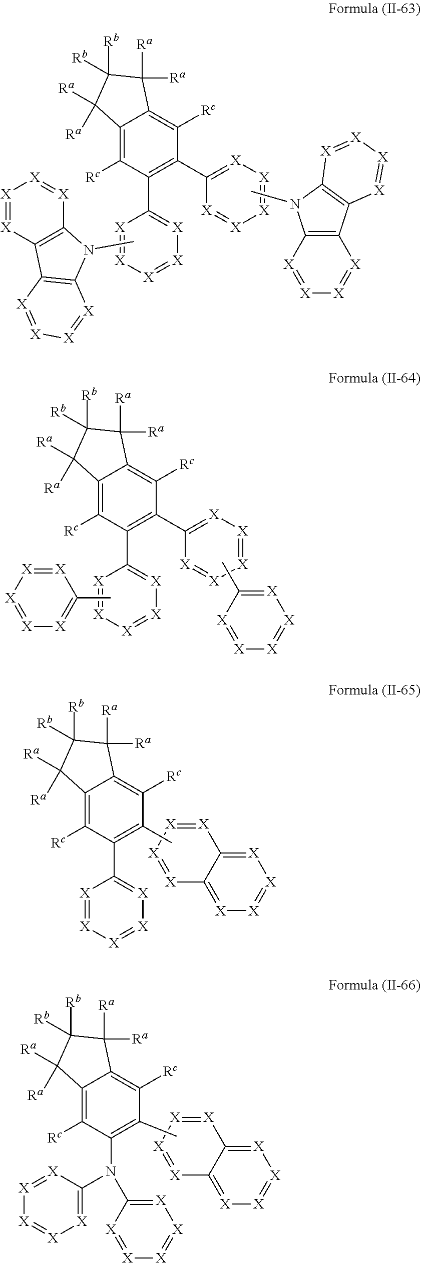 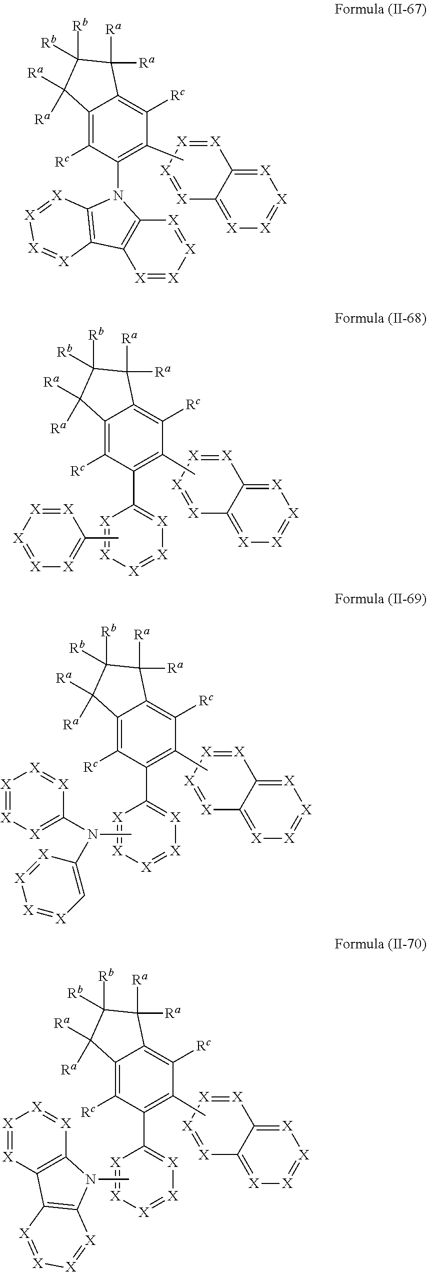 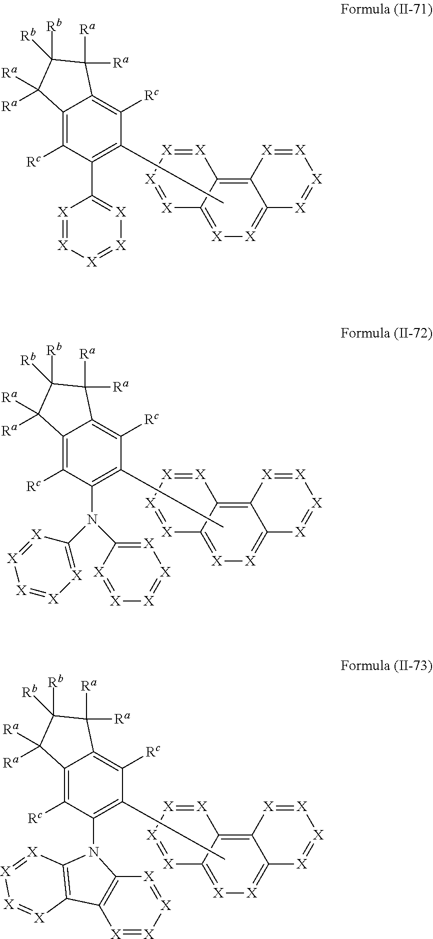 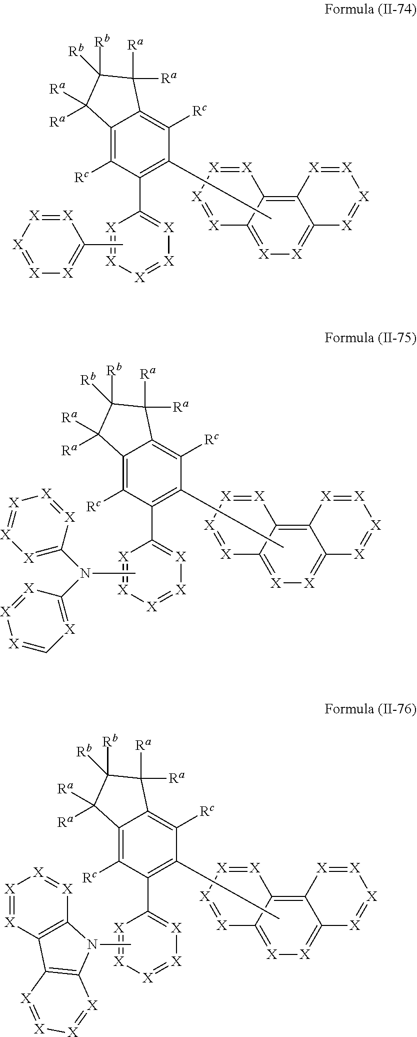 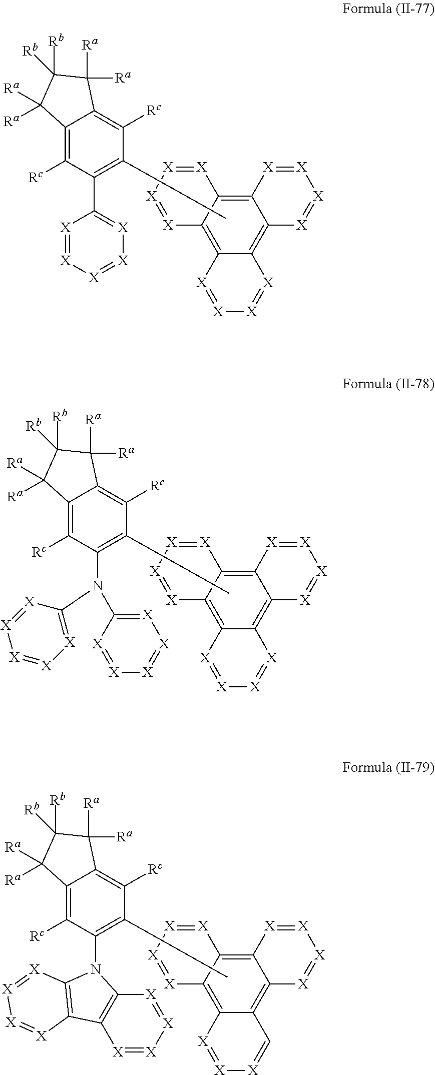 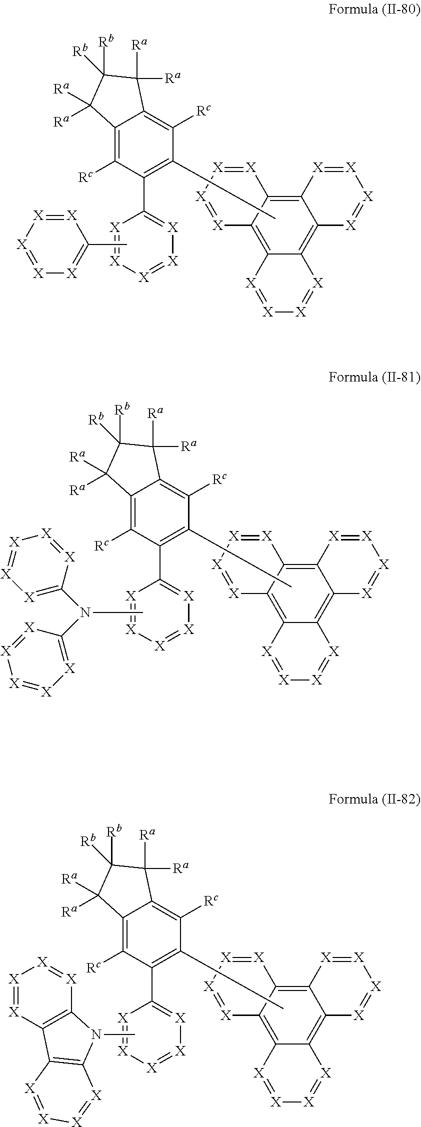 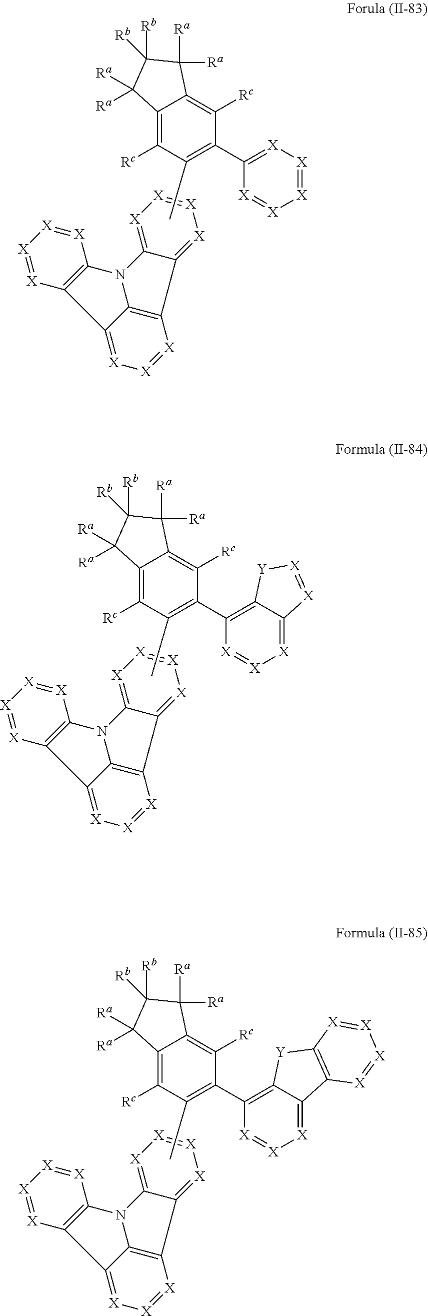 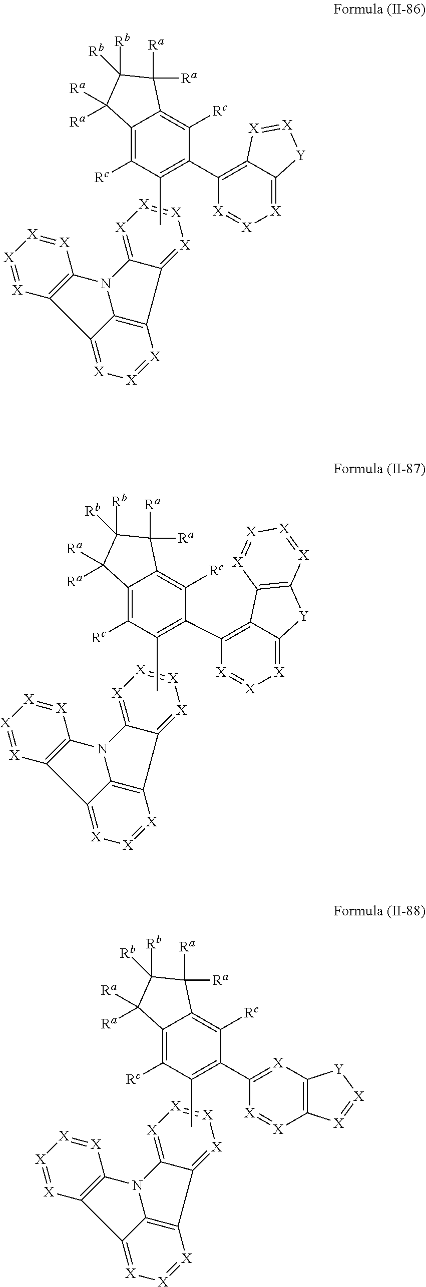 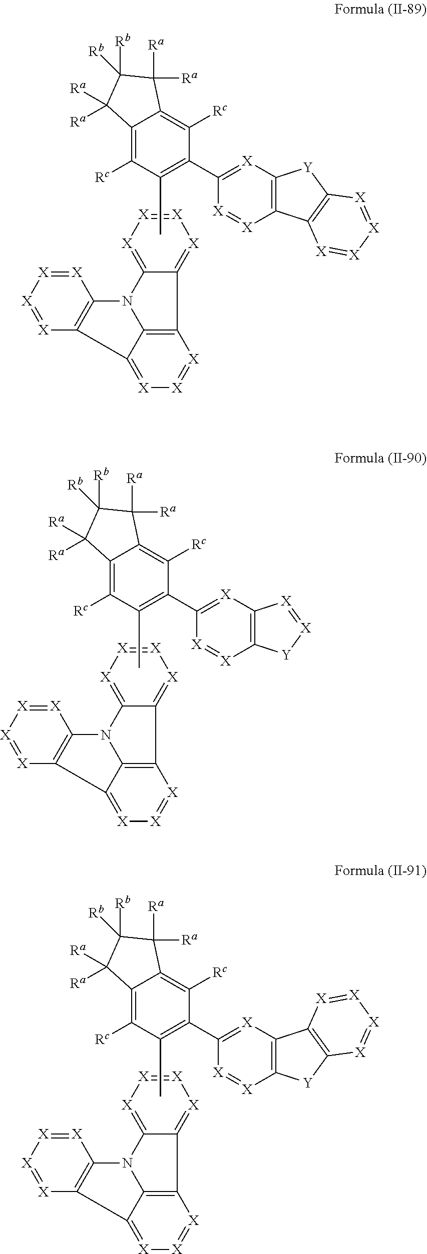 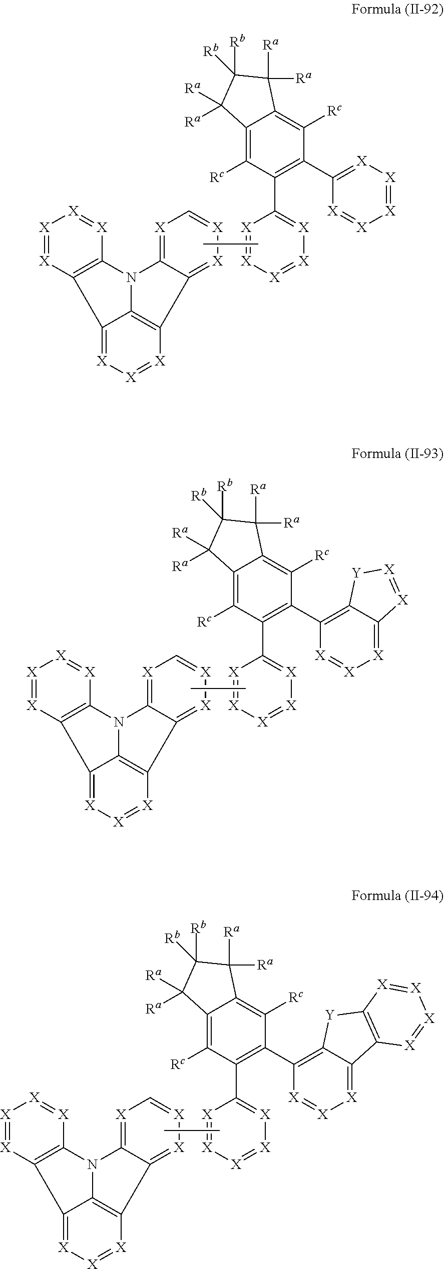 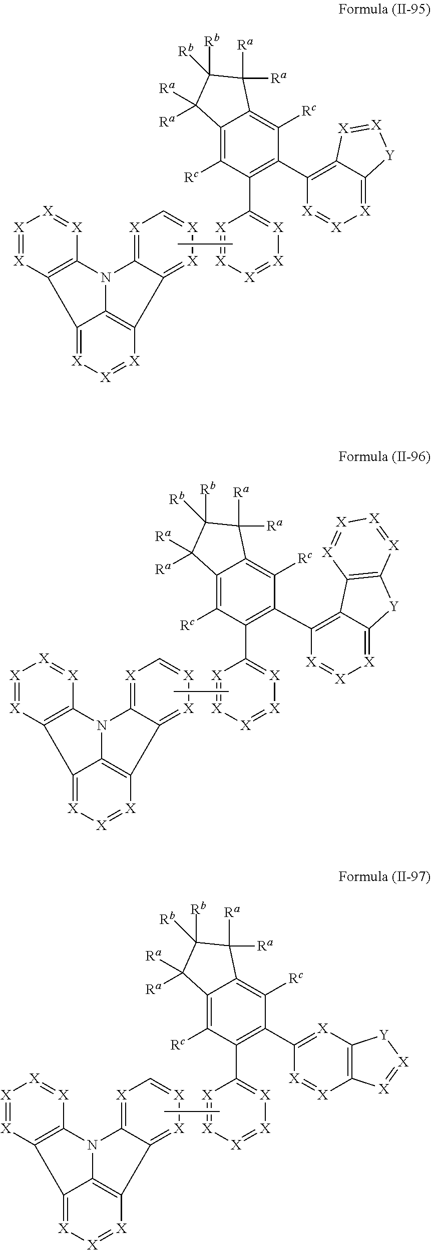 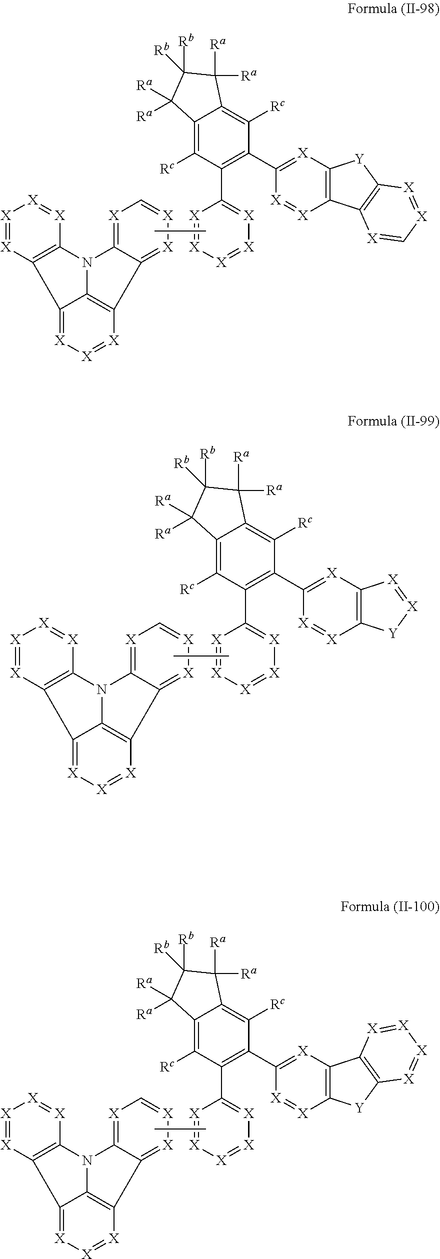 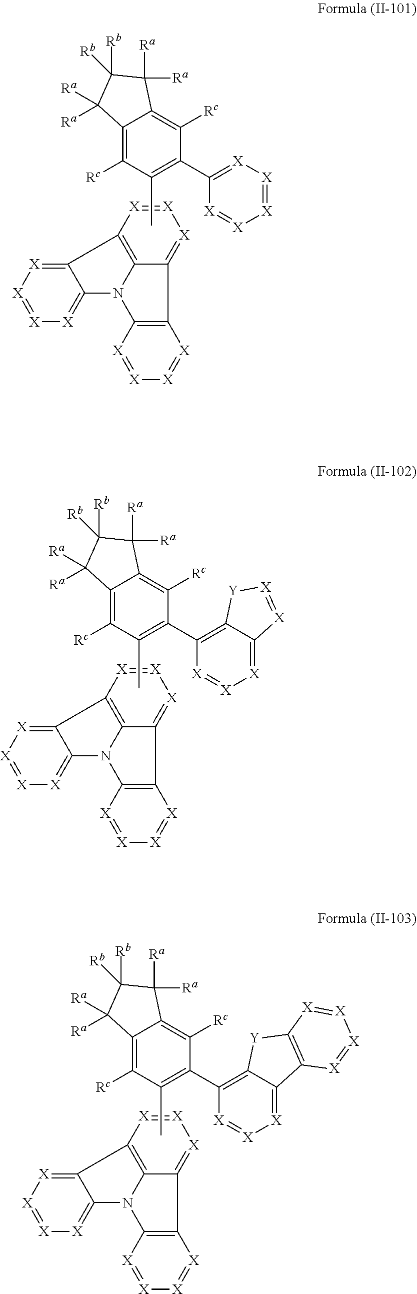 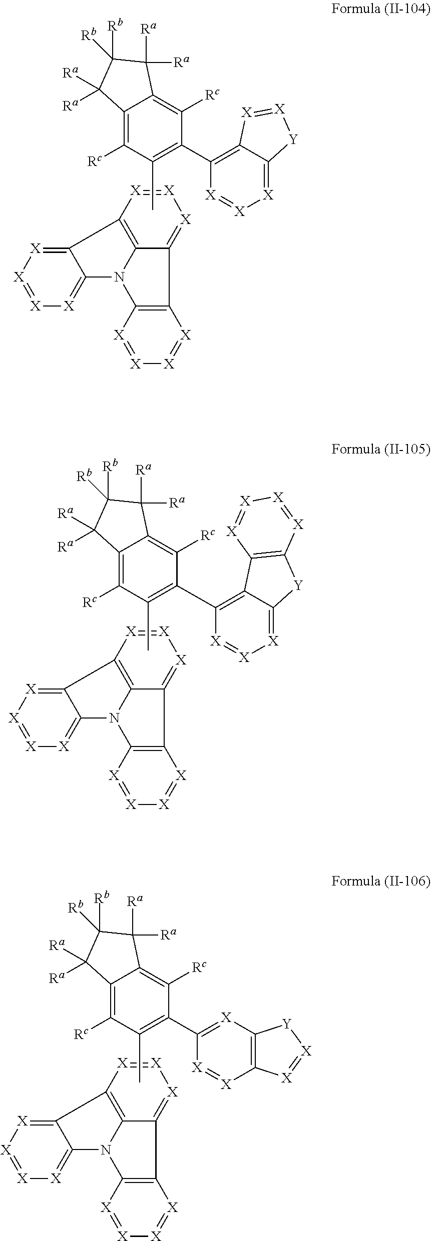 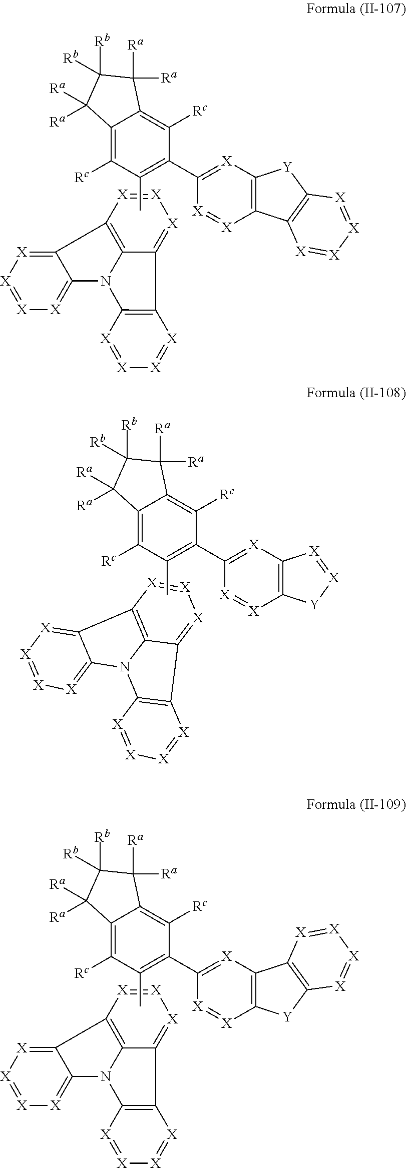 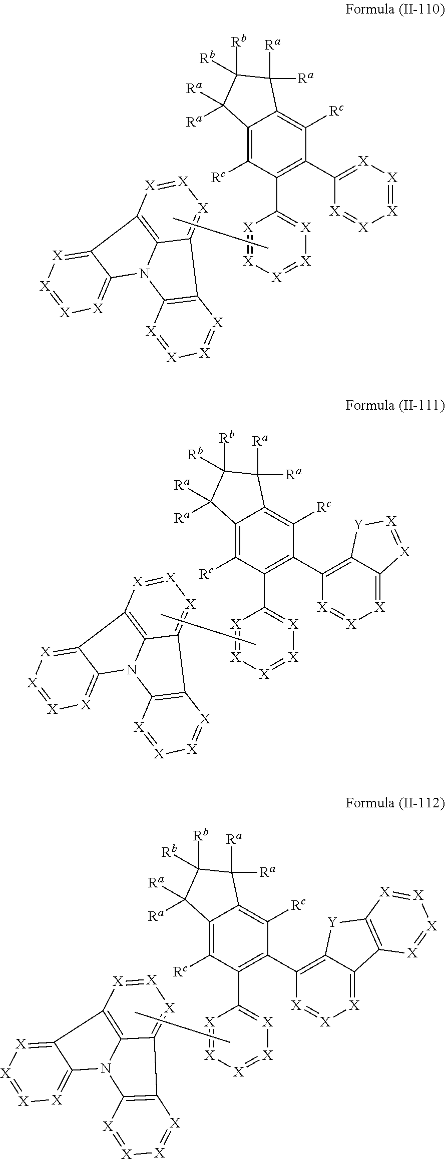 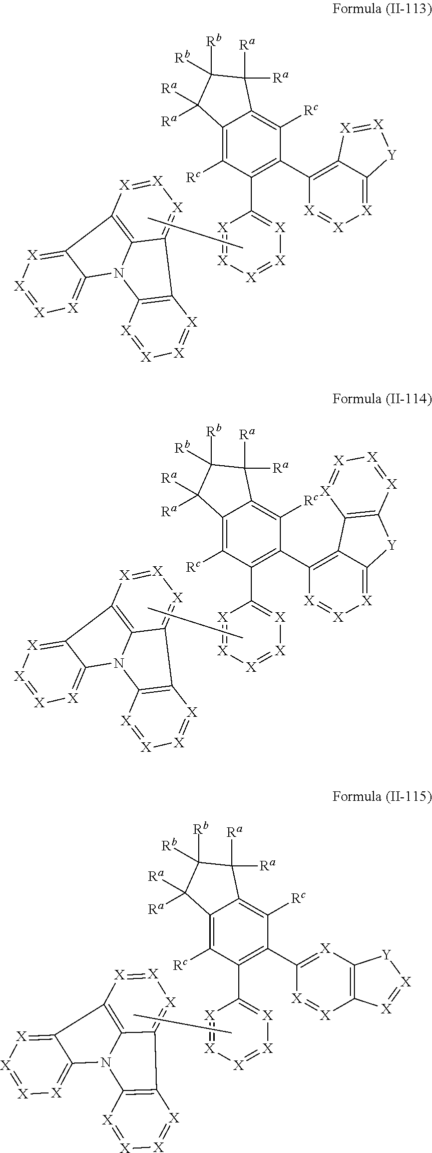 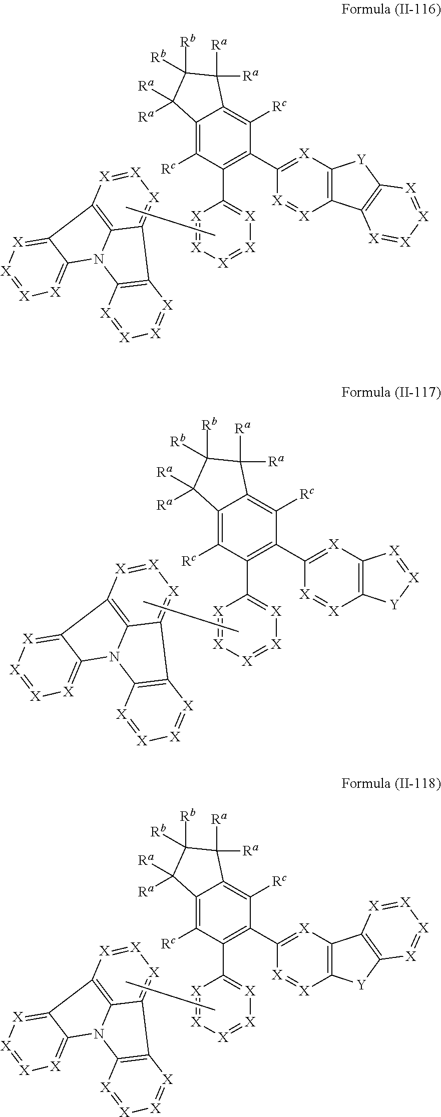 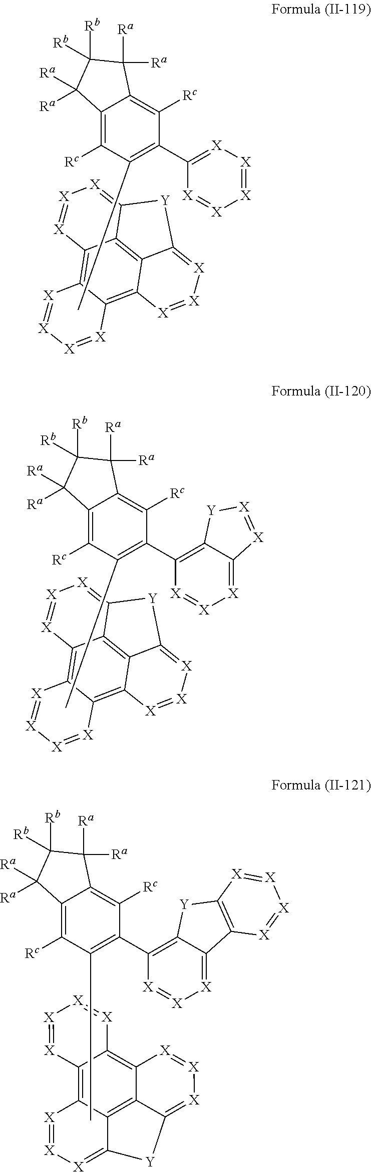 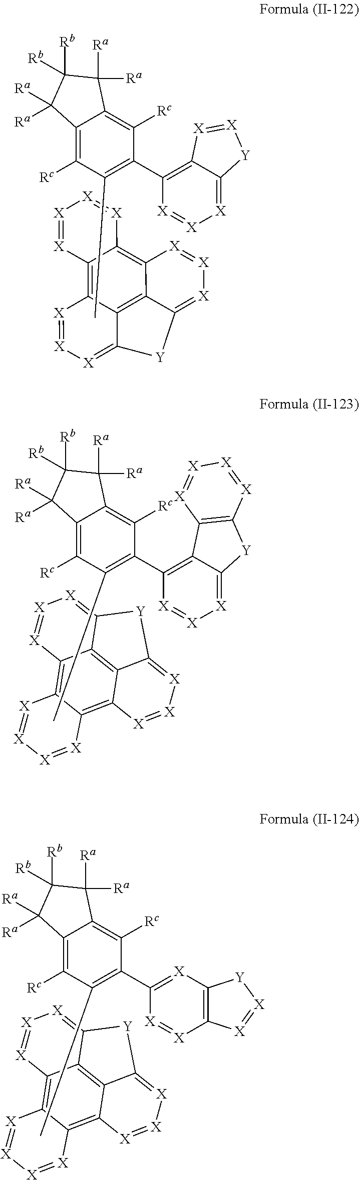 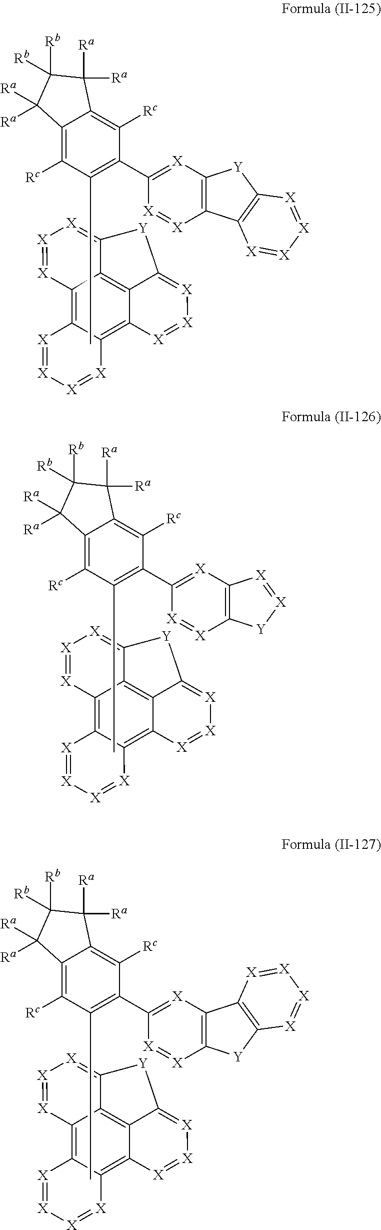 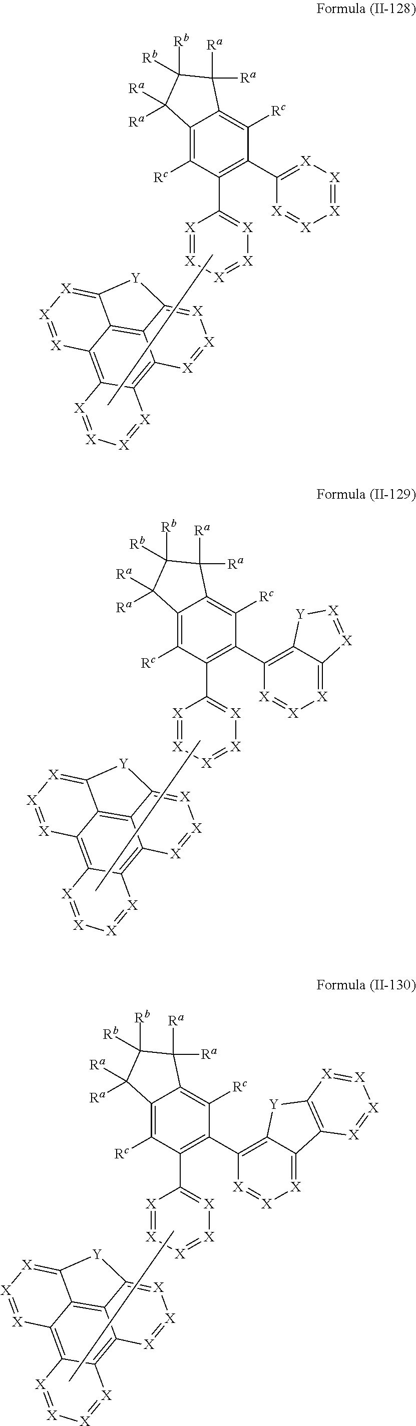 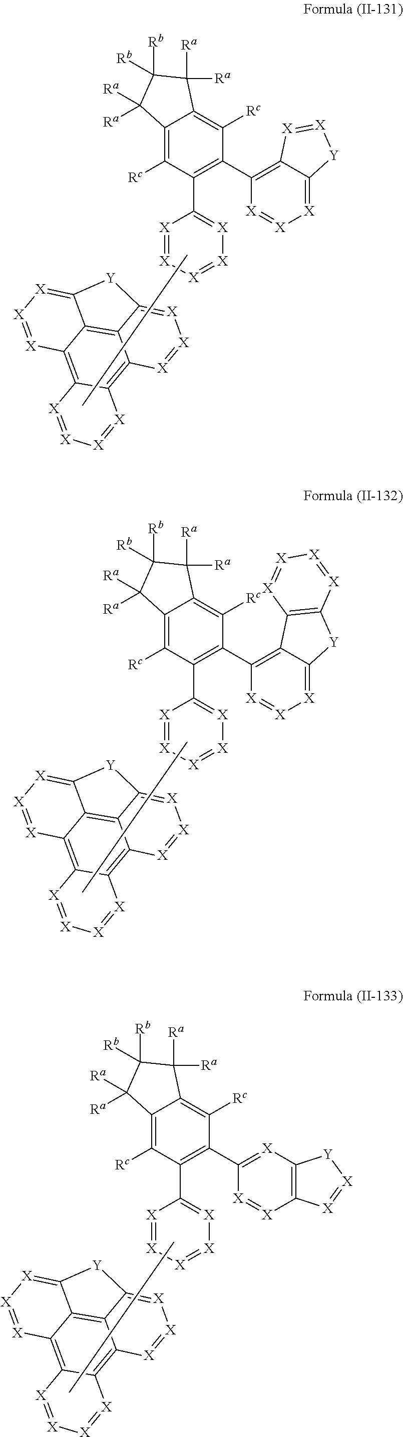 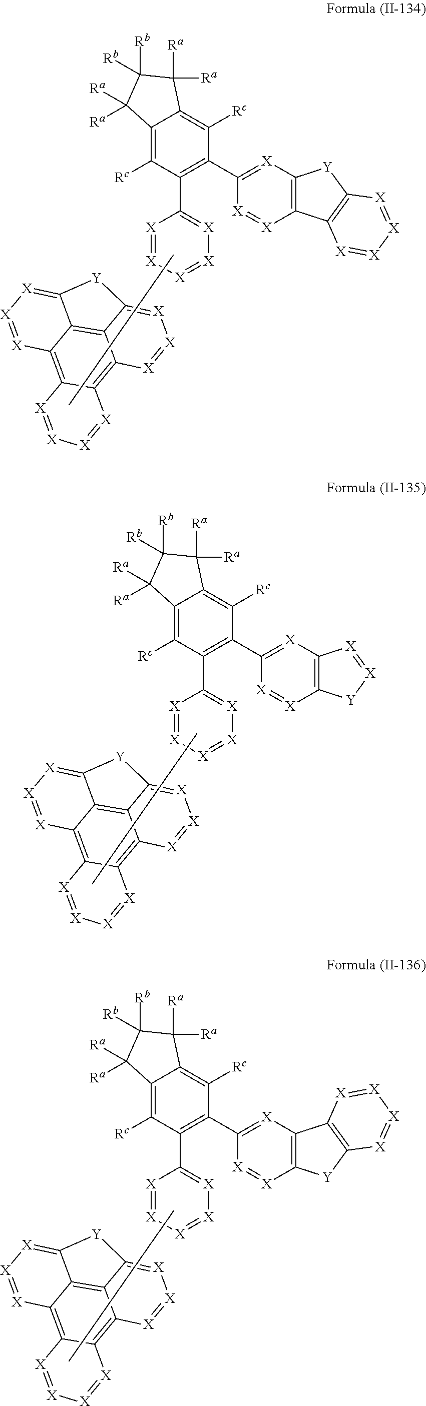 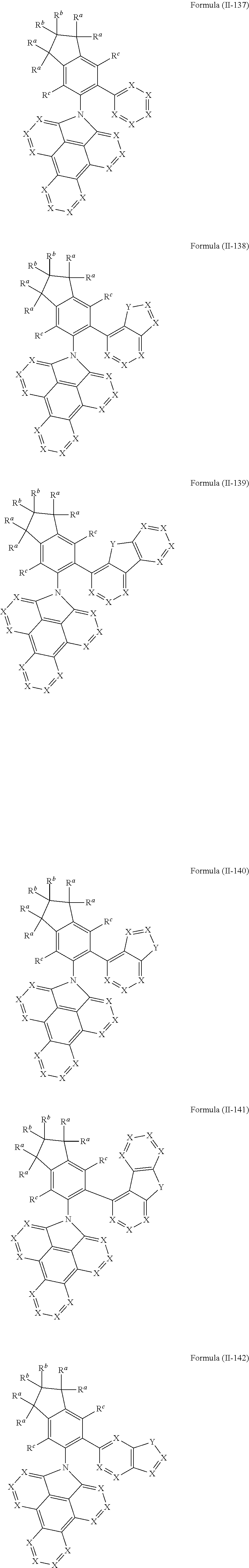 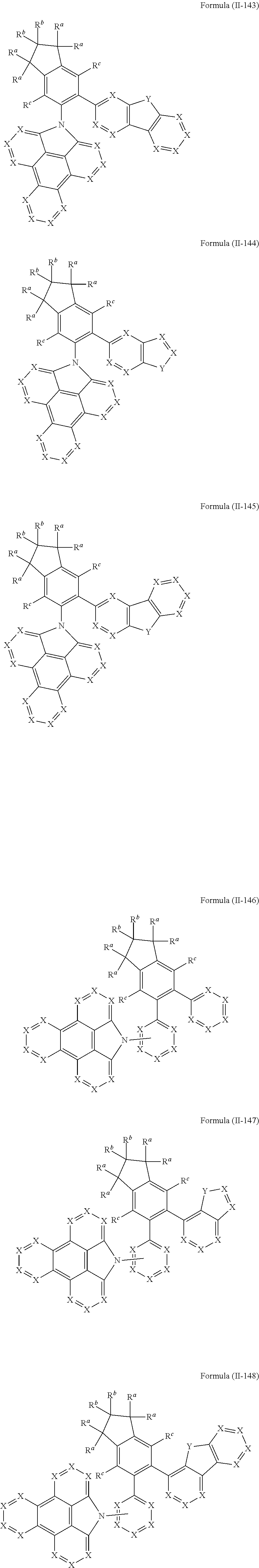 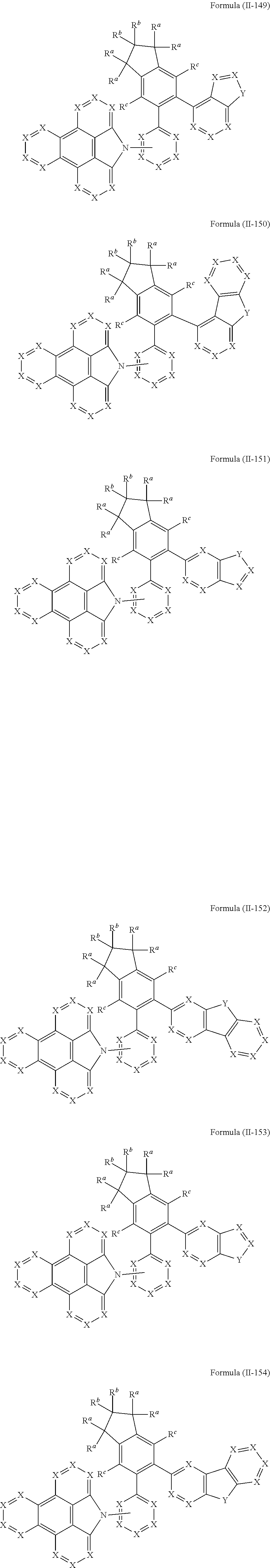
where the symbols R, R^{a}, R^{b} and R^{c} have the definitions given in claim 20, and the further symbols are as follows:
X is the same or different at each instance and is N, CR, or C if a group binds to the structure; and
Y is O, S, NR or C(R)₂.

32. The compound according to claim 20, comprising at least one structure of the formulae (III-1) to (III-60), 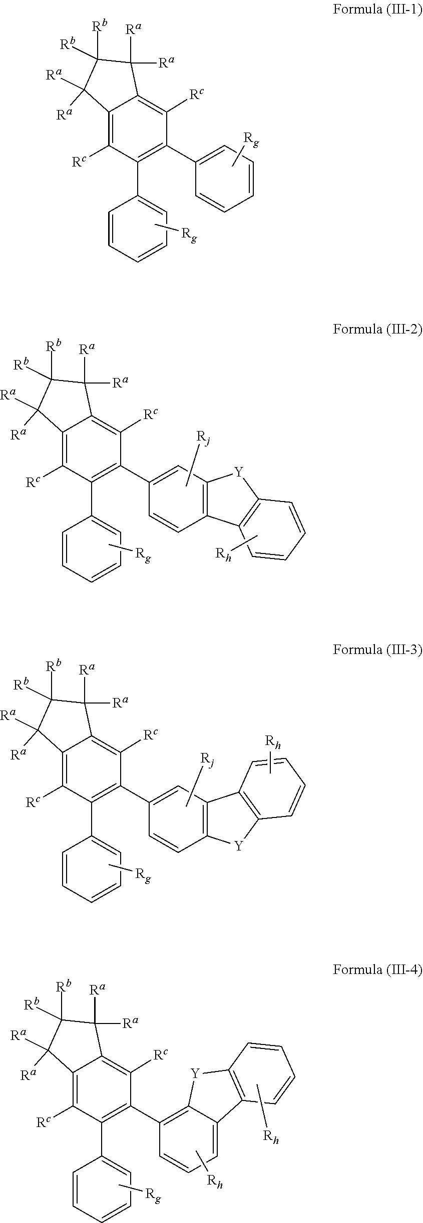 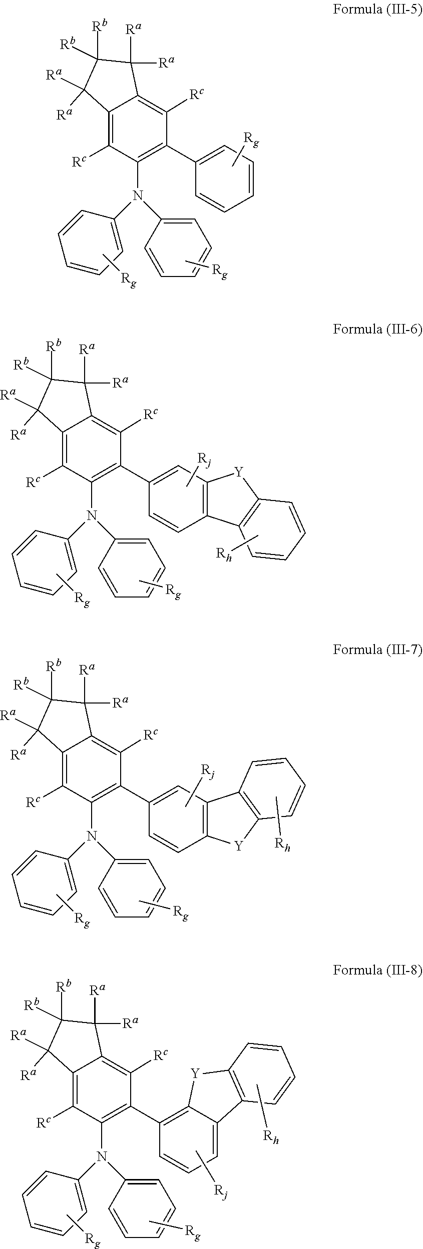 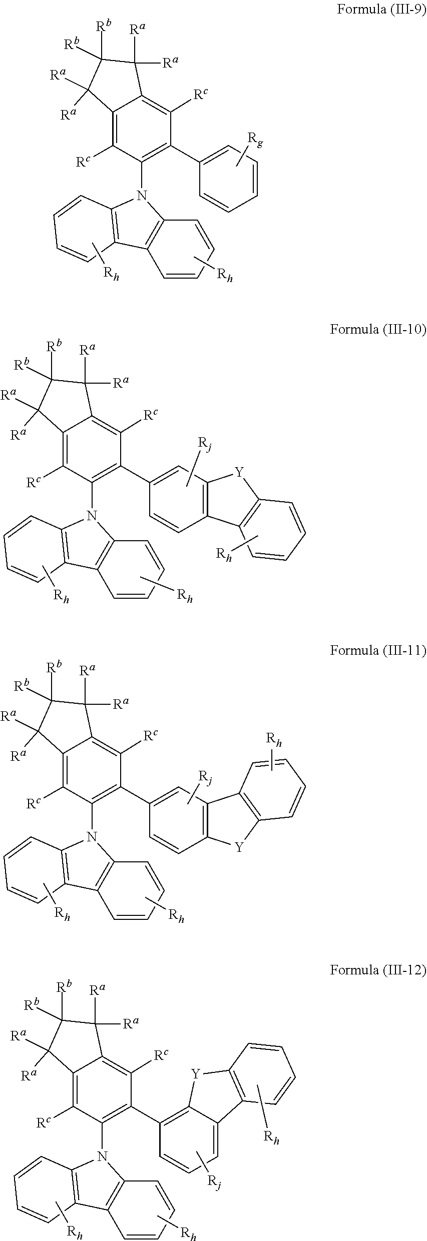 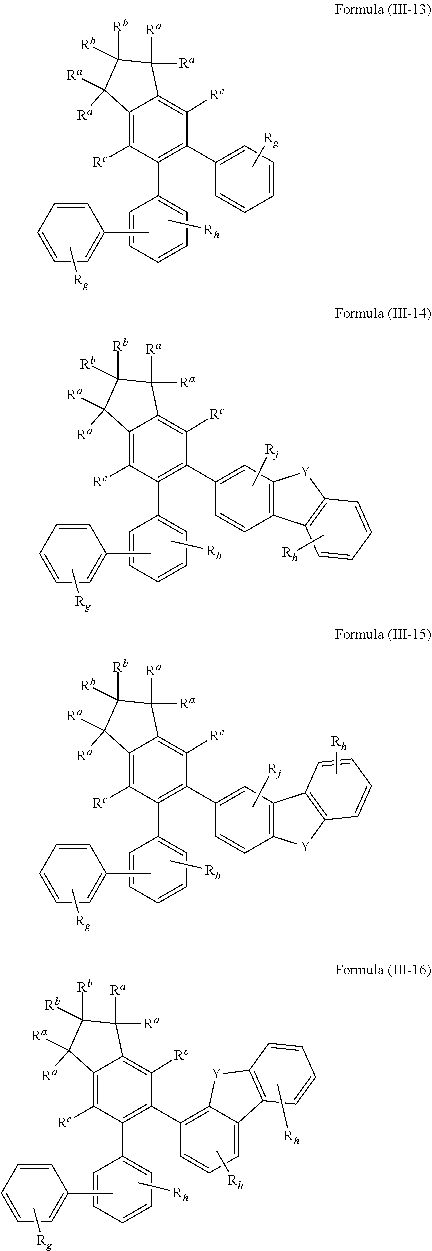 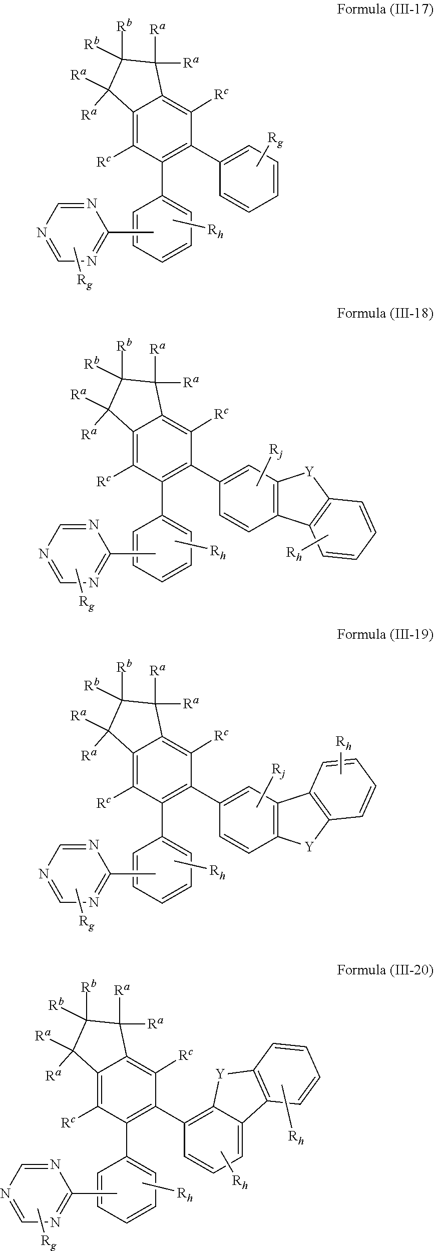 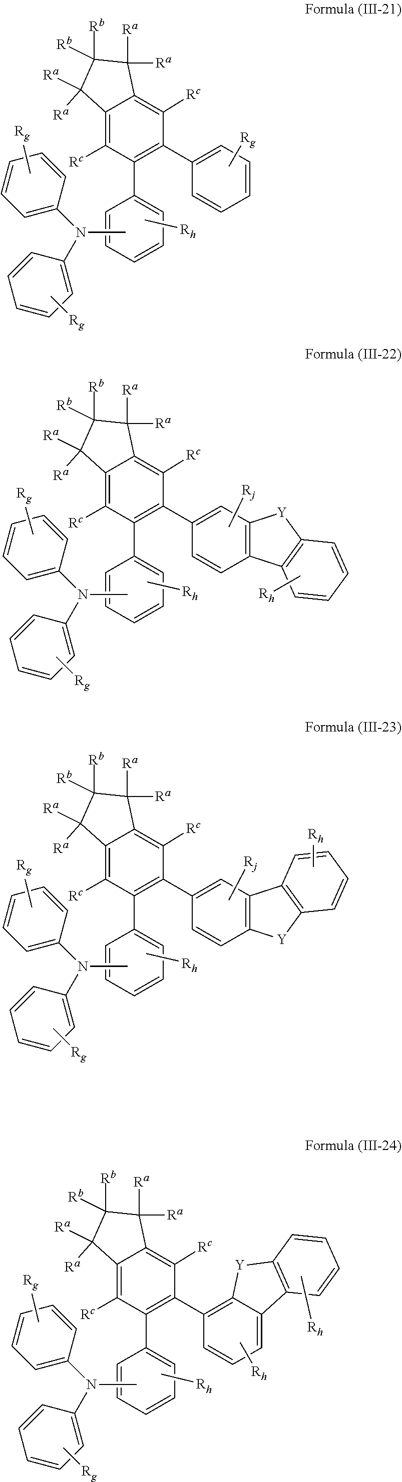 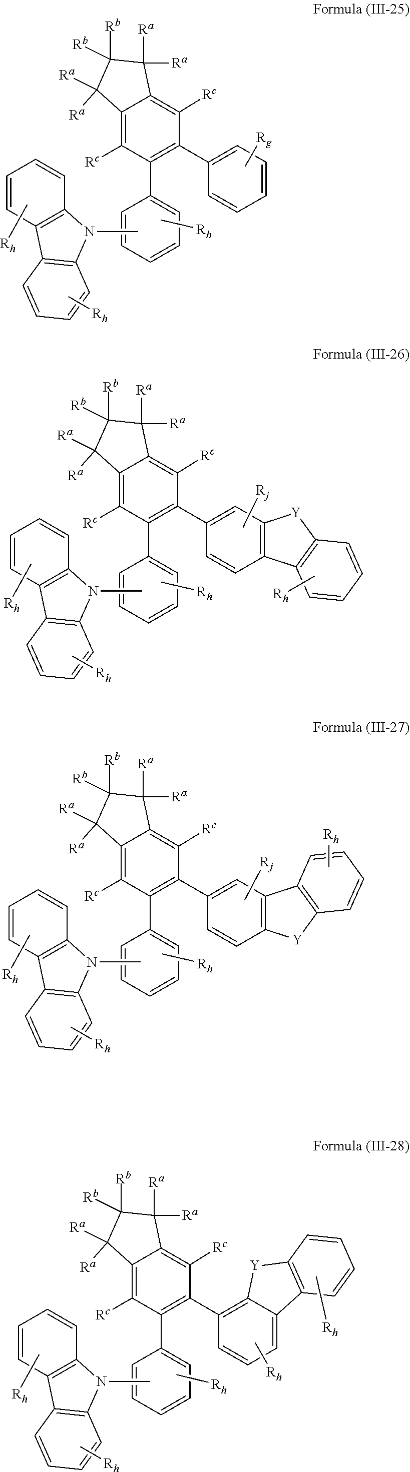 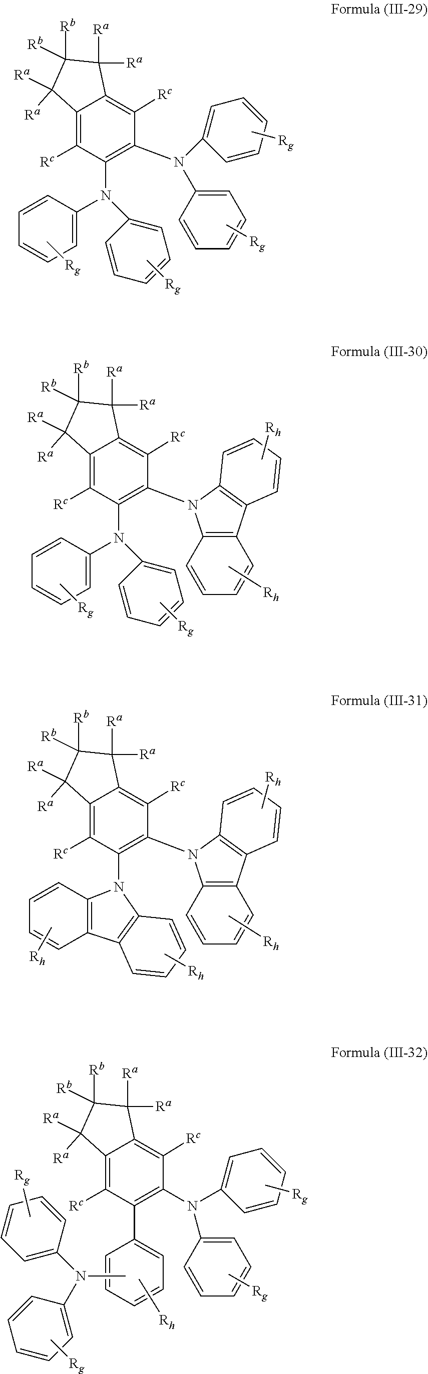 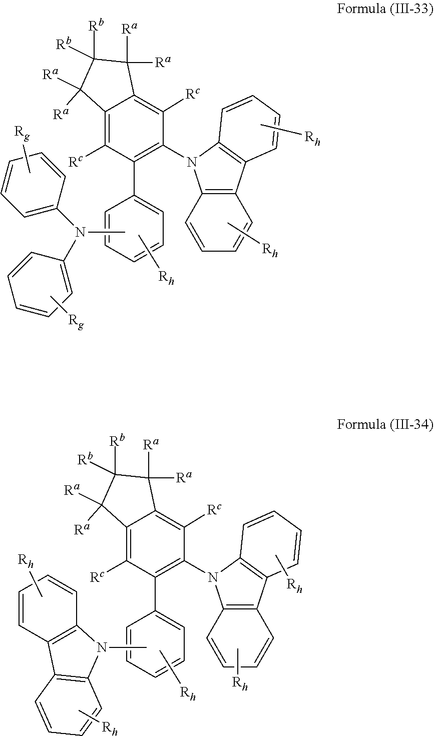 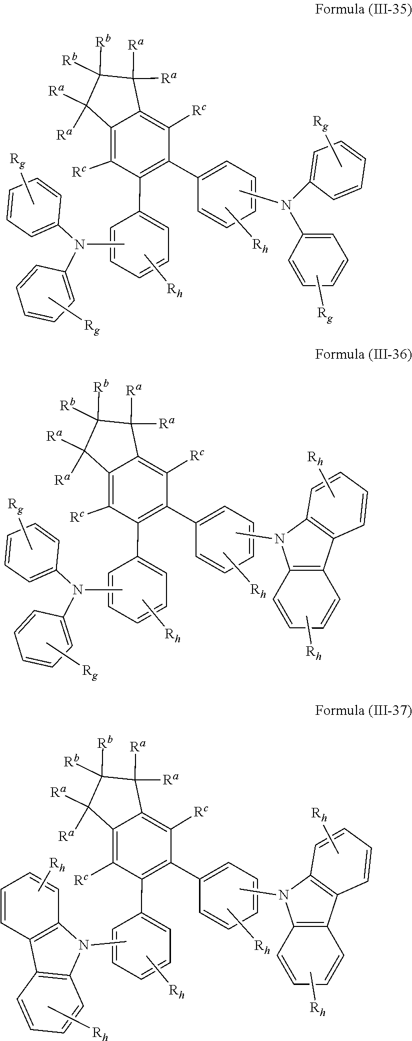 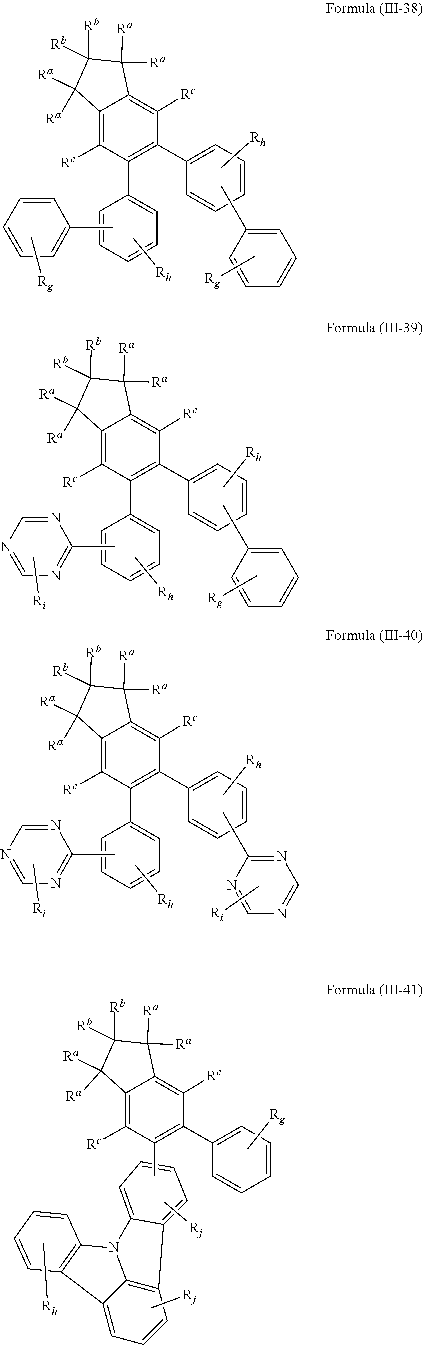 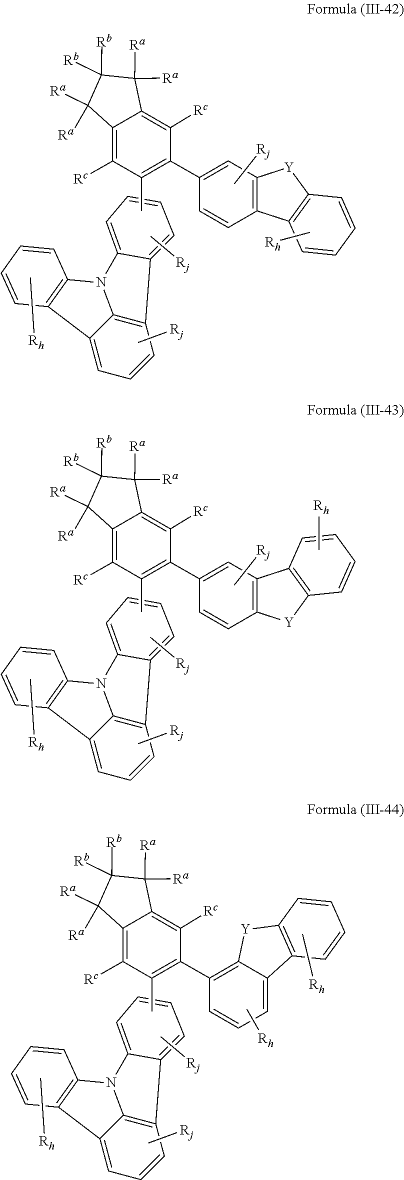 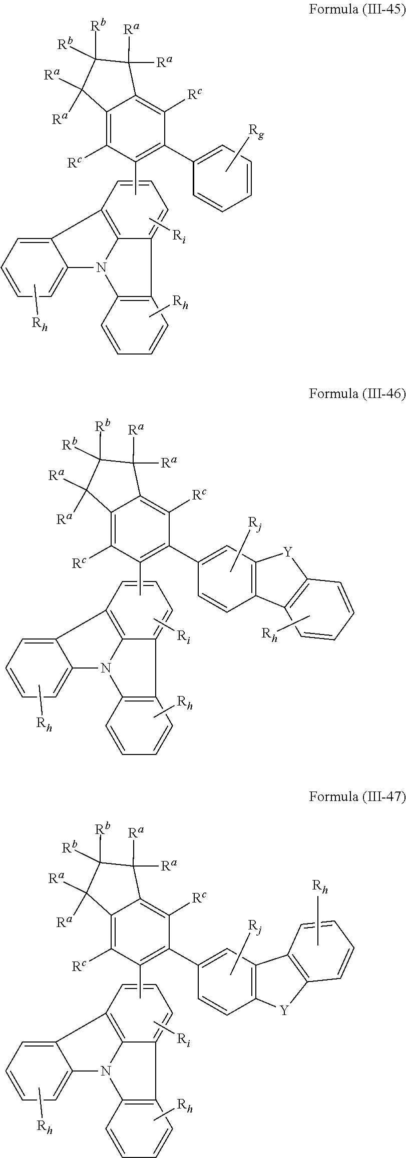 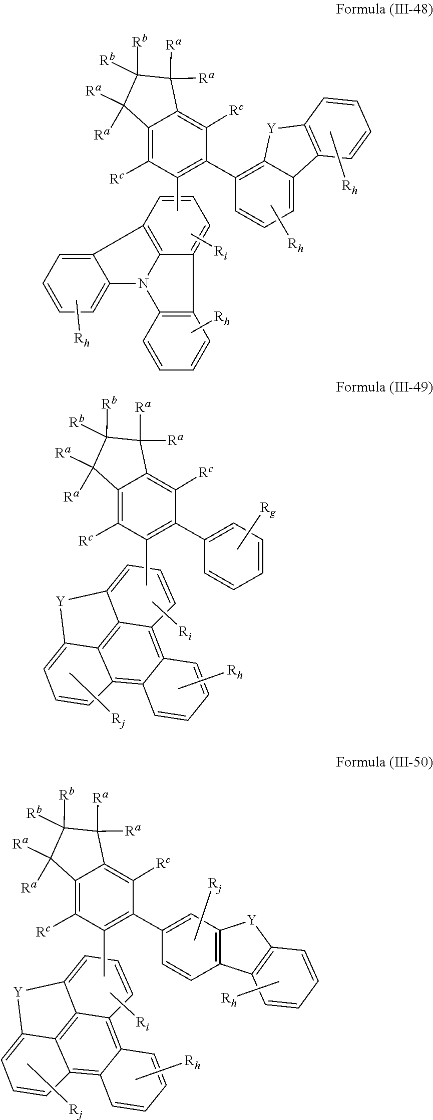 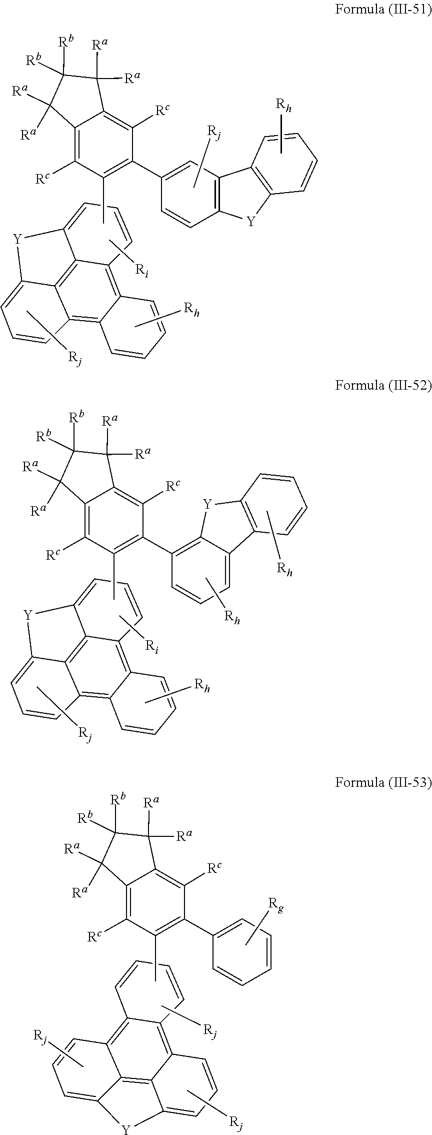 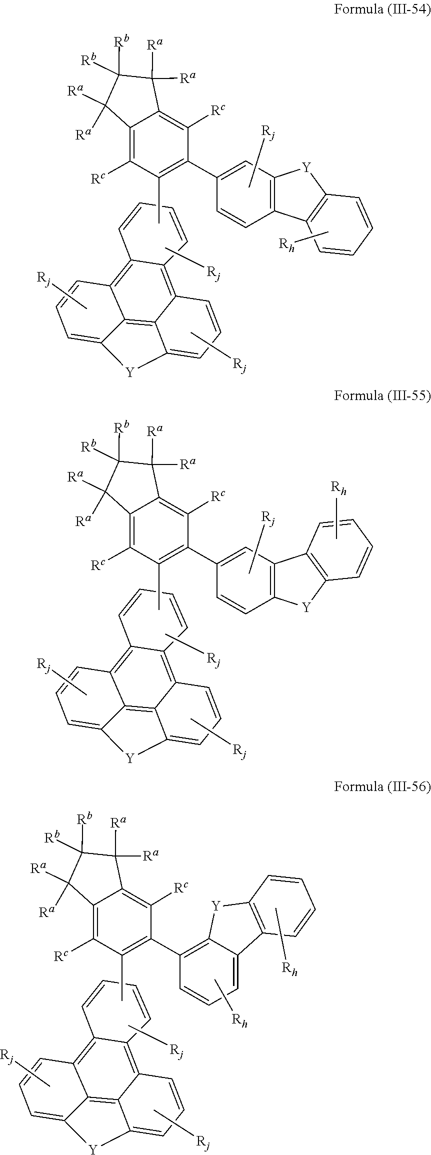 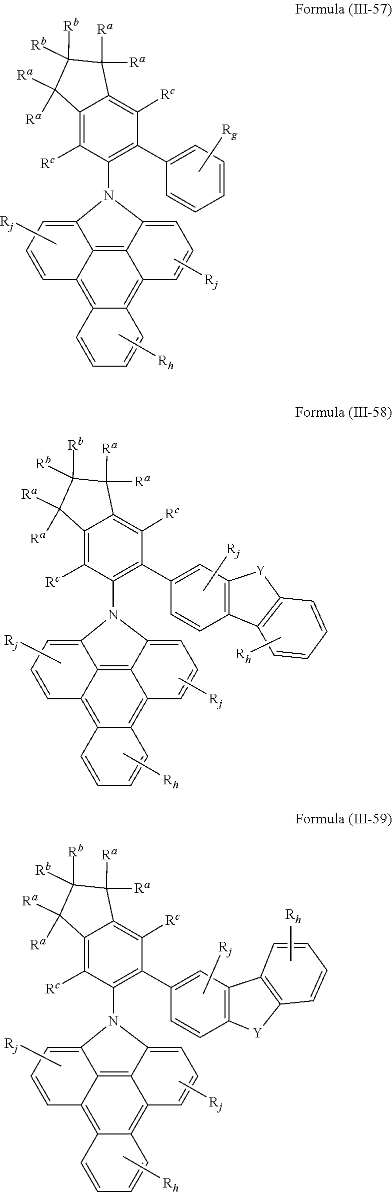 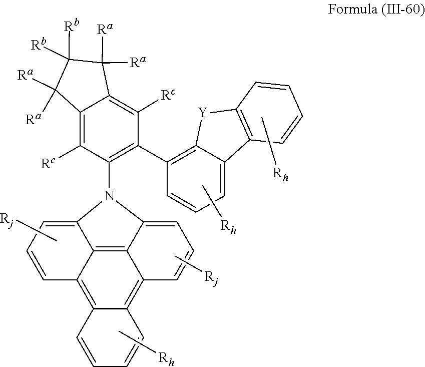
where the symbols R, R^{a}, R^{b} and R^{c} have the definitions given in claim 20, and the symbols used are as follows:
Y is O, S, NR or C(R)₂;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5.

33. An oligomer, polymer or dendrimer containing one or more compounds as claimed in claim 20, wherein, in place of a hydrogen atom or a substituent, there are one or more bonds of the compounds to the polymer, oligomer or dendrimer.

34. A formulation comprising at least one compound as claimed in claim 20 or an oligomer, polymer or dendrimer comprising the compound and at least one further compound.

35. A composition comprising at least one compound as claimed in claim 20 or an oligomer, polymer or dendrimer comprising the compound and at least one further compound selected from the group consisting of fluorescent emitters, phosphorescent emitters, emitters that exhibit TADF, host materials, electron transport materials, electron injection materials, hole conductor materials, hole injection materials, electron blocker materials and hole blocker materials.

36. A process for preparing the compound as claimed in claim 20, wherein a phenyl compound to which a cyclopentyl group is fused is synthesized and at least one aromatic or heteroaromatic radical is introduced.

37. A method comprising providing the compound as claimed in claim 20 or an oligomer, polymer or dendrimer comprising the compound and incorporating the compound or an oligomer, polymer or dendrimer comprising the compound in an electronic device.

38. An electronic device comprising at least one compound as claimed in claim 20 or an oligomer, polymer or dendrimer comprising the compound.
